(19) United States
(12) Patent Application Publication
(10) Pub. No.: US 2022/0144858 A1
(43) Pub. Date: May 12, 2022

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Won-Jang JEONG, Yongin-si (KR); Seong-Jong PARK, Yongin-si (KR); Dong-Jun KIM, Yongin-si (KR); Nam-Jin LEE, Yongin-si (KR)

(73) Assignee: LT MATERIALS CO., LTD., Yongin-si, Gyeonggi-do (KR)

(21) Appl. No.: 17/604,039

(22) PCT Filed: Jul. 21, 2020
(86) PCT No.: PCT/KR2020/009605

§ 371 Date: Oct. 15, 2021

(30) Foreign Application Priority Data
Jul. 26, 2019 (KR) 10-2019-0091146

(51) Int. Cl. C07D 519/00; H01L 51/00; C07F 9/28; C07D 487/04

(52) U.S. Cl. CPC C07D 519/00; H01L 51/0067; H01L 51/0072; H01L 51/0054; H01L 51/5092; H01L 51/0058; C07F 9/28; C07D 487/04; H01L 51/0073

(57) ABSTRACT

The present specification relates to a heterocyclic compound represented by Chemical Formula 1, and an organic light emitting device comprising the same.

### TECHNICAL FIELD

This application claims priority to and the benefits of Korean Patent Application No. 10-2019-0091146, filed with the Korean Intellectual Property Office on Jul. 26, 2019, the entire contents of which are incorporated herein by reference.

The present specification relates to a heterocyclic compound, and an organic light emitting device comprising the same.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### DISCLOSURE

### Technical Problem

The present disclosure is directed to providing a heterocyclic compound, and an organic light emitting device comprising the same.

### Technical Solution

One embodiment of the present application provides a heterocyclic compound represented by the following Chemical Formula 1.

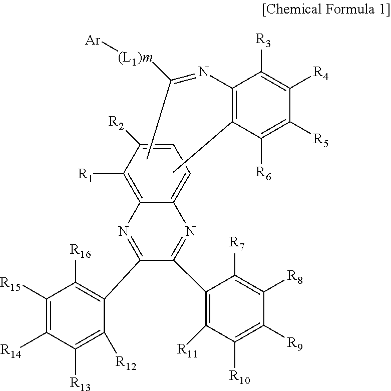

In Chemical Formula 1,

R₁ to R₆ are the same as or different from each other, and each independently selected from the group consisting of hydrogen; deuterium; a halogen group; —CN; a substituted or unsubstituted C1 to C60 alkyl group; a substituted or unsubstituted C6 to C60 aryl group; a substituted or unsubstituted C2 to C60 heteroaryl group; —SiRR′R″; —P(═O)RR′; and an amine group unsubstituted or substituted with a substituted or unsubstituted C1 to C60 alkyl group, a substituted or unsubstituted C6 to C60 aryl group or a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring,

L₁ is a substituted or unsubstituted C6 to C60 arylene group; or a substituted or unsubstituted C2 to C60 heteroarylene group,

Ar is hydrogen; a halogen group; —CN; a substituted or unsubstituted C1 to C60 alkyl group; a substituted or unsubstituted C6 to C60 aryl group; a substituted or unsubstituted C2 to C60 heteroaryl group; —SiRR′R″; or —P(═O)RR′,

R₇ to R₁₆ are the same as or different from each other, and each independently hydrogen; a halogen group; —CN; a substituted or unsubstituted C6 to C60 aryl group; or a substituted or unsubstituted C2 to C60 heteroaryl group,

R, R′ and R″ are the same as or different from each other, and each independently hydrogen; a substituted or unsubstituted C1 to C40 alkyl group; or a substituted or unsubstituted C6 to C40 aryl group, and

m is an integer of 1 to 4, and when m is 2 or greater, the two or more L₁s are the same as or different from each other.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

### Advantageous Effects

A compound described in the present specification can be used as a material of an organic material layer of an organic light emitting device. The compound is capable of performing a role of a hole injection material, a hole transfer material, a hole blocking material, a light emitting material, an electron transfer material, an electron injection material, a charge generation material or the like in an organic light emitting device. Particularly, the compound can be used as an electron transfer layer material, a hole blocking layer material, a charge generation layer material or a hole injection layer material of an organic light emitting device.

When using the compound represented by Chemical Formula 1 in an organic material layer, a driving voltage of a device can be lowered, light efficiency can be enhanced, and lifetime properties of the device can be enhanced by thermal stability of the compound.

The compound represented by Chemical Formula 1 has a core form in which a quinoline group is fused to quinoxaline, and by adding a more electron-friendly heteroatom to the central skeleton of the core structure, an electron transfer ability is enhanced, and as a result, superior device properties are obtained when used in an organic light emitting device later.

In addition, the compound represented by Chemical Formula 1 has a structure having substituents linked to the quinoxaline group with two phenyl groups, and by having two phenyl groups, thermal stability is excellent, and as a result, significantly superior lifetime properties are obtained.

### DESCRIPTION OF DRAWINGS

FIG. 1 to FIG. 4 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of C1 to C60 haloalkyl; C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R₁₀₁R₁₀₂, and R₁₀₁ and R₁₀₂ are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted, it may be represented by any one of the following structural formulae.

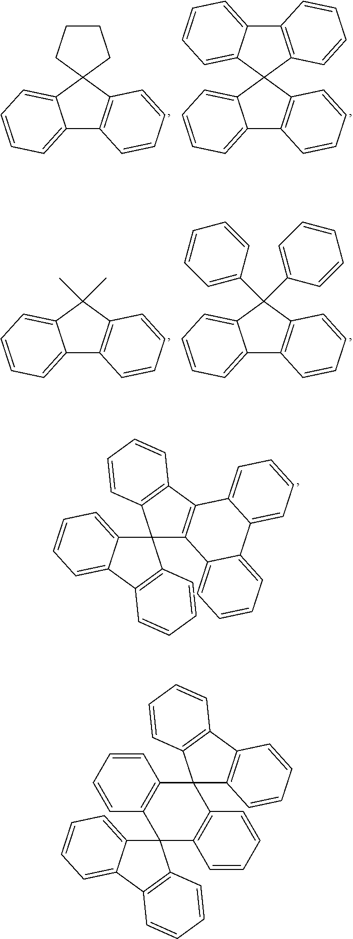

In the present specification, the heteroaryl group comprises S, O, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, the “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

One embodiment of the present application provides a compound represented by Chemical Formula 1.

The compound represented by Chemical Formula 1 has a structure having substituents linked to the quinoxaline group, a core structure, with two phenyl groups, and by having two phenyl groups, thermal stability is excellent, and as a result, significantly superior lifetime properties are obtained.

In other words, when there is no substituent linked to the quinoxaline group with a phenyl group or when there is only one such substituent, reactivity of carbon next to nitrogen of the quinoxaline group is high causing a problem in thermal stability, which results in a phenomenon of significantly reduced lifetime compared to a structure having two phenyl group-linked substituents as in Chemical Formula 1 of the present application.

In addition, Chemical Formula 1 of the present application has a core structure in which a quinoxaline group is fused to a quinoline group, and when another structure such as an imidazole group is fused to the quinoline group, electron distribution is concentrated in the skeleton structure, a core structure, compared to when a quinoxaline group is fused resulting in decreased electron mobility, and in this case, driving voltage increases and efficiency may decrease compared to the compound of Chemical Formula 1 of the present application.

In one embodiment of the present application, Chemical Formula 1 may be represented by the following Chemical Formula 2 or Chemical Formula 3.

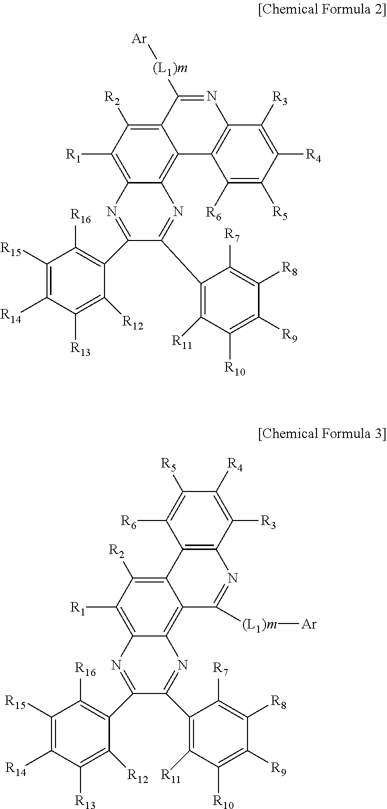

In Chemical Formulae 2 and 3, R₁ to R₁₆, Ar, m and L₁ have the same definitions as in Chemical Formula 1.

In one embodiment of the present application, Chemical Formula 1 may be represented by any one of the following Chemical Formula 4 to Chemical Formula 8.

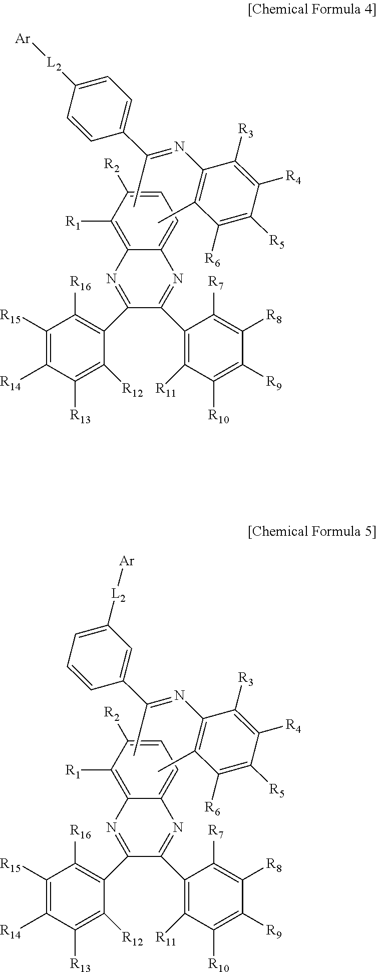

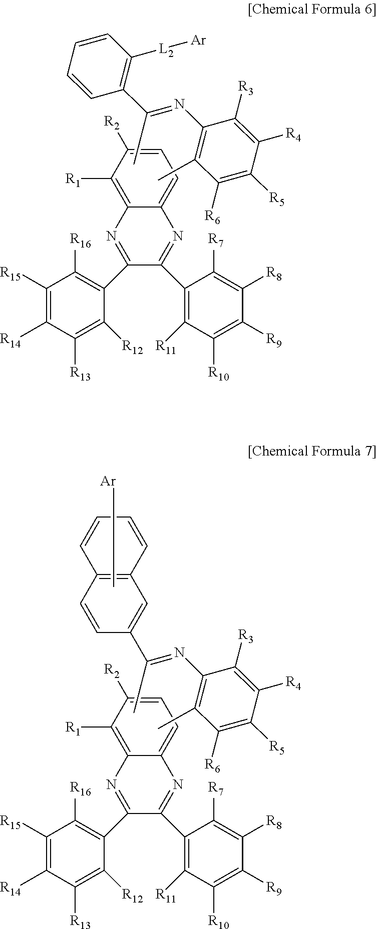

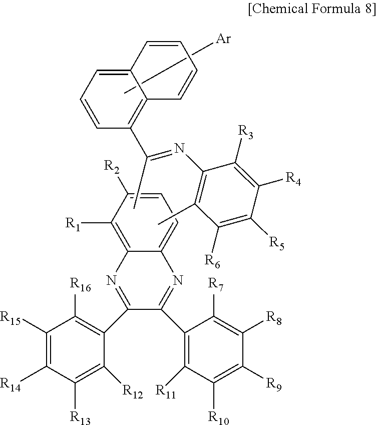

In Chemical Formulae 4 to 8,

R₁ to R₁₆ and Ar have the same definitions as in Chemical Formula 1,

L₂ is a direct bond; a substituted or unsubstituted C6 to C60 arylene group; or a substituted or unsubstituted C2 to C60 heteroarylene group.

In one embodiment of the present application, L₁ may be a substituted or unsubstituted C6 to C60 arylene group; or a substituted or unsubstituted C2 to C60 heteroarylene group.

In another embodiment, L₁ may be a substituted or unsubstituted C6 to C40 arylene group; or a substituted or unsubstituted C2 to C40 heteroarylene group.

In another embodiment, L₁ may be a substituted or unsubstituted monocyclic or polycyclic C6 to C40 arylene group.

In another embodiment, L₁ may be a monocyclic C6 to C20 arylene group.

In another embodiment, L₁ may be a polycyclic C10 to C20 arylene group.

In another embodiment, L₁ may be a phenylene group; a biphenylene group; or a naphthalene group.

In one embodiment of the present application, Ar may be hydrogen; a halogen group; —CN; a substituted or unsubstituted C1 to C60 alkyl group; a substituted or unsubstituted C6 to C60 aryl group; a substituted or unsubstituted C2 to C60 heteroaryl group; —SiRR′ R″; or —P(═O)RR′.

In another embodiment, Ar may be hydrogen; a halogen group; —CN; a substituted or unsubstituted C1 to C40 alkyl group; a substituted or unsubstituted C6 to C40 aryl group; a substituted or unsubstituted C2 to C40 heteroaryl group; —SiRR′ R″; or —P(═O)RR′.

In another embodiment, Ar may be hydrogen; a halogen group; —CN; a C1 to C40 alkyl group unsubstituted or substituted with a halogen group; a C6 to C40 aryl group unsubstituted or substituted with one or more substituents selected from the group consisting of a halogen group, —CN and —CF₃; a C2 to C40 heteroaryl group unsubstituted or substituted with a C6 to C40 aryl group; or —P(═O)RR′.

In another embodiment, Ar may be hydrogen; a halogen group; —CN; a C1 to C20 alkyl group unsubstituted or substituted with a halogen group; a monocyclic or polycyclic C6 to C40 aryl group unsubstituted or substituted with one or more substituents selected from the group consisting of a halogen group, —CN and —CF₃; a monocyclic or polycyclic C2 to C40 heteroaryl group unsubstituted or substituted with a C6 to C40 aryl group; or —P(═O)RR′.

In another embodiment, Ar may be hydrogen; —F; —CN; —CF₃; a phenyl group unsubstituted or substituted with one or more substituents selected from the group consisting of a fluoro group, —CN and —CF₃; a triphenylenyl group; a triazine group unsubstituted or substituted with a phenyl group; a pyrimidine group unsubstituted or substituted with a phenyl group; a phenanthroline group unsubstituted or substituted with a phenyl group; a carbazole group; or —P(═O)RR′.

In one embodiment of the present application, R, R′ and R″ are the same as or different from each other, and may be each independently hydrogen; a substituted or unsubstituted C1 to C40 alkyl group; or a substituted or unsubstituted C6 to C40 aryl group.

In another embodiment, R, R′ and R″ are the same as or different from each other, and may be each independently a substituted or unsubstituted C1 to C20 alkyl group; or a substituted or unsubstituted C6 to C20 aryl group.

In another embodiment, R, R′ and R″ are the same as or different from each other, and may be each independently a C1 to C20 alkyl group; or a C6 to C20 aryl group.

In another embodiment, R, R′ and R″ are the same as or different from each other, and may be each independently a C6 to C20 monocyclic aryl group.

In another embodiment, R, R′ and R″ may be a phenyl group.

In one embodiment of the present application, when Ar is a substituted or unsubstituted C6 to C40 aryl group, it may be any one selected from among the following structural formulae.

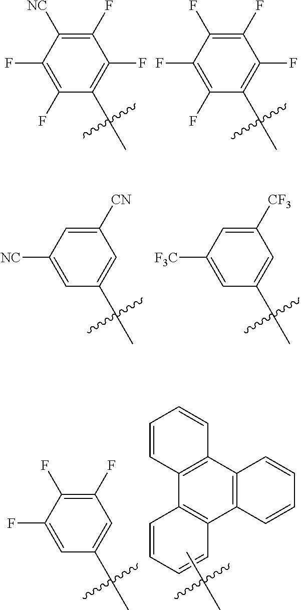

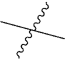

In the structural formulae, means a position linked to L₁ of Chemical Formula 1.

In one embodiment of the present application, R₇ to R₁₆ are the same as or different from each other, and may be each independently hydrogen; a halogen group; —CN; a substituted or unsubstituted C6 to C60 aryl group; or a substituted or unsubstituted C2 to C60 heteroaryl group.

In another embodiment, R₇ to R₁₆ are the same as or different from each other, and may be each independently hydrogen; a halogen group; —CN; a substituted or unsubstituted C6 to C40 aryl group; or a substituted or unsubstituted C2 to C40 heteroaryl group.

In another embodiment, R₇ to R₁₆ are the same as or different from each other, and may be each independently hydrogen; —F; —CN; a C6 to C40 aryl group; or a C2 to C40 heteroaryl group.

In another embodiment, R₇ to R₁₆ are the same as or different from each other, and may be each independently hydrogen; or —CN.

In one embodiment of the present application, R₇ to R₁₆ may be hydrogen.

In one embodiment of the present application, R₁₀ and R₁₃ of R₇ to R₁₆ are —CN, and the rest of the substituents may be hydrogen.

In one embodiment of the present application, R₁ to R₆ are the same as or different from each other, and each independently selected from the group consisting of hydrogen; deuterium; a halogen group; —CN; a substituted or unsubstituted C1 to C60 alkyl group; a substituted or unsubstituted C6 to C60 aryl group; a substituted or unsubstituted C2 to C60 heteroaryl group; —SiRR′ R″; —P(═O)RR′; and an amine group unsubstituted or substituted with a substituted or unsubstituted C1 to C60 alkyl group, a substituted or unsubstituted C6 to C60 aryl group or a substituted or unsubstituted C2 to C60 heteroaryl group, or two or more groups adjacent to each other may bond to each other to form a substituted or unsubstituted C6 to C60 aromatic hydrocarbon ring or a substituted or unsubstituted C2 to C60 heteroring.

In another embodiment, R₁ to R₆ are the same as or different from each other, and each independently selected from the group consisting of hydrogen; a halogen group; —CN; a substituted or unsubstituted C1 to C60 alkyl group; a substituted or unsubstituted C6 to C60 aryl group; a substituted or unsubstituted C2 to C60 heteroaryl group; —P(═O)RR′; and an amine group unsubstituted or substituted with a substituted or unsubstituted C1 to C60 alkyl group, a substituted or unsubstituted C6 to C60 aryl group or a substituted or unsubstituted C2 to C60 heteroaryl group, or two or more groups adjacent to each other may bond to each other to form a substituted or unsubstituted C6 to C60 aromatic hydrocarbon ring or a substituted or unsubstituted C2 to C60 heteroring.

In another embodiment, R₁ to R₆ are the same as or different from each other, and may be each independently selected from the group consisting of hydrogen; a halogen group; —CN; a substituted or unsubstituted C1 to C40 alkyl group; a substituted or unsubstituted C6 to C40 aryl group; a substituted or unsubstituted C2 to C40 heteroaryl group; and —P(═O)RR′.

In another embodiment, R₁ to R₆ are the same as or different from each other, and may be each independently selected from the group consisting of hydrogen; a halogen group; —CN; a C1 to C40 alkyl group unsubstituted or substituted with a halogen group; a C6 to C40 aryl group unsubstituted or substituted with one or more substituents selected from the group consisting of —CN, —F, —P(═O)RR′ and a C2 to C40 heteroaryl group; a C2 to C40 heteroaryl group unsubstituted or substituted with one or more substituents selected from the group consisting of —CN, —F, —P(═O)RR′ and a C6 to C40 aryl group; and —P(═O)RR′.

In one embodiment of the present application, when Ar of Chemical Formula 1 is hydrogen, at least one of R₁ to R₆ may be represented by -(L₃)p-(Z₁)q, and the rest are hydrogen; or a substituted or unsubstituted C6 to C60 aryl group,

L₃ is a direct bond; a substituted or unsubstituted C6 to C60 arylene group; or a substituted or unsubstituted C2 to C60 heteroarylene group,

Z₁ is a halogen group; —CN; a substituted or unsubstituted C1 to C60 alkyl group; a substituted or unsubstituted C6 to C60 aryl group; a substituted or unsubstituted C2 to C60 heteroaryl group; —SiRR′R″; or —P(═O)RR′,

R and R′ have the same definitions as in Chemical Formula 1,

p is an integer of 0 to 3, and

q is an integer of 1 to 3.

In one embodiment of the present application, when Ar of Chemical Formula 1 is hydrogen, one of R₁ to R₆ may be represented by -(L₃)p-(Z₁)q.

In one embodiment of the present application, when Ar of Chemical Formula 1 is hydrogen, R₁ of R₁ to R₆ may be represented by -(L₃)p-(Z₁)q, and the rest may be hydrogen; or a phenyl group.

In one embodiment of the present application, when Ar of Chemical Formula 1 is hydrogen, R₂ of R₁ to R₆ may be represented by -(L₃)p-(Z₁)q, and the rest may be hydrogen; or a phenyl group.

In one embodiment of the present application, when Ar of Chemical Formula 1 is hydrogen, R₃ of R₁ to R₆ may be represented by -(L₃)p-(Z₁)q, and the rest may be hydrogen; or a phenyl group.

In one embodiment of the present application, when Ar of Chemical Formula 1 is hydrogen, R₄ of R₁ to R₆ may be represented by -(L₃)p-(Z₁)q, and the rest may be hydrogen; or a phenyl group.

In one embodiment of the present application, when Ar of Chemical Formula 1 is hydrogen, R₅ of R₁ to R₆ may be represented by -(L₃)p-(Z₁)q, and the rest may be hydrogen; or a phenyl group.

In one embodiment of the present application, when Ar of Chemical Formula 1 is hydrogen, R₆ of R₁ to R₆ may be represented by -(L₃)p-(Z₁)q, and the rest may be hydrogen; or a phenyl group.

In one embodiment of the present application, L₃ may be a direct bond; or a substituted or unsubstituted C6 to C60 arylene group.

In another embodiment, L₃ may be a direct bond; or a substituted or unsubstituted C6 to C40 arylene group.

In another embodiment, L₃ may be a direct bond; or a C6 to C40 arylene group.

In another embodiment, L₃ may be a direct bond; or a phenylene group.

In one embodiment of the present application, Z₁ may be a halogen group; —CN; a substituted or unsubstituted C1 to C60 alkyl group; a substituted or unsubstituted C6 to C60 aryl group; a substituted or unsubstituted C2 to C60 heteroaryl group; —SiRR′ R″; or —P(═O)RR′.

In another embodiment, Z₁ may be a halogen group; —CN; a substituted or unsubstituted C1 to C40 alkyl group; a substituted or unsubstituted C6 to C40 aryl group; a substituted or unsubstituted C2 to C40 heteroaryl group; —SiRR′ R″; or —P(═O)RR′.

In another embodiment, Z₁ may be a halogen group; —CN; a C1 to C40 alkyl group unsubstituted or substituted with a halogen group; a C6 to C40 aryl group unsubstituted or substituted with one or more substituents selected from the group consisting of a halogen group, —CN and —CF₃; a C2 to C40 heteroaryl group unsubstituted or substituted with a C6 to C40 aryl group; or —P(═O)RR′.

In another embodiment Z₁ may be a halogen group; —CN; a C1 to C20 alkyl group unsubstituted or substituted with a halogen group; a monocyclic or polycyclic C6 to C40 aryl group unsubstituted or substituted with one or more substituents selected from the group consisting of a halogen group, —CN and —CF₃; a monocyclic or polycyclic C2 to C40 heteroaryl group unsubstituted or substituted with a C6 to C40 aryl group; or —P(═O)RR′.

In another embodiment, Z₁ may be —F; —CN; —CF₃; a phenyl group unsubstituted or substituted with one or more substituents selected from the group consisting of a fluoro group, —CN and —CF₃; a triphenylenyl group; a triazine group unsubstituted or substituted with a phenyl group; a pyrimidine group unsubstituted or substituted with a phenyl group; a phenanthroline group unsubstituted or substituted with a phenyl group; a carbazole group; or —P(═O)RR′.

In the heterocyclic compound provided in one embodiment of the present application, Chemical Formula 1 is represented by any one of the following compounds.

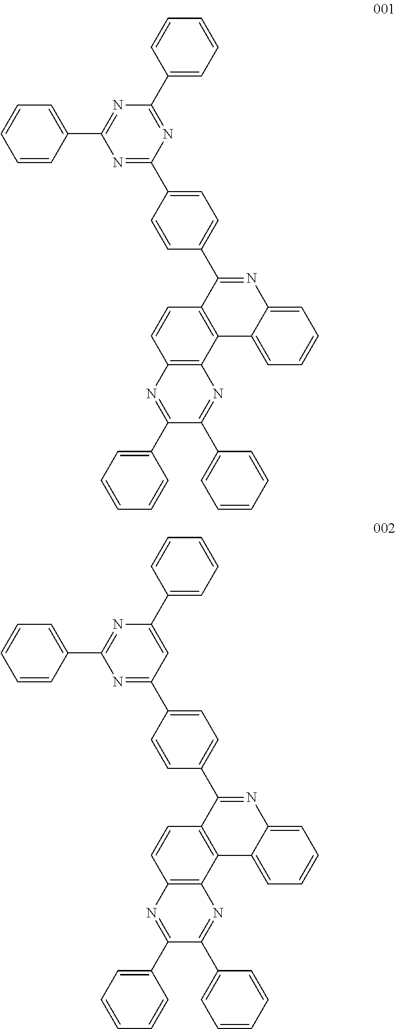

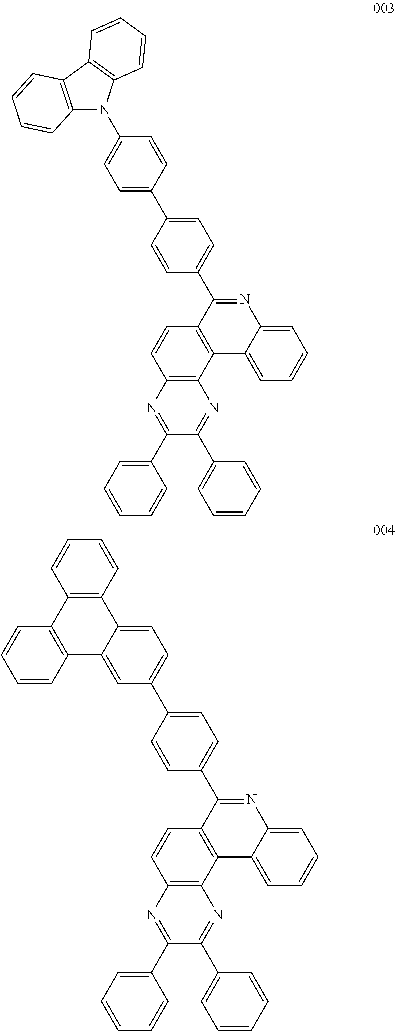

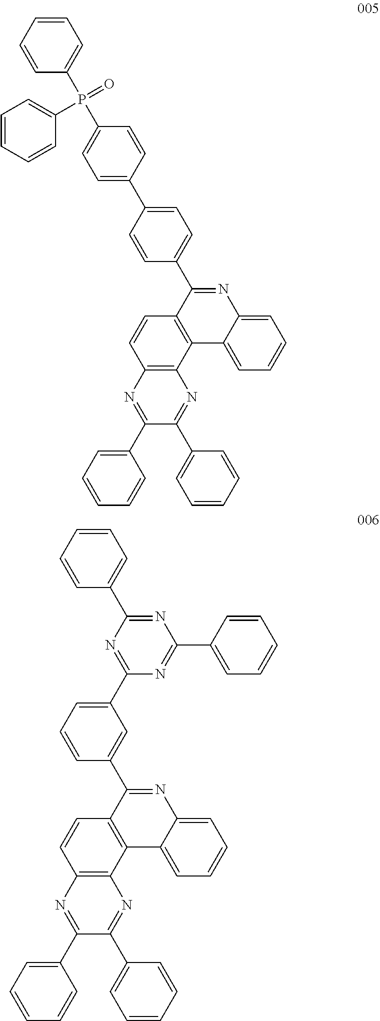

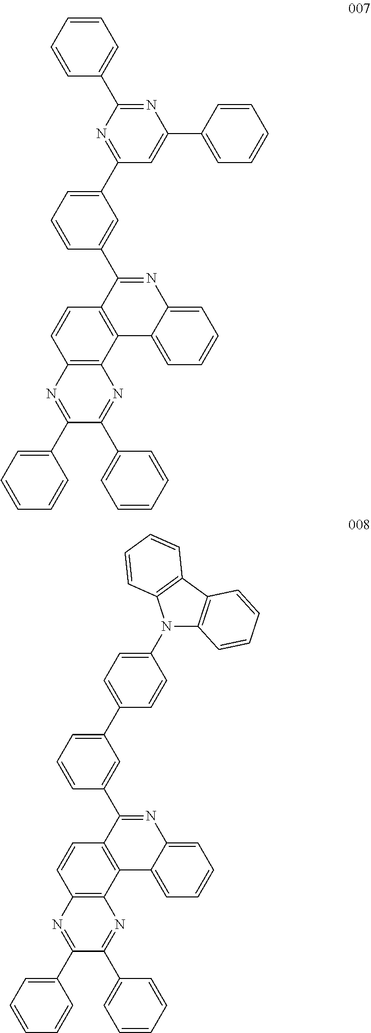

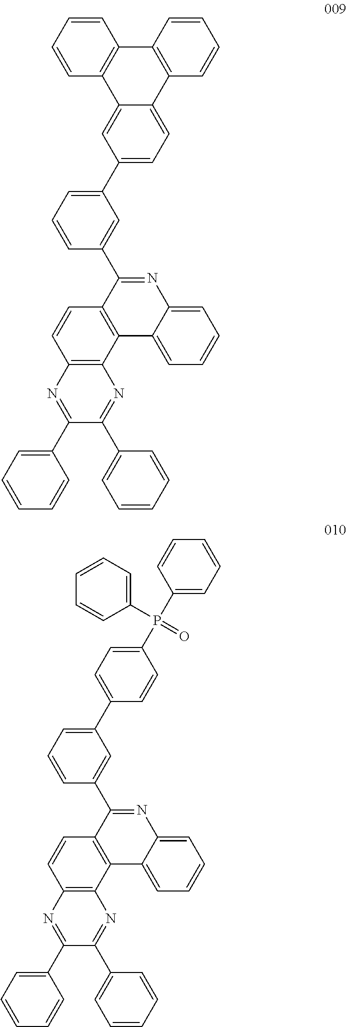

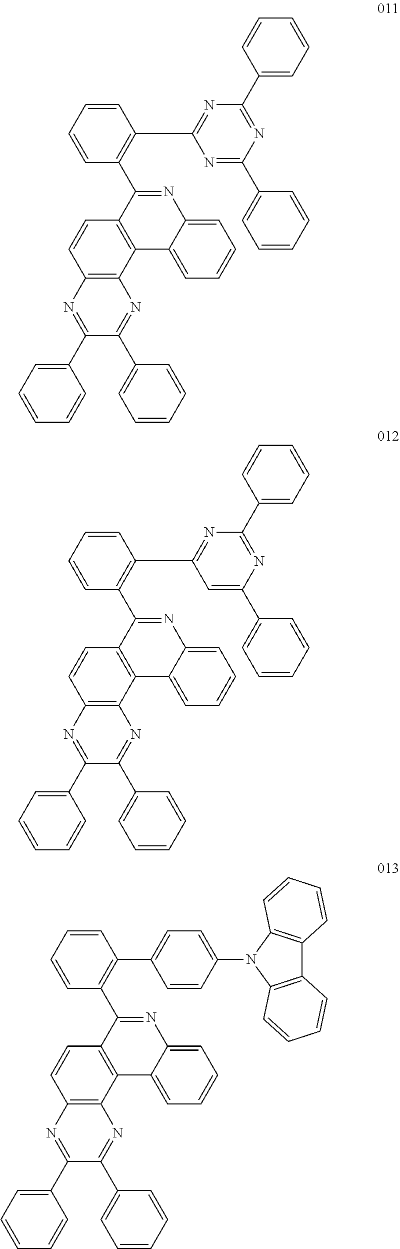 [SMILES: c1ccc(-c2nc(-c3ccccc3)nc(-c3ccccc3-c3nc4ccccc4c4c3ccc3nc(-c5ccccc5)c(-c5ccccc5)nc34)n2)cc1 ; c1ccc(-c2cc(-c3ccccc3-c3nc4ccccc4c4c3ccc3nc(-c5ccccc5)c(-c5ccccc5)nc34)nc(-c3ccccc3)n2)cc1 ; c1ccc(-c2nc3ccc4c(-c5ccccc5-c5ccc(-n6c7ccccc7c7ccccc76)cc5)nc5ccccc5c4c3nc2-c2ccccc2)cc1]

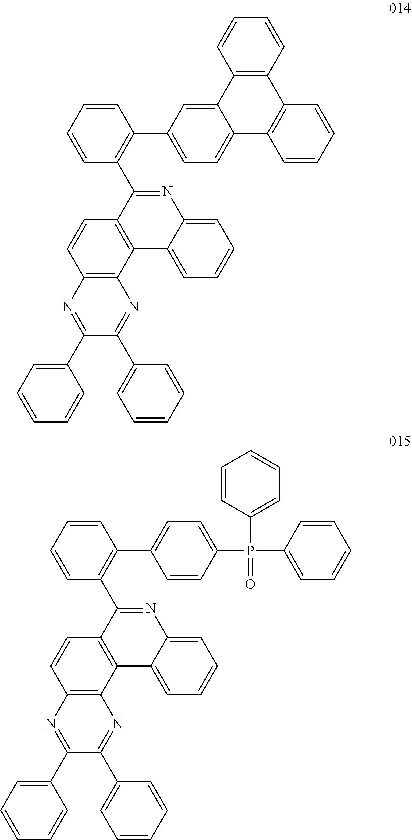 [SMILES: c1ccc(-c2nc3ccc4c(-c5ccccc5-c5ccc6c7ccccc7c7ccccc7c6c5)nc5ccccc5c4c3nc2-c2ccccc2)cc1 ; O=P(c1ccccc1)(c1ccccc1)c1ccc(-c2ccccc2-c2nc3ccccc3c3c2ccc2nc(-c4ccccc4)c(-c4ccccc4)nc23)cc1]

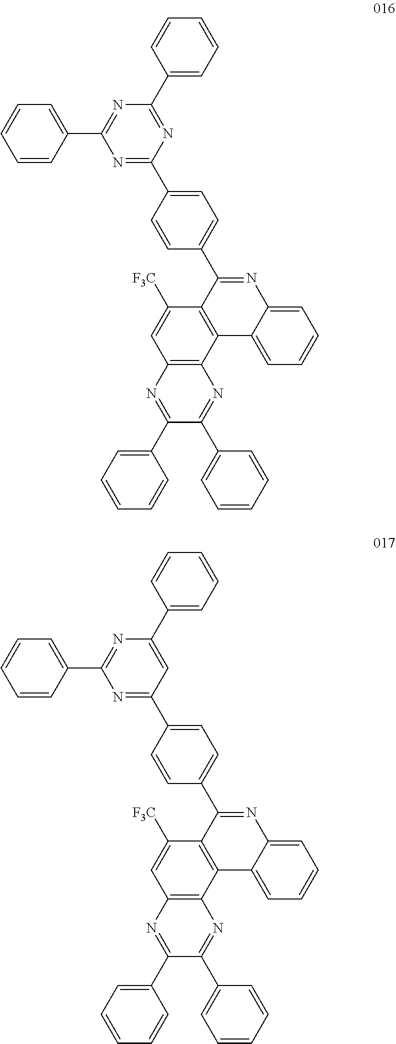 [SMILES: FC(F)(F)c1cc2nc(-c3ccccc3)c(-c3ccccc3)nc2c2c1c(-c1ccc(-c3nc(-c4ccccc4)nc(-c4ccccc4)n3)cc1)nc1ccccc12 ; FC(F)(F)c1cc2nc(-c3ccccc3)c(-c3ccccc3)nc2c2c1c(-c1ccc(-c3cc(-c4ccccc4)nc(-c4ccccc4)n3)cc1)nc1ccccc12]

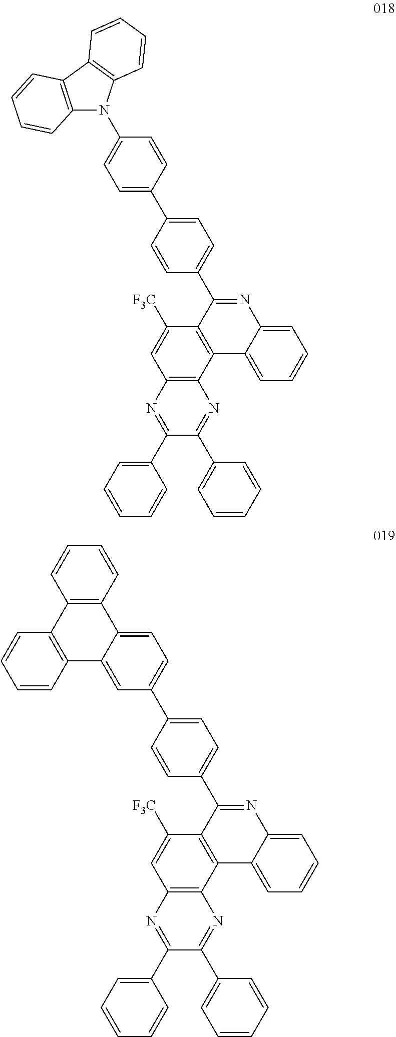

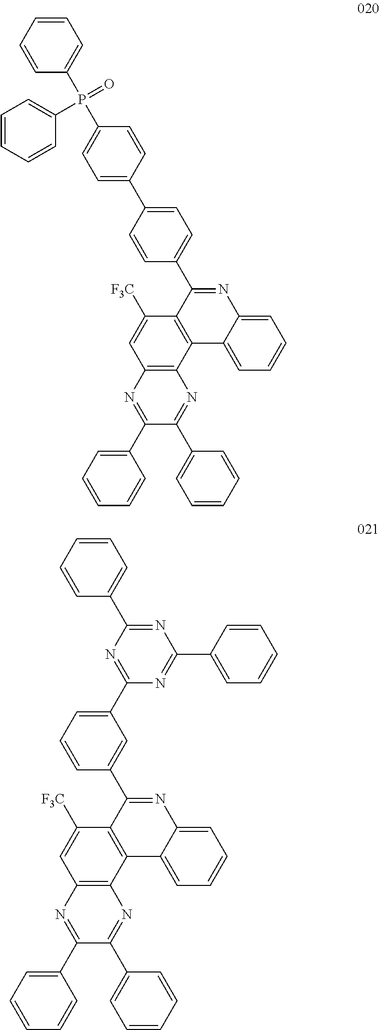

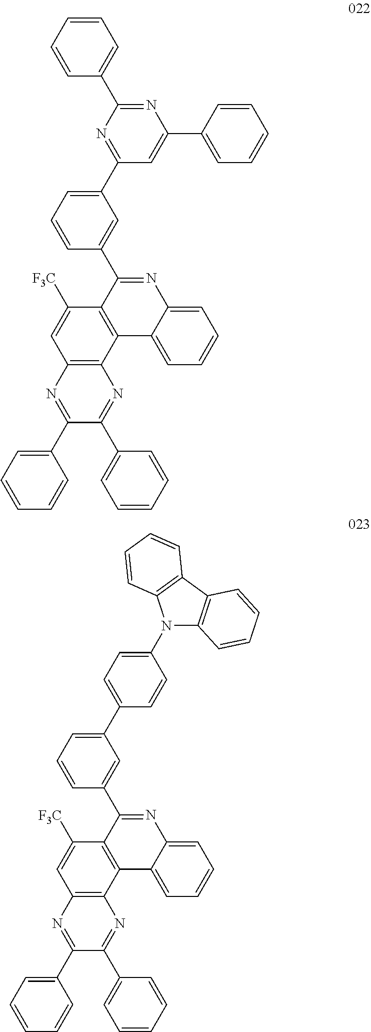

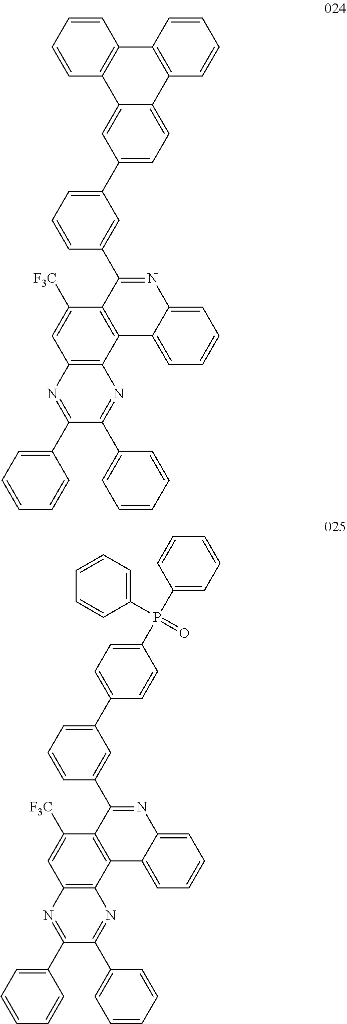

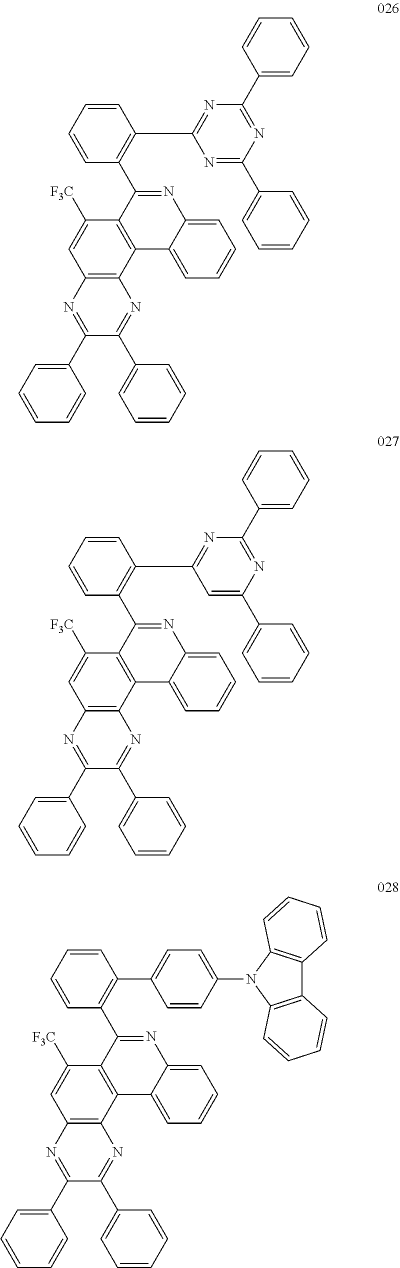 [SMILES: FC(F)(F)c1cc2nc(-c3ccccc3)c(-c3ccccc3)nc2c2c1c(-c1ccccc1-c1nc(-c3ccccc3)nc(-c3ccccc3)n1)nc1ccccc12 ; FC(F)(F)c1cc2nc(-c3ccccc3)c(-c3ccccc3)nc2c2c1c(-c1ccccc1-c1cc(-c3ccccc3)nc(-c3ccccc3)n1)nc1ccccc12 ; FC(F)(F)c1cc2nc(-c3ccccc3)c(-c3ccccc3)nc2c2c1c(-c1ccccc1-c1ccc(-n3c4ccccc4c4ccccc43)cc1)nc1ccccc12]

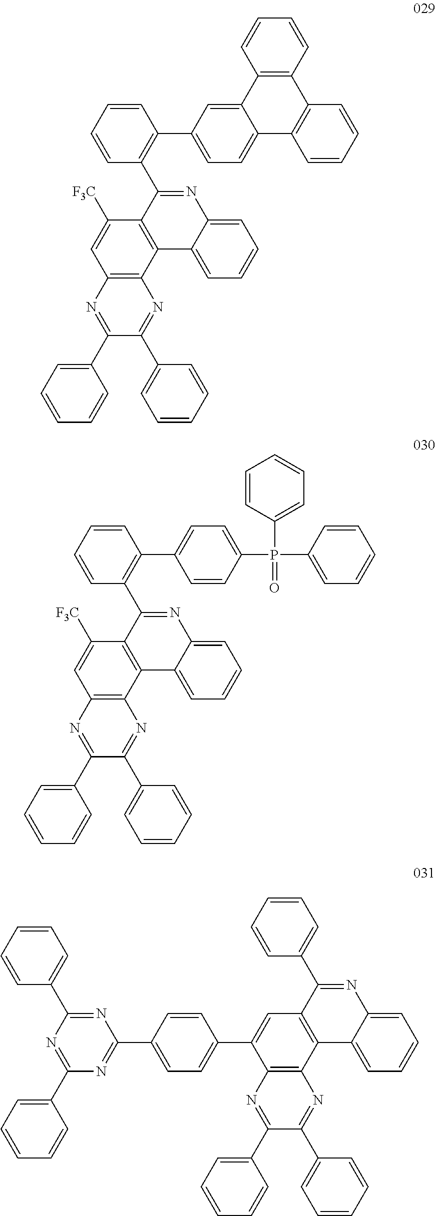 [SMILES: FC(F)(F)c1cc2nc(-c3ccccc3)c(-c3ccccc3)nc2c2c1c(-c1ccccc1-c1ccc3c4ccccc4c4ccccc4c3c1)nc1ccccc12 ; O=P(c1ccccc1)(c1ccccc1)c1ccc(-c2ccccc2-c2nc3ccccc3c3c2c(C(F)(F)F)cc2nc(-c4ccccc4)c(-c4ccccc4)nc23)cc1 ; c1ccc(-c2nc(-c3ccccc3)nc(-c3ccc(-c4cc5c(-c6ccccc6)nc6ccccc6c5c5nc(-c6ccccc6)c(-c6ccccc6)nc45)cc3)n2)cc1]

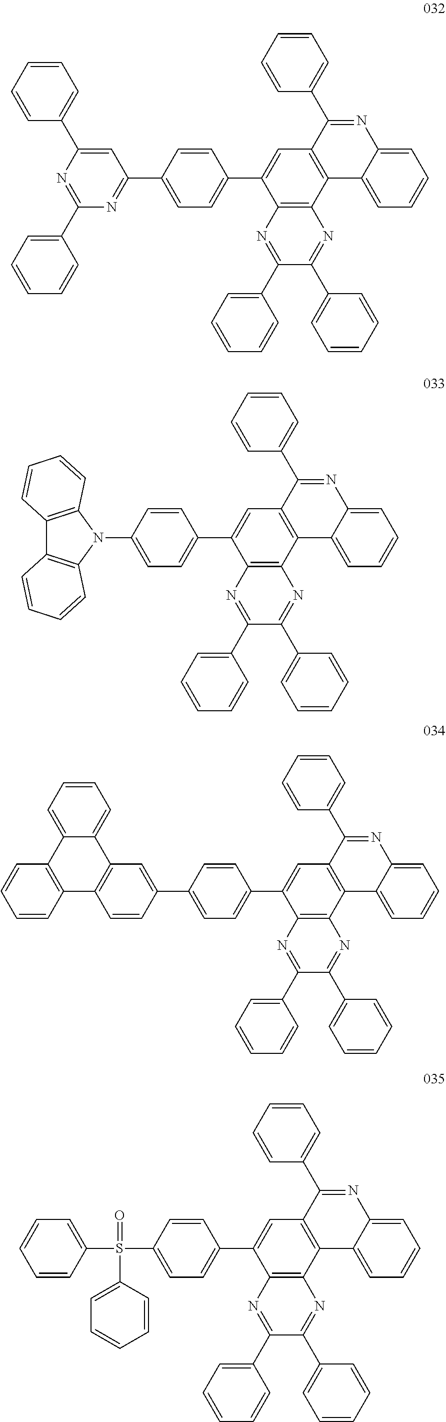

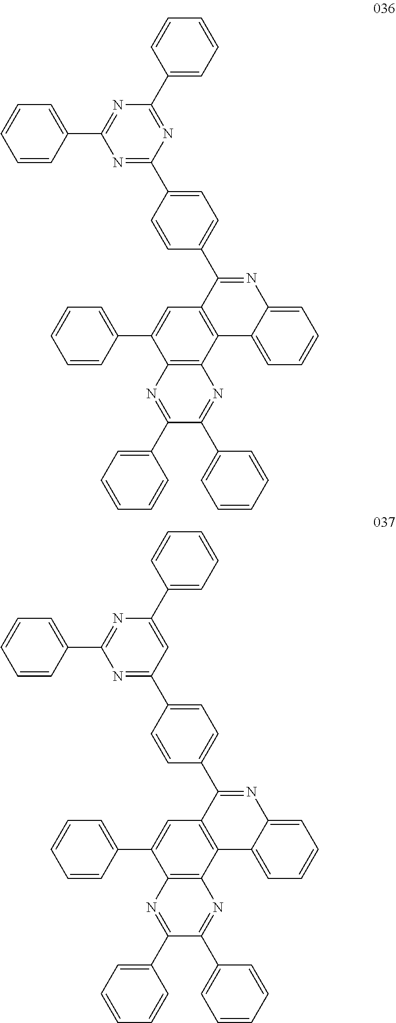

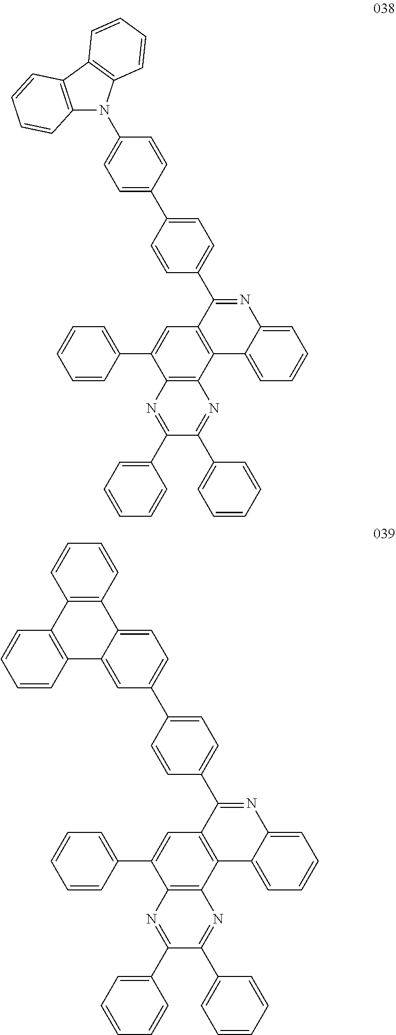

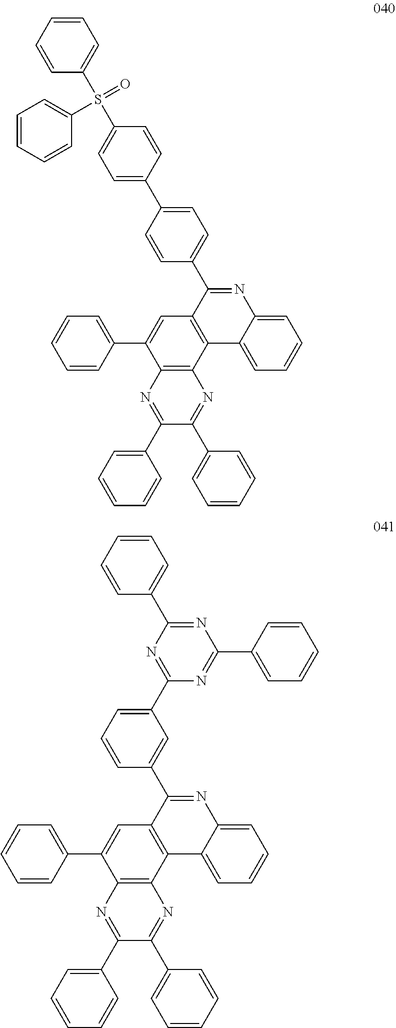

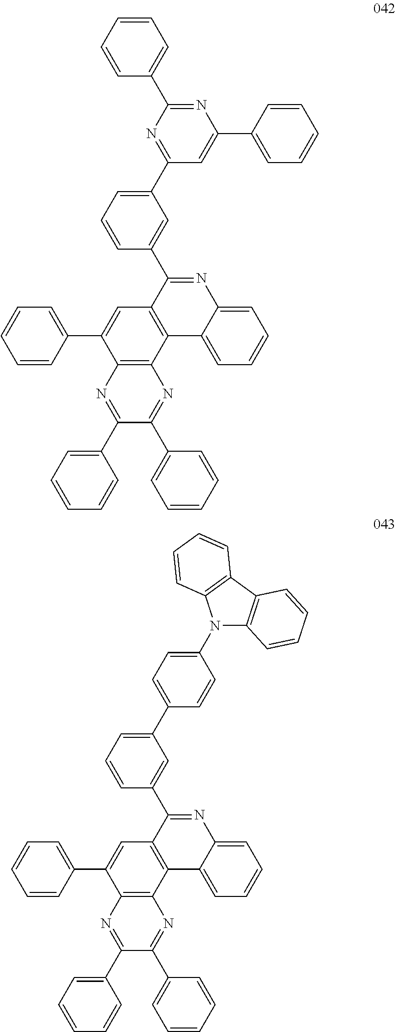

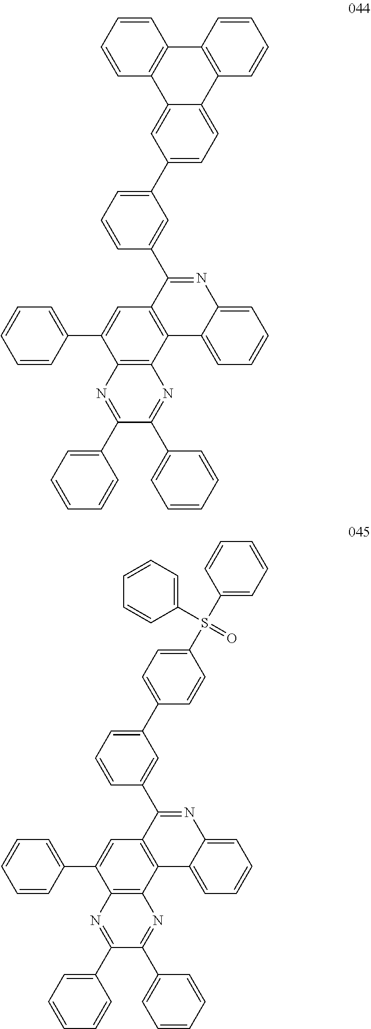

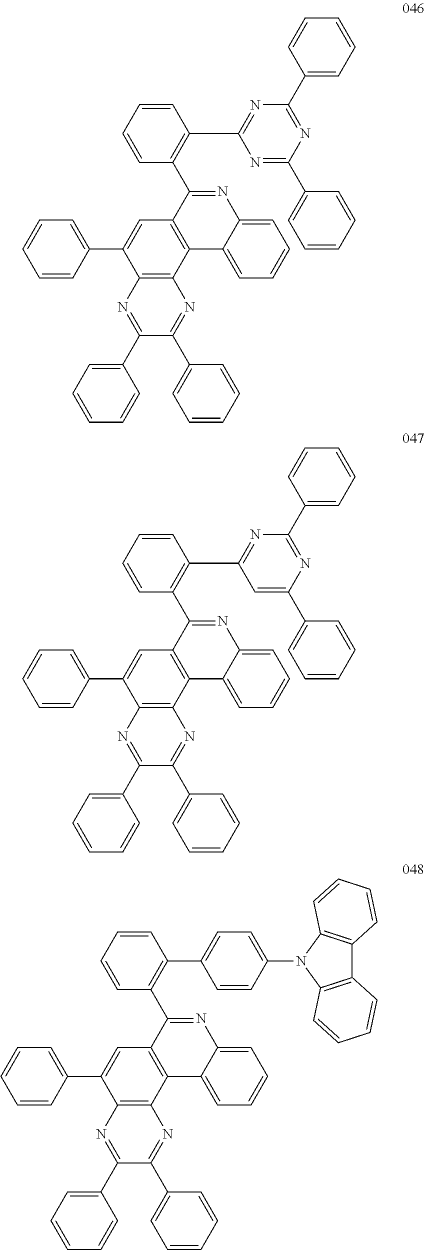 [SMILES: c1ccc(-c2nc(-c3ccccc3)nc(-c3ccccc3-c3nc4ccccc4c4c3cc(-c3ccccc3)c3nc(-c5ccccc5)c(-c5ccccc5)nc34)n2)cc1 ; c1ccc(-c2cc(-c3ccccc3-c3nc4ccccc4c4c3cc(-c3ccccc3)c3nc(-c5ccccc5)c(-c5ccccc5)nc34)nc(-c3ccccc3)n2)cc1 ; c1ccc(-c2nc3c(-c4ccccc4)cc4c(-c5ccccc5-c5ccc(-n6c7ccccc7c7ccccc76)cc5)nc5ccccc5c4c3nc2-c2ccccc2)cc1]

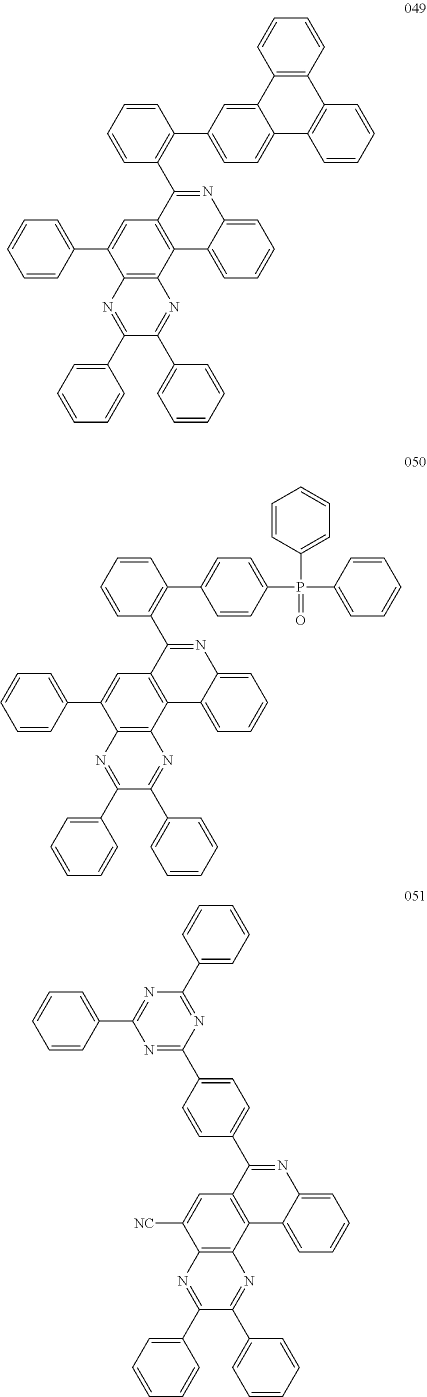

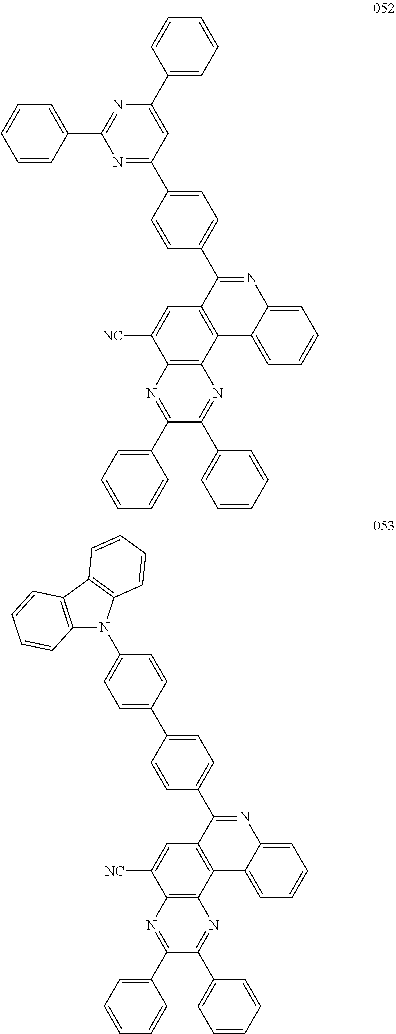

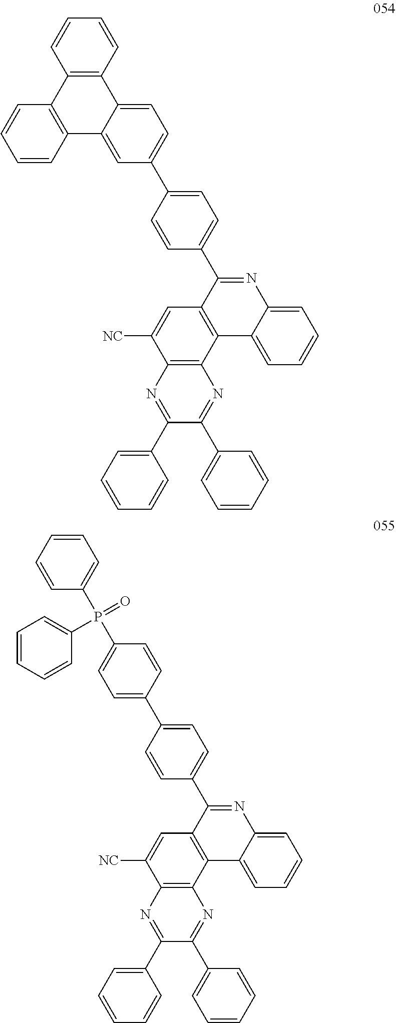

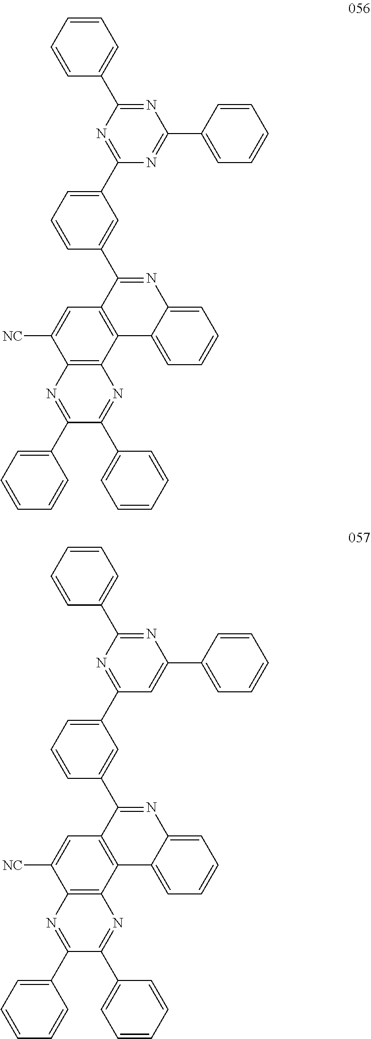

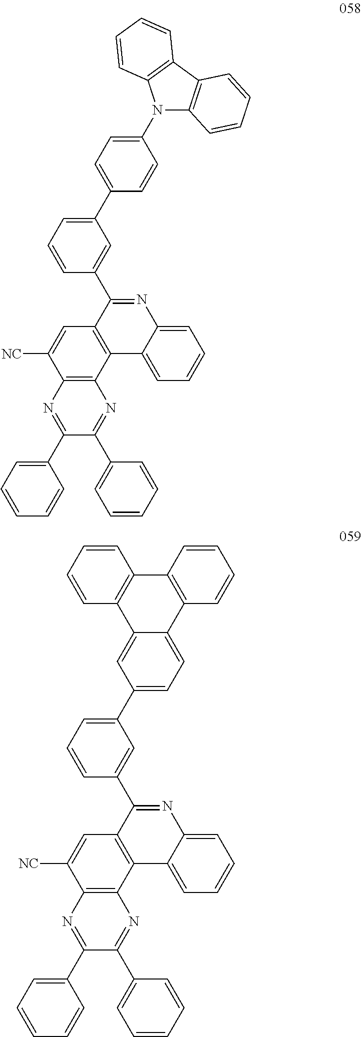

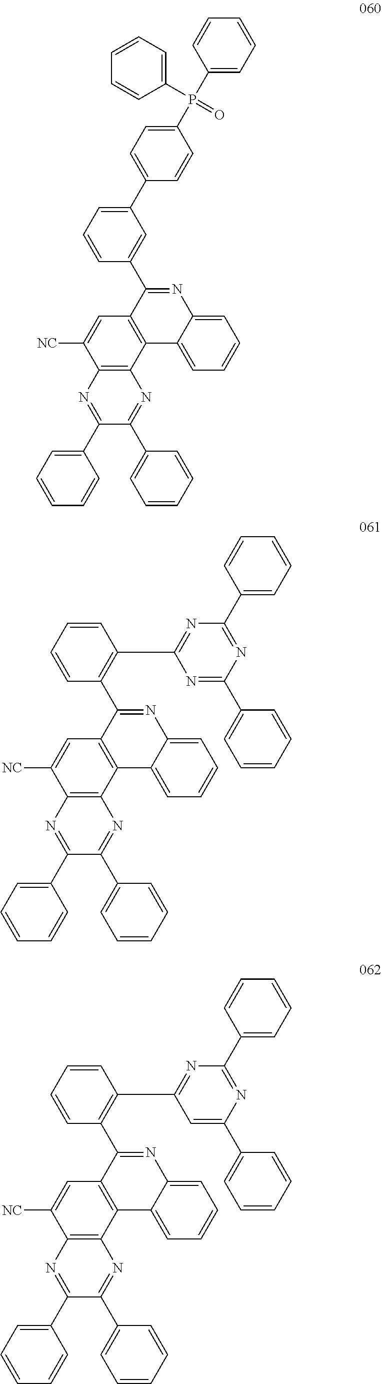 [SMILES: N#Cc1cc2c(-c3cccc(-c4ccc(P(=O)(c5ccccc5)c5ccccc5)cc4)c3)nc3ccccc3c2c2nc(-c3ccccc3)c(-c3ccccc3)nc12 ; N#Cc1cc2c(-c3ccccc3-c3nc(-c4ccccc4)nc(-c4ccccc4)n3)nc3ccccc3c2c2nc(-c3ccccc3)c(-c3ccccc3)nc12 ; N#Cc1cc2c(-c3ccccc3-c3cc(-c4ccccc4)nc(-c4ccccc4)n3)nc3ccccc3c2c2nc(-c3ccccc3)c(-c3ccccc3)nc12]

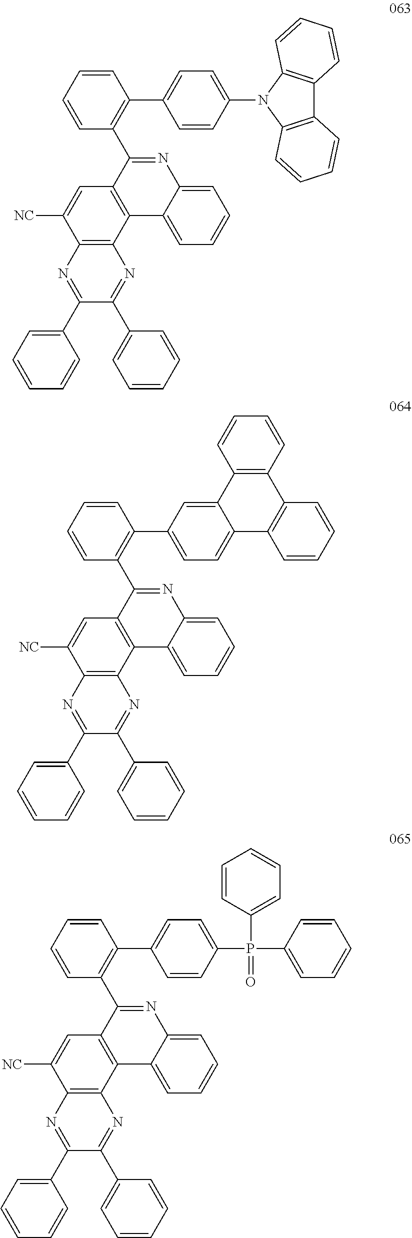 [SMILES: N#Cc1cc2c(-c3ccccc3-c3ccc(-n4c5ccccc5c5ccccc54)cc3)nc3ccccc3c2c2nc(-c3ccccc3)c(-c3ccccc3)nc12 ; N#Cc1cc2c(-c3ccccc3-c3ccc4c5ccccc5c5ccccc5c4c3)nc3ccccc3c2c2nc(-c3ccccc3)c(-c3ccccc3)nc12 ; N#Cc1cc2c(-c3ccccc3-c3ccc(P(=O)(c4ccccc4)c4ccccc4)cc3)nc3ccccc3c2c2nc(-c3ccccc3)c(-c3ccccc3)nc12]

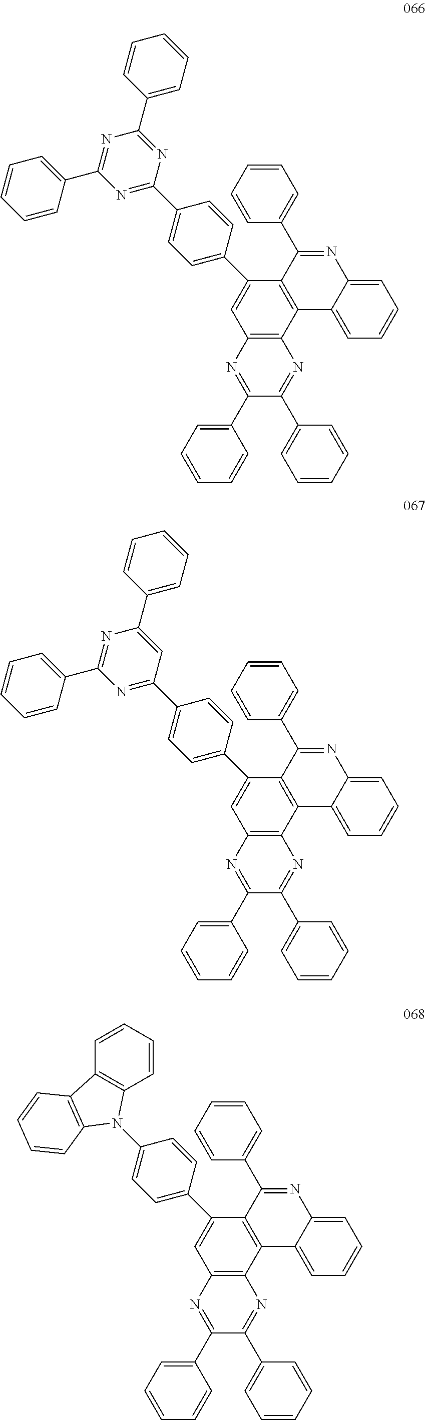 [SMILES: c1ccc(-c2nc(-c3ccccc3)nc(-c3ccc(-c4cc5nc(-c6ccccc6)c(-c6ccccc6)nc5c5c4c(-c4ccccc4)nc4ccccc45)cc3)n2)cc1 ; c1ccc(-c2cc(-c3ccc(-c4cc5nc(-c6ccccc6)c(-c6ccccc6)nc5c5c4c(-c4ccccc4)nc4ccccc45)cc3)nc(-c3ccccc3)n2)cc1 ; c1ccc(-c2nc3cc(-c4ccc(-n5c6ccccc6c6ccccc65)cc4)c4c(-c5ccccc5)nc5ccccc5c4c3nc2-c2ccccc2)cc1]

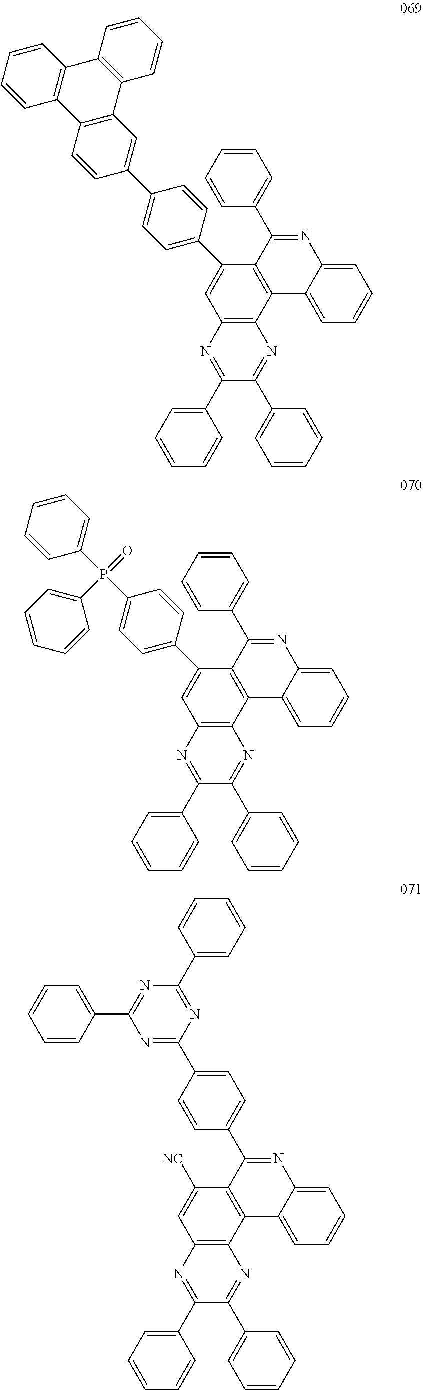

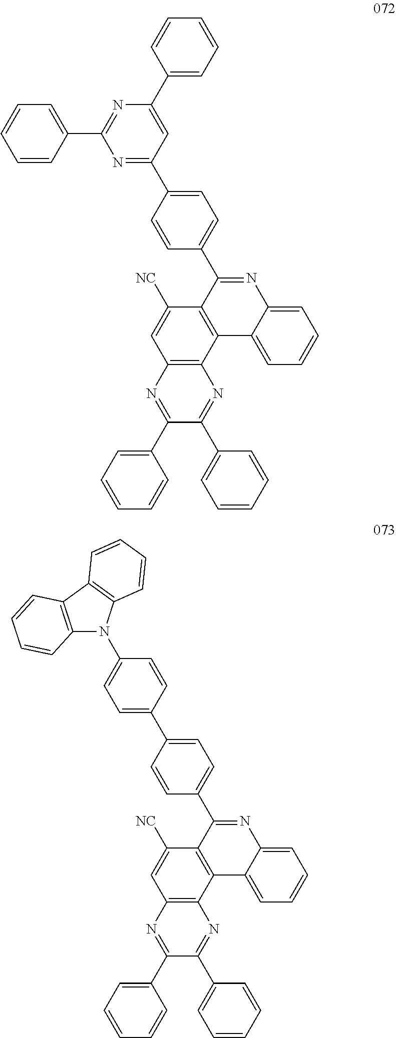

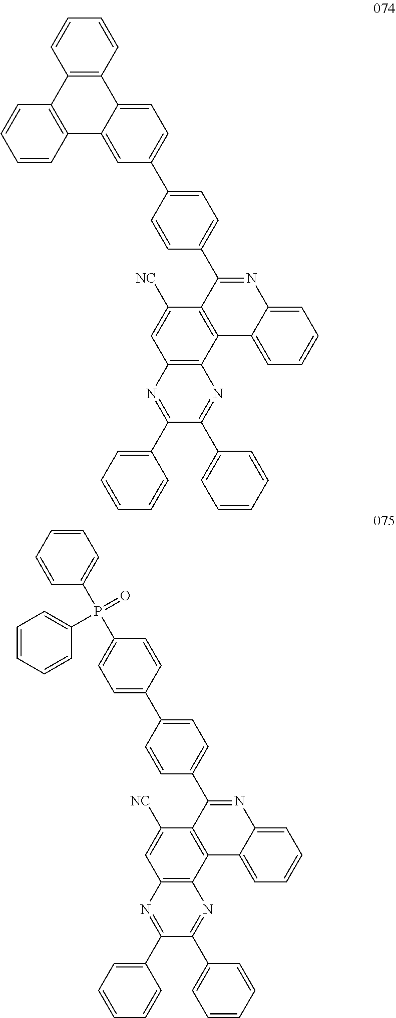

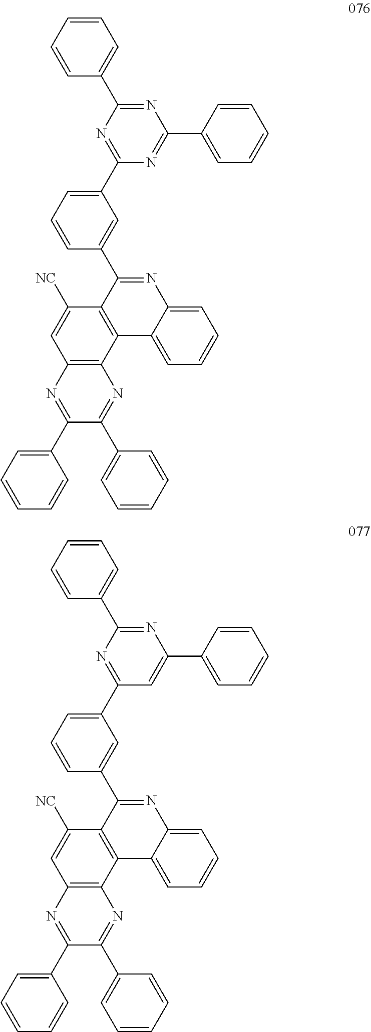

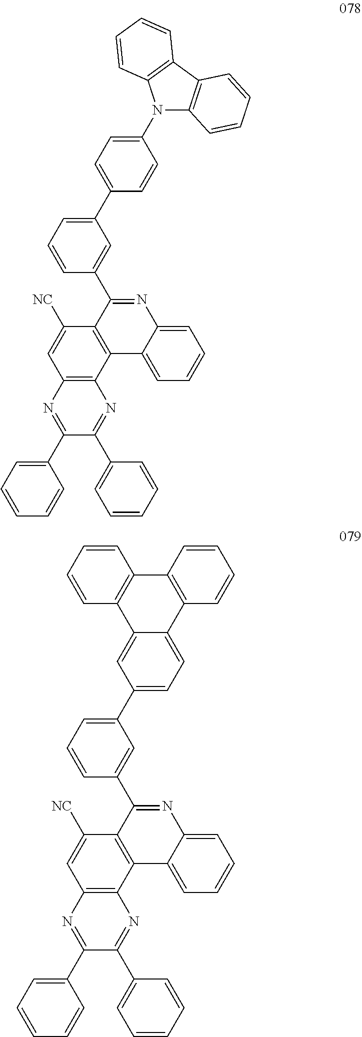

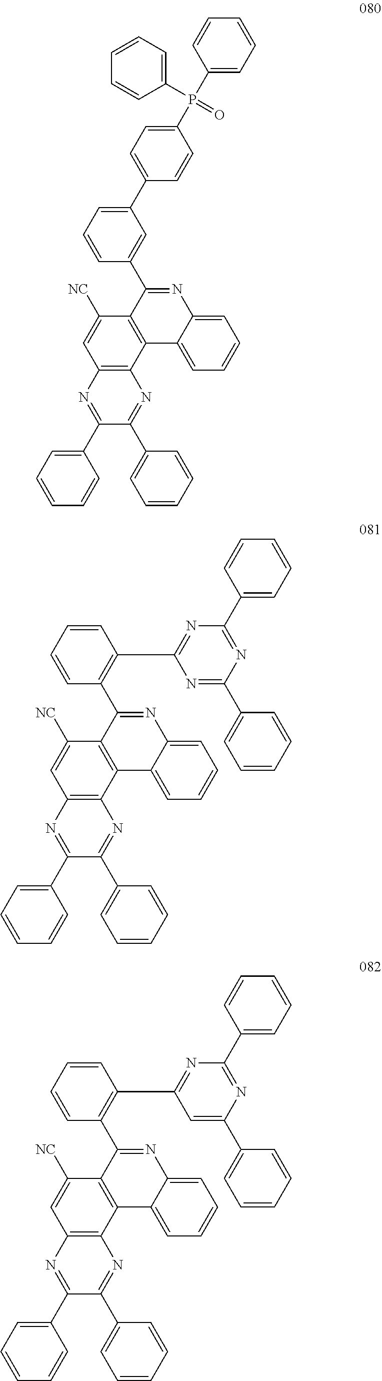 [SMILES: N#Cc1cc2nc(-c3ccccc3)c(-c3ccccc3)nc2c2c1c(-c1cccc(-c3ccc(P(=O)(c4ccccc4)c4ccccc4)cc3)c1)nc1ccccc12 ; N#Cc1cc2nc(-c3ccccc3)c(-c3ccccc3)nc2c2c1c(-c1ccccc1-c1nc(-c3ccccc3)nc(-c3ccccc3)n1)nc1ccccc12 ; N#Cc1cc2nc(-c3ccccc3)c(-c3ccccc3)nc2c2c1c(-c1ccccc1-c1cc(-c3ccccc3)nc(-c3ccccc3)n1)nc1ccccc12]

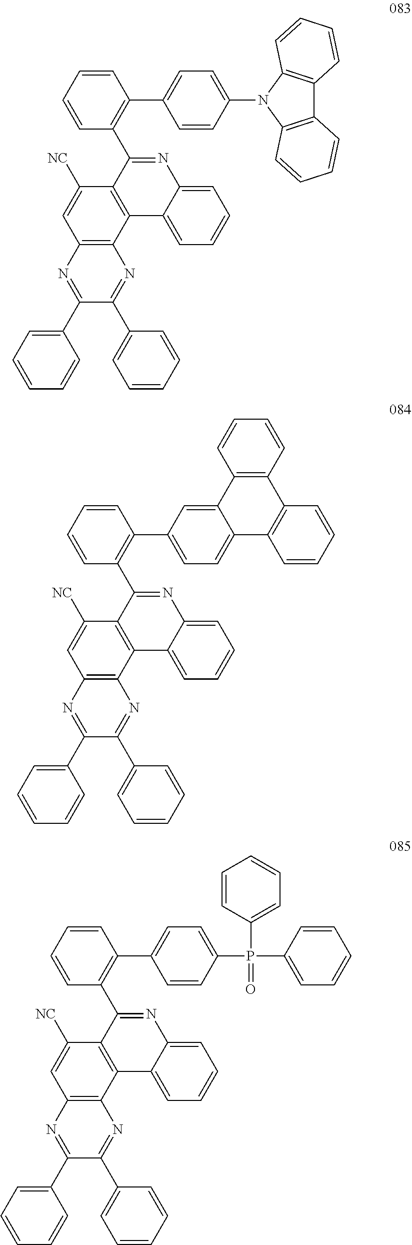 [SMILES: N#Cc1cc2nc(-c3ccccc3)c(-c3ccccc3)nc2c2c1c(-c1ccccc1-c1ccc(-n3c4ccccc4c4ccccc43)cc1)nc1ccccc12 ; N#Cc1cc2nc(-c3ccccc3)c(-c3ccccc3)nc2c2c1c(-c1ccccc1-c1ccc3c4ccccc4c4ccccc4c3c1)nc1ccccc12 ; N#Cc1cc2nc(-c3ccccc3)c(-c3ccccc3)nc2c2c1c(-c1ccccc1-c1ccc(P(=O)(c3ccccc3)c3ccccc3)cc1)nc1ccccc12]

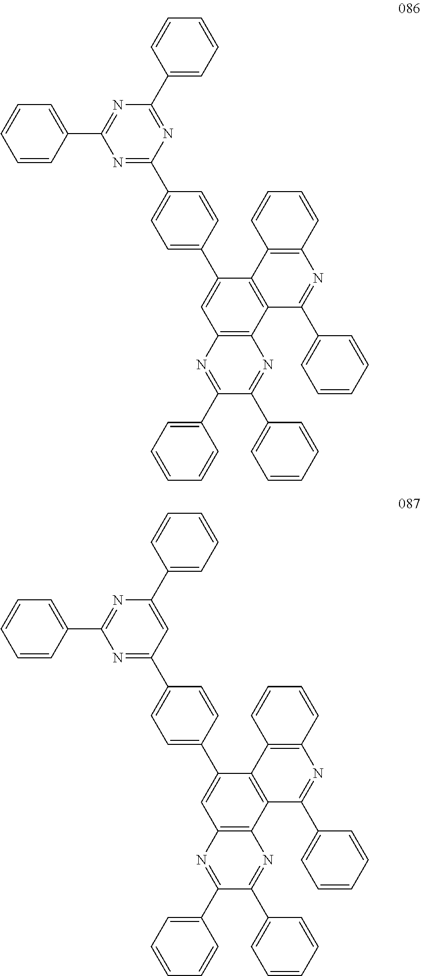

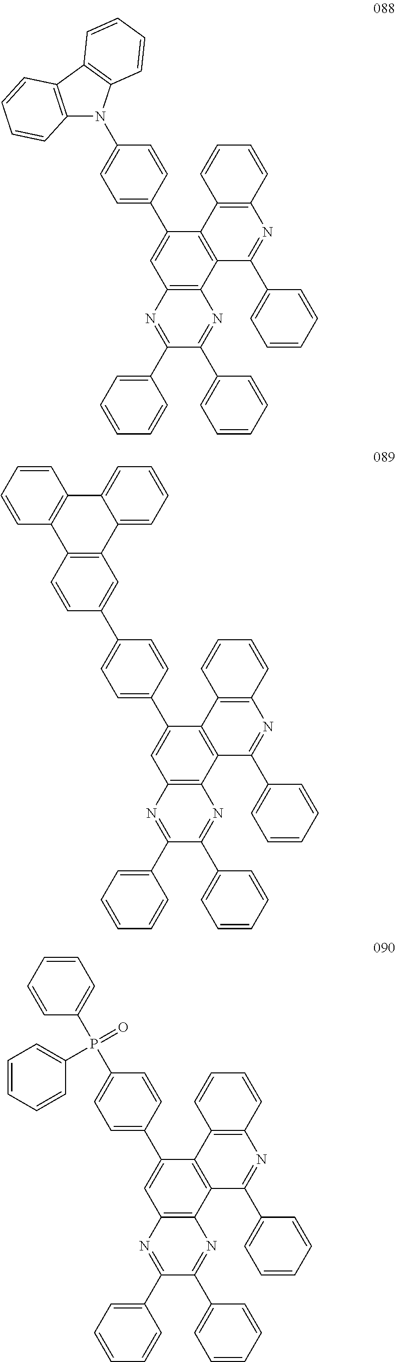

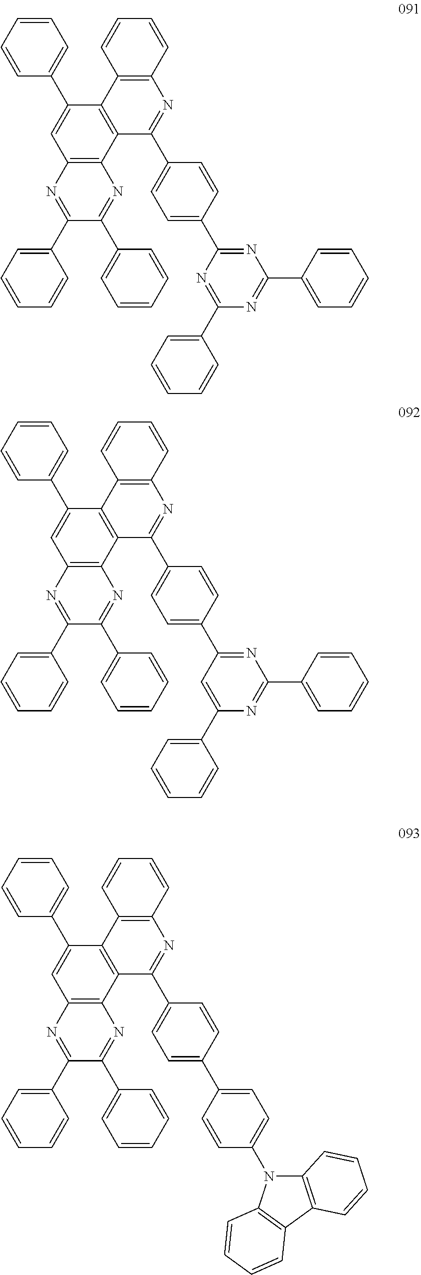

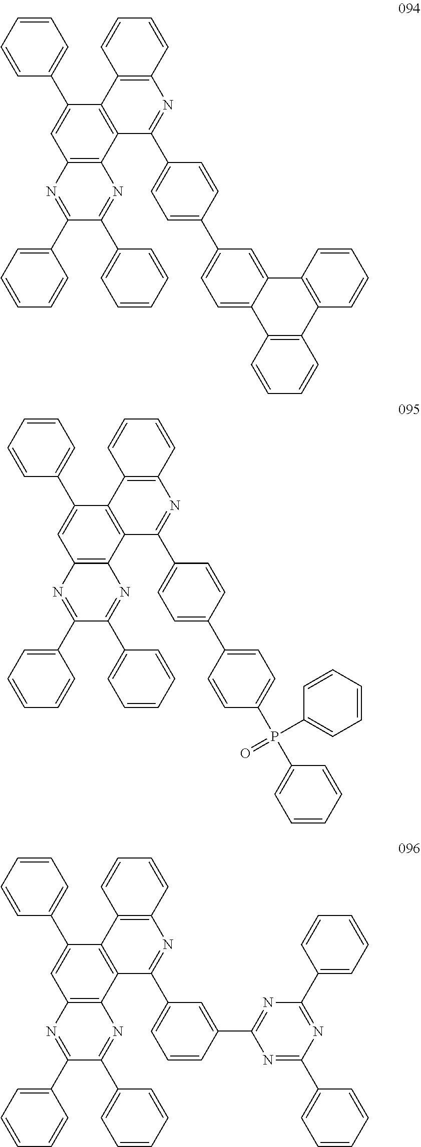 [SMILES: c1ccc(-c2nc3cc(-c4ccccc4)c4c5ccccc5nc(-c5ccc(-c6ccc7c8ccccc8c8ccccc8c7c6)cc5)c4c3nc2-c2ccccc2)cc1 ; O=P(c1ccccc1)(c1ccccc1)c1ccc(-c2ccc(-c3nc4ccccc4c4c(-c5ccccc5)cc5nc(-c6ccccc6)c(-c6ccccc6)nc5c34)cc2)cc1 ; c1ccc(-c2nc(-c3ccccc3)nc(-c3cccc(-c4nc5ccccc5c5c(-c6ccccc6)cc6nc(-c7ccccc7)c(-c7ccccc7)nc6c45)c3)n2)cc1]

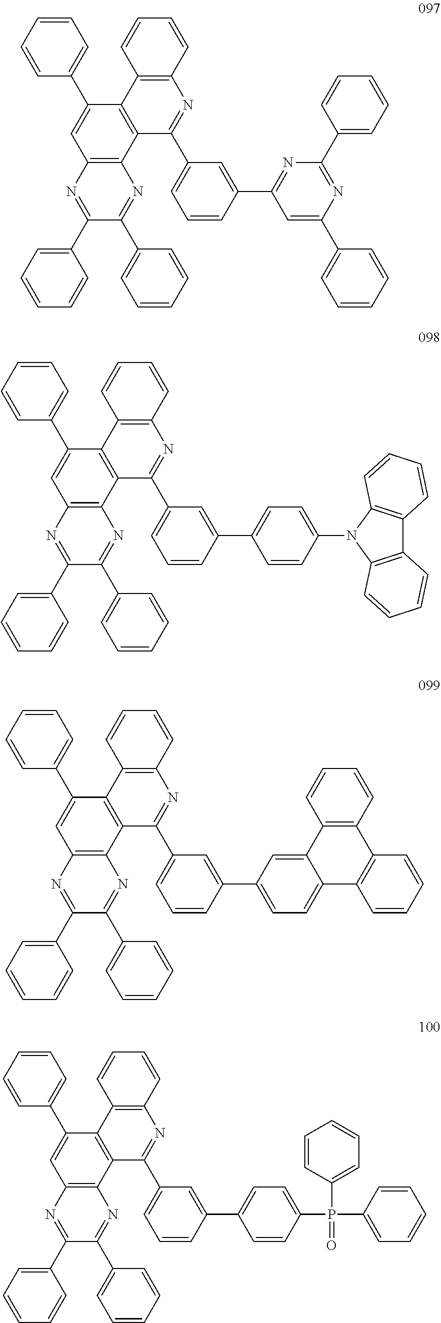 [SMILES: c1ccc(-c2cc(-c3cccc(-c4nc5ccccc5c5c(-c6ccccc6)cc6nc(-c7ccccc7)c(-c7ccccc7)nc6c45)c3)nc(-c3ccccc3)n2)cc1 ; c1ccc(-c2nc3cc(-c4ccccc4)c4c5ccccc5nc(-c5cccc(-c6ccc(-n7c8ccccc8c8ccccc87)cc6)c5)c4c3nc2-c2ccccc2)cc1 ; c1ccc(-c2nc3cc(-c4ccccc4)c4c5ccccc5nc(-c5cccc(-c6ccc7c8ccccc8c8ccccc8c7c6)c5)c4c3nc2-c2ccccc2)cc1 ; O=P(c1ccccc1)(c1ccccc1)c1ccc(-c2cccc(-c3nc4ccccc4c4c(-c5ccccc5)cc5nc(-c6ccccc6)c(-c6ccccc6)nc5c34)c2)cc1]

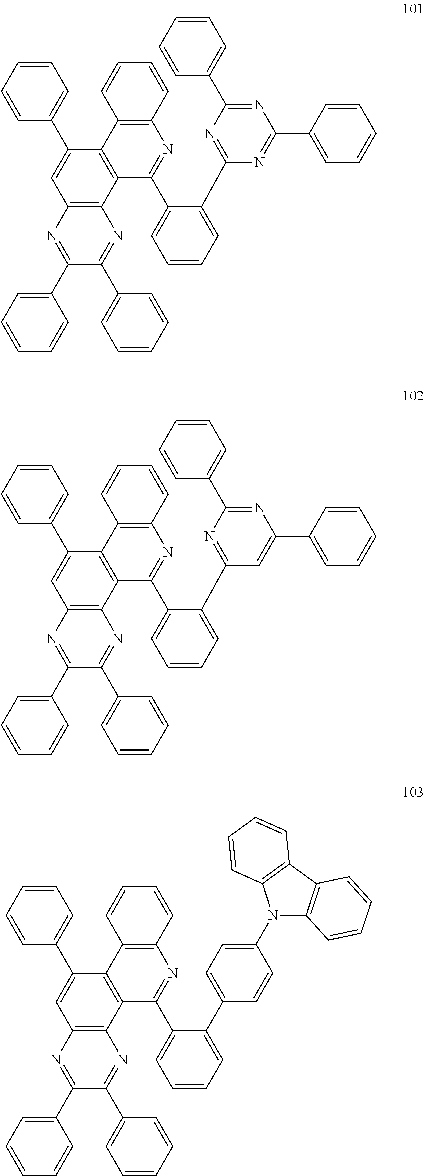 [SMILES: c1ccc(-c2nc(-c3ccccc3)nc(-c3ccccc3-c3nc4ccccc4c4c(-c5ccccc5)cc5nc(-c6ccccc6)c(-c6ccccc6)nc5c34)n2)cc1 ; c1ccc(-c2cc(-c3ccccc3-c3nc4ccccc4c4c(-c5ccccc5)cc5nc(-c6ccccc6)c(-c6ccccc6)nc5c34)nc(-c3ccccc3)n2)cc1 ; c1ccc(-c2nc3cc(-c4ccccc4)c4c5ccccc5nc(-c5ccccc5-c5ccc(-n6c7ccccc7c7ccccc76)cc5)c4c3nc2-c2ccccc2)cc1]

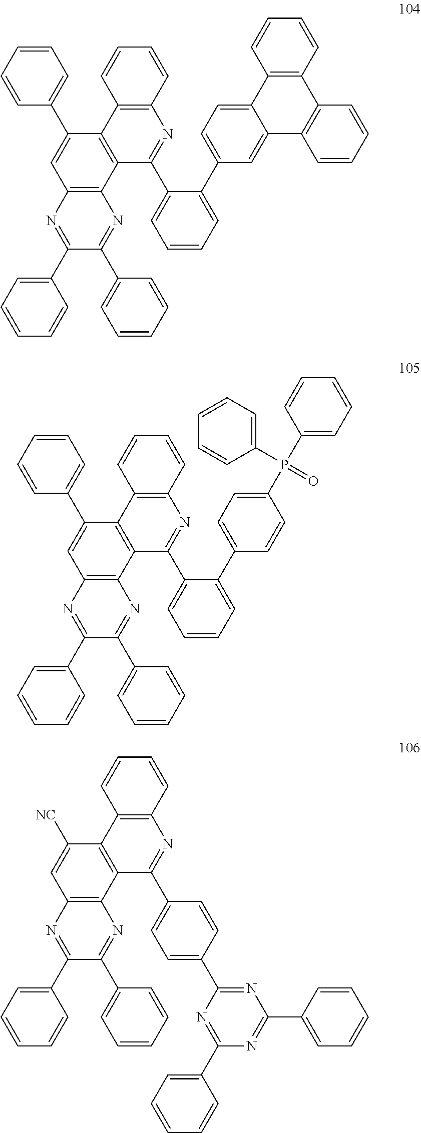

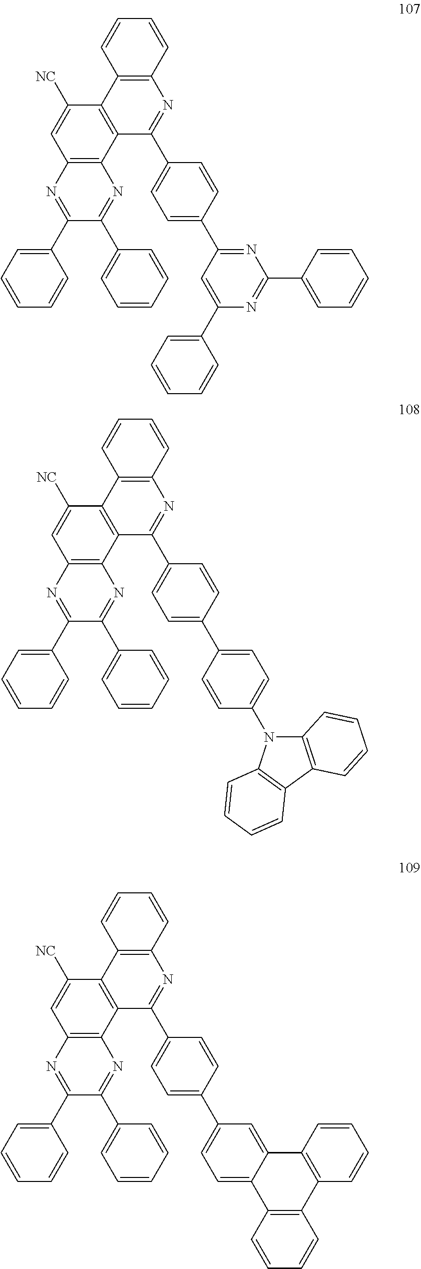

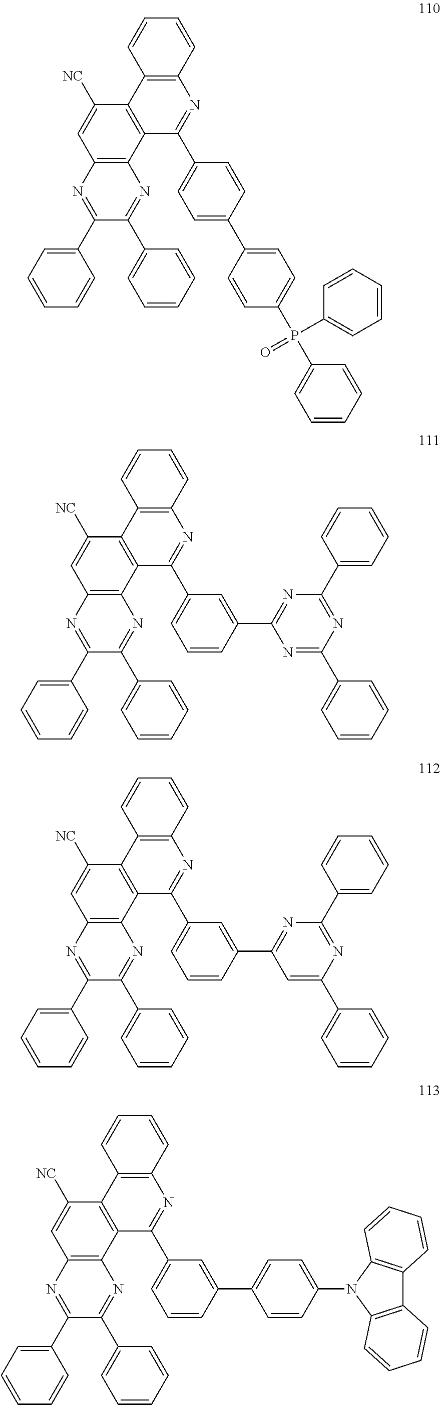

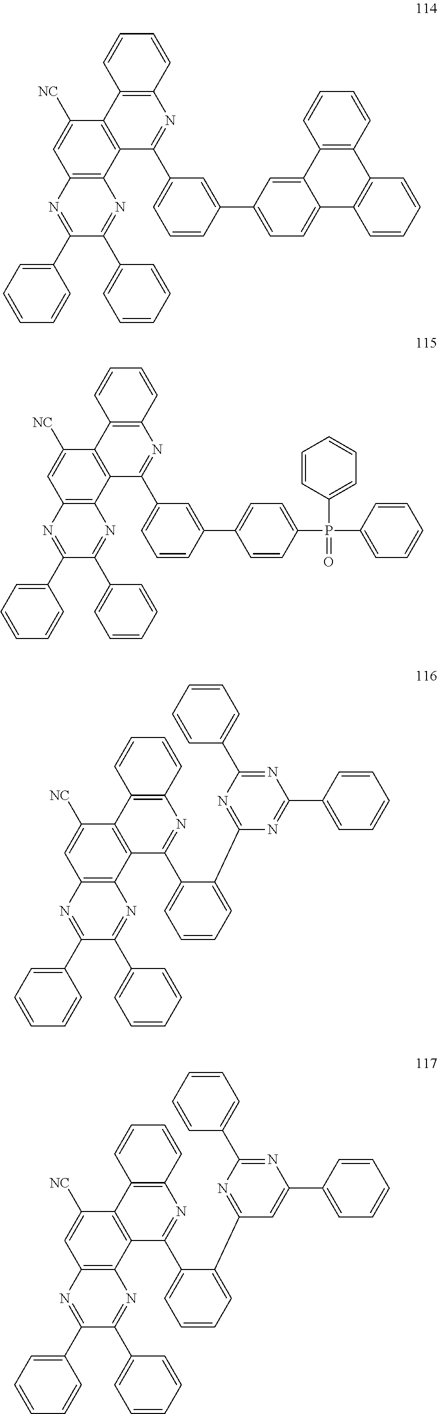 [SMILES: N#Cc1cc2nc(-c3ccccc3)c(-c3ccccc3)nc2c2c(-c3cccc(-c4ccc5c6ccccc6c6ccccc6c5c4)c3)nc3ccccc3c12 ; N#Cc1cc2nc(-c3ccccc3)c(-c3ccccc3)nc2c2c(-c3cccc(-c4ccc(P(=O)(c5ccccc5)c5ccccc5)cc4)c3)nc3ccccc3c12 ; N#Cc1cc2nc(-c3ccccc3)c(-c3ccccc3)nc2c2c(-c3ccccc3-c3nc(-c4ccccc4)nc(-c4ccccc4)n3)nc3ccccc3c12 ; N#Cc1cc2nc(-c3ccccc3)c(-c3ccccc3)nc2c2c(-c3ccccc3-c3cc(-c4ccccc4)nc(-c4ccccc4)n3)nc3ccccc3c12]

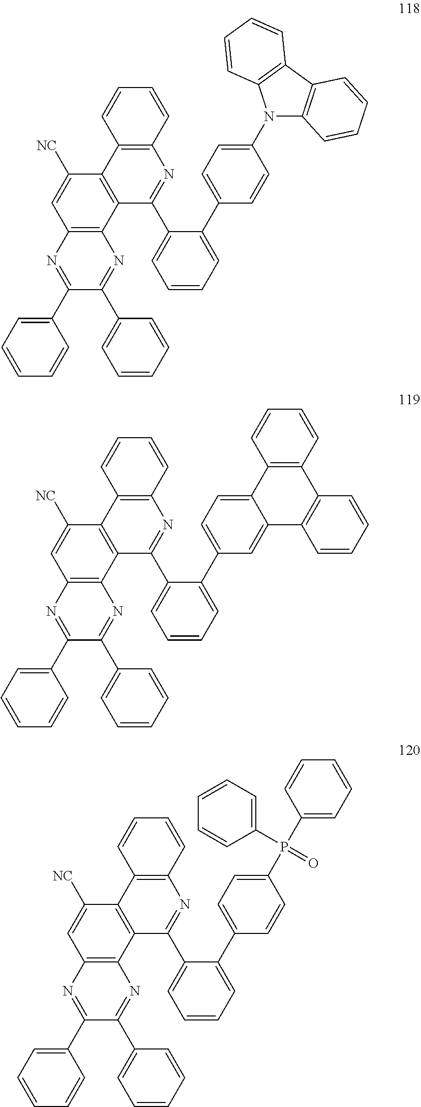 [SMILES: N#Cc1cc2nc(-c3ccccc3)c(-c3ccccc3)nc2c2c(-c3ccccc3-c3ccc(-n4c5ccccc5c5ccccc54)cc3)nc3ccccc3c12 ; N#Cc1cc2nc(-c3ccccc3)c(-c3ccccc3)nc2c2c(-c3ccccc3-c3ccc4c5ccccc5c5ccccc5c4c3)nc3ccccc3c12 ; N#Cc1cc2nc(-c3ccccc3)c(-c3ccccc3)nc2c2c(-c3ccccc3-c3ccc(P(=O)(c4ccccc4)c4ccccc4)cc3)nc3ccccc3c12]

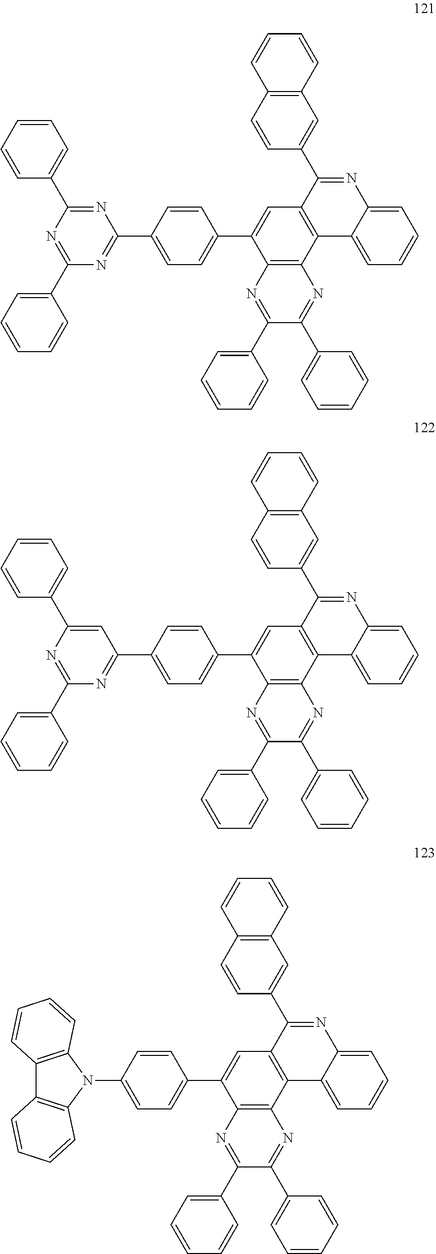

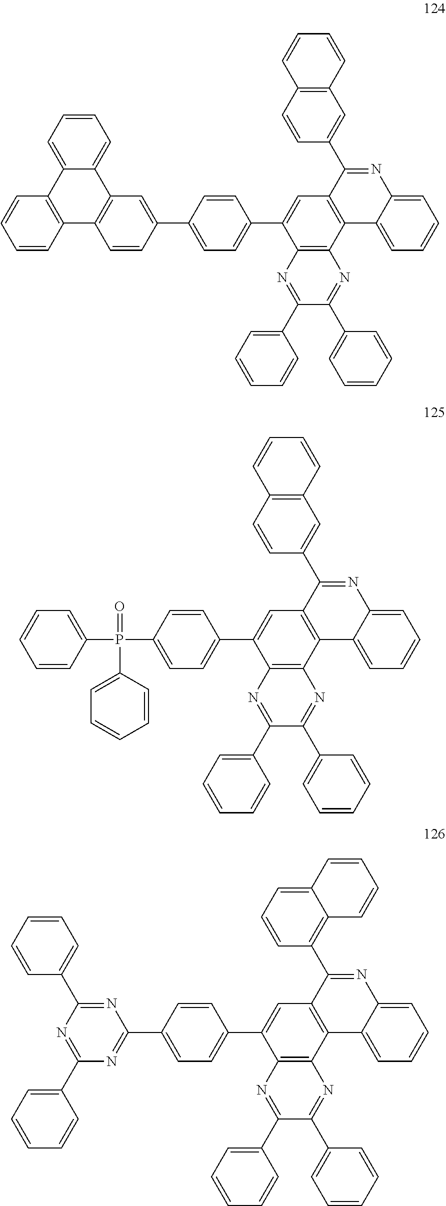 [SMILES: c1ccc(-c2nc3c(-c4ccc(-c5ccc6c7ccccc7c7ccccc7c6c5)cc4)cc4c(-c5ccc6ccccc6c5)nc5ccccc5c4c3nc2-c2ccccc2)cc1 ; O=P(c1ccccc1)(c1ccccc1)c1ccc(-c2cc3c(-c4ccc5ccccc5c4)nc4ccccc4c3c3nc(-c4ccccc4)c(-c4ccccc4)nc23)cc1 ; c1ccc(-c2nc(-c3ccccc3)nc(-c3ccc(-c4cc5c(-c6cccc7ccccc67)nc6ccccc6c5c5nc(-c6ccccc6)c(-c6ccccc6)nc45)cc3)n2)cc1]

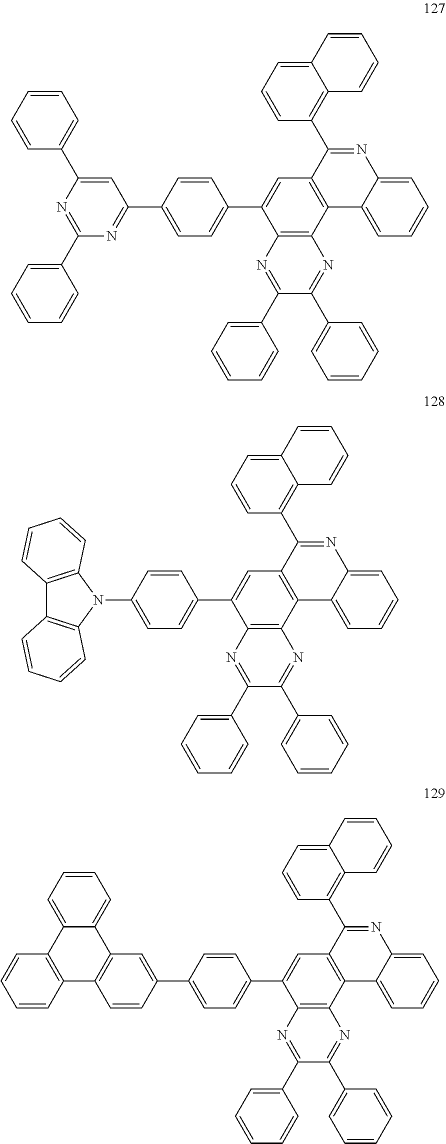

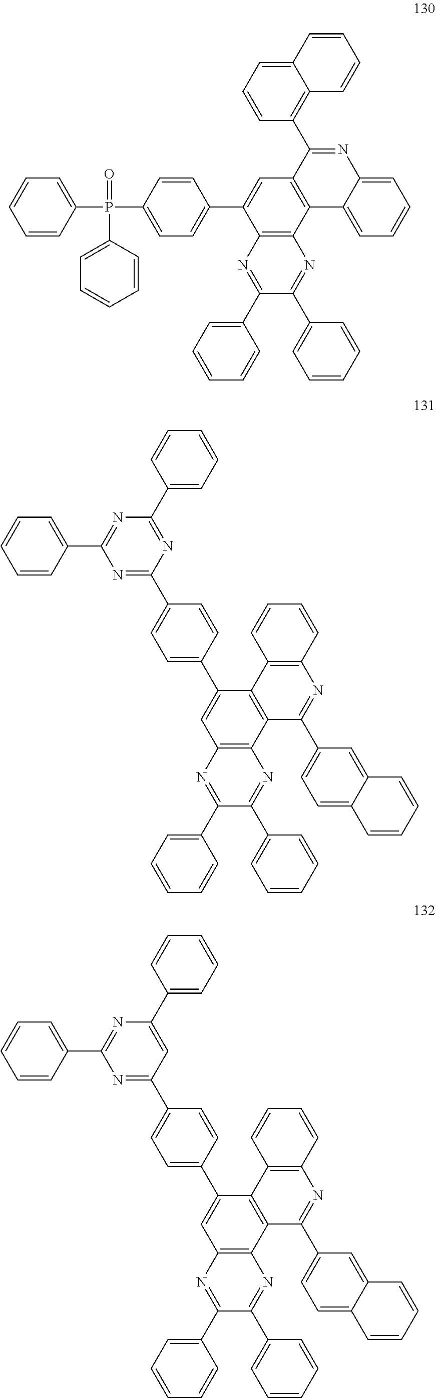

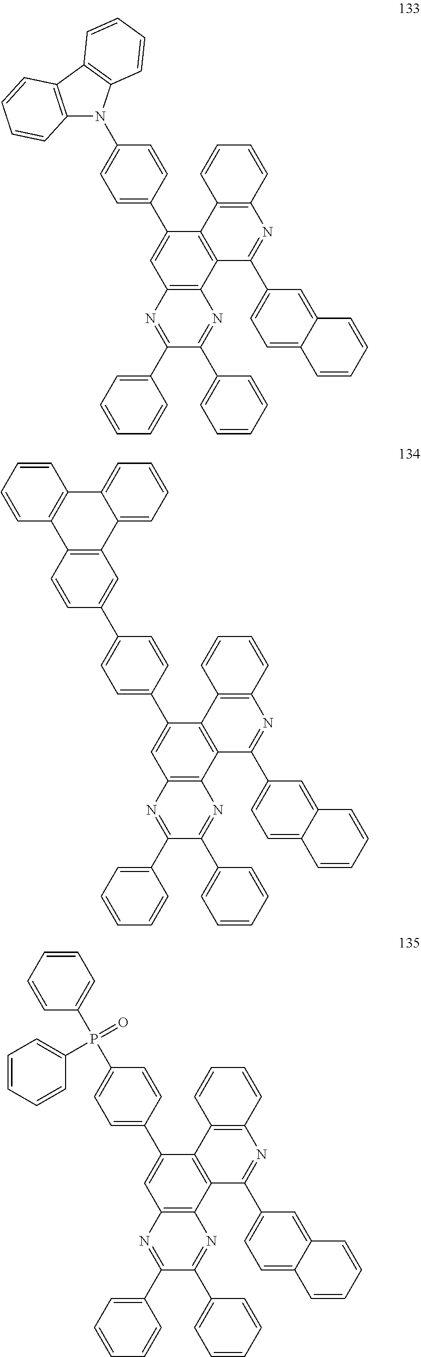

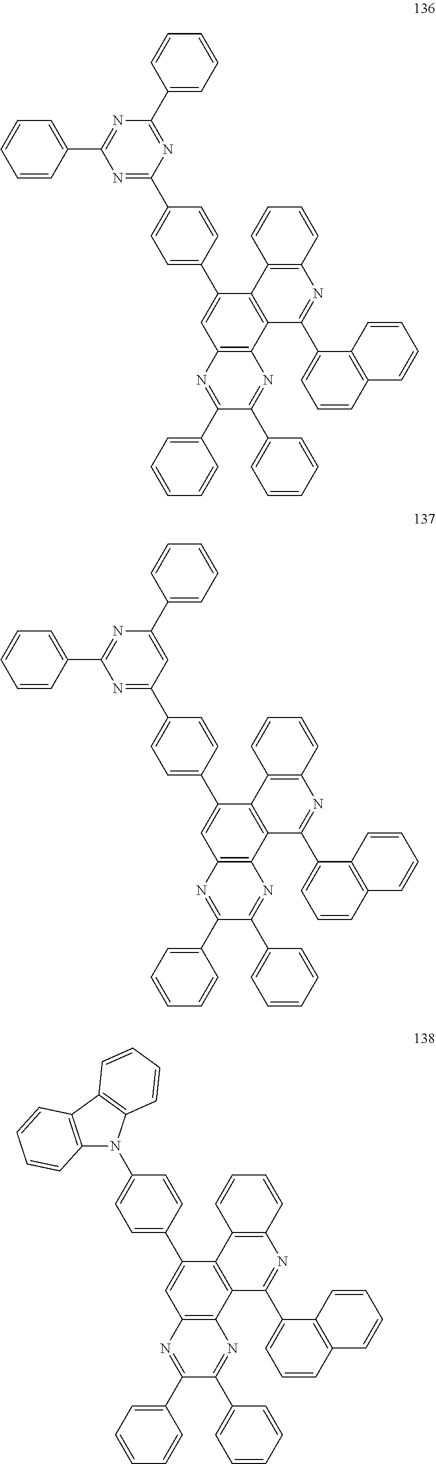 [SMILES: c1ccc(-c2nc(-c3ccccc3)nc(-c3ccc(-c4cc5nc(-c6ccccc6)c(-c6ccccc6)nc5c5c(-c6cccc7ccccc67)nc6ccccc6c45)cc3)n2)cc1 ; c1ccc(-c2cc(-c3ccc(-c4cc5nc(-c6ccccc6)c(-c6ccccc6)nc5c5c(-c6cccc7ccccc67)nc6ccccc6c45)cc3)nc(-c3ccccc3)n2)cc1 ; c1ccc(-c2nc3cc(-c4ccc(-n5c6ccccc6c6ccccc65)cc4)c4c5ccccc5nc(-c5cccc6ccccc56)c4c3nc2-c2ccccc2)cc1]

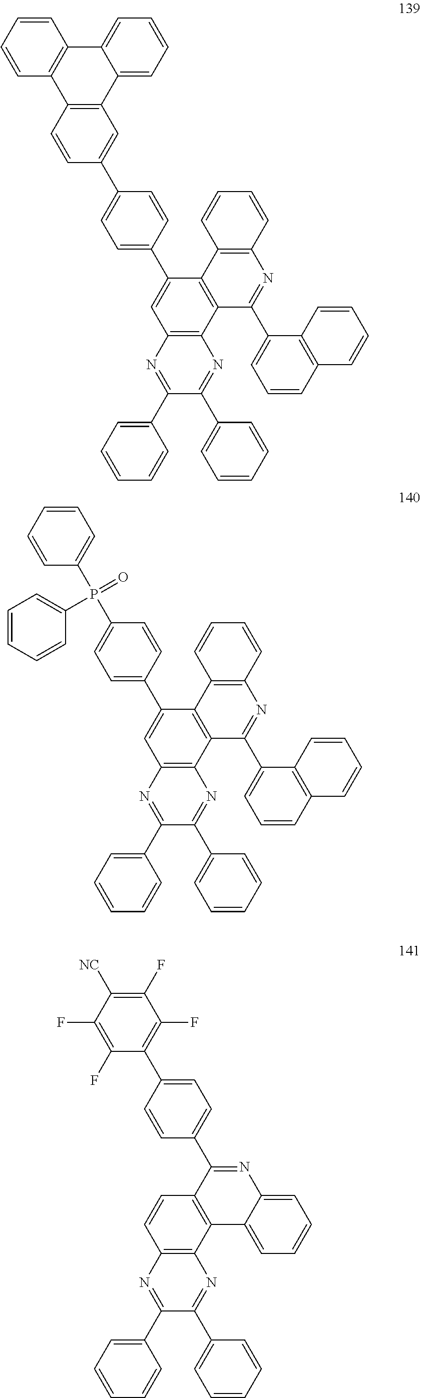 [SMILES: c1ccc(-c2nc3cc(-c4ccc(-c5ccc6c7ccccc7c7ccccc7c6c5)cc4)c4c5ccccc5nc(-c5cccc6ccccc56)c4c3nc2-c2ccccc2)cc1 ; O=P(c1ccccc1)(c1ccccc1)c1ccc(-c2cc3nc(-c4ccccc4)c(-c4ccccc4)nc3c3c(-c4cccc5ccccc45)nc4ccccc4c23)cc1 ; N#Cc1c(F)c(F)c(-c2ccc(-c3nc4ccccc4c4c3ccc3nc(-c5ccccc5)c(-c5ccccc5)nc34)cc2)c(F)c1F]

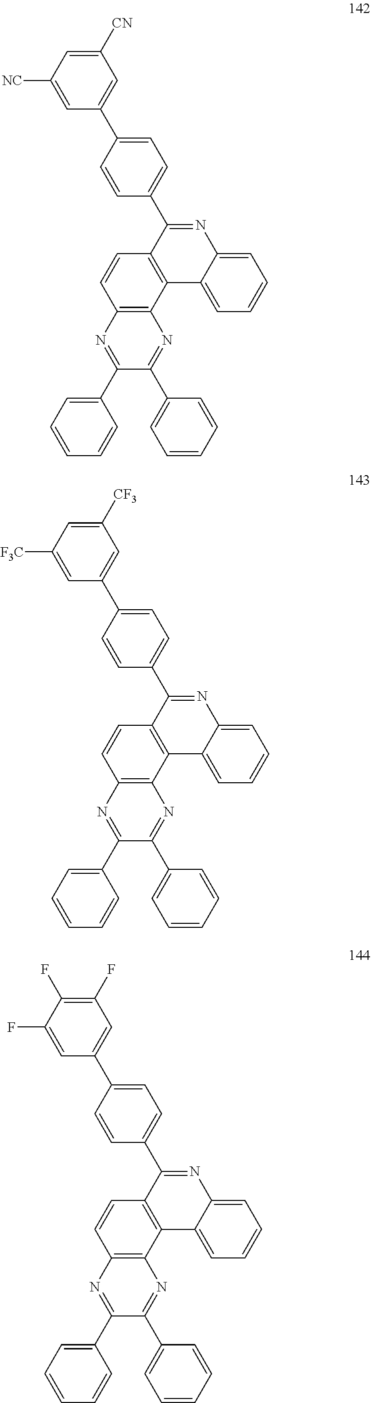

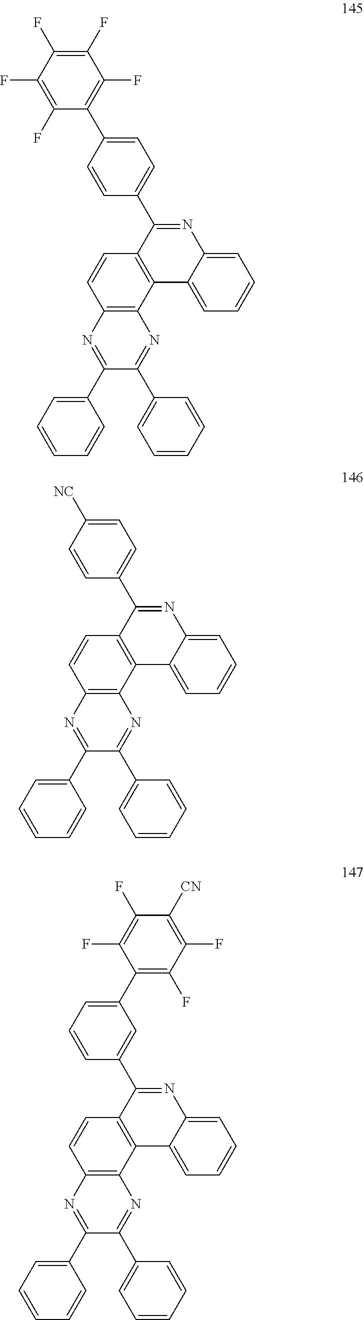

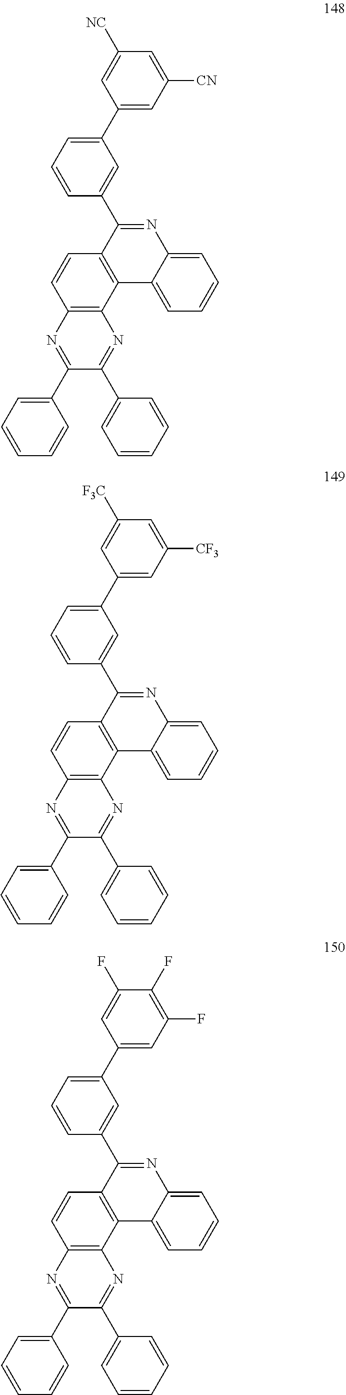

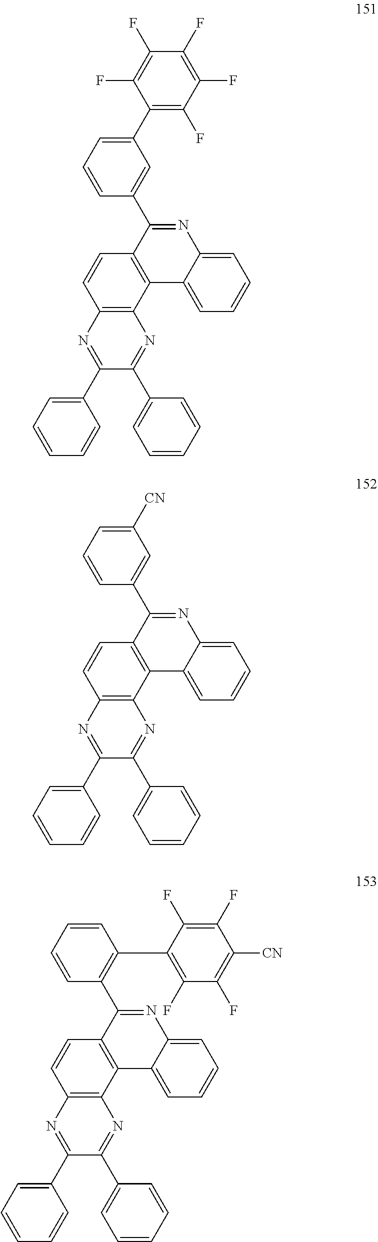

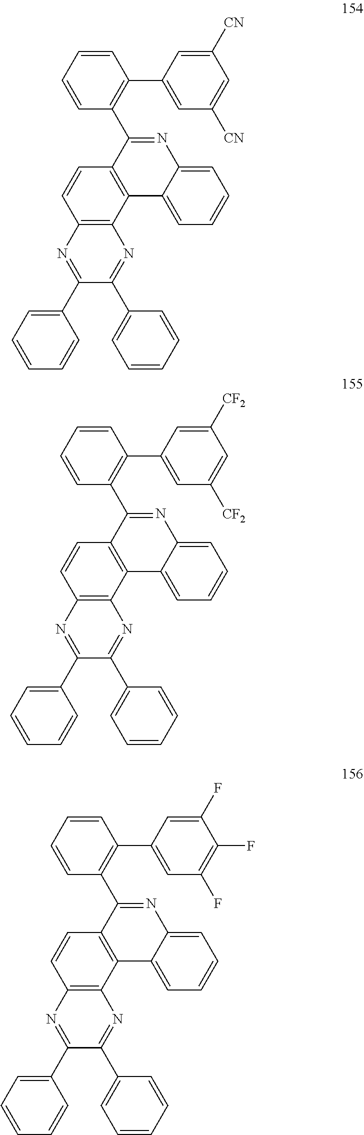

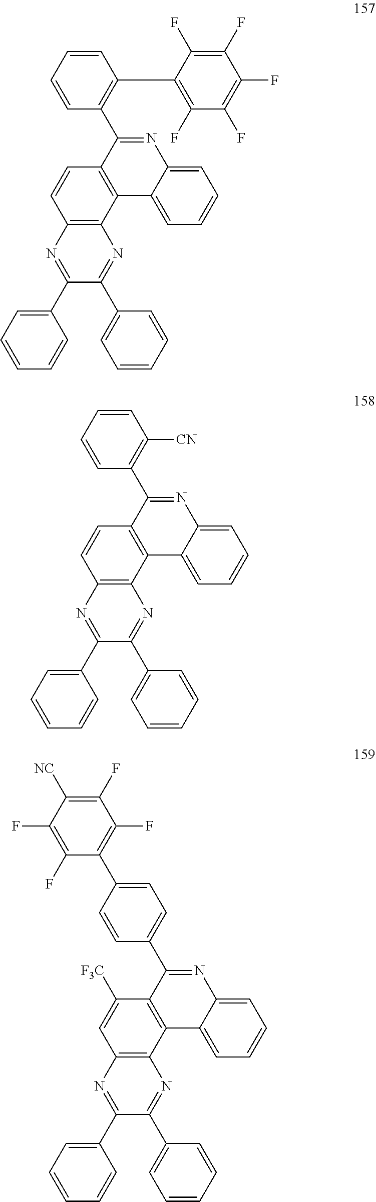

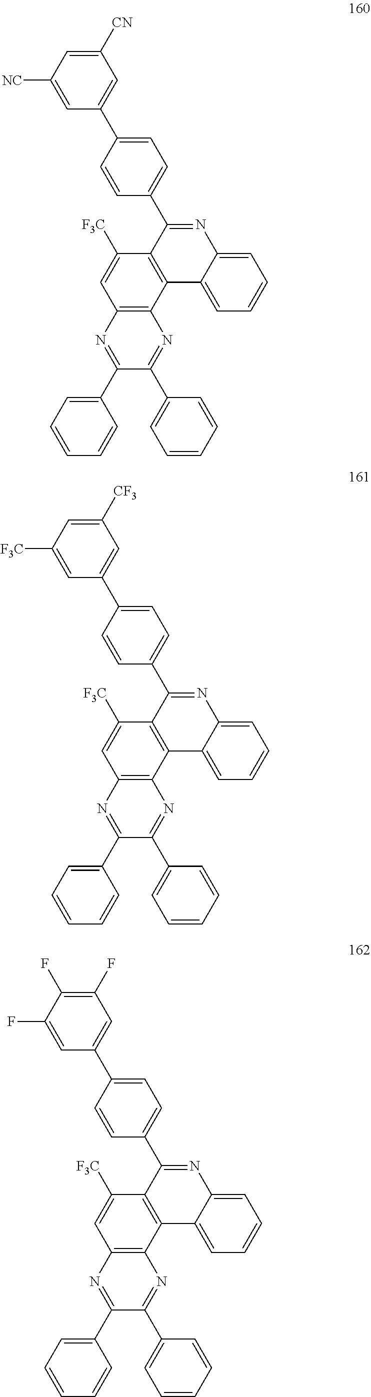

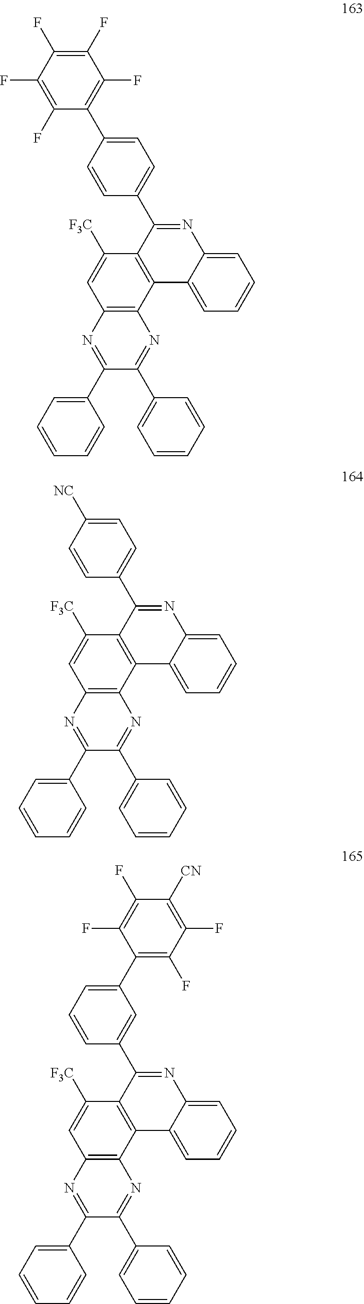

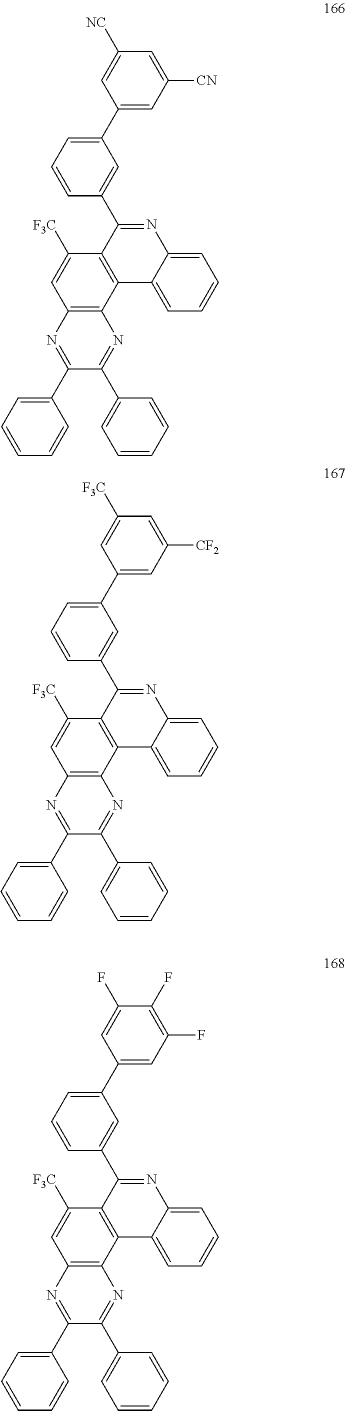

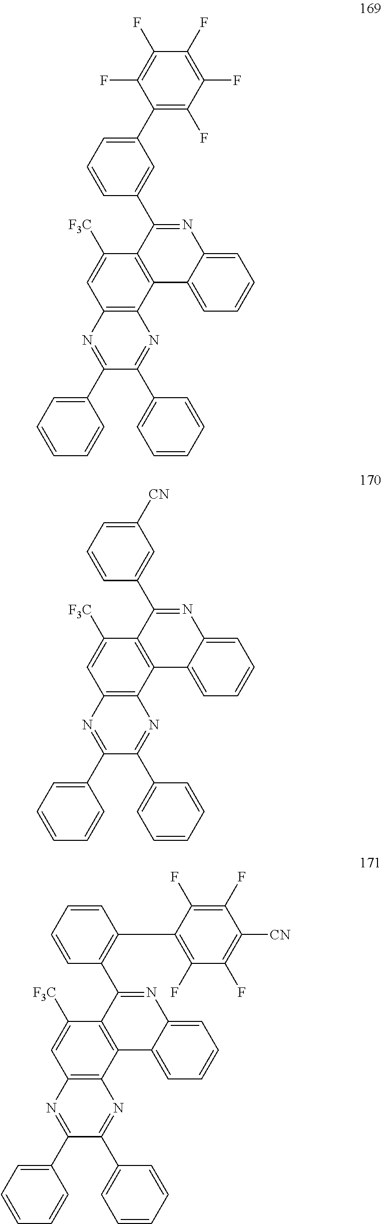

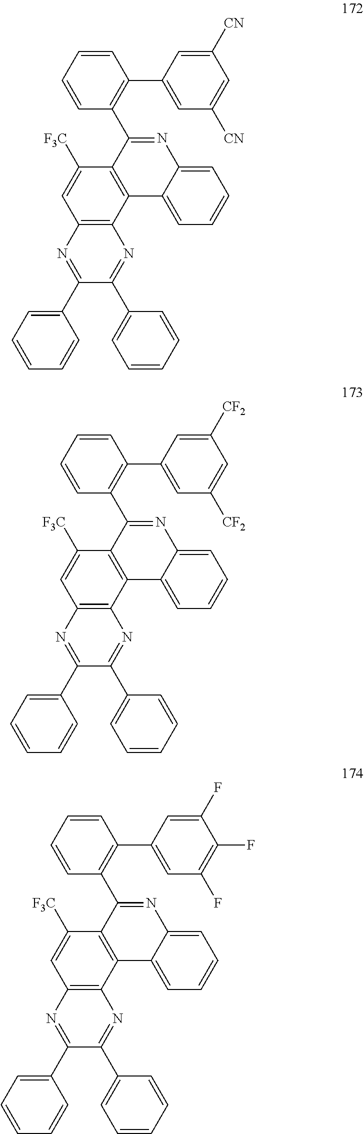

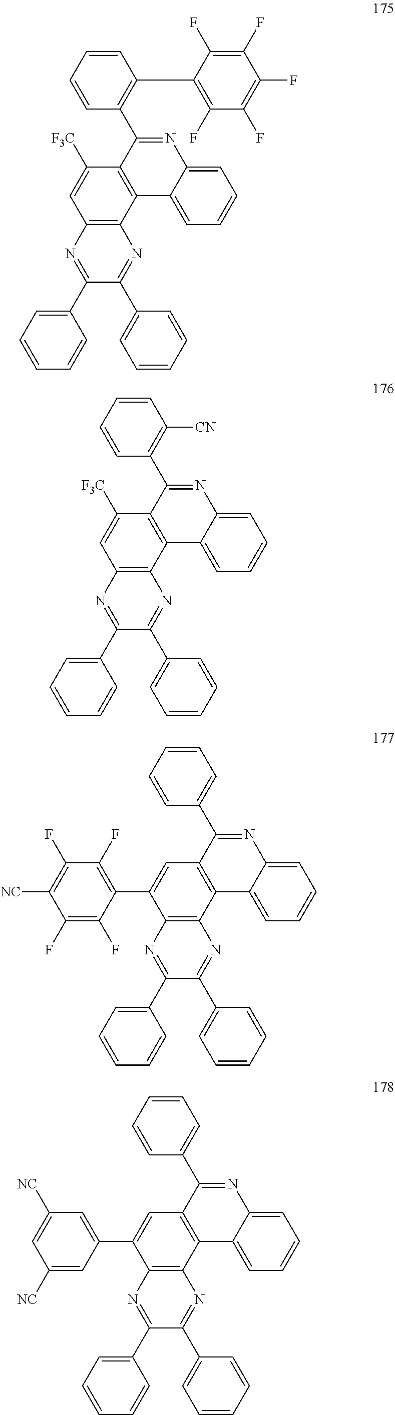

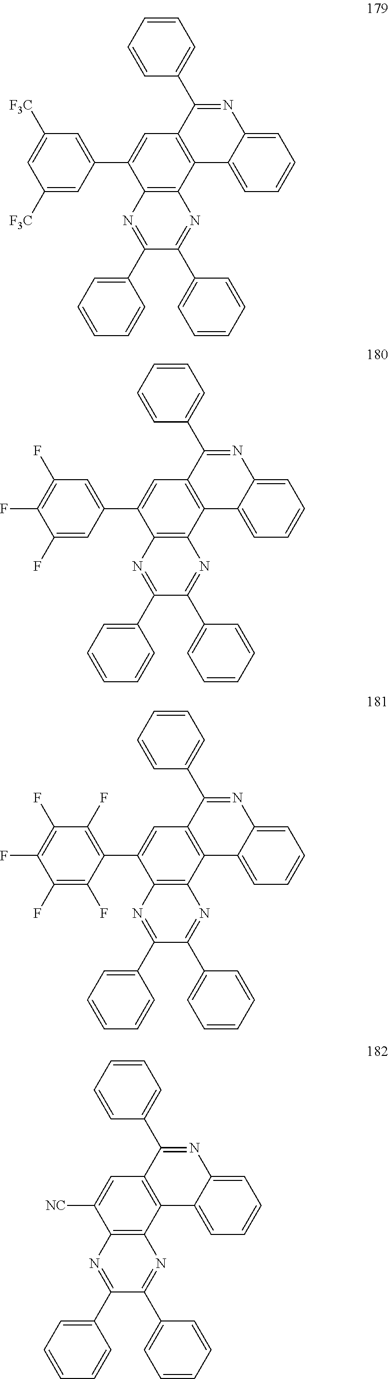

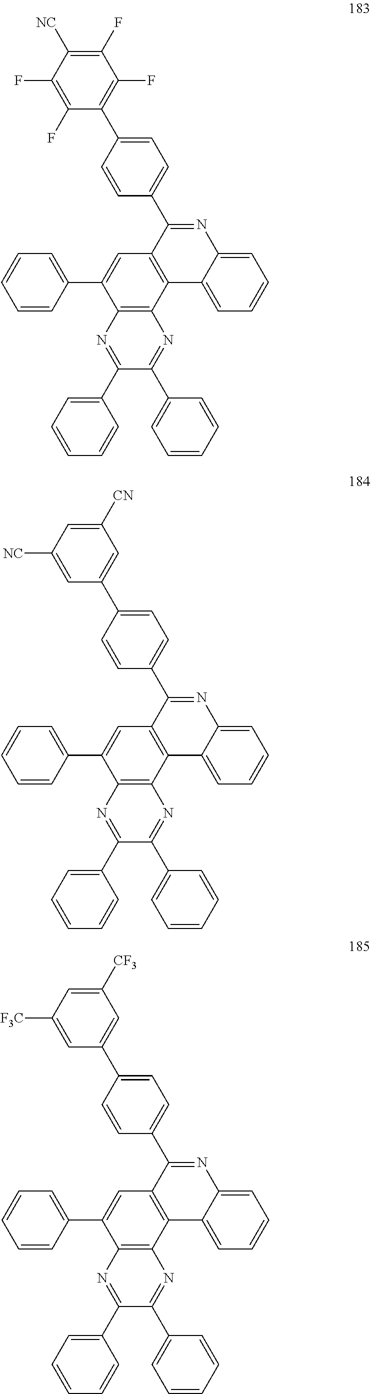

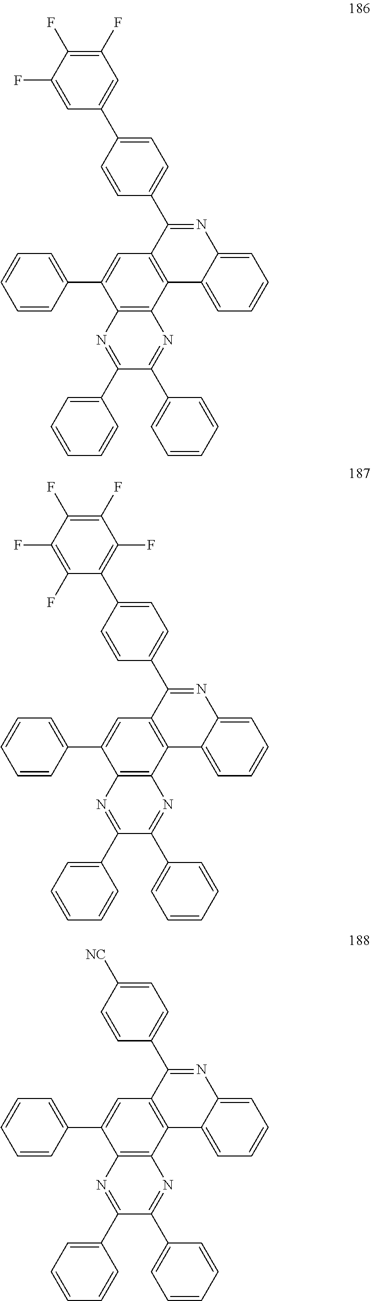

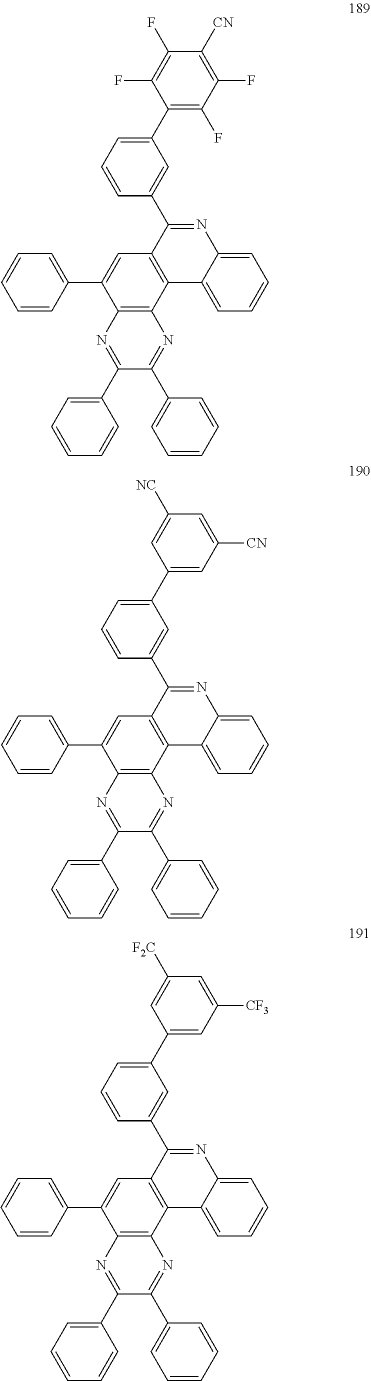

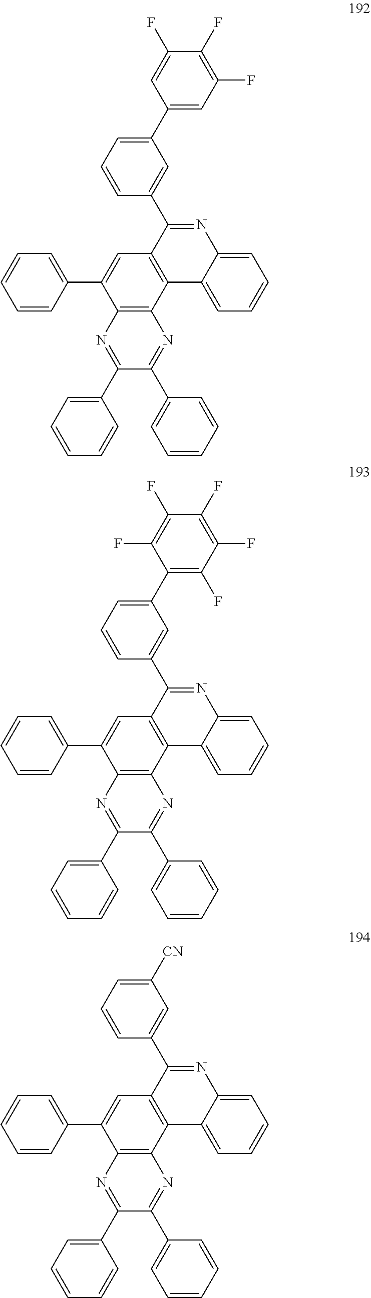

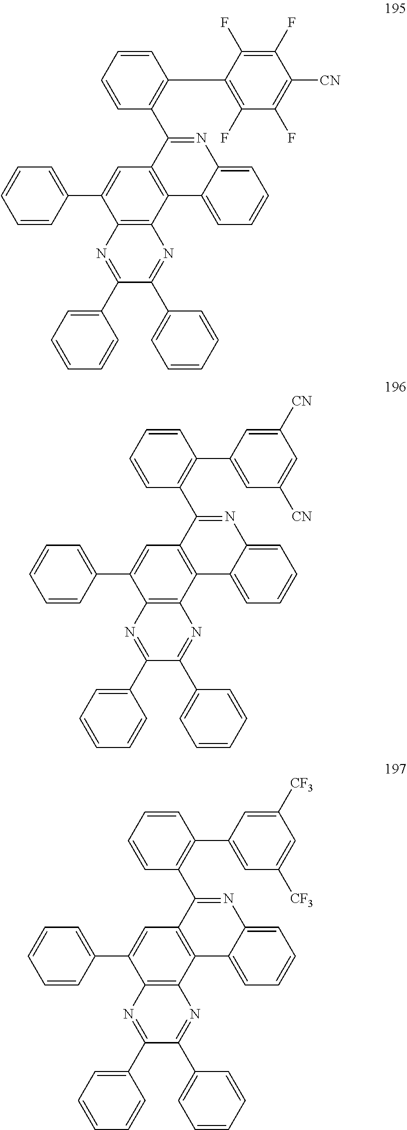 [SMILES: N#Cc1c(F)c(F)c(-c2ccccc2-c2nc3ccccc3c3c2cc(-c2ccccc2)c2nc(-c4ccccc4)c(-c4ccccc4)nc23)c(F)c1F ; N#Cc1cc(C#N)cc(-c2ccccc2-c2nc3ccccc3c3c2cc(-c2ccccc2)c2nc(-c4ccccc4)c(-c4ccccc4)nc23)c1 ; FC(F)(F)c1cc(-c2ccccc2-c2nc3ccccc3c3c2cc(-c2ccccc2)c2nc(-c4ccccc4)c(-c4ccccc4)nc23)cc(C(F)(F)F)c1]

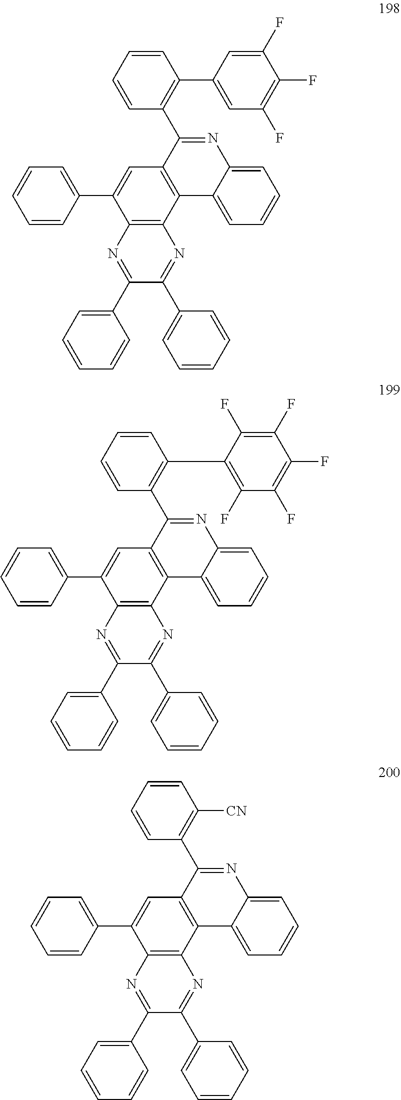

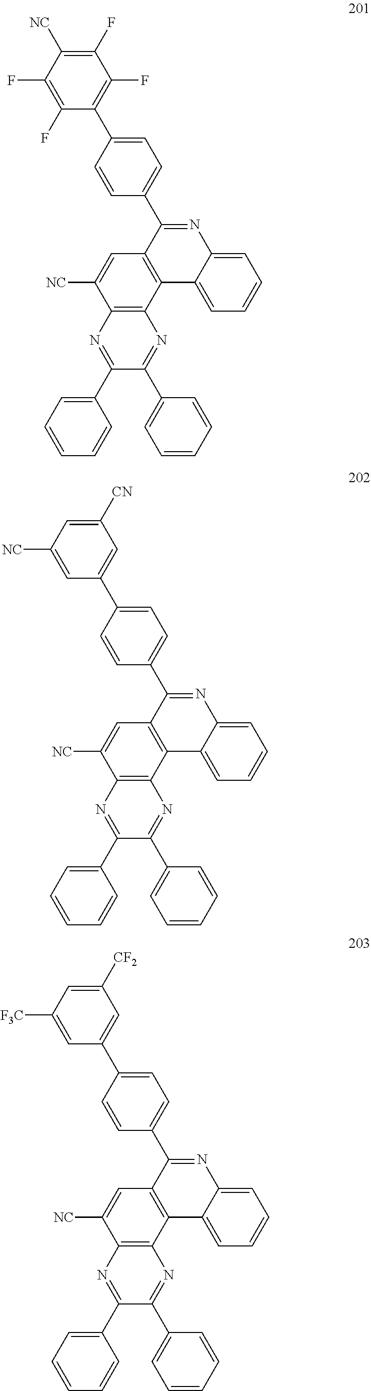

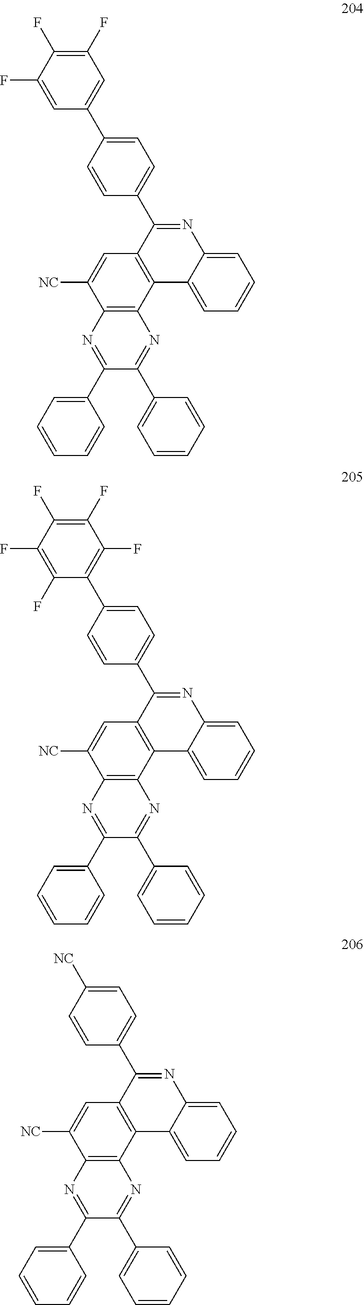

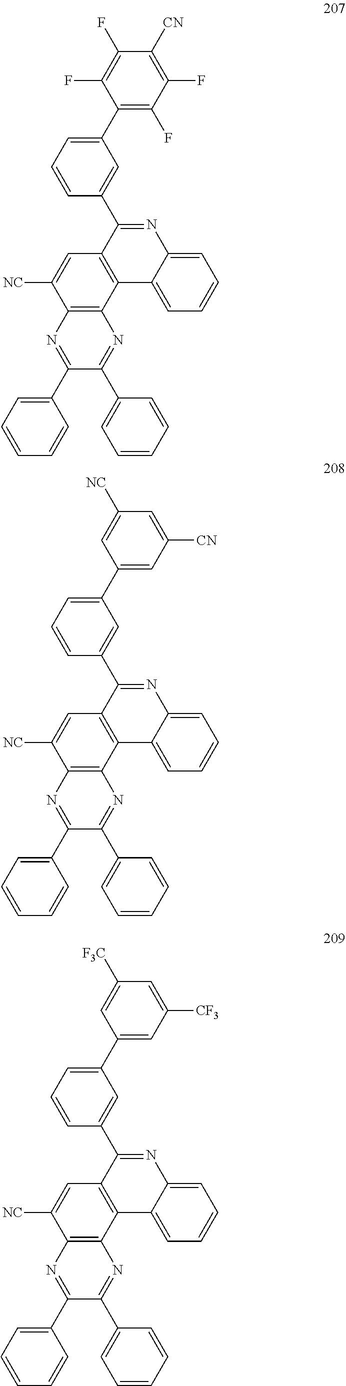

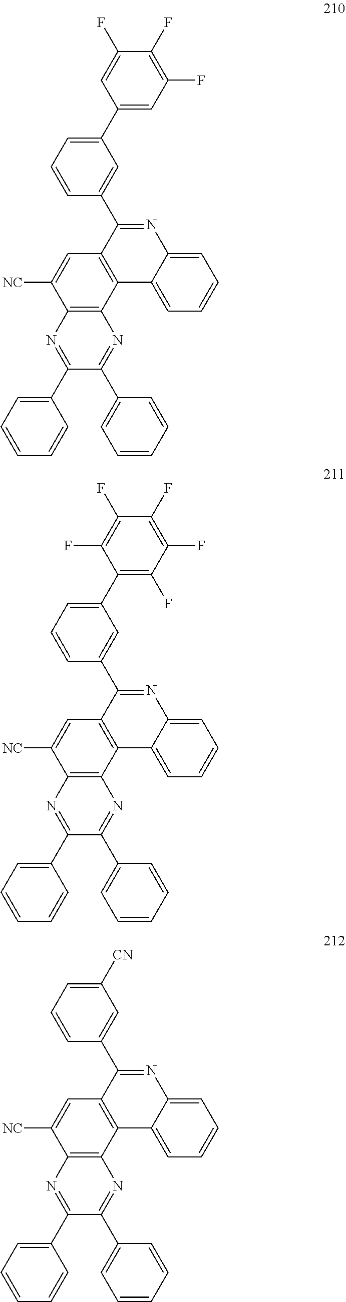

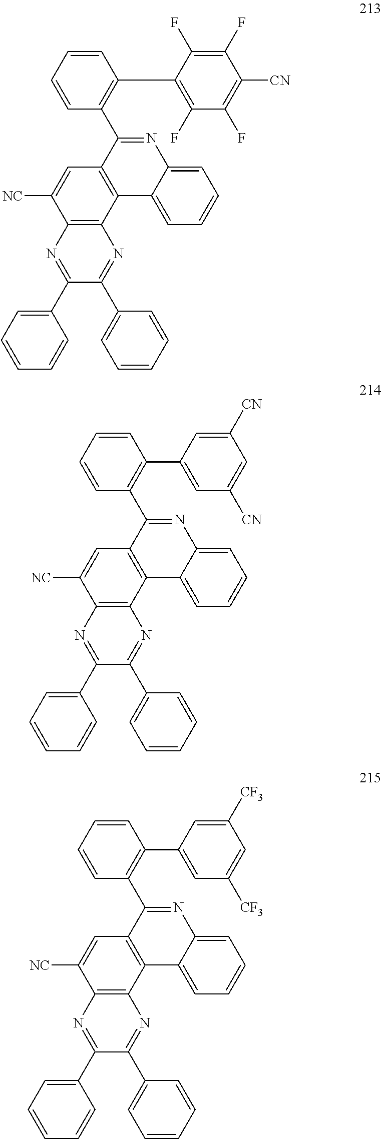 [SMILES: N#Cc1c(F)c(F)c(-c2ccccc2-c2nc3ccccc3c3c2cc(C#N)c2nc(-c4ccccc4)c(-c4ccccc4)nc23)c(F)c1F ; N#Cc1cc(C#N)cc(-c2ccccc2-c2nc3ccccc3c3c2cc(C#N)c2nc(-c4ccccc4)c(-c4ccccc4)nc23)c1 ; N#Cc1cc2c(-c3ccccc3-c3cc(C(F)(F)F)cc(C(F)(F)F)c3)nc3ccccc3c2c2nc(-c3ccccc3)c(-c3ccccc3)nc12]

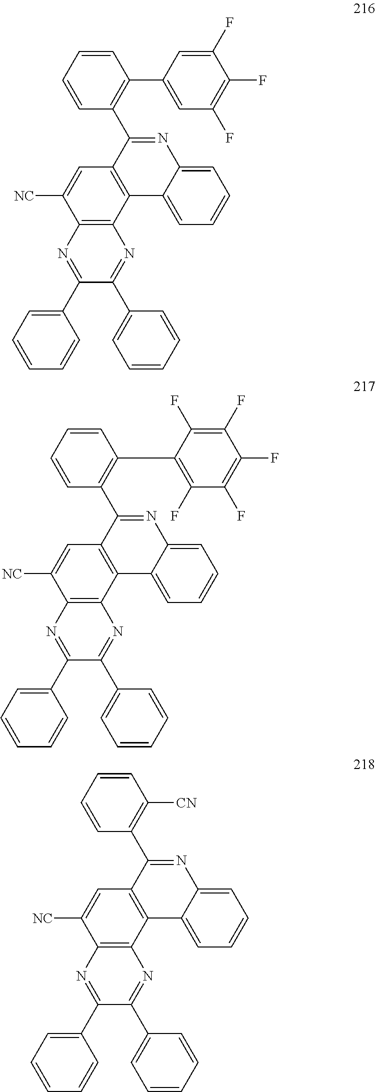

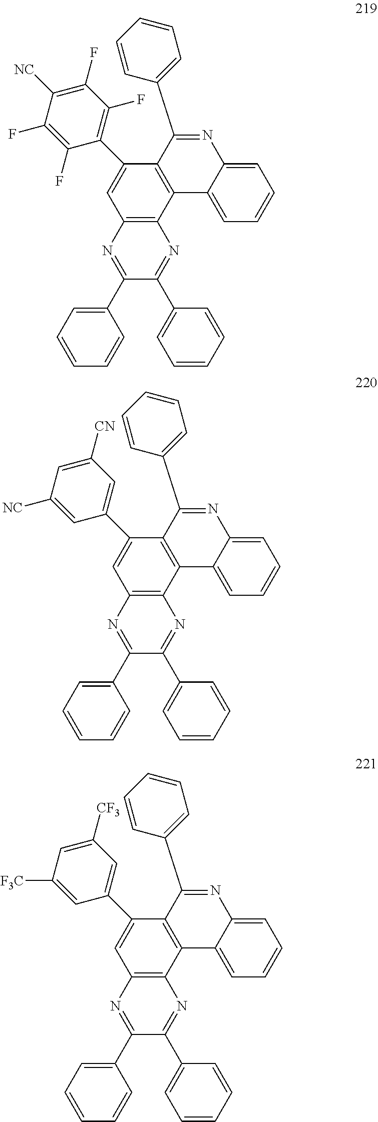

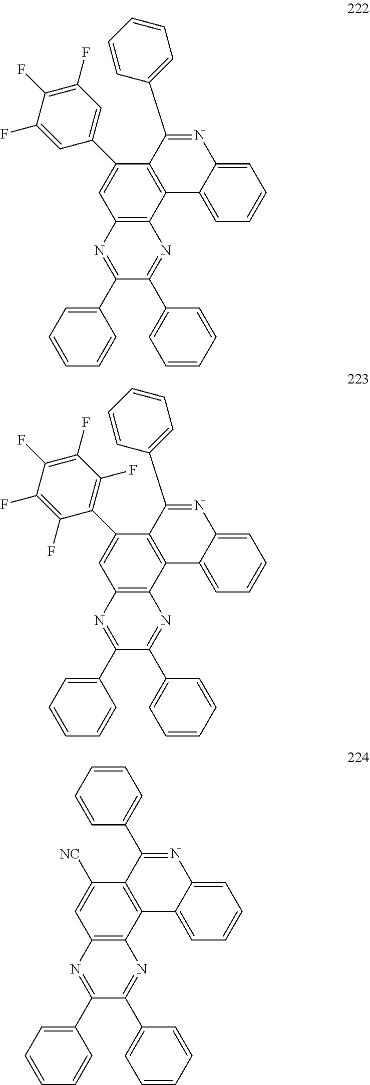

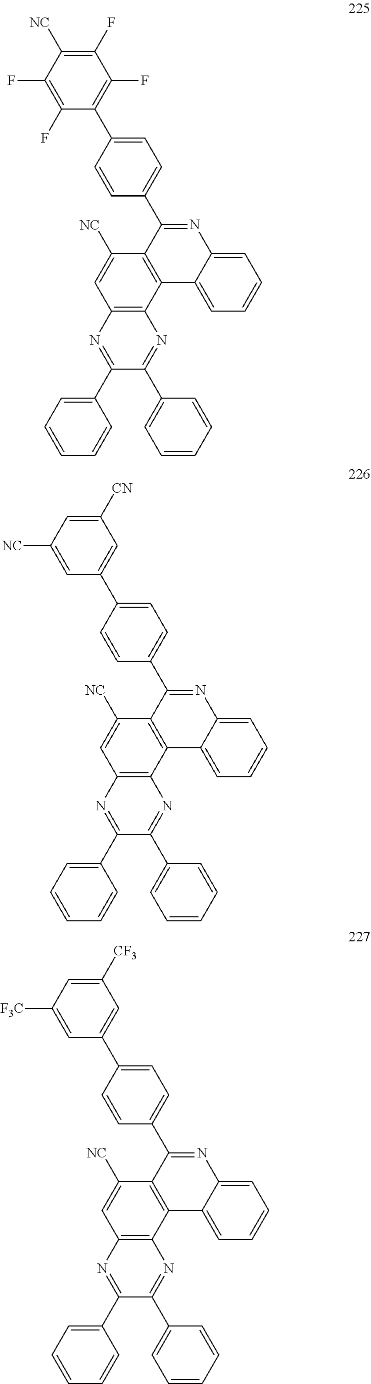

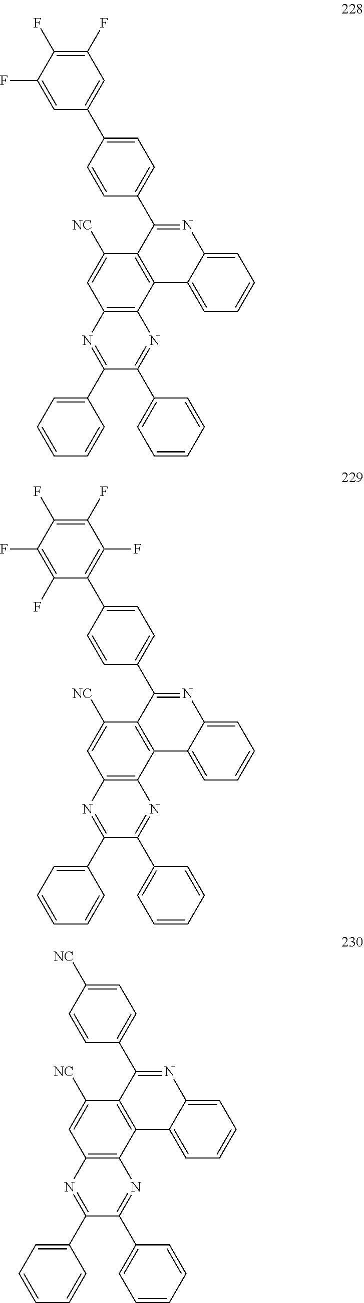

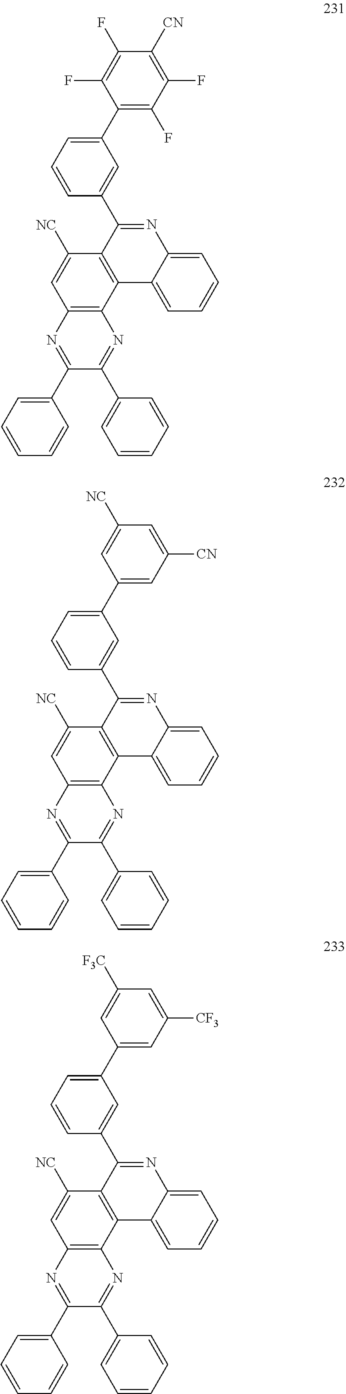

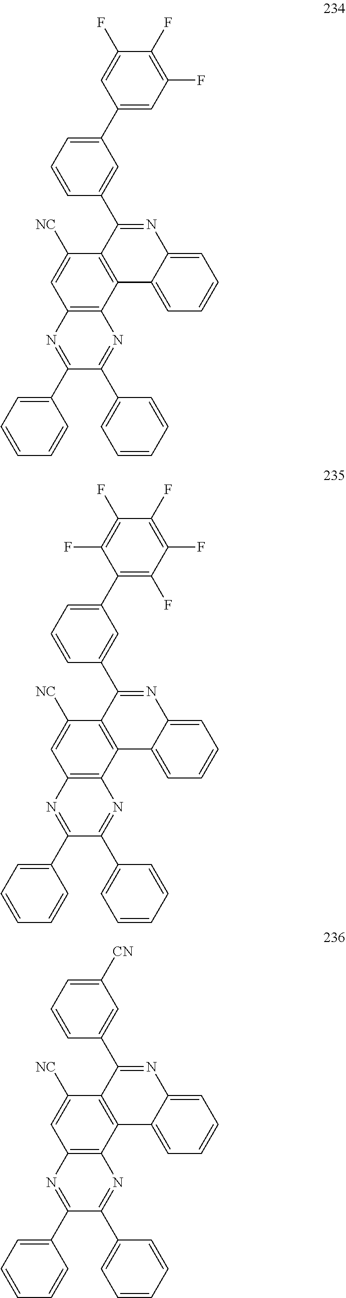

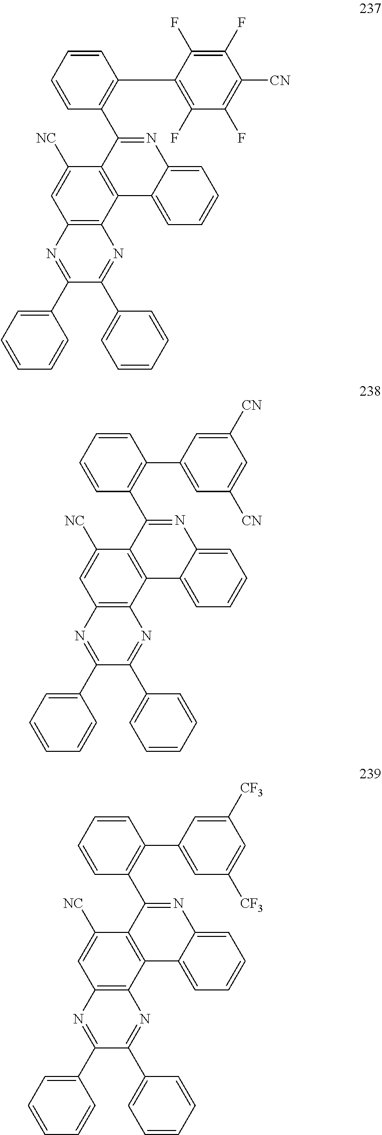 [SMILES: N#Cc1c(F)c(F)c(-c2ccccc2-c2nc3ccccc3c3c2c(C#N)cc2nc(-c4ccccc4)c(-c4ccccc4)nc23)c(F)c1F ; N#Cc1cc(C#N)cc(-c2ccccc2-c2nc3ccccc3c3c2c(C#N)cc2nc(-c4ccccc4)c(-c4ccccc4)nc23)c1 ; N#Cc1cc2nc(-c3ccccc3)c(-c3ccccc3)nc2c2c1c(-c1ccccc1-c1cc(C(F)(F)F)cc(C(F)(F)F)c1)nc1ccccc12]

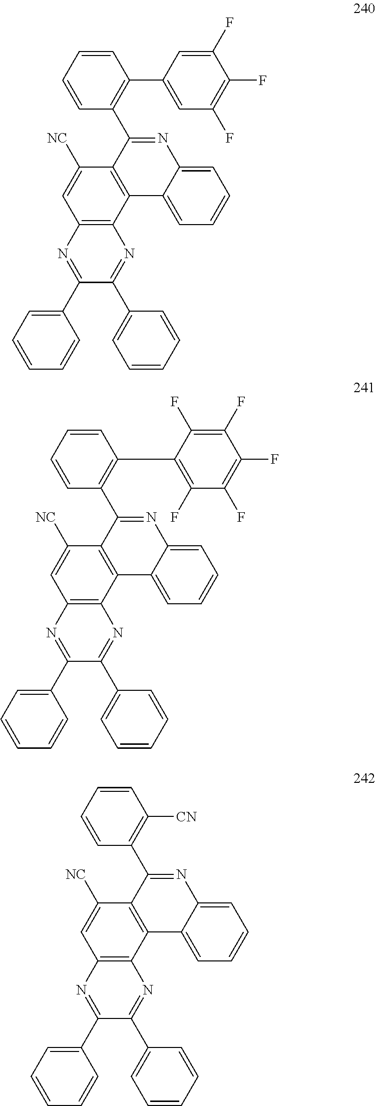

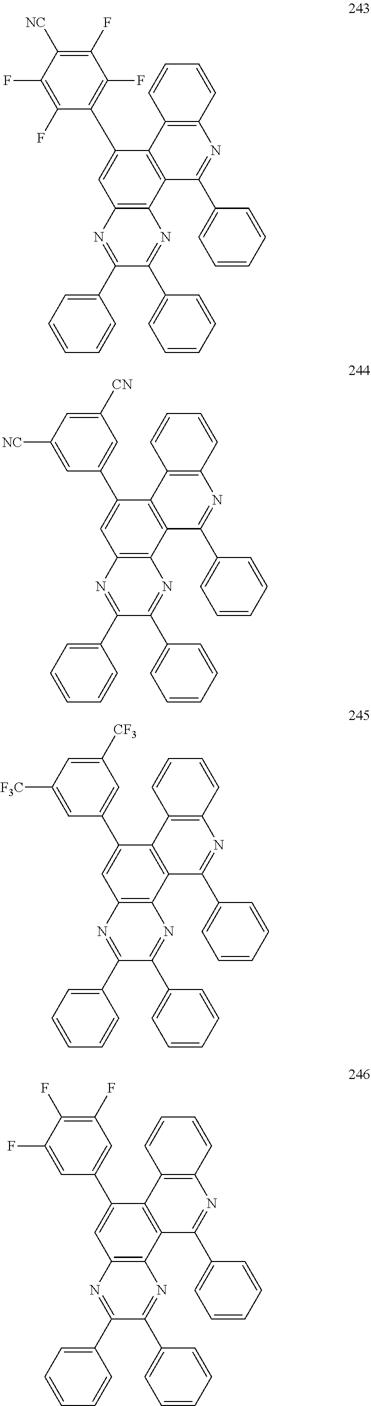

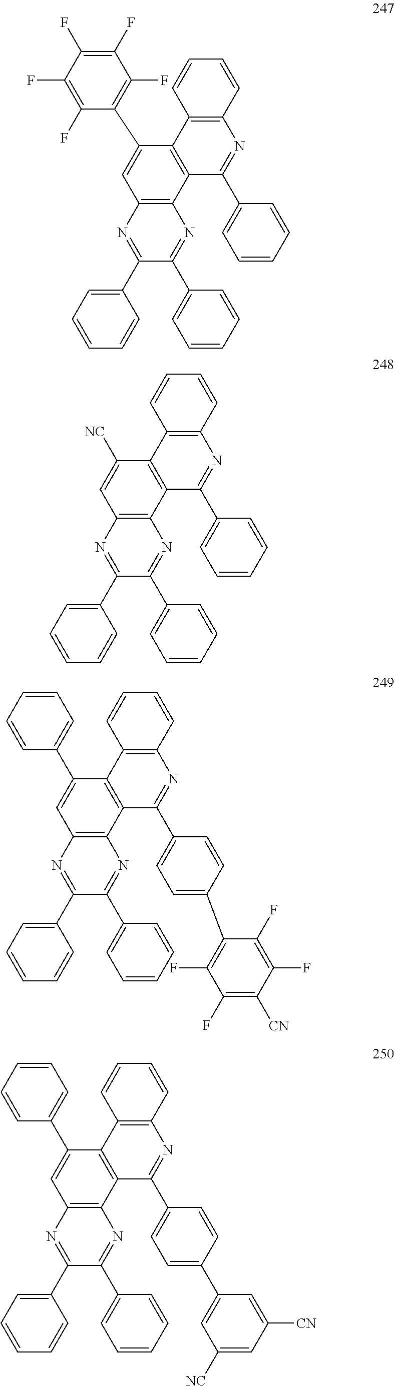

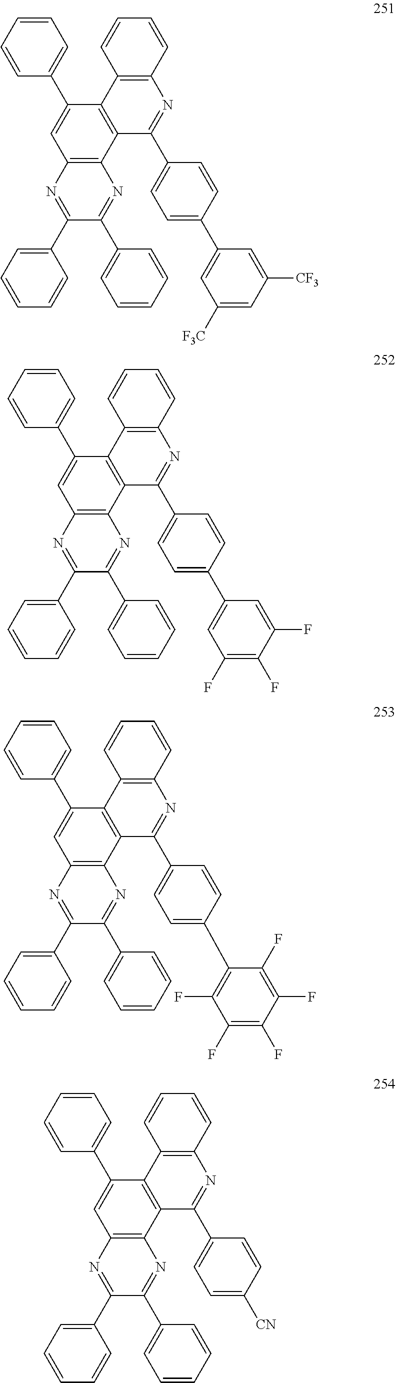

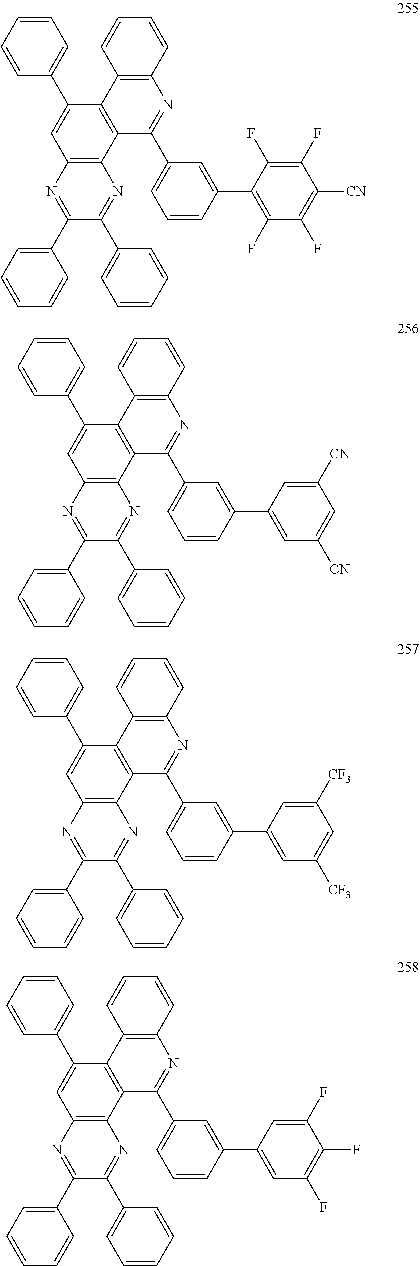

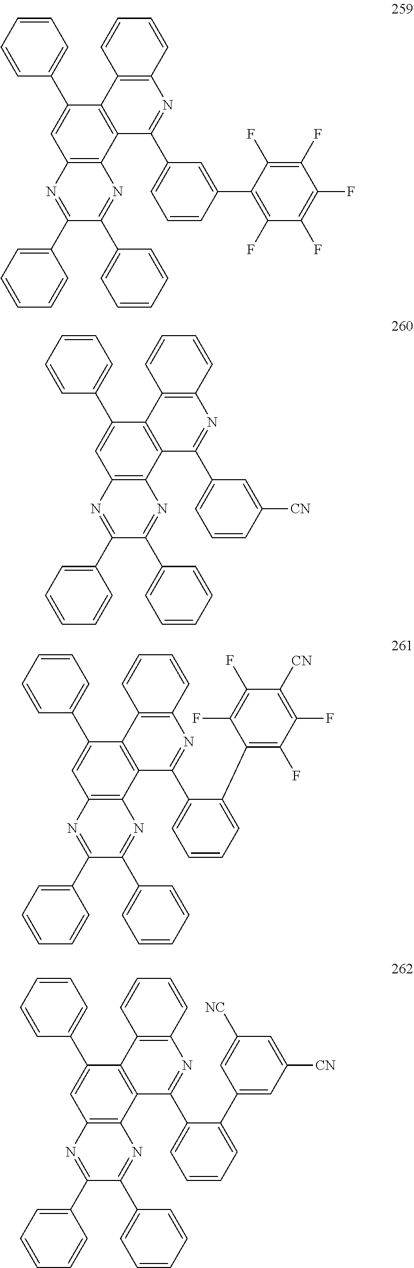

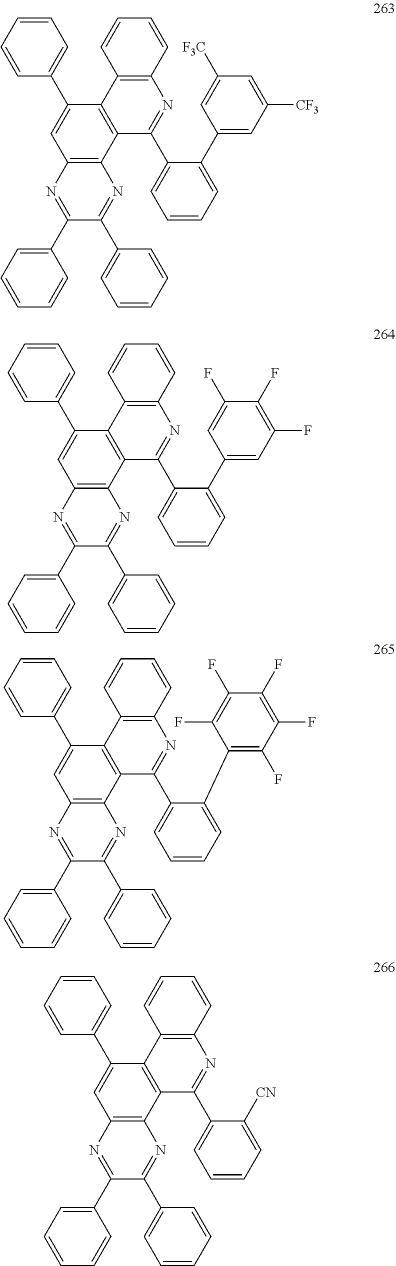

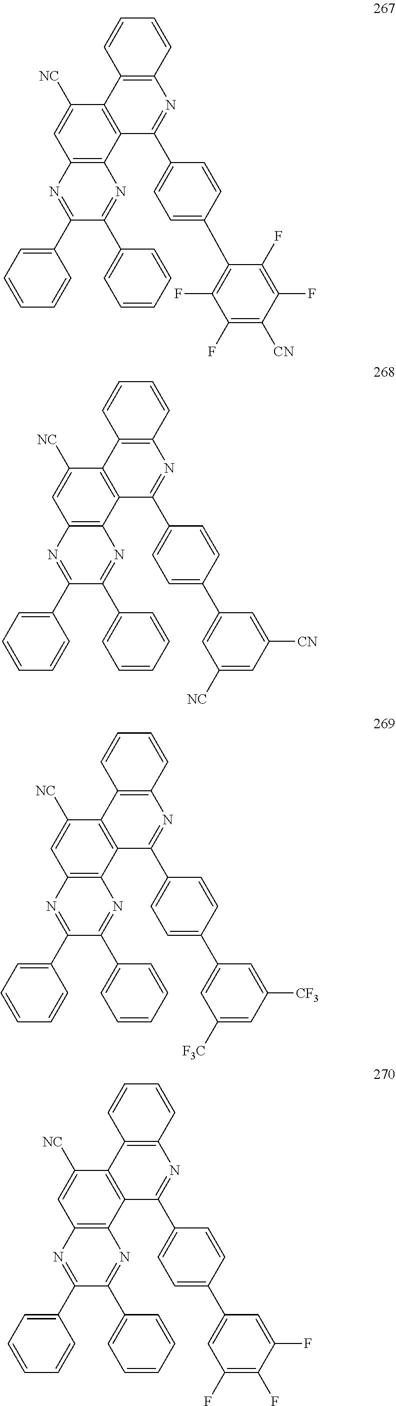 [SMILES: N#Cc1c(F)c(F)c(-c2ccc(-c3nc4ccccc4c4c(C#N)cc5nc(-c6ccccc6)c(-c6ccccc6)nc5c34)cc2)c(F)c1F ; N#Cc1cc(C#N)cc(-c2ccc(-c3nc4ccccc4c4c(C#N)cc5nc(-c6ccccc6)c(-c6ccccc6)nc5c34)cc2)c1 ; N#Cc1cc2nc(-c3ccccc3)c(-c3ccccc3)nc2c2c(-c3ccc(-c4cc(C(F)(F)F)cc(C(F)(F)F)c4)cc3)nc3ccccc3c12 ; N#Cc1cc2nc(-c3ccccc3)c(-c3ccccc3)nc2c2c(-c3ccc(-c4cc(F)c(F)c(F)c4)cc3)nc3ccccc3c12]

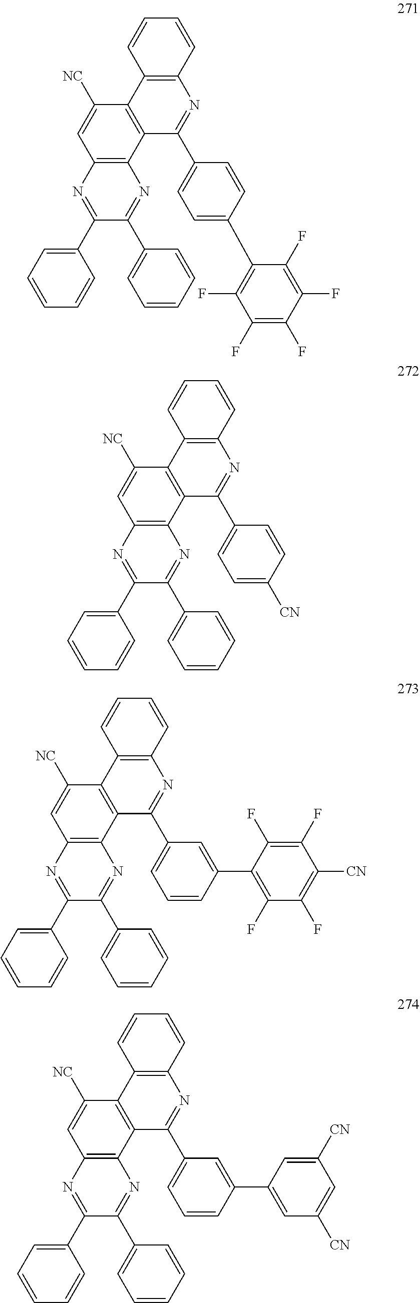

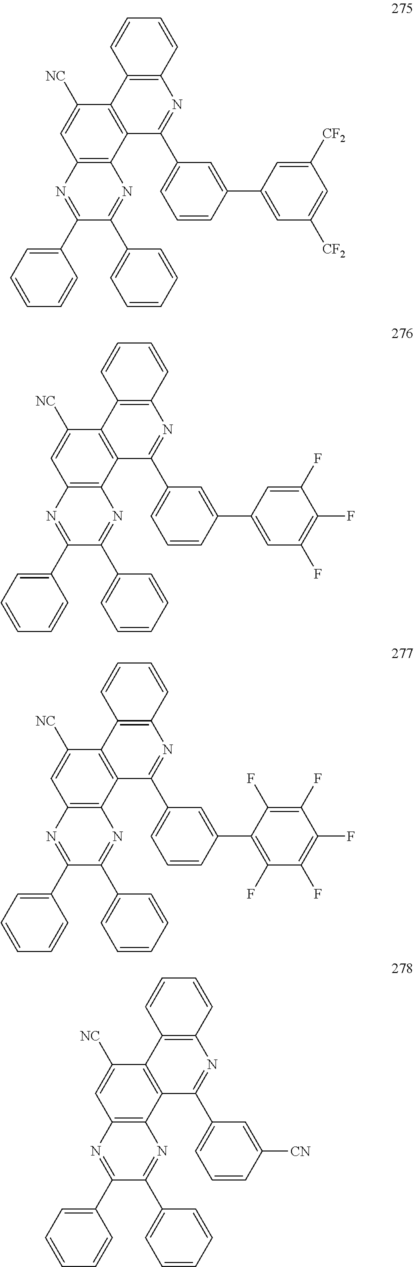

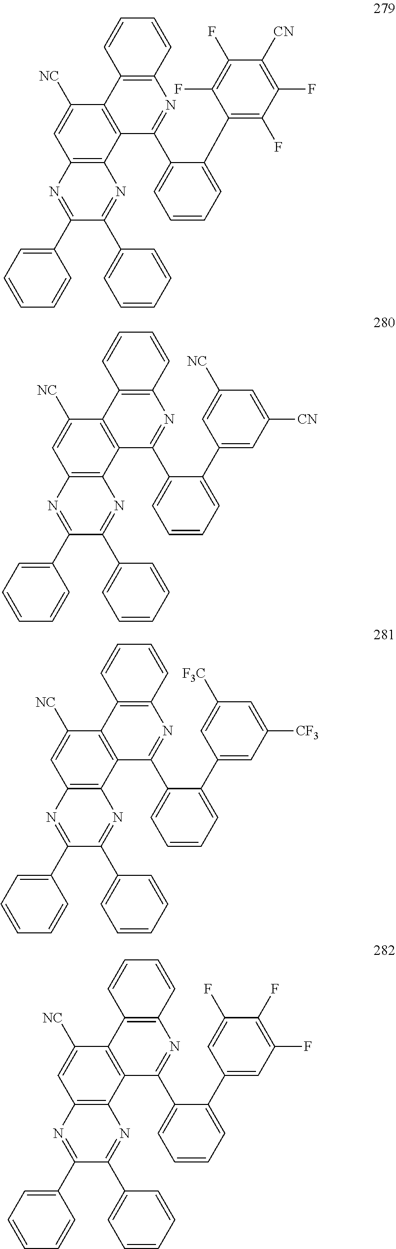

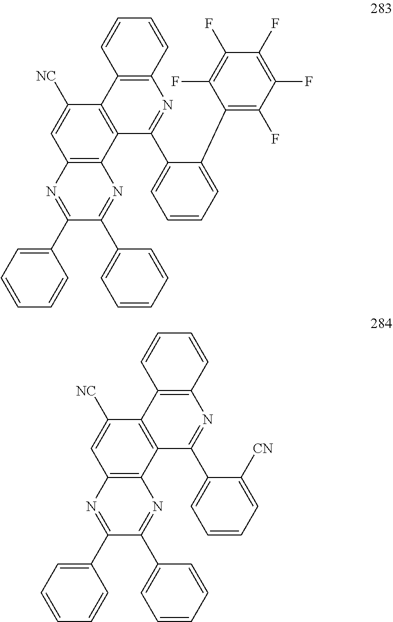

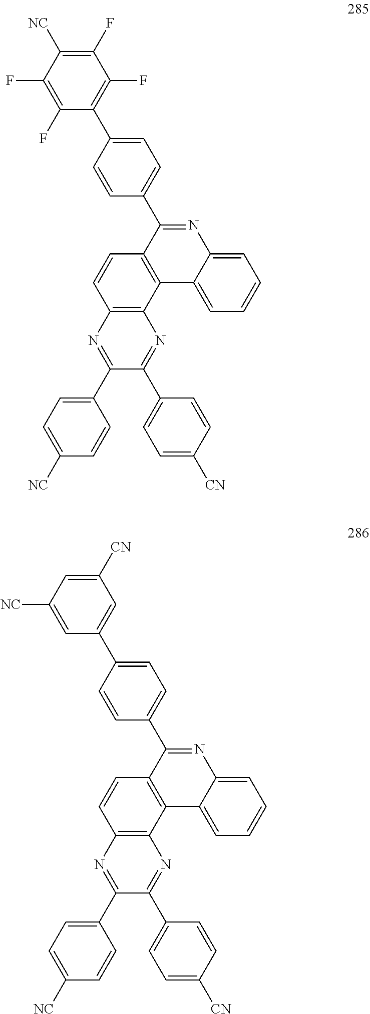 [SMILES: N#Cc1ccc(-c2nc3ccc4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3ccc4c(-c5ccc(-c6cc(C#N)cc(C#N)c6)cc5)nc5ccccc5c4c3nc2-c2ccc(C#N)cc2)cc1]

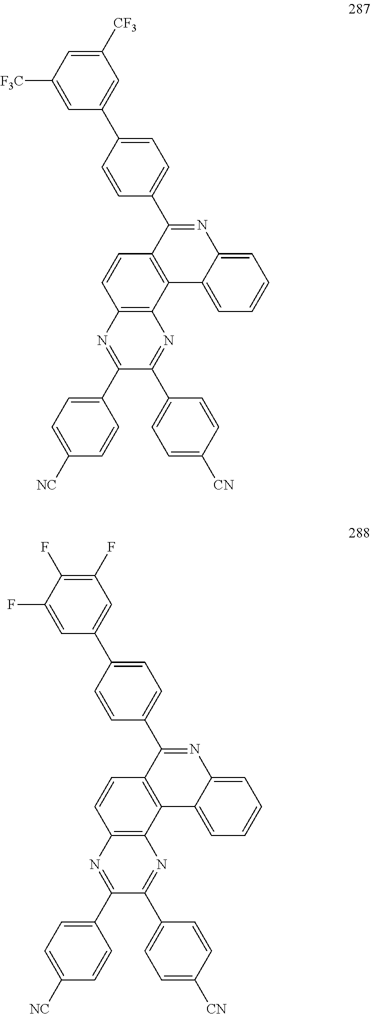 [SMILES: N#Cc1ccc(-c2nc3ccc4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3ccc4c(-c5ccc(-c6cc(F)c(F)c(F)c6)cc5)nc5ccccc5c4c3nc2-c2ccc(C#N)cc2)cc1]

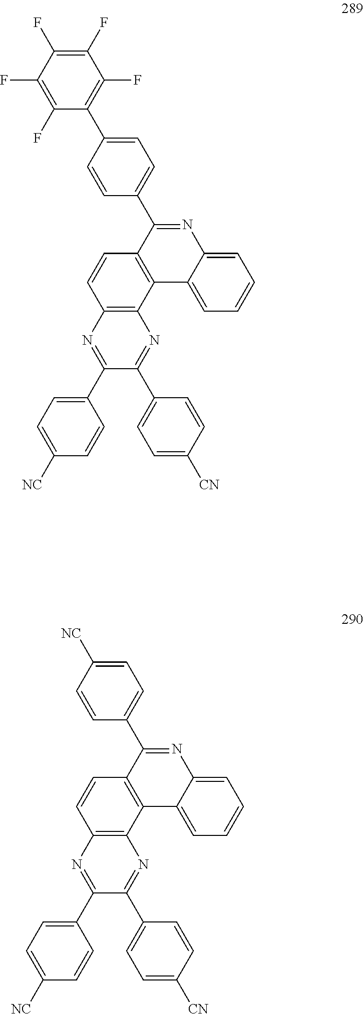 [SMILES: N#Cc1ccc(-c2nc3ccc4c(-c5ccc(-c6c(F)c(F)c(F)c(F)c6F)cc5)nc5ccccc5c4c3nc2-c2ccc(C#N)cc2)cc1 ; N#Cc1ccc(-c2nc3ccc4c(-c5ccc(C#N)cc5)nc5ccccc5c4c3nc2-c2ccc(C#N)cc2)cc1]

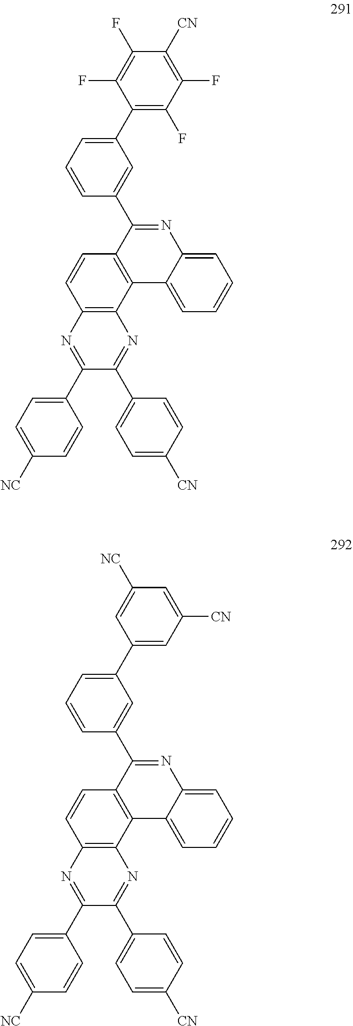 [SMILES: N#Cc1ccc(-c2nc3ccc4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3ccc4c(-c5cccc(-c6cc(C#N)cc(C#N)c6)c5)nc5ccccc5c4c3nc2-c2ccc(C#N)cc2)cc1]

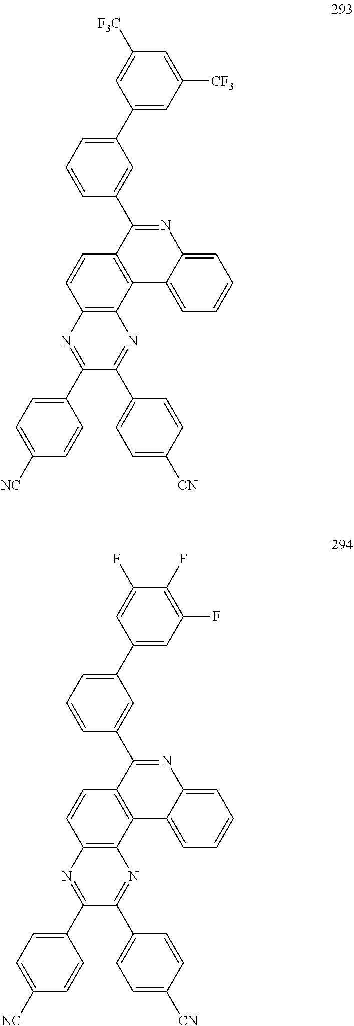 [SMILES: N#Cc1ccc(-c2nc3ccc4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3ccc4c(-c5cccc(-c6cc(F)c(F)c(F)c6)c5)nc5ccccc5c4c3nc2-c2ccc(C#N)cc2)cc1]

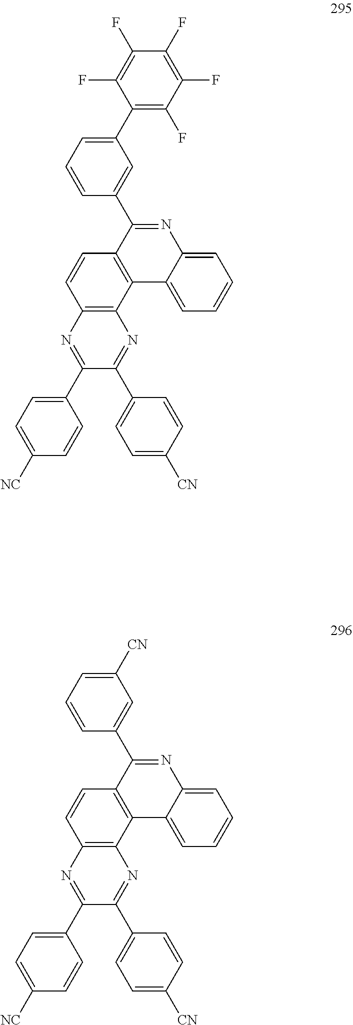 [SMILES: N#Cc1ccc(-c2nc3ccc4c(-c5cccc(-c6c(F)c(F)c(F)c(F)c6F)c5)nc5ccccc5c4c3nc2-c2ccc(C#N)cc2)cc1 ; N#Cc1ccc(-c2nc3ccc4c(-c5cccc(C#N)c5)nc5ccccc5c4c3nc2-c2ccc(C#N)cc2)cc1]

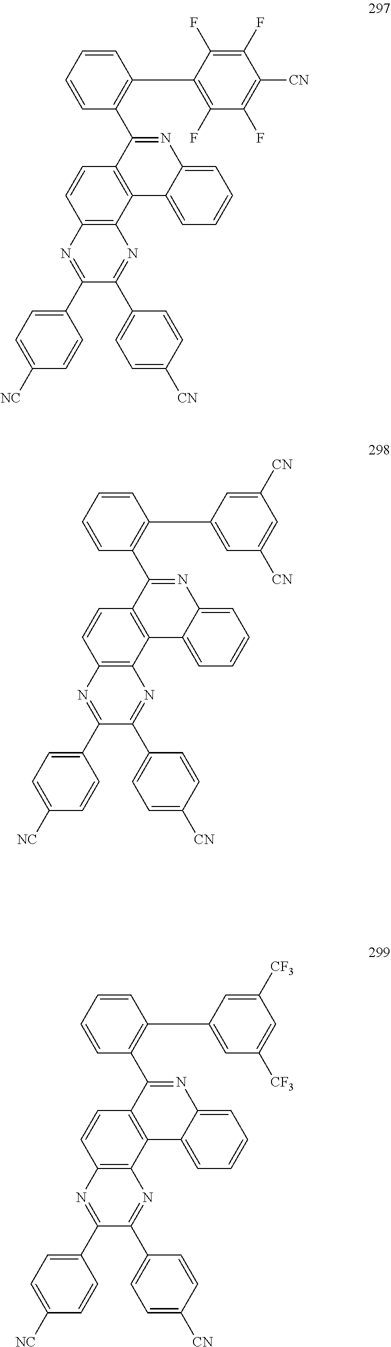 [SMILES: N#Cc1ccc(-c2nc3ccc4c(-c5ccccc5-c5c(F)c(F)c(C#N)c(F)c5F)nc5ccccc5c4c3nc2-c2ccc(C#N)cc2)cc1 ; N#Cc1ccc(-c2nc3ccc4c(-c5ccccc5-c5cc(C#N)cc(C#N)c5)nc5ccccc5c4c3nc2-c2ccc(C#N)cc2)cc1 ; N#Cc1ccc(-c2nc3ccc4c(-c5ccccc5-c5cc(C(F)(F)F)cc(C(F)(F)F)c5)nc5ccccc5c4c3nc2-c2ccc(C#N)cc2)cc1]

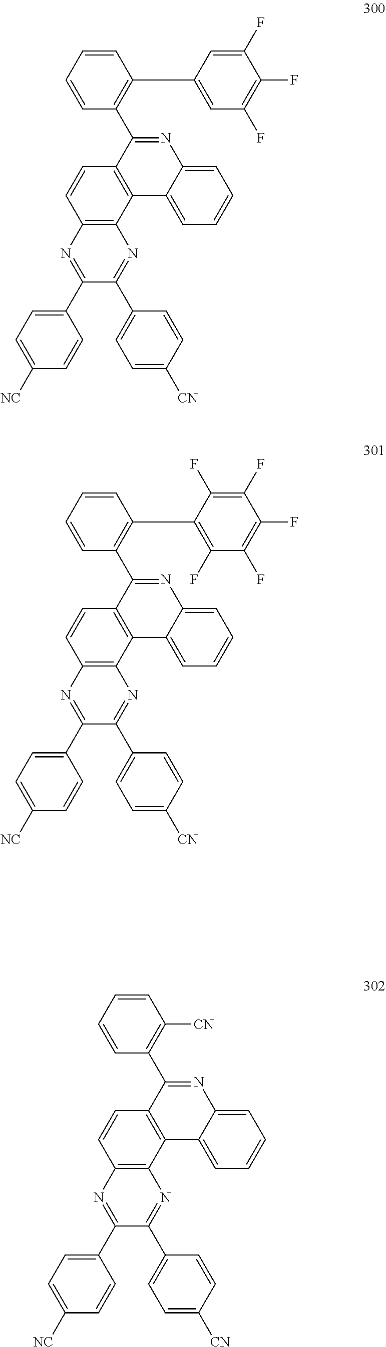 [SMILES: N#Cc1ccc(-c2nc3ccc4c(-c5ccccc5-c5cc(F)c(F)c(F)c5)nc5ccccc5c4c3nc2-c2ccc(C#N)cc2)cc1 ; N#Cc1ccc(-c2nc3ccc4c(-c5ccccc5-c5c(F)c(F)c(F)c(F)c5F)nc5ccccc5c4c3nc2-c2ccc(C#N)cc2)cc1 ; N#Cc1ccc(-c2nc3ccc4c(-c5ccccc5C#N)nc5ccccc5c4c3nc2-c2ccc(C#N)cc2)cc1]

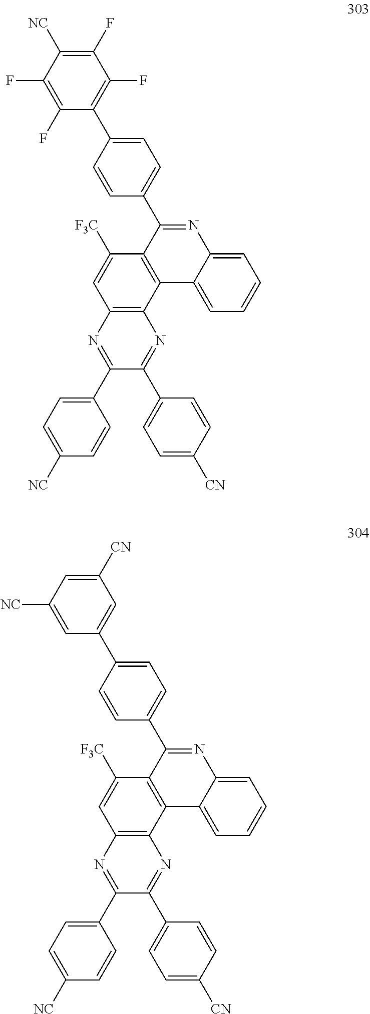 [SMILES: N#Cc1ccc(-c2nc3cc(C(F)(F)F)c4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3cc(C(F)(F)F)c4c(-c5ccc(-c6cc(C#N)cc(C#N)c6)cc5)nc5ccccc5c4c3nc2-c2ccc(C#N)cc2)cc1]

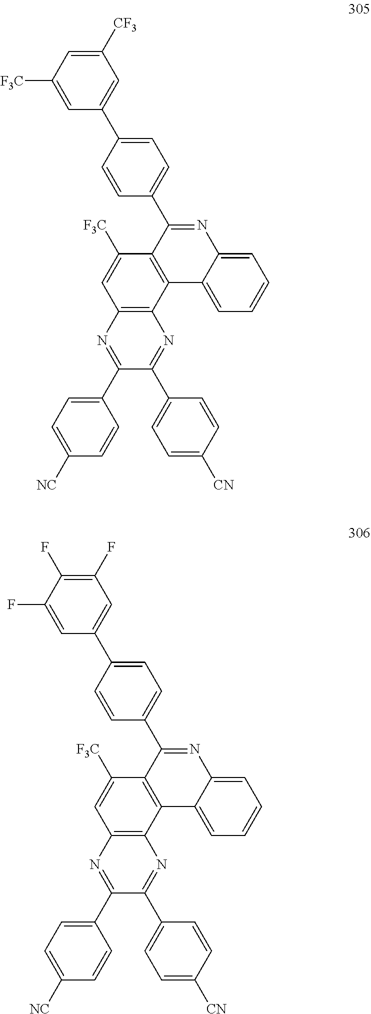 [SMILES: N#Cc1ccc(-c2nc3cc(C(F)(F)F)c4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3cc(C(F)(F)F)c4c(-c5ccc(-c6cc(F)c(F)c(F)c6)cc5)nc5ccccc5c4c3nc2-c2ccc(C#N)cc2)cc1]

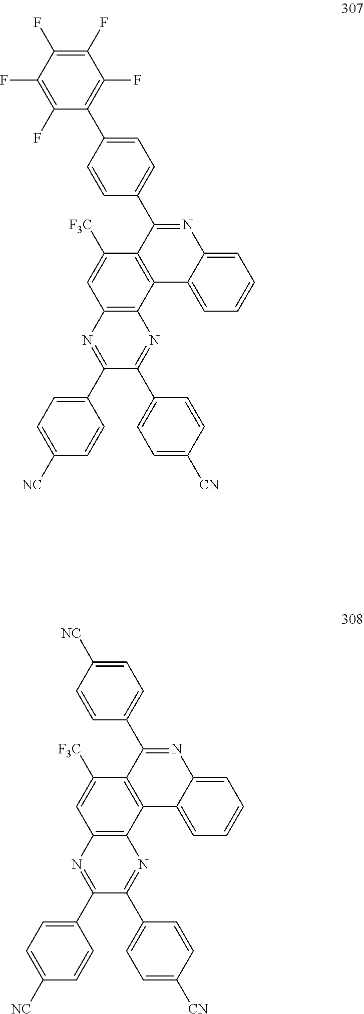 [SMILES: N#Cc1ccc(-c2nc3cc(C(F)(F)F)c4c(-c5ccc(-c6c(F)c(F)c(F)c(F)c6F)cc5)nc5ccccc5c4c3nc2-c2ccc(C#N)cc2)cc1 ; N#Cc1ccc(-c2nc3cc(C(F)(F)F)c4c(-c5ccc(C#N)cc5)nc5ccccc5c4c3nc2-c2ccc(C#N)cc2)cc1]

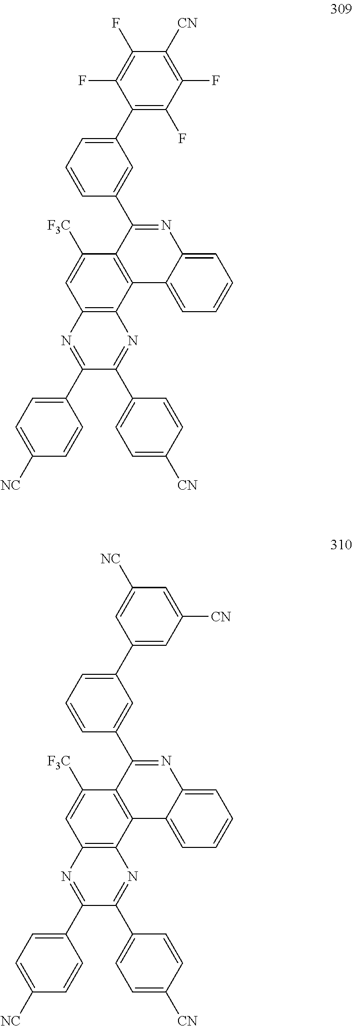 [SMILES: N#Cc1ccc(-c2nc3cc(C(F)(F)F)c4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3cc(C(F)(F)F)c4c(-c5cccc(-c6cc(C#N)cc(C#N)c6)c5)nc5ccccc5c4c3nc2-c2ccc(C#N)cc2)cc1]

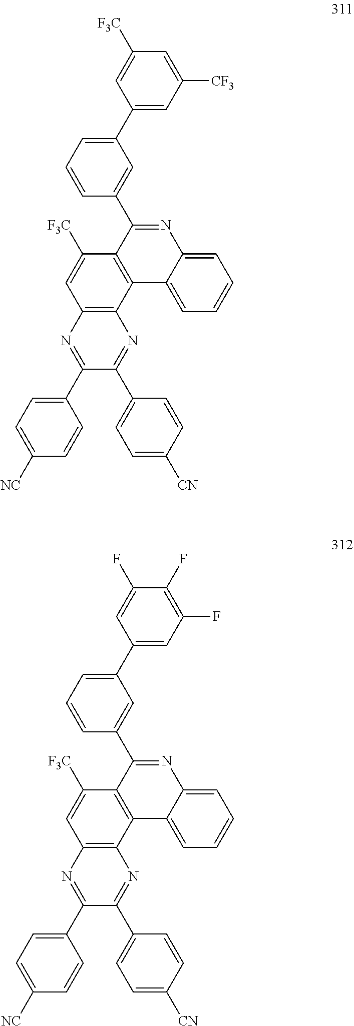 [SMILES: N#Cc1ccc(-c2nc3cc(C(F)(F)F)c4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3cc(C(F)(F)F)c4c(-c5cccc(-c6cc(F)c(F)c(F)c6)c5)nc5ccccc5c4c3nc2-c2ccc(C#N)cc2)cc1]

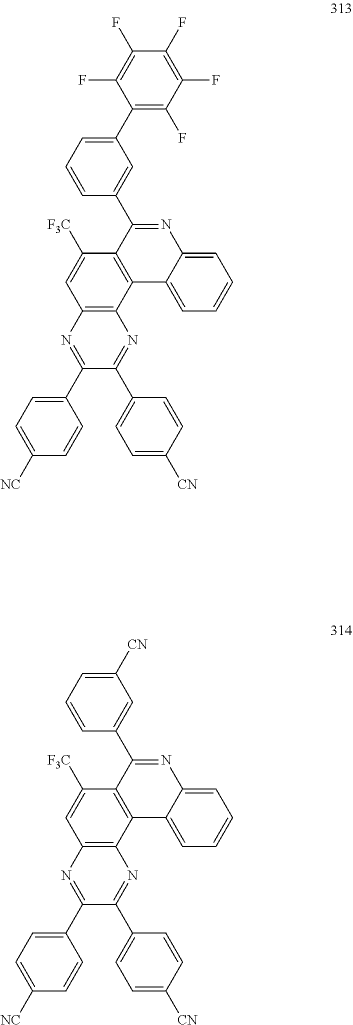 [SMILES: N#Cc1ccc(-c2nc3cc(C(F)(F)F)c4c(-c5cccc(-c6c(F)c(F)c(F)c(F)c6F)c5)nc5ccccc5c4c3nc2-c2ccc(C#N)cc2)cc1 ; N#Cc1ccc(-c2nc3cc(C(F)(F)F)c4c(-c5cccc(C#N)c5)nc5ccccc5c4c3nc2-c2ccc(C#N)cc2)cc1]

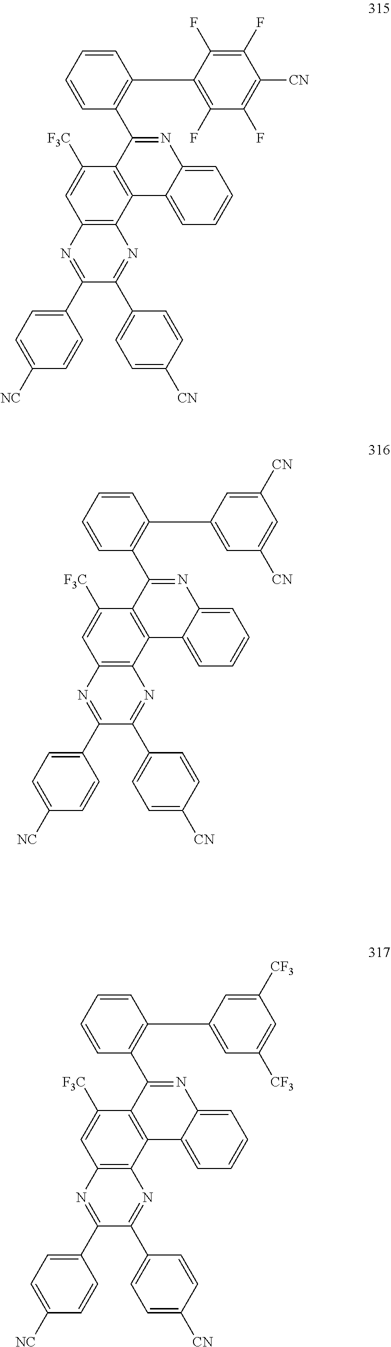 [SMILES: N#Cc1ccc(-c2nc3cc(C(F)(F)F)c4c(-c5ccccc5-c5c(F)c(F)c(C#N)c(F)c5F)nc5ccccc5c4c3nc2-c2ccc(C#N)cc2)cc1 ; N#Cc1ccc(-c2nc3cc(C(F)(F)F)c4c(-c5ccccc5-c5cc(C#N)cc(C#N)c5)nc5ccccc5c4c3nc2-c2ccc(C#N)cc2)cc1 ; N#Cc1ccc(-c2nc3cc(C(F)(F)F)c4c(-c5ccccc5-c5cc(C(F)(F)F)cc(C(F)(F)F)c5)nc5ccccc5c4c3nc2-c2ccc(C#N)cc2)cc1]

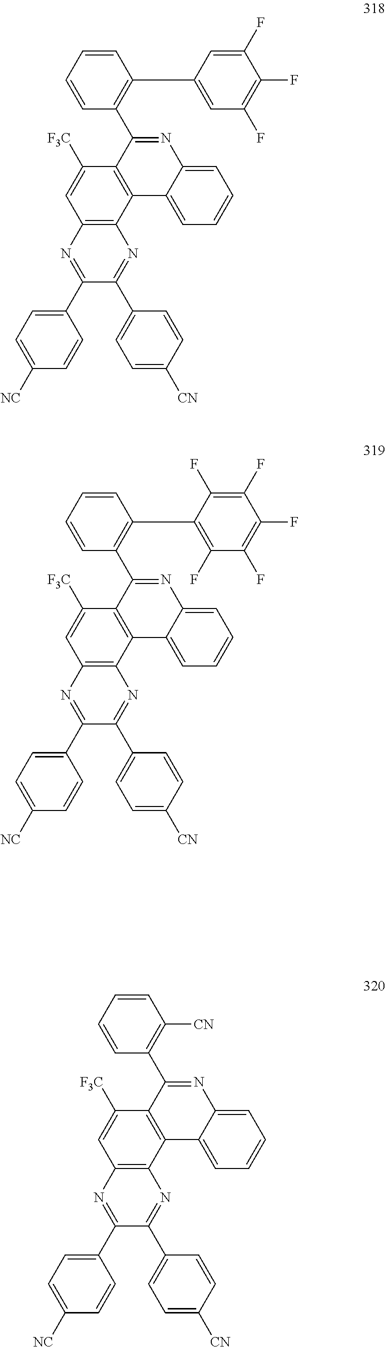 [SMILES: N#Cc1ccc(-c2nc3cc(C(F)(F)F)c4c(-c5ccccc5-c5cc(F)c(F)c(F)c5)nc5ccccc5c4c3nc2-c2ccc(C#N)cc2)cc1 ; N#Cc1ccc(-c2nc3cc(C(F)(F)F)c4c(-c5ccccc5-c5c(F)c(F)c(F)c(F)c5F)nc5ccccc5c4c3nc2-c2ccc(C#N)cc2)cc1 ; N#Cc1ccc(-c2nc3cc(C(F)(F)F)c4c(-c5ccccc5C#N)nc5ccccc5c4c3nc2-c2ccc(C#N)cc2)cc1]

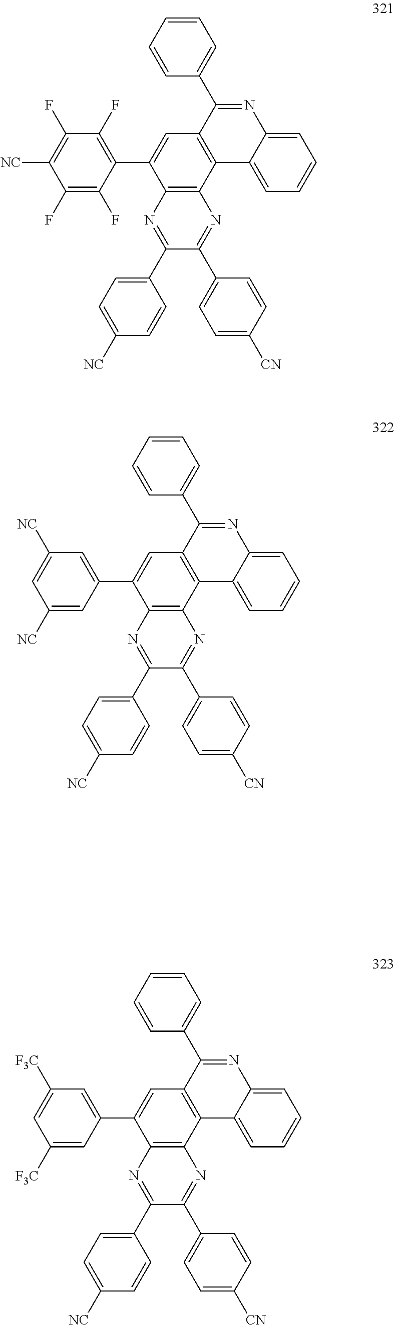 [SMILES: N#Cc1ccc(-c2nc3c(-c4c(F)c(F)c(C#N)c(F)c4F)cc4c(-c5ccccc5)nc5ccccc5c4c3nc2-c2ccc(C#N)cc2)cc1 ; N#Cc1ccc(-c2nc3c(-c4cc(C#N)cc(C#N)c4)cc4c(-c5ccccc5)nc5ccccc5c4c3nc2-c2ccc(C#N)cc2)cc1 ; N#Cc1ccc(-c2nc3c(-c4cc(C(F)(F)F)cc(C(F)(F)F)c4)cc4c(-c5ccccc5)nc5ccccc5c4c3nc2-c2ccc(C#N)cc2)cc1]

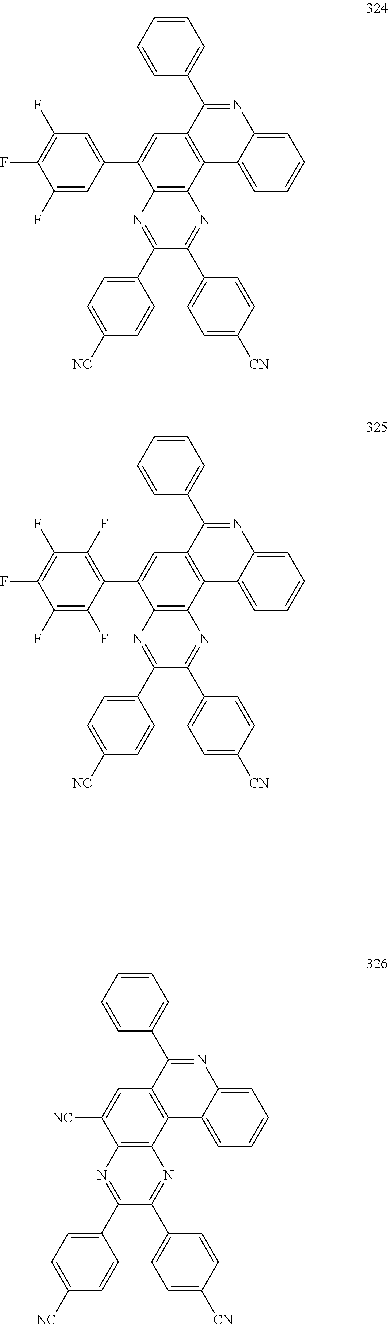 [SMILES: N#Cc1ccc(-c2nc3c(-c4cc(F)c(F)c(F)c4)cc4c(-c5ccccc5)nc5ccccc5c4c3nc2-c2ccc(C#N)cc2)cc1 ; N#Cc1ccc(-c2nc3c(-c4c(F)c(F)c(F)c(F)c4F)cc4c(-c5ccccc5)nc5ccccc5c4c3nc2-c2ccc(C#N)cc2)cc1 ; N#Cc1ccc(-c2nc3c(C#N)cc4c(-c5ccccc5)nc5ccccc5c4c3nc2-c2ccc(C#N)cc2)cc1]

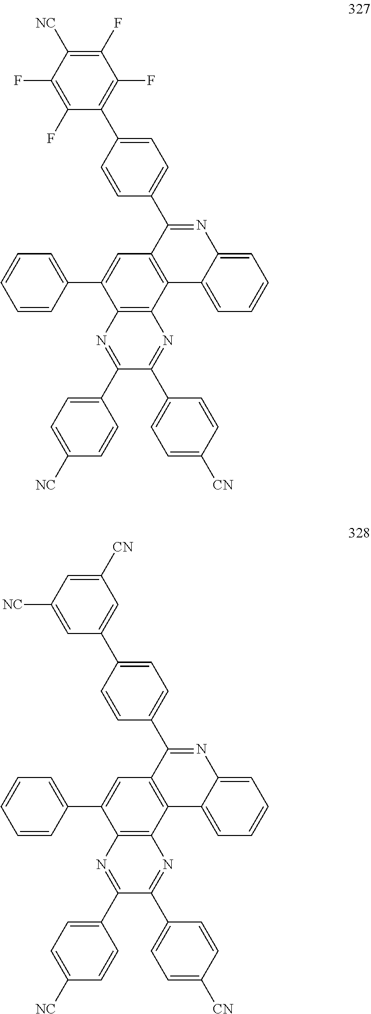 [SMILES: N#Cc1ccc(-c2nc3c(-c4ccccc4)cc4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3c(-c4ccccc4)cc4c(-c5ccc(-c6cc(C#N)cc(C#N)c6)cc5)nc5ccccc5c4c3nc2-c2ccc(C#N)cc2)cc1]

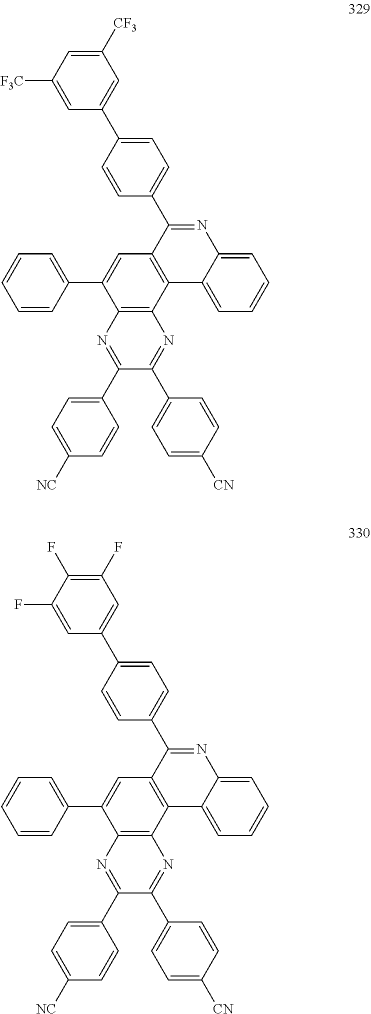 [SMILES: N#Cc1ccc(-c2nc3c(-c4ccccc4)cc4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3c(-c4ccccc4)cc4c(-c5ccc(-c6cc(F)c(F)c(F)c6)cc5)nc5ccccc5c4c3nc2-c2ccc(C#N)cc2)cc1]

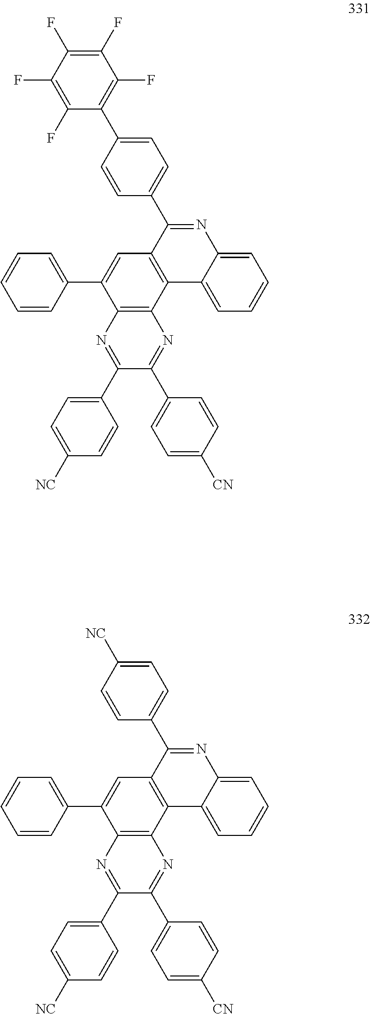 [SMILES: N#Cc1ccc(-c2nc3c(-c4ccccc4)cc4c(-c5ccc(-c6c(F)c(F)c(F)c(F)c6F)cc5)nc5ccccc5c4c3nc2-c2ccc(C#N)cc2)cc1 ; N#Cc1ccc(-c2nc3c(-c4ccccc4)cc4c(-c5ccc(C#N)cc5)nc5ccccc5c4c3nc2-c2ccc(C#N)cc2)cc1]

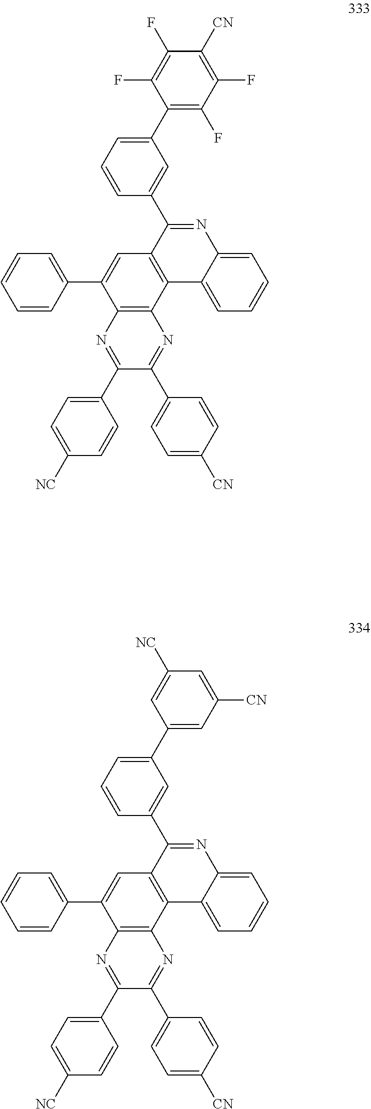 [SMILES: N#Cc1ccc(-c2nc3c(-c4ccccc4)cc4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3c(-c4ccccc4)cc4c(-c5cccc(-c6cc(C#N)cc(C#N)c6)c5)nc5ccccc5c4c3nc2-c2ccc(C#N)cc2)cc1]

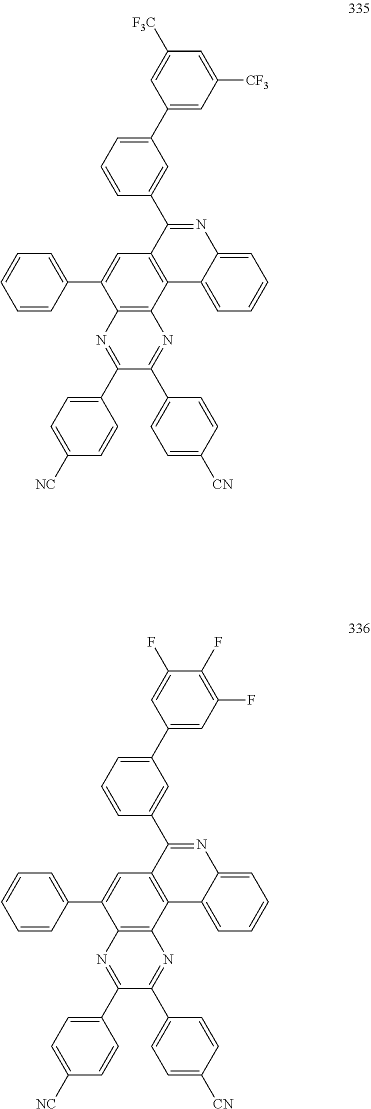 [SMILES: N#Cc1ccc(-c2nc3c(-c4ccccc4)cc4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3c(-c4ccccc4)cc4c(-c5cccc(-c6cc(F)c(F)c(F)c6)c5)nc5ccccc5c4c3nc2-c2ccc(C#N)cc2)cc1]

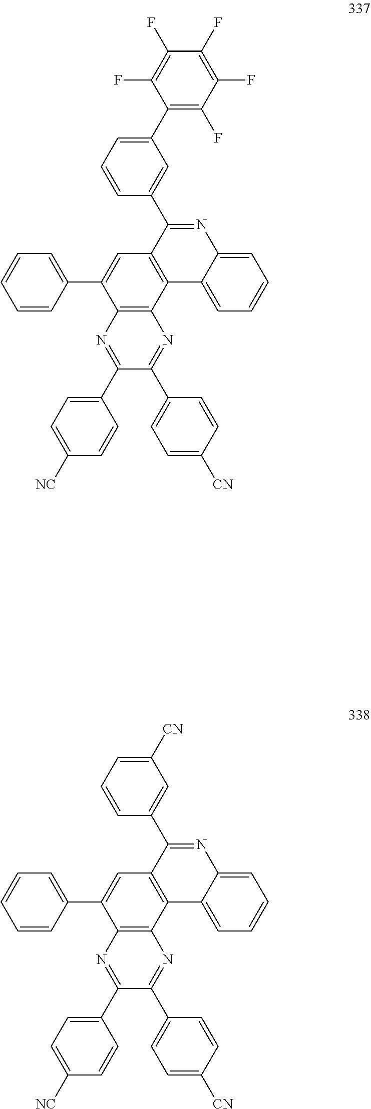 [SMILES: N#Cc1ccc(-c2nc3c(-c4ccccc4)cc4c(-c5cccc(-c6c(F)c(F)c(F)c(F)c6F)c5)nc5ccccc5c4c3nc2-c2ccc(C#N)cc2)cc1 ; N#Cc1ccc(-c2nc3c(-c4ccccc4)cc4c(-c5cccc(C#N)c5)nc5ccccc5c4c3nc2-c2ccc(C#N)cc2)cc1]

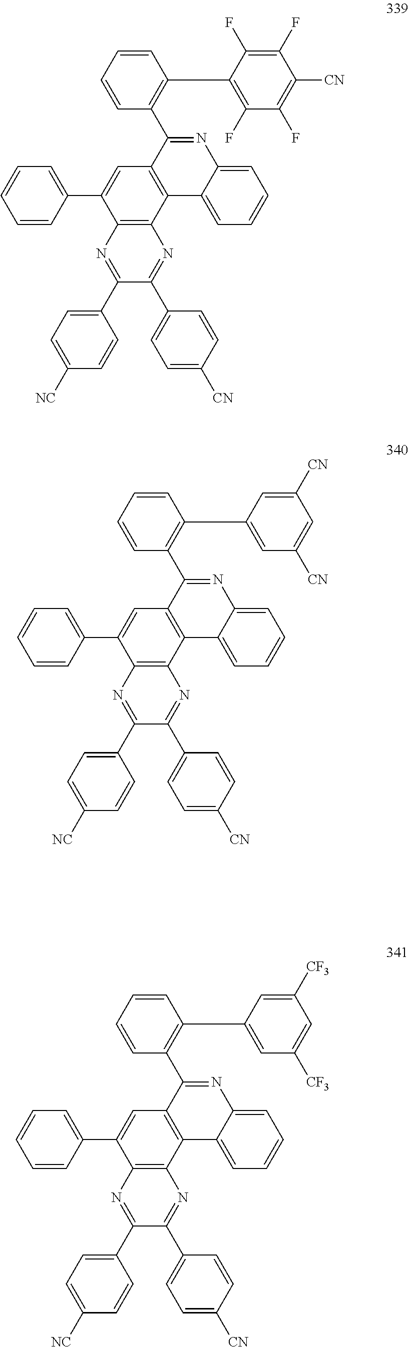 [SMILES: N#Cc1ccc(-c2nc3c(-c4ccccc4)cc4c(-c5ccccc5-c5c(F)c(F)c(C#N)c(F)c5F)nc5ccccc5c4c3nc2-c2ccc(C#N)cc2)cc1 ; N#Cc1ccc(-c2nc3c(-c4ccccc4)cc4c(-c5ccccc5-c5cc(C#N)cc(C#N)c5)nc5ccccc5c4c3nc2-c2ccc(C#N)cc2)cc1 ; N#Cc1ccc(-c2nc3c(-c4ccccc4)cc4c(-c5ccccc5-c5cc(C(F)(F)F)cc(C(F)(F)F)c5)nc5ccccc5c4c3nc2-c2ccc(C#N)cc2)cc1]

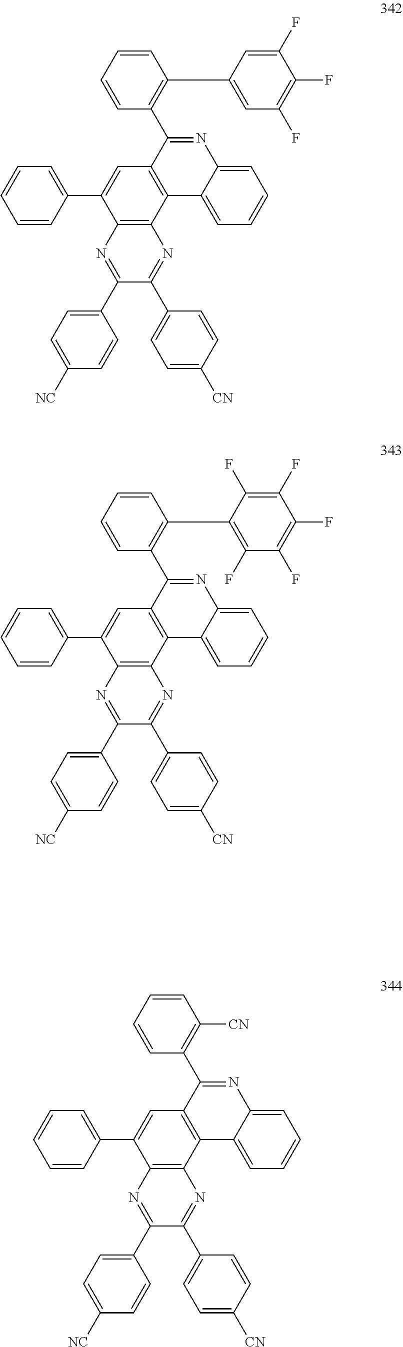 [SMILES: N#Cc1ccc(-c2nc3c(-c4ccccc4)cc4c(-c5ccccc5-c5cc(F)c(F)c(F)c5)nc5ccccc5c4c3nc2-c2ccc(C#N)cc2)cc1 ; N#Cc1ccc(-c2nc3c(-c4ccccc4)cc4c(-c5ccccc5-c5c(F)c(F)c(F)c(F)c5F)nc5ccccc5c4c3nc2-c2ccc(C#N)cc2)cc1 ; N#Cc1ccc(-c2nc3c(-c4ccccc4)cc4c(-c5ccccc5C#N)nc5ccccc5c4c3nc2-c2ccc(C#N)cc2)cc1]

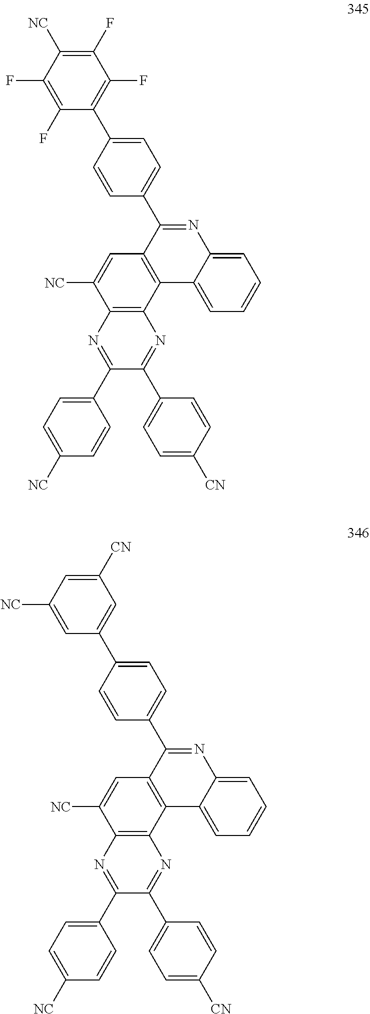 [SMILES: N#Cc1ccc(-c2nc3c(C#N)cc4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3c(C#N)cc4c(-c5ccc(-c6cc(C#N)cc(C#N)c6)cc5)nc5ccccc5c4c3nc2-c2ccc(C#N)cc2)cc1]

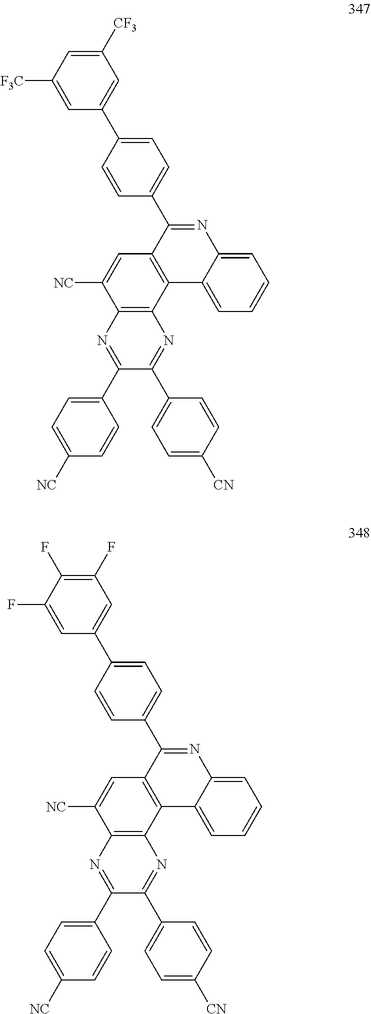 [SMILES: N#Cc1ccc(-c2nc3c(C#N)cc4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3c(C#N)cc4c(-c5ccc(-c6cc(F)c(F)c(F)c6)cc5)nc5ccccc5c4c3nc2-c2ccc(C#N)cc2)cc1]

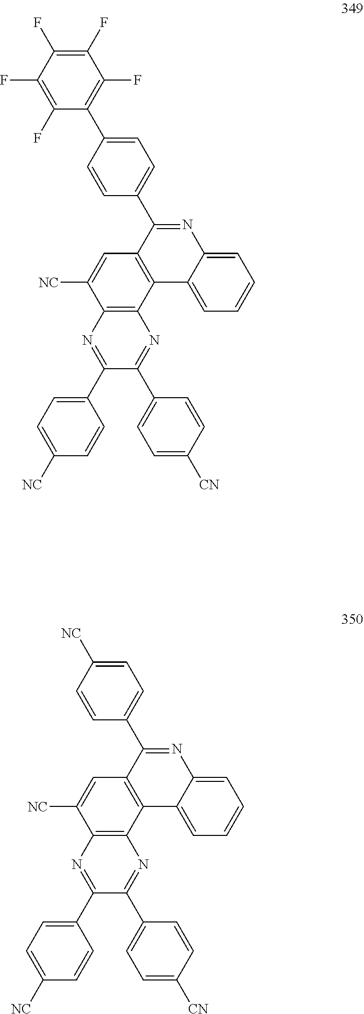 [SMILES: N#Cc1ccc(-c2nc3c(C#N)cc4c(-c5ccc(-c6c(F)c(F)c(F)c(F)c6F)cc5)nc5ccccc5c4c3nc2-c2ccc(C#N)cc2)cc1 ; N#Cc1ccc(-c2nc3c(C#N)cc4c(-c5ccc(C#N)cc5)nc5ccccc5c4c3nc2-c2ccc(C#N)cc2)cc1]

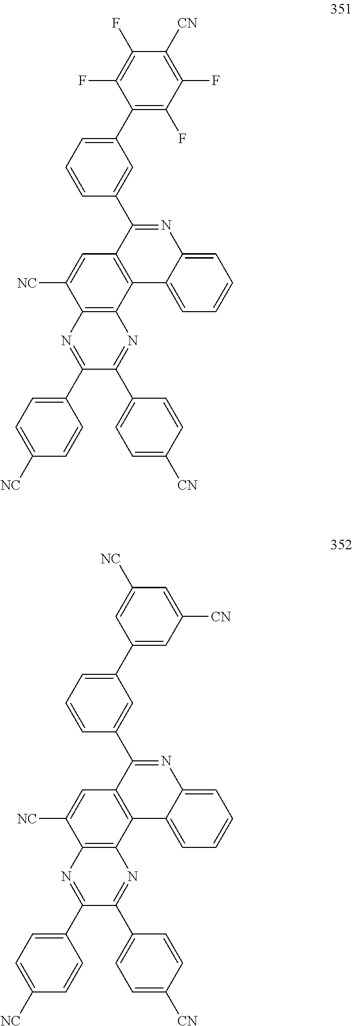 [SMILES: N#Cc1ccc(-c2nc3c(C#N)cc4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3c(C#N)cc4c(-c5cccc(-c6cc(C#N)cc(C#N)c6)c5)nc5ccccc5c4c3nc2-c2ccc(C#N)cc2)cc1]

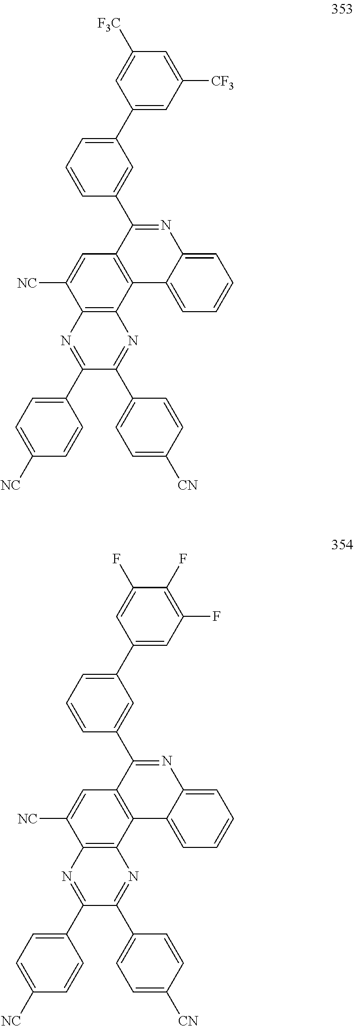 [SMILES: N#Cc1ccc(-c2nc3c(C#N)cc4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3c(C#N)cc4c(-c5cccc(-c6cc(F)c(F)c(F)c6)c5)nc5ccccc5c4c3nc2-c2ccc(C#N)cc2)cc1]

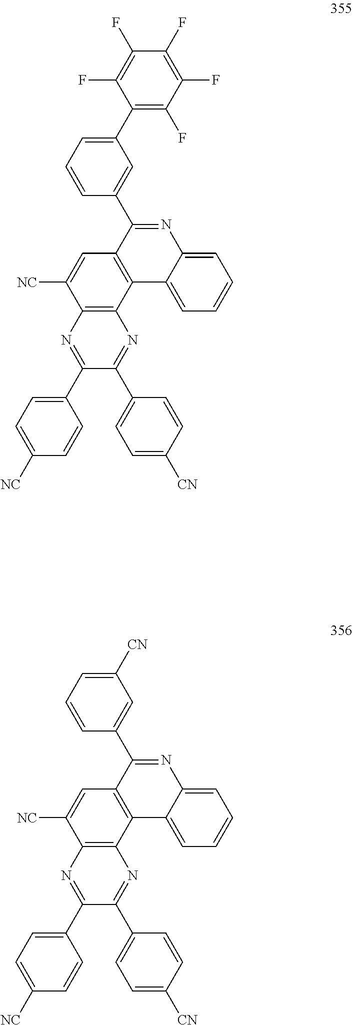 [SMILES: N#Cc1ccc(-c2nc3c(C#N)cc4c(-c5cccc(-c6c(F)c(F)c(F)c(F)c6F)c5)nc5ccccc5c4c3nc2-c2ccc(C#N)cc2)cc1 ; N#Cc1ccc(-c2nc3c(C#N)cc4c(-c5cccc(C#N)c5)nc5ccccc5c4c3nc2-c2ccc(C#N)cc2)cc1]

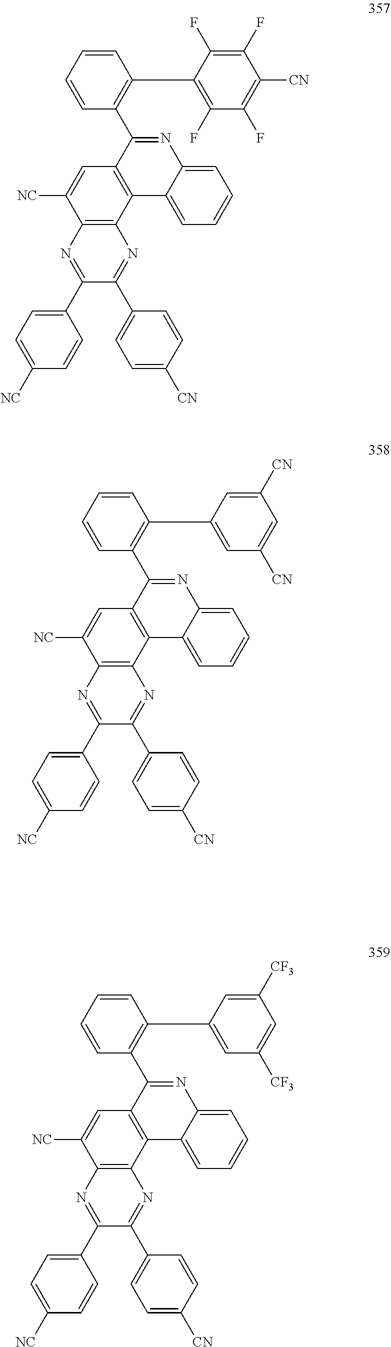 [SMILES: N#Cc1ccc(-c2nc3c(C#N)cc4c(-c5ccccc5-c5c(F)c(F)c(C#N)c(F)c5F)nc5ccccc5c4c3nc2-c2ccc(C#N)cc2)cc1 ; N#Cc1ccc(-c2nc3c(C#N)cc4c(-c5ccccc5-c5cc(C#N)cc(C#N)c5)nc5ccccc5c4c3nc2-c2ccc(C#N)cc2)cc1 ; N#Cc1ccc(-c2nc3c(C#N)cc4c(-c5ccccc5-c5cc(C(F)(F)F)cc(C(F)(F)F)c5)nc5ccccc5c4c3nc2-c2ccc(C#N)cc2)cc1]

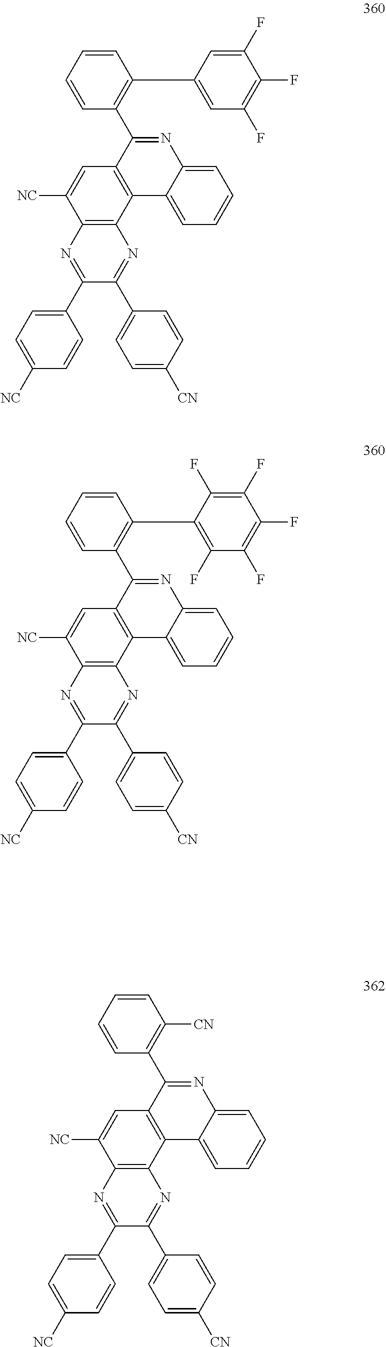 [SMILES: N#Cc1ccc(-c2nc3c(C#N)cc4c(-c5ccccc5-c5cc(F)c(F)c(F)c5)nc5ccccc5c4c3nc2-c2ccc(C#N)cc2)cc1 ; N#Cc1ccc(-c2nc3c(C#N)cc4c(-c5ccccc5-c5c(F)c(F)c(F)c(F)c5F)nc5ccccc5c4c3nc2-c2ccc(C#N)cc2)cc1 ; N#Cc1ccc(-c2nc3c(C#N)cc4c(-c5ccccc5C#N)nc5ccccc5c4c3nc2-c2ccc(C#N)cc2)cc1]

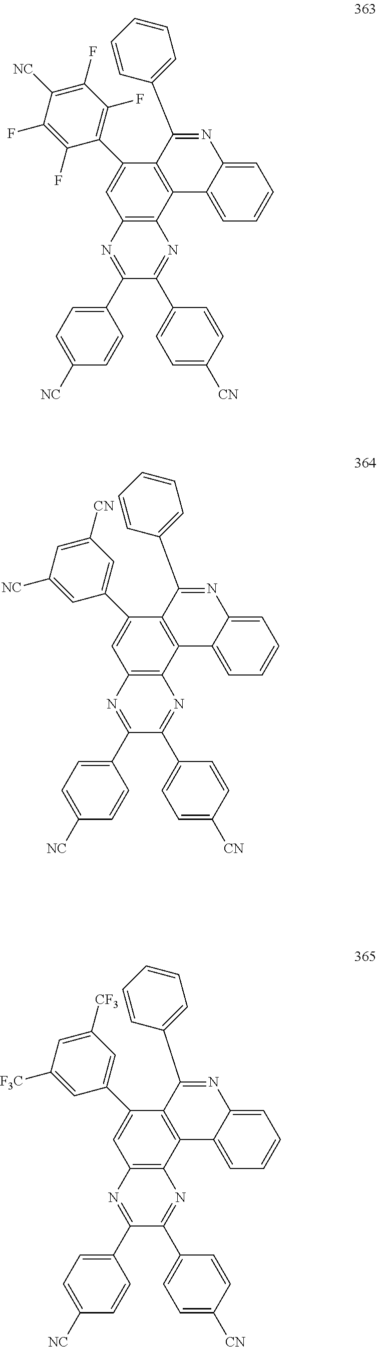 [SMILES: N#Cc1ccc(-c2nc3cc(-c4c(F)c(F)c(C#N)c(F)c4F)c4c(-c5ccccc5)nc5ccccc5c4c3nc2-c2ccc(C#N)cc2)cc1 ; N#Cc1ccc(-c2nc3cc(-c4cc(C#N)cc(C#N)c4)c4c(-c5ccccc5)nc5ccccc5c4c3nc2-c2ccc(C#N)cc2)cc1 ; N#Cc1ccc(-c2nc3cc(-c4cc(C(F)(F)F)cc(C(F)(F)F)c4)c4c(-c5ccccc5)nc5ccccc5c4c3nc2-c2ccc(C#N)cc2)cc1]

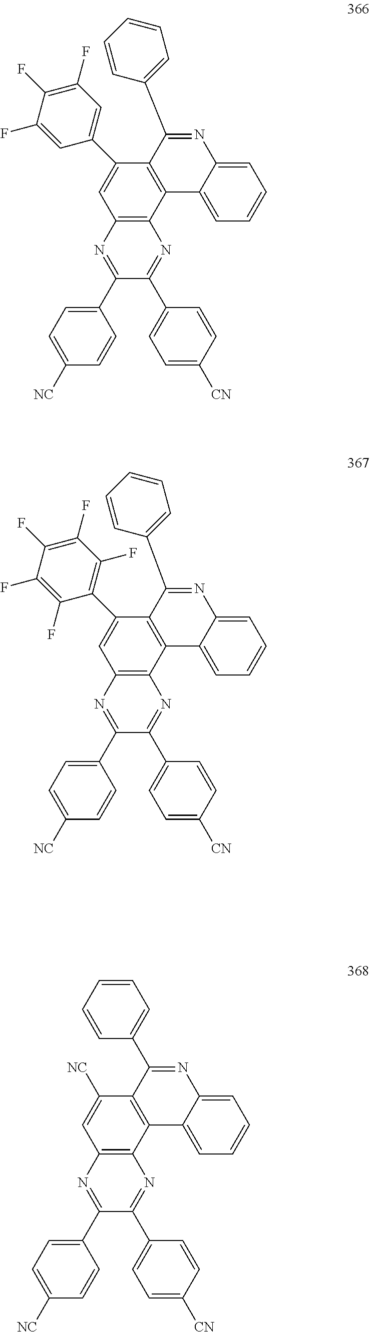 [SMILES: N#Cc1ccc(-c2nc3cc(-c4cc(F)c(F)c(F)c4)c4c(-c5ccccc5)nc5ccccc5c4c3nc2-c2ccc(C#N)cc2)cc1 ; N#Cc1ccc(-c2nc3cc(-c4c(F)c(F)c(F)c(F)c4F)c4c(-c5ccccc5)nc5ccccc5c4c3nc2-c2ccc(C#N)cc2)cc1 ; N#Cc1ccc(-c2nc3cc(C#N)c4c(-c5ccccc5)nc5ccccc5c4c3nc2-c2ccc(C#N)cc2)cc1]

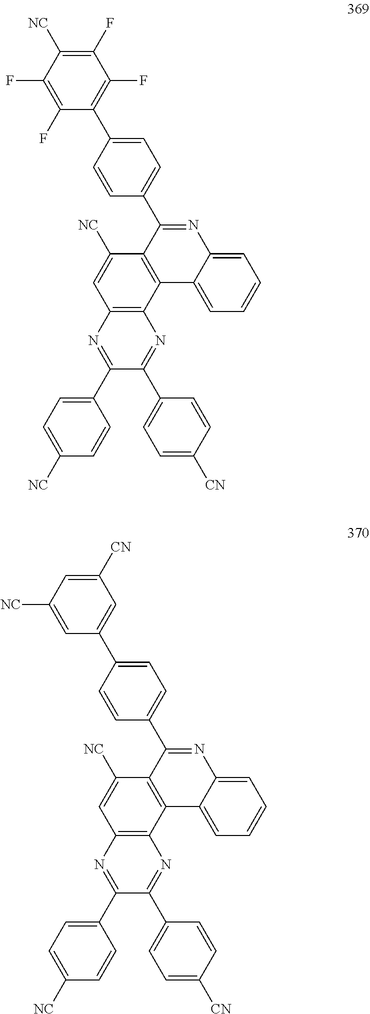 [SMILES: N#Cc1ccc(-c2nc3cc(C#N)c4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3cc(C#N)c4c(-c5ccc(-c6cc(C#N)cc(C#N)c6)cc5)nc5ccccc5c4c3nc2-c2ccc(C#N)cc2)cc1]

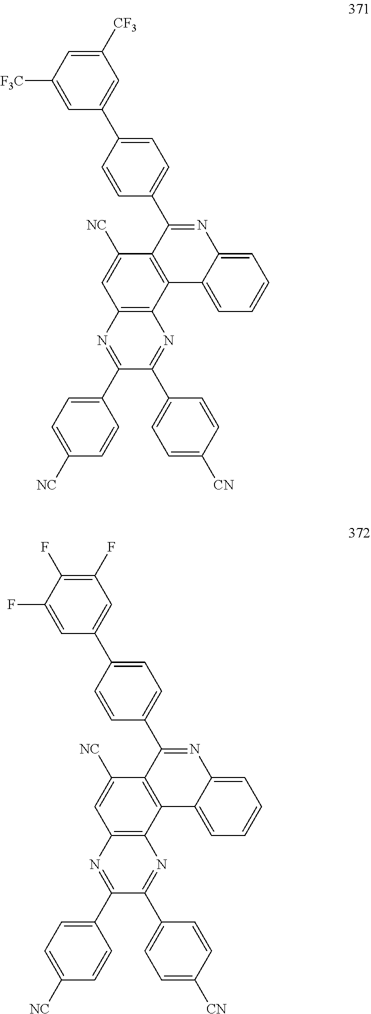 [SMILES: N#Cc1ccc(-c2nc3cc(C#N)c4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3cc(C#N)c4c(-c5ccc(-c6cc(F)c(F)c(F)c6)cc5)nc5ccccc5c4c3nc2-c2ccc(C#N)cc2)cc1]

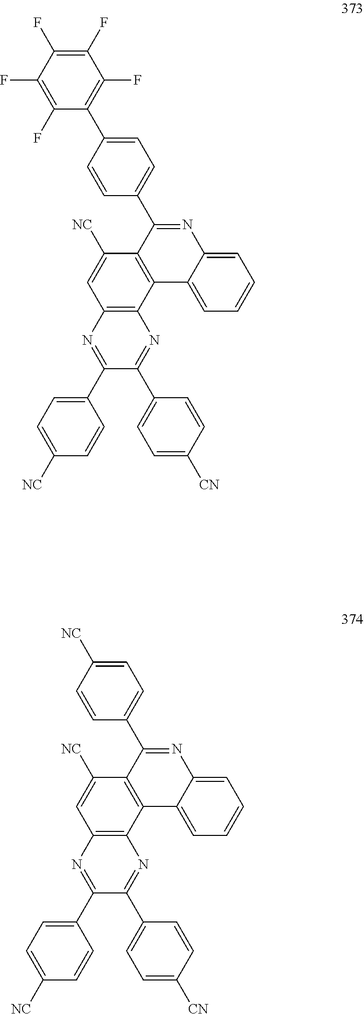 [SMILES: N#Cc1ccc(-c2nc3cc(C#N)c4c(-c5ccc(-c6c(F)c(F)c(F)c(F)c6F)cc5)nc5ccccc5c4c3nc2-c2ccc(C#N)cc2)cc1 ; N#Cc1ccc(-c2nc3cc(C#N)c4c(-c5ccc(C#N)cc5)nc5ccccc5c4c3nc2-c2ccc(C#N)cc2)cc1]

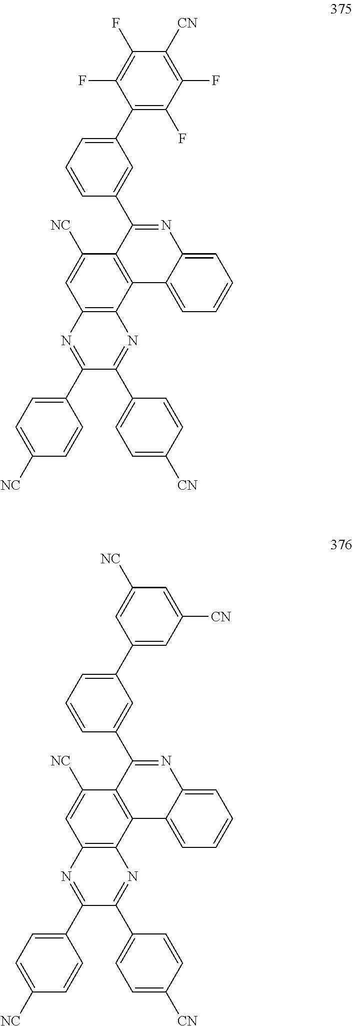 [SMILES: N#Cc1ccc(-c2nc3cc(C#N)c4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3cc(C#N)c4c(-c5cccc(-c6cc(C#N)cc(C#N)c6)c5)nc5ccccc5c4c3nc2-c2ccc(C#N)cc2)cc1]

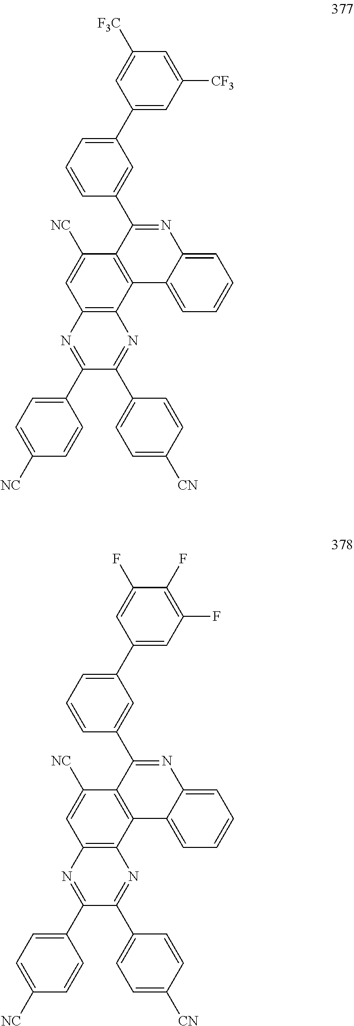 [SMILES: N#Cc1ccc(-c2nc3cc(C#N)c4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3cc(C#N)c4c(-c5cccc(-c6cc(F)c(F)c(F)c6)c5)nc5ccccc5c4c3nc2-c2ccc(C#N)cc2)cc1]

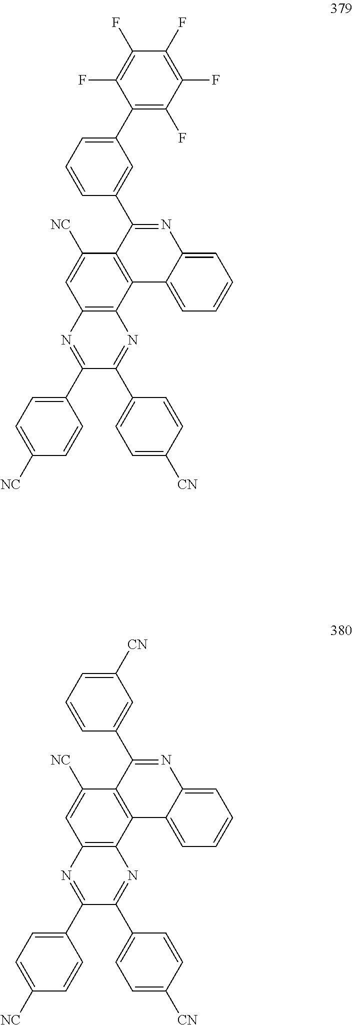 [SMILES: N#Cc1ccc(-c2nc3cc(C#N)c4c(-c5cccc(-c6c(F)c(F)c(F)c(F)c6F)c5)nc5ccccc5c4c3nc2-c2ccc(C#N)cc2)cc1 ; N#Cc1ccc(-c2nc3cc(C#N)c4c(-c5cccc(C#N)c5)nc5ccccc5c4c3nc2-c2ccc(C#N)cc2)cc1]

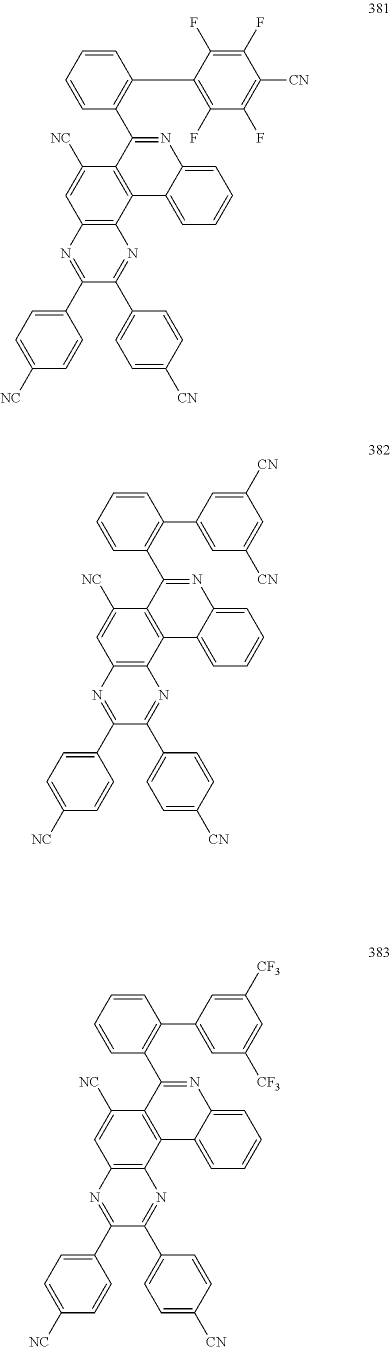 [SMILES: N#Cc1ccc(-c2nc3cc(C#N)c4c(-c5ccccc5-c5c(F)c(F)c(C#N)c(F)c5F)nc5ccccc5c4c3nc2-c2ccc(C#N)cc2)cc1 ; N#Cc1ccc(-c2nc3cc(C#N)c4c(-c5ccccc5-c5cc(C#N)cc(C#N)c5)nc5ccccc5c4c3nc2-c2ccc(C#N)cc2)cc1 ; N#Cc1ccc(-c2nc3cc(C#N)c4c(-c5ccccc5-c5cc(C(F)(F)F)cc(C(F)(F)F)c5)nc5ccccc5c4c3nc2-c2ccc(C#N)cc2)cc1]

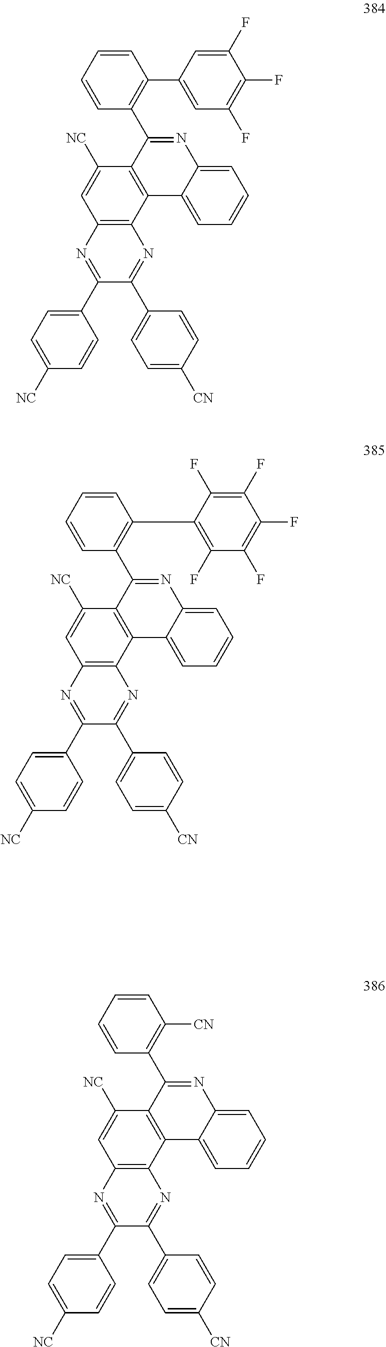 [SMILES: N#Cc1ccc(-c2nc3cc(C#N)c4c(-c5ccccc5-c5cc(F)c(F)c(F)c5)nc5ccccc5c4c3nc2-c2ccc(C#N)cc2)cc1 ; N#Cc1ccc(-c2nc3cc(C#N)c4c(-c5ccccc5-c5c(F)c(F)c(F)c(F)c5F)nc5ccccc5c4c3nc2-c2ccc(C#N)cc2)cc1 ; N#Cc1ccc(-c2nc3cc(C#N)c4c(-c5ccccc5C#N)nc5ccccc5c4c3nc2-c2ccc(C#N)cc2)cc1]

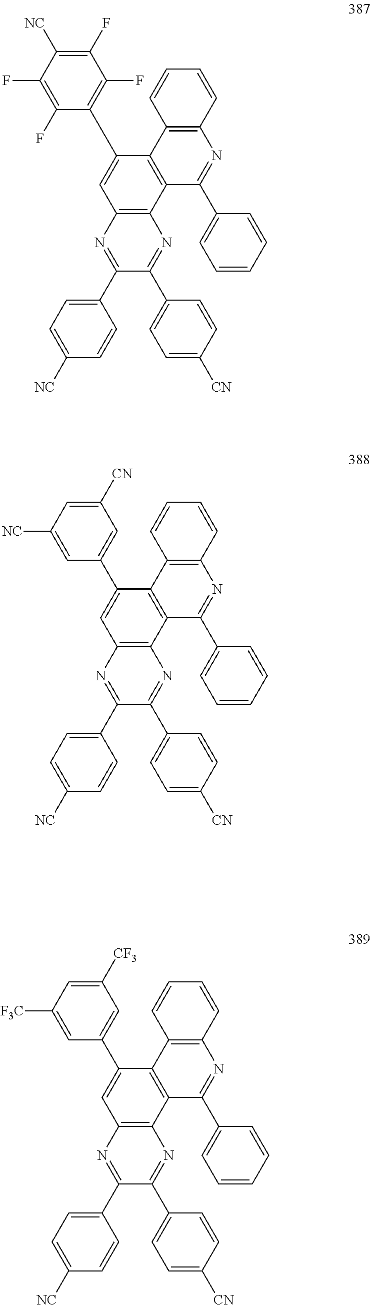 [SMILES: N#Cc1ccc(-c2nc3cc(-c4c(F)c(F)c(C#N)c(F)c4F)c4c5ccccc5nc(-c5ccccc5)c4c3nc2-c2ccc(C#N)cc2)cc1 ; N#Cc1ccc(-c2nc3cc(-c4cc(C#N)cc(C#N)c4)c4c5ccccc5nc(-c5ccccc5)c4c3nc2-c2ccc(C#N)cc2)cc1 ; N#Cc1ccc(-c2nc3cc(-c4cc(C(F)(F)F)cc(C(F)(F)F)c4)c4c5ccccc5nc(-c5ccccc5)c4c3nc2-c2ccc(C#N)cc2)cc1]

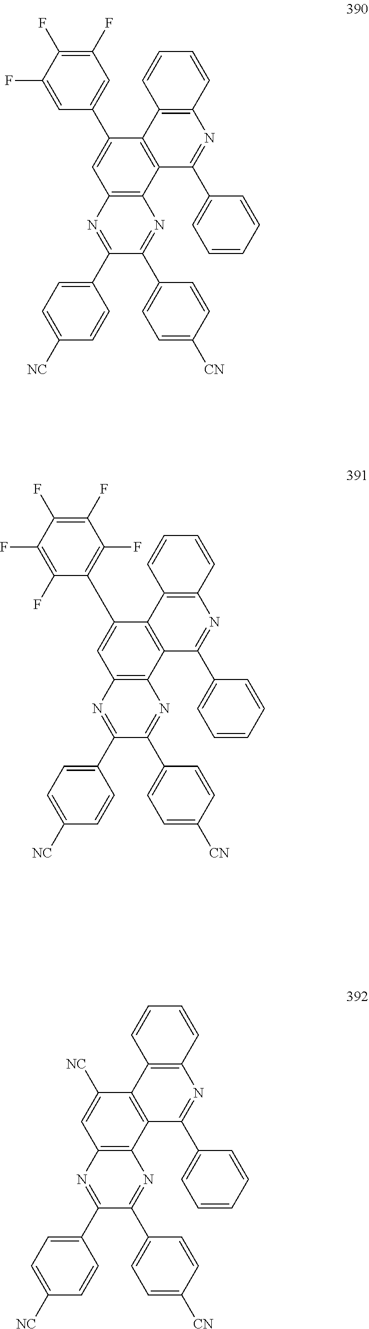 [SMILES: N#Cc1ccc(-c2nc3cc(-c4cc(F)c(F)c(F)c4)c4c5ccccc5nc(-c5ccccc5)c4c3nc2-c2ccc(C#N)cc2)cc1 ; N#Cc1ccc(-c2nc3cc(-c4c(F)c(F)c(F)c(F)c4F)c4c5ccccc5nc(-c5ccccc5)c4c3nc2-c2ccc(C#N)cc2)cc1 ; N#Cc1ccc(-c2nc3cc(C#N)c4c5ccccc5nc(-c5ccccc5)c4c3nc2-c2ccc(C#N)cc2)cc1]

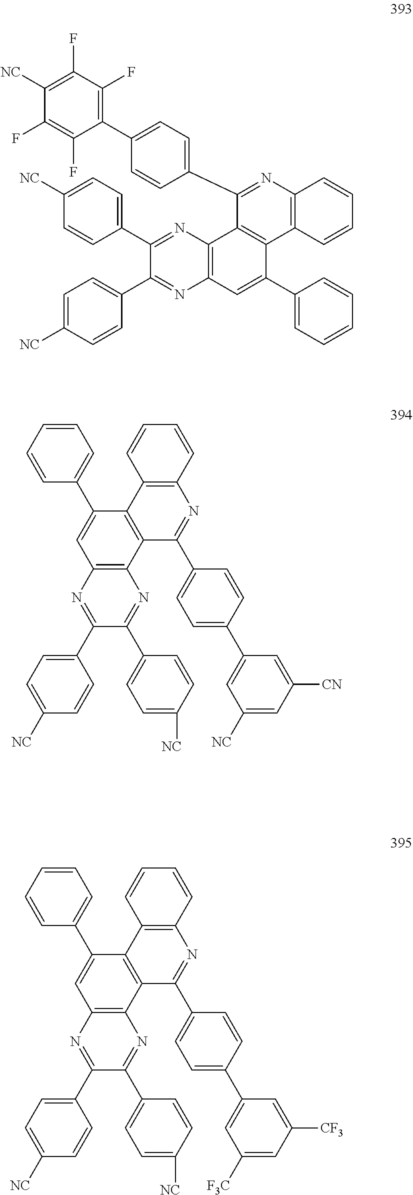 [SMILES: N#Cc1ccc(-c2nc3cc(-c4ccccc4)c4c5ccccc5nc(-c5ccc(-c6c(F)c(F)c(C#N)c(F)c6F)cc5)c4c3nc2-c2ccc(C#N)cc2)cc1 ; N#Cc1ccc(-c2nc3cc(-c4ccccc4)c4c5ccccc5nc(-c5ccc(-c6cc(C#N)cc(C#N)c6)cc5)c4c3nc2-c2ccc(C#N)cc2)cc1 ; N#Cc1ccc(-c2nc3cc(-c4ccccc4)c4c5ccccc5nc(-c5ccc(-c6cc(C(F)(F)F)cc(C(F)(F)F)c6)cc5)c4c3nc2-c2ccc(C#N)cc2)cc1]

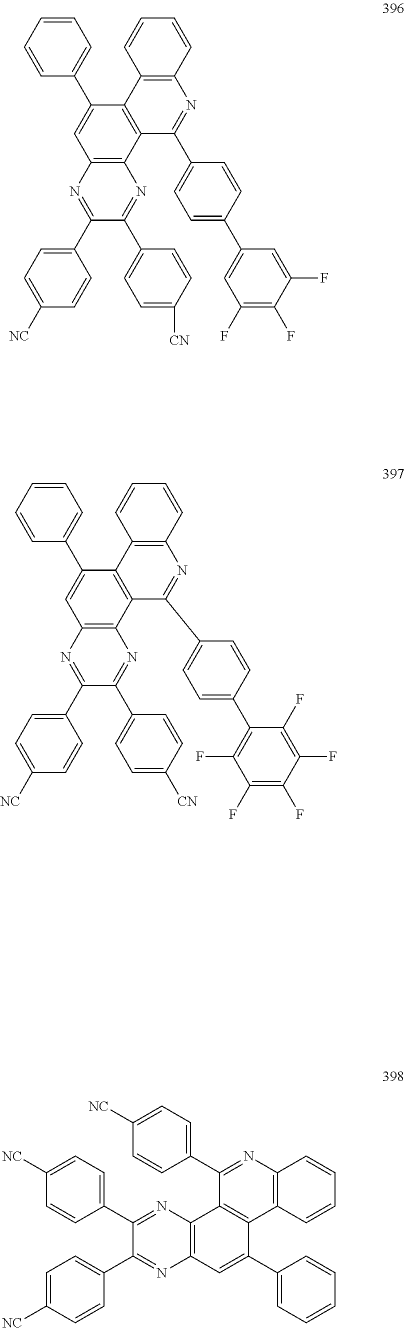 [SMILES: N#Cc1ccc(-c2nc3cc(-c4ccccc4)c4c5ccccc5nc(-c5ccc(-c6cc(F)c(F)c(F)c6)cc5)c4c3nc2-c2ccc(C#N)cc2)cc1 ; N#Cc1ccc(-c2nc3cc(-c4ccccc4)c4c5ccccc5nc(-c5ccc(-c6c(F)c(F)c(F)c(F)c6F)cc5)c4c3nc2-c2ccc(C#N)cc2)cc1 ; N#Cc1ccc(-c2nc3cc(-c4ccccc4)c4c5ccccc5nc(-c5ccc(C#N)cc5)c4c3nc2-c2ccc(C#N)cc2)cc1]

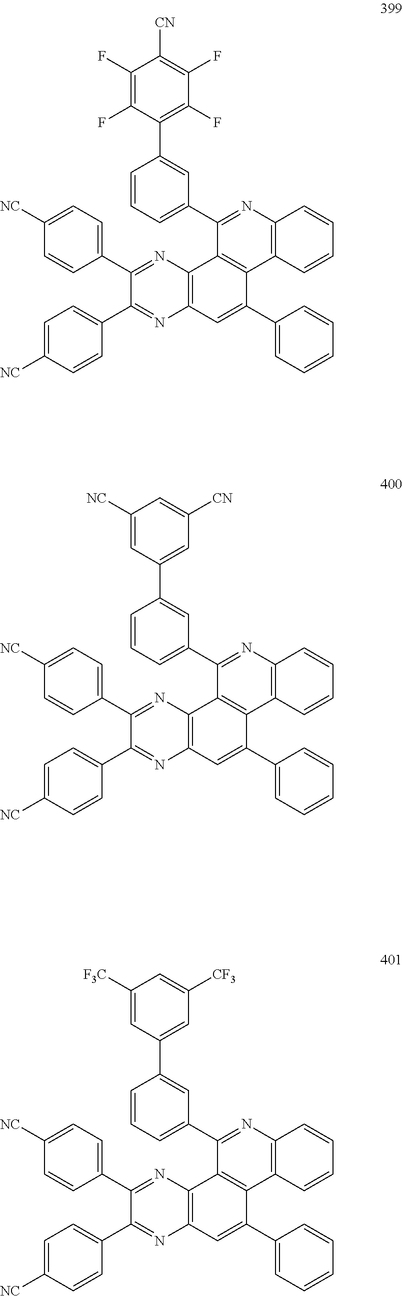 [SMILES: N#Cc1ccc(-c2nc3cc(-c4ccccc4)c4c5ccccc5nc(-c5cccc(-c6c(F)c(F)c(C#N)c(F)c6F)c5)c4c3nc2-c2ccc(C#N)cc2)cc1 ; N#Cc1ccc(-c2nc3cc(-c4ccccc4)c4c5ccccc5nc(-c5cccc(-c6cc(C#N)cc(C#N)c6)c5)c4c3nc2-c2ccc(C#N)cc2)cc1 ; N#Cc1ccc(-c2nc3cc(-c4ccccc4)c4c5ccccc5nc(-c5cccc(-c6cc(C(F)(F)F)cc(C(F)(F)F)c6)c5)c4c3nc2-c2ccc(C#N)cc2)cc1]

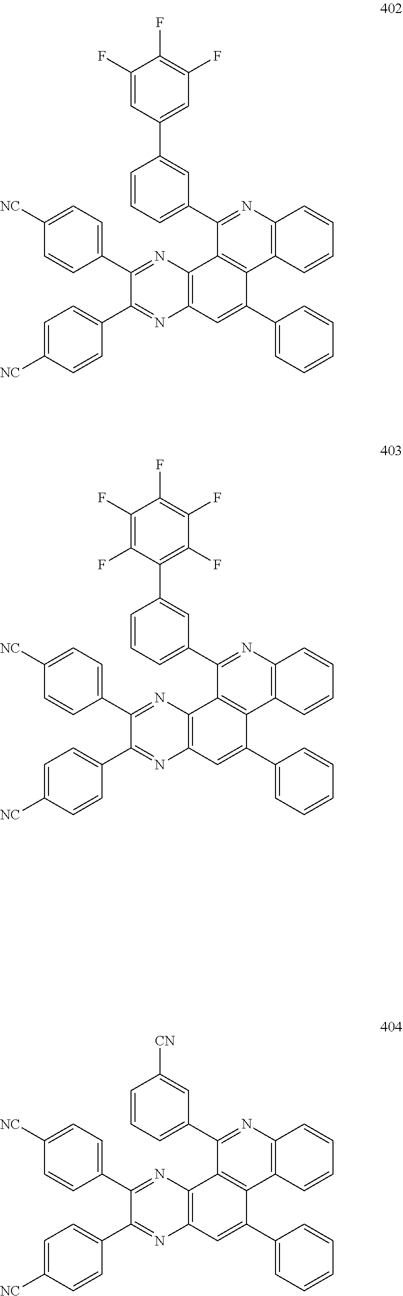 [SMILES: N#Cc1ccc(-c2nc3cc(-c4ccccc4)c4c5ccccc5nc(-c5cccc(-c6cc(F)c(F)c(F)c6)c5)c4c3nc2-c2ccc(C#N)cc2)cc1 ; N#Cc1ccc(-c2nc3cc(-c4ccccc4)c4c5ccccc5nc(-c5cccc(-c6c(F)c(F)c(F)c(F)c6F)c5)c4c3nc2-c2ccc(C#N)cc2)cc1 ; N#Cc1ccc(-c2nc3cc(-c4ccccc4)c4c5ccccc5nc(-c5cccc(C#N)c5)c4c3nc2-c2ccc(C#N)cc2)cc1]

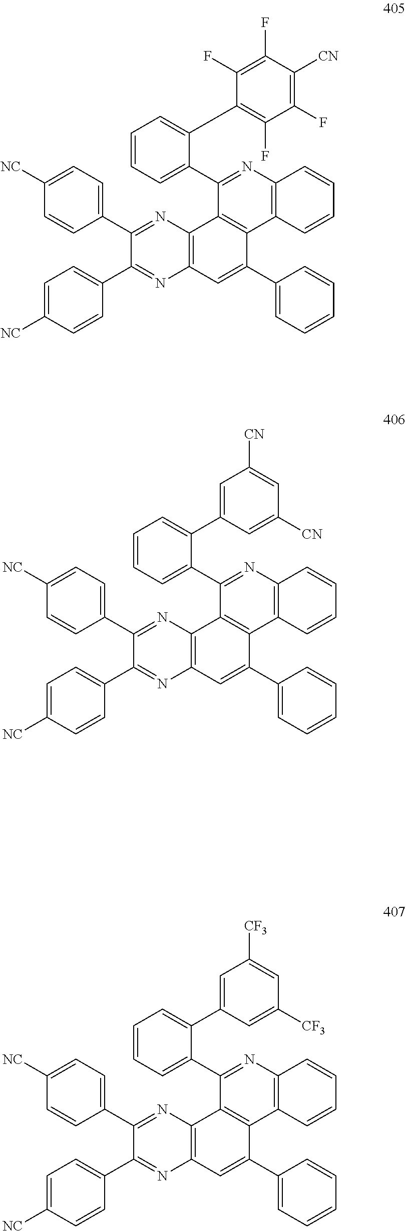 [SMILES: N#Cc1ccc(-c2nc3cc(-c4ccccc4)c4c5ccccc5nc(-c5ccccc5-c5c(F)c(F)c(C#N)c(F)c5F)c4c3nc2-c2ccc(C#N)cc2)cc1 ; N#Cc1ccc(-c2nc3cc(-c4ccccc4)c4c5ccccc5nc(-c5ccccc5-c5cc(C#N)cc(C#N)c5)c4c3nc2-c2ccc(C#N)cc2)cc1 ; N#Cc1ccc(-c2nc3cc(-c4ccccc4)c4c5ccccc5nc(-c5ccccc5-c5cc(C(F)(F)F)cc(C(F)(F)F)c5)c4c3nc2-c2ccc(C#N)cc2)cc1]

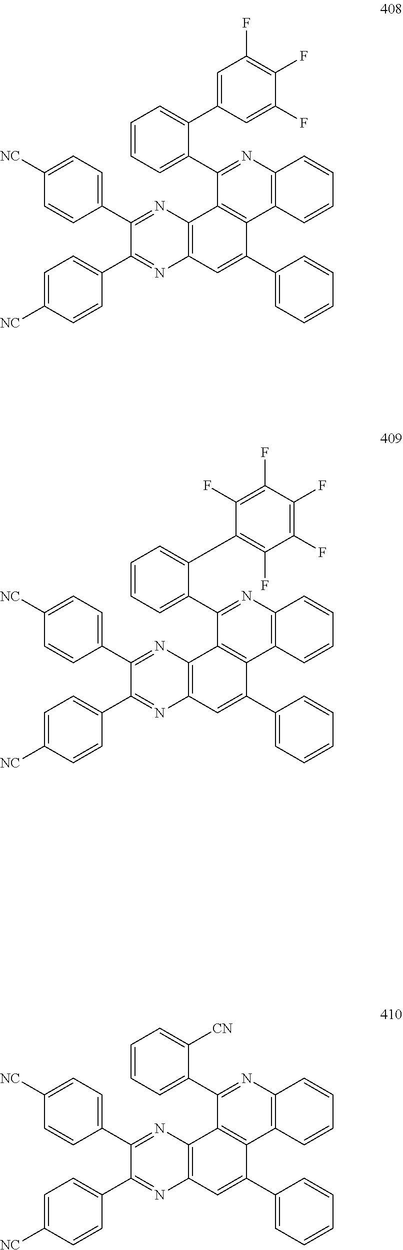 [SMILES: N#Cc1ccc(-c2nc3cc(-c4ccccc4)c4c5ccccc5nc(-c5ccccc5-c5cc(F)c(F)c(F)c5)c4c3nc2-c2ccc(C#N)cc2)cc1 ; N#Cc1ccc(-c2nc3cc(-c4ccccc4)c4c5ccccc5nc(-c5ccccc5-c5c(F)c(F)c(F)c(F)c5F)c4c3nc2-c2ccc(C#N)cc2)cc1 ; N#Cc1ccc(-c2nc3cc(-c4ccccc4)c4c5ccccc5nc(-c5ccccc5C#N)c4c3nc2-c2ccc(C#N)cc2)cc1]

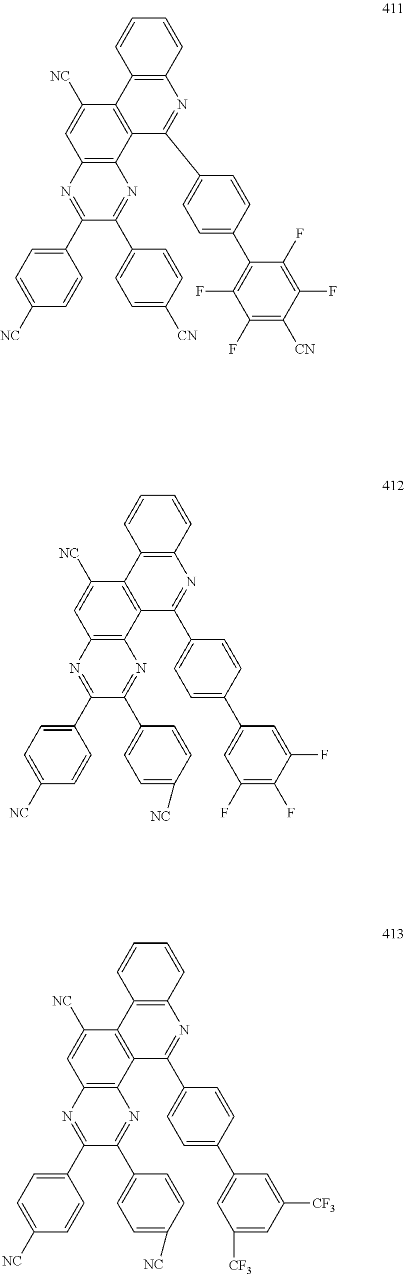 [SMILES: N#Cc1ccc(-c2nc3cc(C#N)c4c5ccccc5nc(-c5ccc(-c6c(F)c(F)c(C#N)c(F)c6F)cc5)c4c3nc2-c2ccc(C#N)cc2)cc1 ; N#Cc1ccc(-c2nc3cc(C#N)c4c5ccccc5nc(-c5ccc(-c6cc(F)c(F)c(F)c6)cc5)c4c3nc2-c2ccc(C#N)cc2)cc1 ; N#Cc1ccc(-c2nc3cc(C#N)c4c5ccccc5nc(-c5ccc(-c6cc(C(F)(F)F)cc(C(F)(F)F)c6)cc5)c4c3nc2-c2ccc(C#N)cc2)cc1]

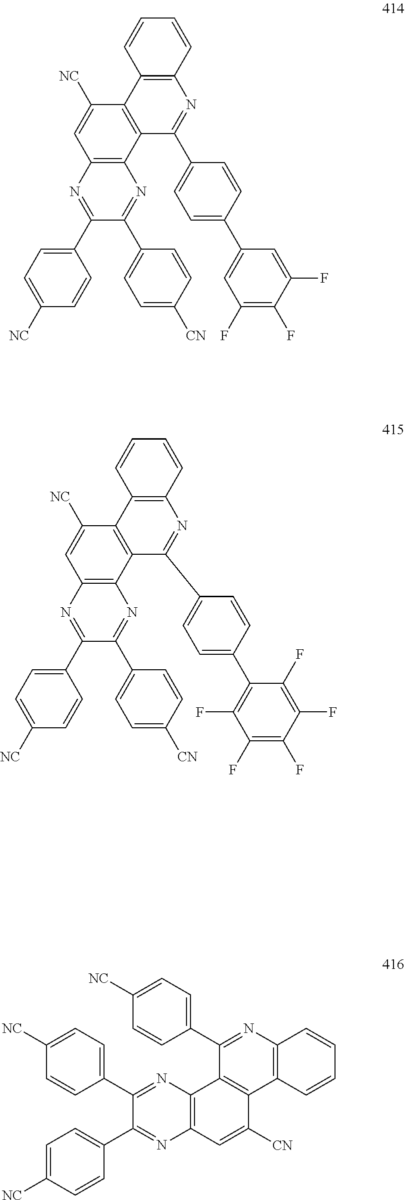 [SMILES: N#Cc1ccc(-c2nc3cc(C#N)c4c5ccccc5nc(-c5ccc(-c6cc(F)c(F)c(F)c6)cc5)c4c3nc2-c2ccc(C#N)cc2)cc1 ; N#Cc1ccc(-c2nc3cc(C#N)c4c5ccccc5nc(-c5ccc(-c6c(F)c(F)c(F)c(F)c6F)cc5)c4c3nc2-c2ccc(C#N)cc2)cc1 ; N#Cc1ccc(-c2nc3cc(C#N)c4c5ccccc5nc(-c5ccc(C#N)cc5)c4c3nc2-c2ccc(C#N)cc2)cc1]

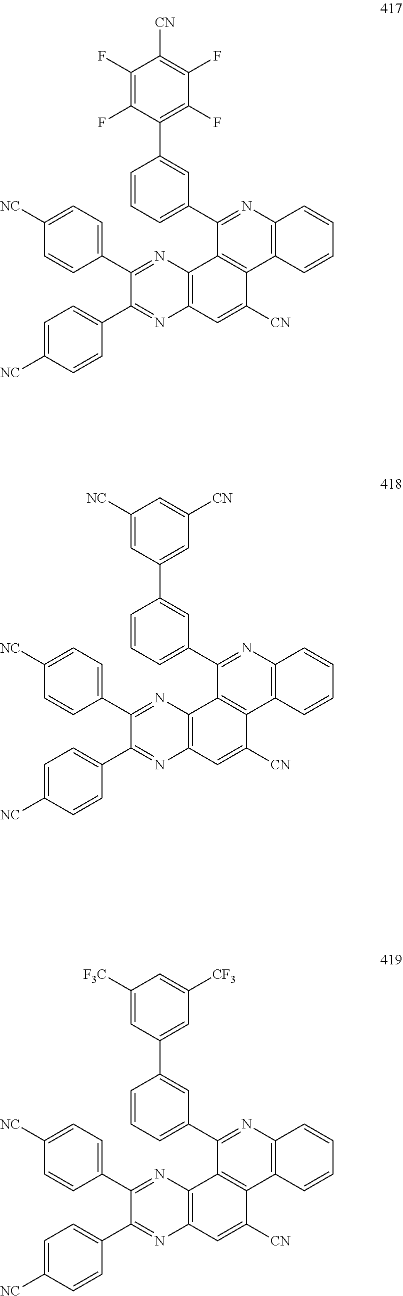 [SMILES: N#Cc1ccc(-c2nc3cc(C#N)c4c5ccccc5nc(-c5cccc(-c6c(F)c(F)c(C#N)c(F)c6F)c5)c4c3nc2-c2ccc(C#N)cc2)cc1 ; N#Cc1ccc(-c2nc3cc(C#N)c4c5ccccc5nc(-c5cccc(-c6cc(C#N)cc(C#N)c6)c5)c4c3nc2-c2ccc(C#N)cc2)cc1 ; N#Cc1ccc(-c2nc3cc(C#N)c4c5ccccc5nc(-c5cccc(-c6cc(C(F)(F)F)cc(C(F)(F)F)c6)c5)c4c3nc2-c2ccc(C#N)cc2)cc1]

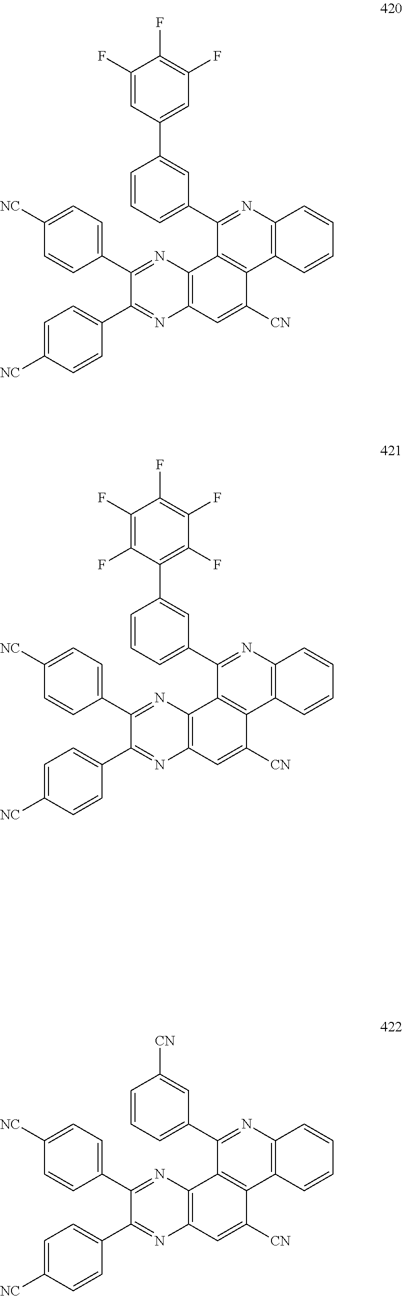 [SMILES: N#Cc1ccc(-c2nc3cc(C#N)c4c5ccccc5nc(-c5cccc(-c6cc(F)c(F)c(F)c6)c5)c4c3nc2-c2ccc(C#N)cc2)cc1 ; N#Cc1ccc(-c2nc3cc(C#N)c4c5ccccc5nc(-c5cccc(-c6c(F)c(F)c(F)c(F)c6F)c5)c4c3nc2-c2ccc(C#N)cc2)cc1 ; N#Cc1ccc(-c2nc3cc(C#N)c4c5ccccc5nc(-c5cccc(C#N)c5)c4c3nc2-c2ccc(C#N)cc2)cc1]

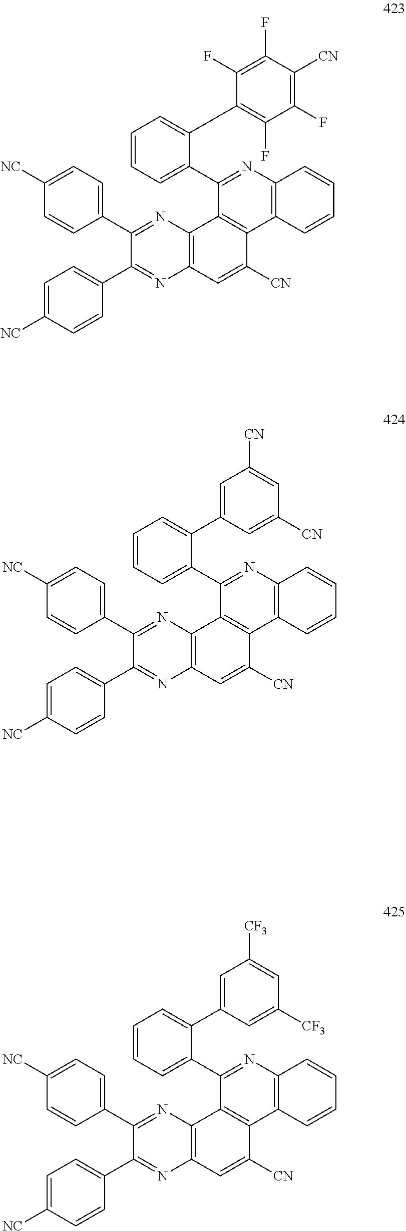 [SMILES: N#Cc1ccc(-c2nc3cc(C#N)c4c5ccccc5nc(-c5ccccc5-c5c(F)c(F)c(C#N)c(F)c5F)c4c3nc2-c2ccc(C#N)cc2)cc1 ; N#Cc1ccc(-c2nc3cc(C#N)c4c5ccccc5nc(-c5ccccc5-c5cc(C#N)cc(C#N)c5)c4c3nc2-c2ccc(C#N)cc2)cc1 ; N#Cc1ccc(-c2nc3cc(C#N)c4c5ccccc5nc(-c5ccccc5-c5cc(C(F)(F)F)cc(C(F)(F)F)c5)c4c3nc2-c2ccc(C#N)cc2)cc1]

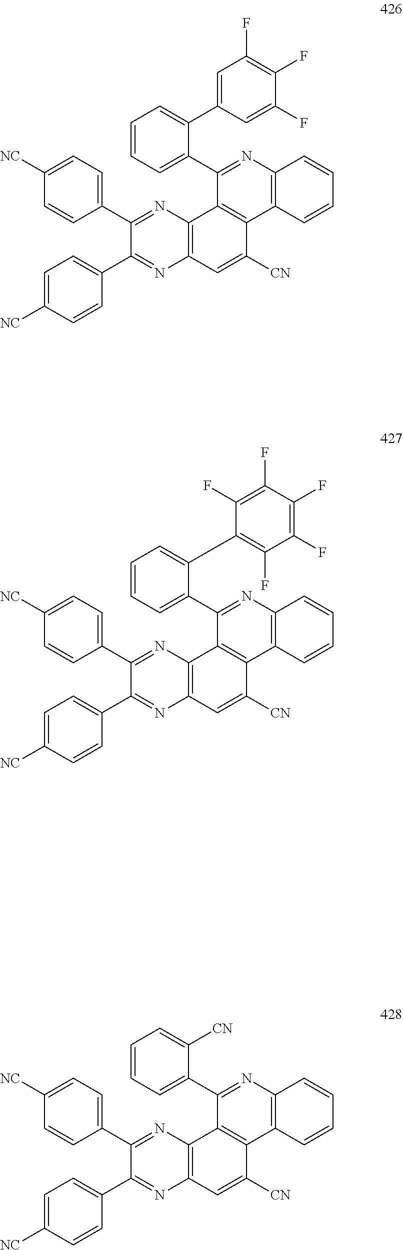 [SMILES: N#Cc1ccc(-c2nc3cc(C#N)c4c5ccccc5nc(-c5ccccc5-c5cc(F)c(F)c(F)c5)c4c3nc2-c2ccc(C#N)cc2)cc1 ; N#Cc1ccc(-c2nc3cc(C#N)c4c5ccccc5nc(-c5ccccc5-c5c(F)c(F)c(F)c(F)c5F)c4c3nc2-c2ccc(C#N)cc2)cc1 ; N#Cc1ccc(-c2nc3cc(C#N)c4c5ccccc5nc(-c5ccccc5C#N)c4c3nc2-c2ccc(C#N)cc2)cc1]

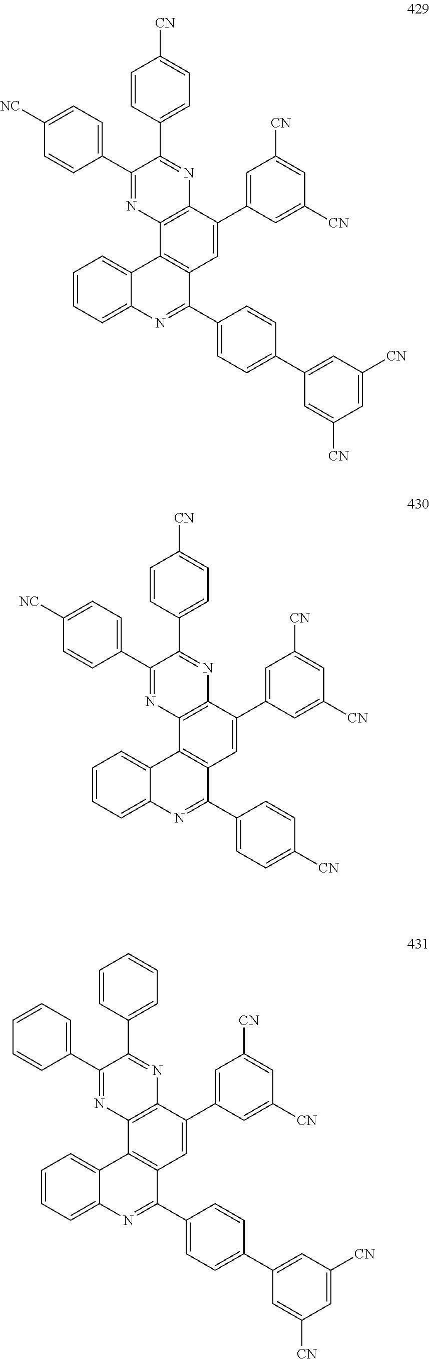 [SMILES: N#Cc1ccc(-c2nc3c(-c4cc(C#N)cc(C#N)c4)cc4c(-c5ccc(-c6cc(C#N)cc(C#N)c6)cc5)nc5ccccc5c4c3nc2-c2ccc(C#N)cc2)cc1 ; N#Cc1ccc(-c2nc3c(-c4cc(C#N)cc(C#N)c4)cc4c(-c5ccc(C#N)cc5)nc5ccccc5c4c3nc2-c2ccc(C#N)cc2)cc1 ; N#Cc1cc(C#N)cc(-c2ccc(-c3nc4ccccc4c4c3cc(-c3cc(C#N)cc(C#N)c3)c3nc(-c5ccccc5)c(-c5ccccc5)nc34)cc2)c1]

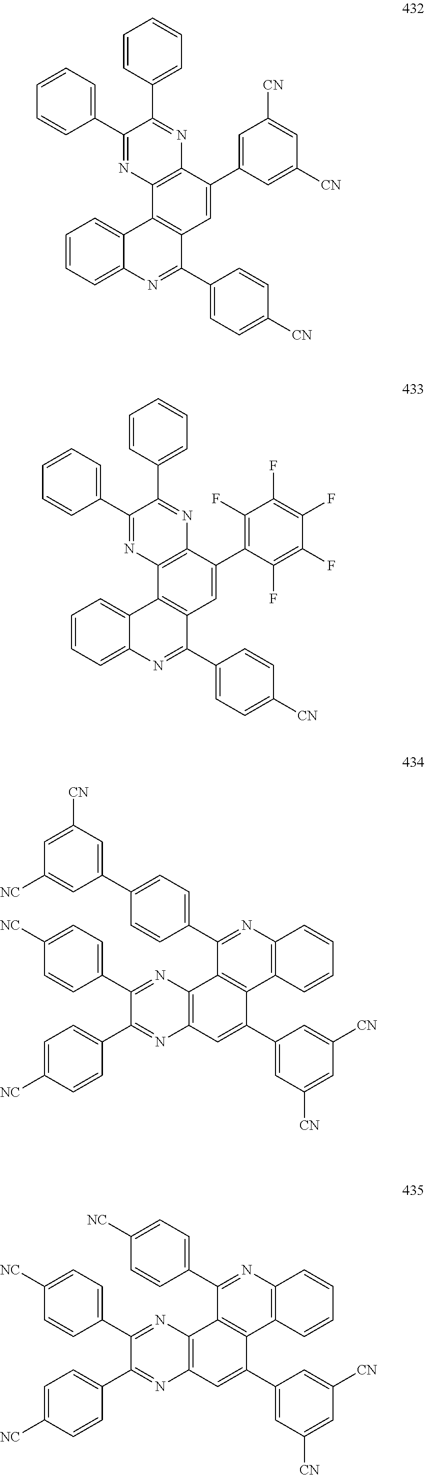 [SMILES: N#Cc1ccc(-c2nc3ccccc3c3c2cc(-c2cc(C#N)cc(C#N)c2)c2nc(-c4ccccc4)c(-c4ccccc4)nc23)cc1 ; N#Cc1ccc(-c2nc3ccccc3c3c2cc(-c2c(F)c(F)c(F)c(F)c2F)c2nc(-c4ccccc4)c(-c4ccccc4)nc23)cc1 ; N#Cc1ccc(-c2nc3cc(-c4cc(C#N)cc(C#N)c4)c4c5ccccc5nc(-c5ccc(-c6cc(C#N)cc(C#N)c6)cc5)c4c3nc2-c2ccc(C#N)cc2)cc1 ; N#Cc1ccc(-c2nc3cc(-c4cc(C#N)cc(C#N)c4)c4c5ccccc5nc(-c5ccc(C#N)cc5)c4c3nc2-c2ccc(C#N)cc2)cc1]

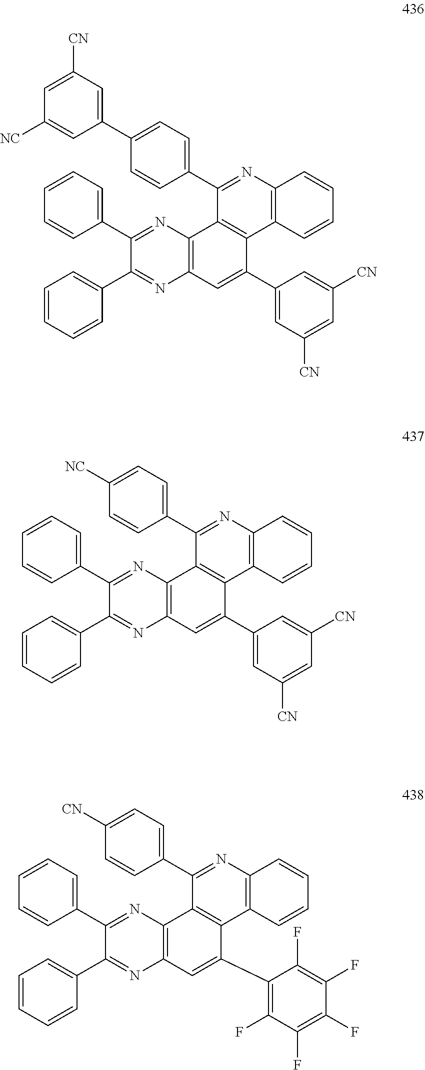

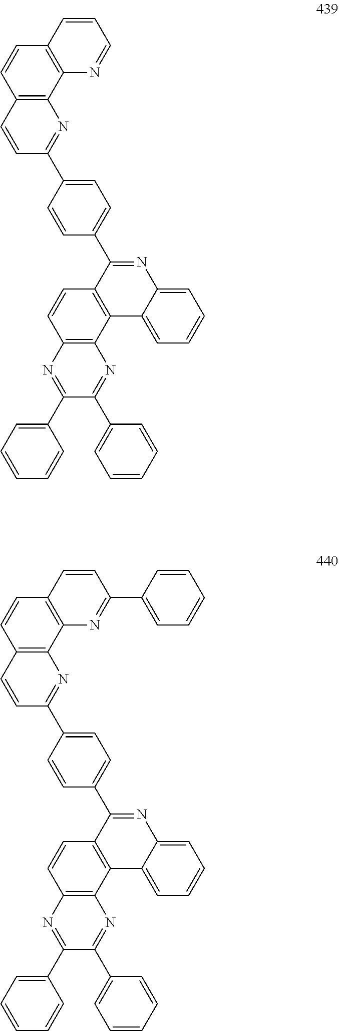 [SMILES: c1ccc(-c2nc3ccc4c(-c5ccc(-c6ccc7ccc8cccnc8c7n6)cc5)nc5ccccc5c4c3nc2-c2ccccc2)cc1 ; c1ccc(-c2ccc3ccc4ccc(-c5ccc(-c6nc7ccccc7c7c6ccc6nc(-c8ccccc8)c(-c8ccccc8)nc67)cc5)nc4c3n2)cc1]

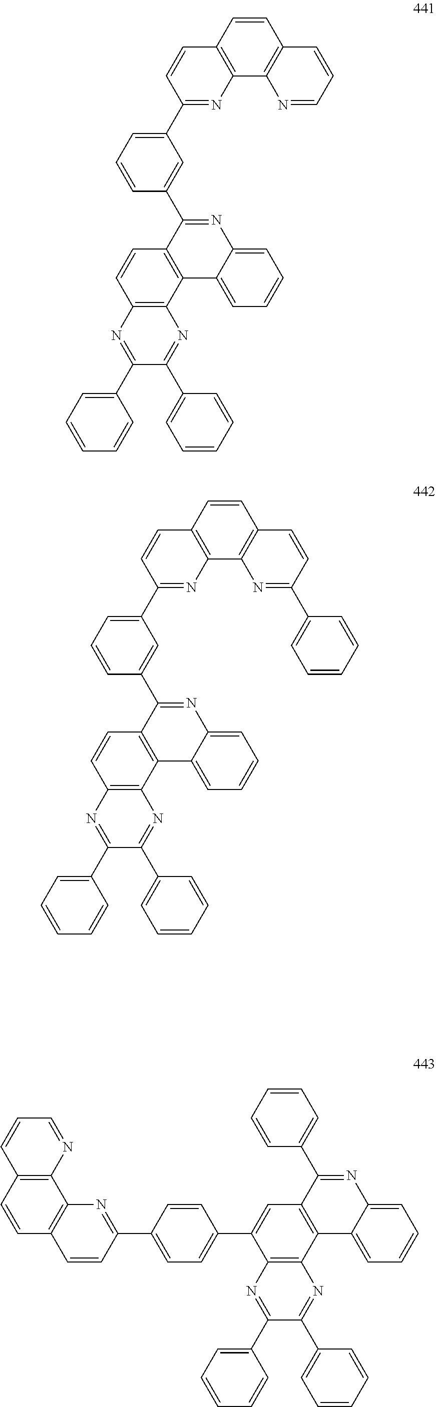 [SMILES: c1ccc(-c2nc3ccc4c(-c5cccc(-c6ccc7ccc8cccnc8c7n6)c5)nc5ccccc5c4c3nc2-c2ccccc2)cc1 ; c1ccc(-c2ccc3ccc4ccc(-c5cccc(-c6nc7ccccc7c7c6ccc6nc(-c8ccccc8)c(-c8ccccc8)nc67)c5)nc4c3n2)cc1 ; c1ccc(-c2nc3c(-c4ccc(-c5ccc6ccc7cccnc7c6n5)cc4)cc4c(-c5ccccc5)nc5ccccc5c4c3nc2-c2ccccc2)cc1]

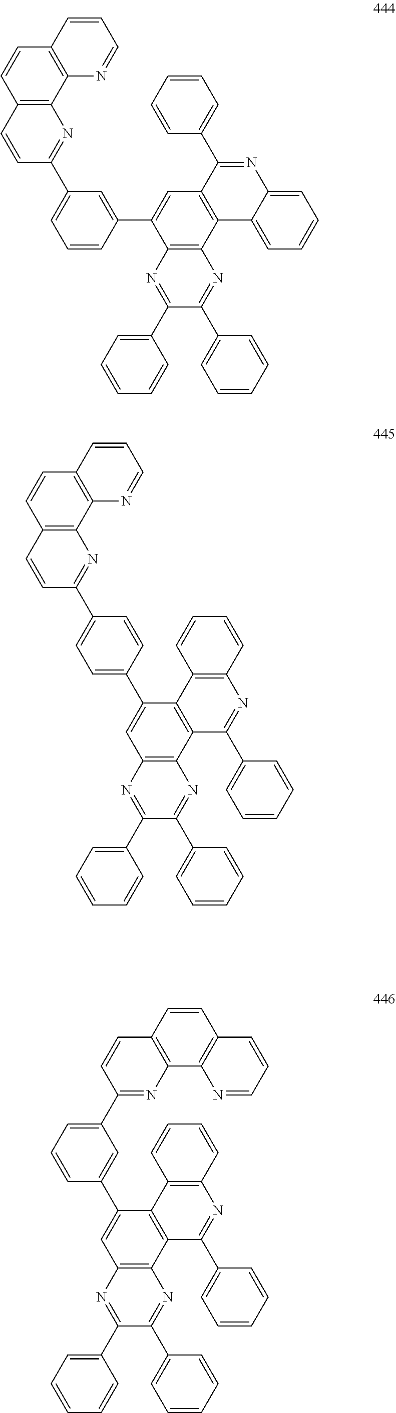 [SMILES: c1ccc(-c2nc3c(-c4cccc(-c5ccc6ccc7cccnc7c6n5)c4)cc4c(-c5ccccc5)nc5ccccc5c4c3nc2-c2ccccc2)cc1 ; c1ccc(-c2nc3cc(-c4ccc(-c5ccc6ccc7cccnc7c6n5)cc4)c4c5ccccc5nc(-c5ccccc5)c4c3nc2-c2ccccc2)cc1 ; c1ccc(-c2nc3cc(-c4cccc(-c5ccc6ccc7cccnc7c6n5)c4)c4c5ccccc5nc(-c5ccccc5)c4c3nc2-c2ccccc2)cc1]

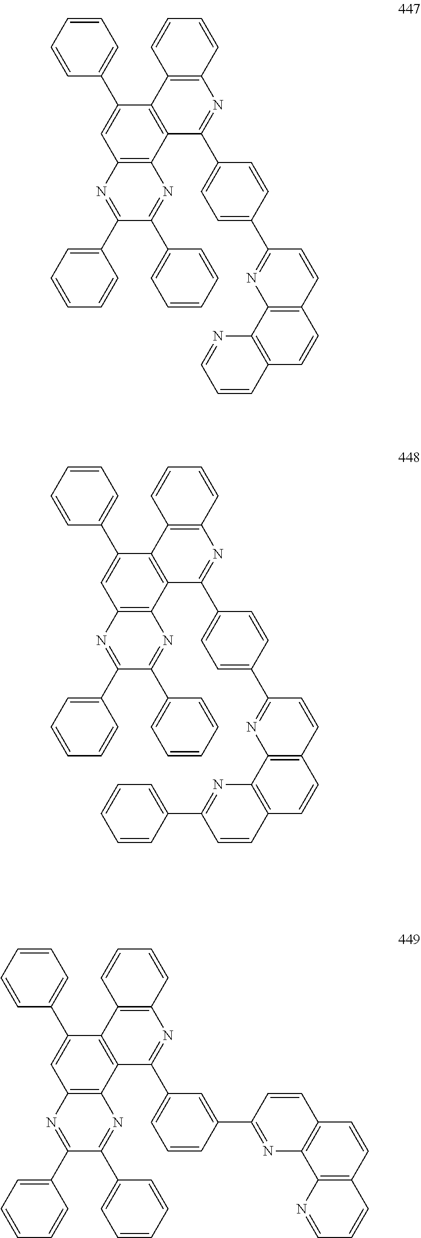 [SMILES: c1ccc(-c2nc3cc(-c4ccccc4)c4c5ccccc5nc(-c5ccc(-c6ccc7ccc8cccnc8c7n6)cc5)c4c3nc2-c2ccccc2)cc1 ; c1ccc(-c2ccc3ccc4ccc(-c5ccc(-c6nc7ccccc7c7c(-c8ccccc8)cc8nc(-c9ccccc9)c(-c9ccccc9)nc8c67)cc5)nc4c3n2)cc1 ; c1ccc(-c2nc3cc(-c4ccccc4)c4c5ccccc5nc(-c5cccc(-c6ccc7ccc8cccnc8c7n6)c5)c4c3nc2-c2ccccc2)cc1]

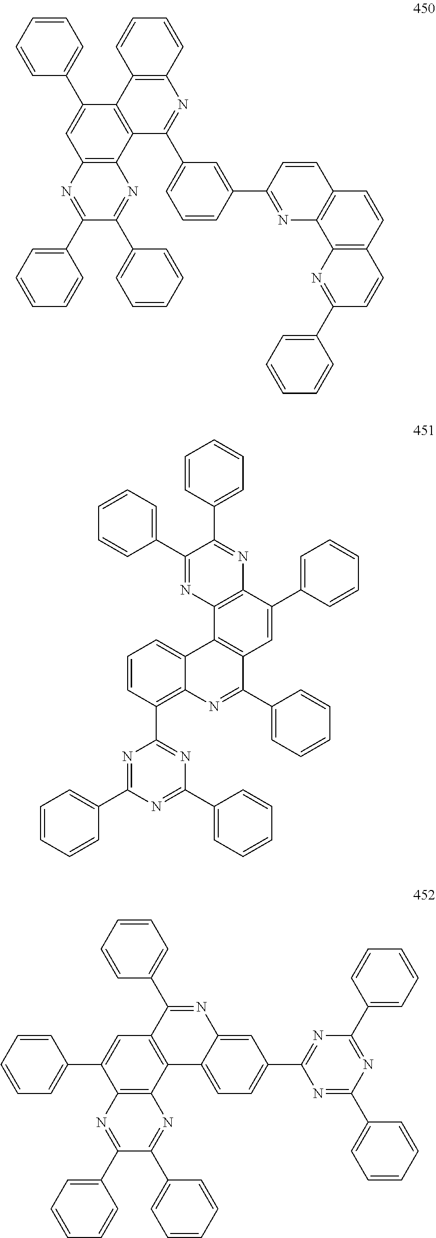 [SMILES: c1ccc(-c2ccc3ccc4ccc(-c5cccc(-c6nc7ccccc7c7c(-c8ccccc8)cc8nc(-c9ccccc9)c(-c9ccccc9)nc8c67)c5)nc4c3n2)cc1 ; c1ccc(-c2nc(-c3ccccc3)nc(-c3cccc4c3nc(-c3ccccc3)c3cc(-c5ccccc5)c5nc(-c6ccccc6)c(-c6ccccc6)nc5c34)n2)cc1 ; c1ccc(-c2nc(-c3ccccc3)nc(-c3ccc4c(c3)nc(-c3ccccc3)c3cc(-c5ccccc5)c5nc(-c6ccccc6)c(-c6ccccc6)nc5c34)n2)cc1]

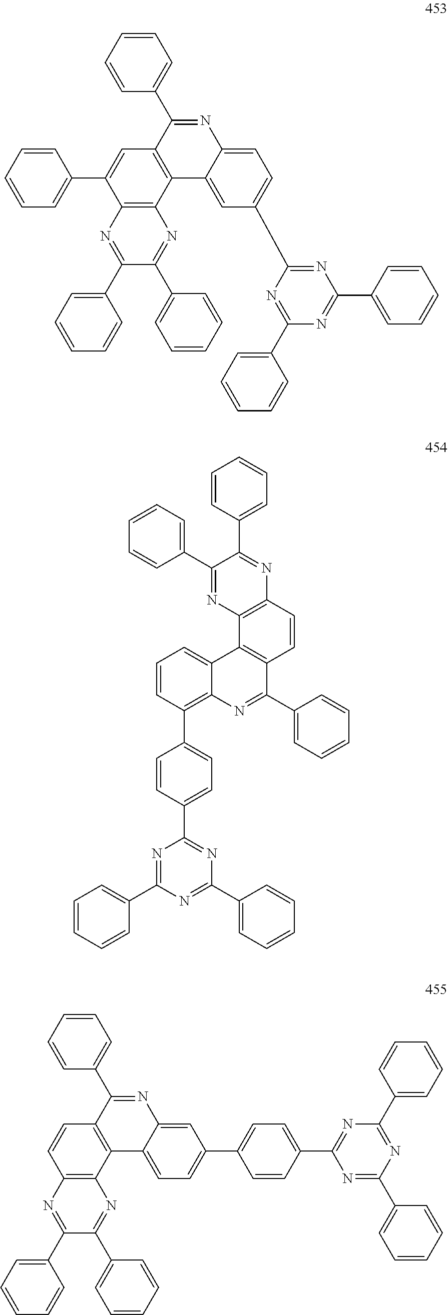 [SMILES: c1ccc(-c2nc(-c3ccccc3)nc(-c3ccc4nc(-c5ccccc5)c5cc(-c6ccccc6)c6nc(-c7ccccc7)c(-c7ccccc7)nc6c5c4c3)n2)cc1 ; c1ccc(-c2nc(-c3ccccc3)nc(-c3ccc(-c4cccc5c4nc(-c4ccccc4)c4ccc6nc(-c7ccccc7)c(-c7ccccc7)nc6c45)cc3)n2)cc1 ; c1ccc(-c2nc(-c3ccccc3)nc(-c3ccc(-c4ccc5c(c4)nc(-c4ccccc4)c4ccc6nc(-c7ccccc7)c(-c7ccccc7)nc6c45)cc3)n2)cc1]

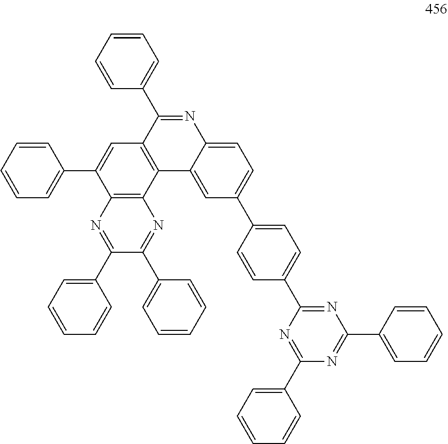 [SMILES: c1ccc(-c2nc(-c3ccccc3)nc(-c3ccc(-c4ccc5nc(-c6ccccc6)c6cc(-c7ccccc7)c7nc(-c8ccccc8)c(-c8ccccc8)nc7c6c5c4)cc3)n2)cc1]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the compound has a high glass transition temperature (Tg), and has excellent thermal stability. Such an increase in the thermal stability becomes an important factor providing driving stability to a device.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In one embodiment of the present application, the first electrode may be an anode, and the second electrode may be a cathode.

In another embodiment, the first electrode may be a cathode, and the second electrode may be an anode.

Specific details on the heterocyclic compound represented by Chemical Formula 1 are the same as the descriptions provided above.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound according to Chemical Formula 1 may be used as a material of the green organic light emitting device.

In one embodiment of the present application, the organic light emitting device may be a red organic light emitting device, and the heterocyclic compound according to Chemical Formula 1 may be used as a material of the red organic light emitting device.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, or may also be formed in a multilayer structure in which two or more organic material layers are laminated. For example, the organic light emitting device according to one embodiment of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise a smaller number of organic material layers.

In the organic light emitting device of the present disclosure, the organic material layer comprises an electron injection layer or an electron transfer layer, and the electron injection layer or the electron transfer layer may comprise the heterocyclic compound.

In the organic light emitting device of the present disclosure, the organic material layer comprises an electron transfer layer, and the electron transfer layer may comprise the heterocyclic compound.

In another organic light emitting device, the organic material layer comprises an electron blocking layer or a hole blocking layer, and the electron blocking layer or the hole blocking layer may comprise the heterocyclic compound.

In another organic light emitting device, the organic material layer comprises a hole blocking layer, and the hole blocking layer may comprise the heterocyclic compound.

In one embodiment of the present application, the organic material layer comprises a hole injection layer, and the hole injection layer may comprise the heterocyclic compound.

In another organic light emitting device, the organic material layer comprises an electron transfer layer, a light emitting layer or a hole blocking layer, and the electron transfer layer, the light emitting layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

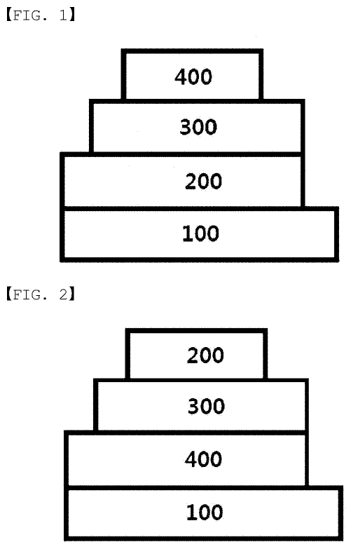

FIG. 1 to FIG. 4 illustrate a lamination order of electrodes and organic material layers of an organic light emitting device according to one embodiment of the present application. However, the scope of the present application is not limited to these diagrams, and structures of organic light emitting devices known in the art may also be used in the present application.

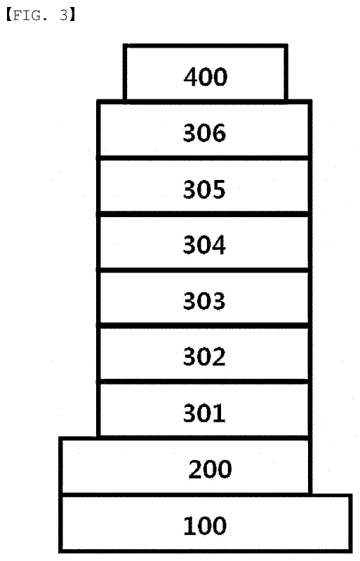

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

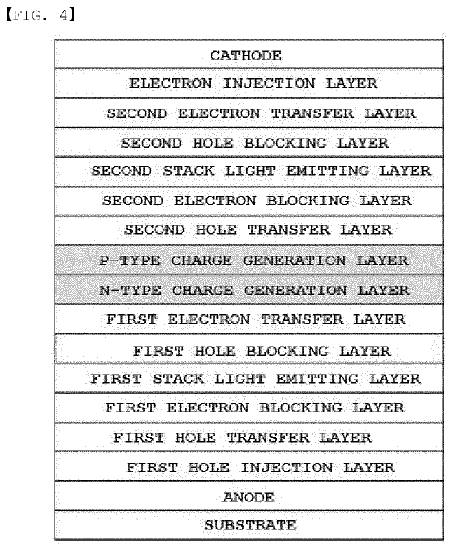

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer comprising Chemical Formula 1 may further comprise other materials as necessary.

In addition, the organic light emitting device according to one embodiment of the present application comprises an anode, a cathode, and two or more stacks provided between the anode and the cathode, wherein the two or more stacks each independently comprise a light emitting layer, a charge generation layer is included between the two or more stacks, and the charge generation layer comprises the heterocyclic compound represented by Chemical Formula 1.

In addition, the organic light emitting device according to one embodiment of the present application comprises an anode, a first stack provided on the anode and comprising a first light emitting layer, a charge generation layer provided on the first stack, a second stack provided on the charge generation layer and comprising a second light emitting layer, and a cathode provided on the second stack. Herein, the charge generation layer may comprise the heterocyclic compound represented by Chemical Formula 1. In addition, the first stack and the second stack may each independently further comprise one or more types of the hole injection layer, the hole transfer layer, the hole blocking layer, the electron transfer layer, the electron injection layer and the like described above.

In the organic light emitting device provided in one embodiment of the present application, the charge generation layer is an N-type charge generation layer, and the charge generation layer comprises the heterocyclic compound.

The charge generation layer may be an N-type charge generation layer, and the charge generation layer may further comprise a dopant known in the art in addition to the heterocyclic compound represented by Chemical Formula 1.

As the organic light emitting device according to one embodiment of the present application, an organic light emitting device having a 2-stack tandem structure is schematically illustrated in FIG. 4.

Herein, the first electron blocking layer, the first hole blocking layer, the second hole blocking layer and the like described in FIG. 4 may not be included in some cases.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### PREPARATION EXAMPLE

### [Preparation Example 1] Preparation of Intermediate A1

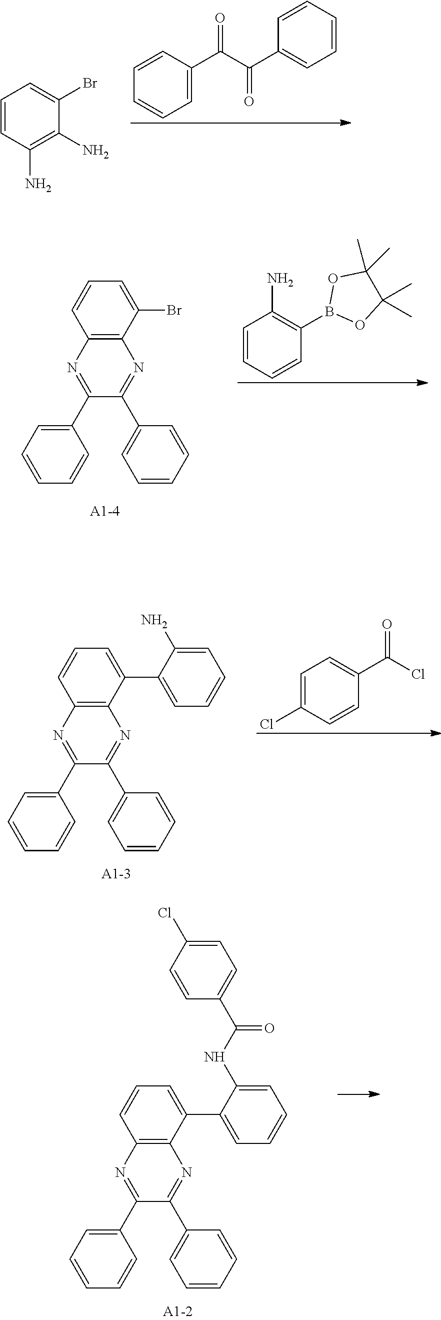

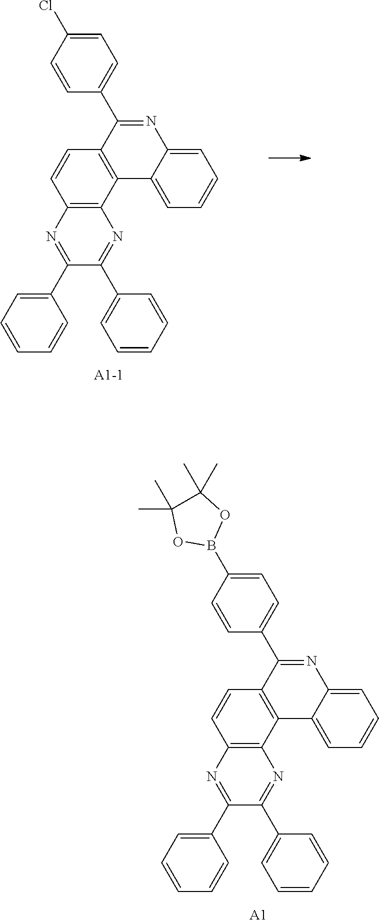

### Preparation Example 1-1 (Preparation of Intermediate A1-4)

To a one-neck round bottom flask, 3-bromobenzene-1,2-diamine (50 g, 267.32 mmol), diphenylethanedione (56.2 g, 267.32 mmol), vanadyl sulfate (4.61 g, 8.02 mmol) and EtOH (800 ml) were introduced, and stirred for 6 hours. After the reaction was finished, the result was extracted with methylene chloride (MC) and H₂O, and, after removing the solvent, purified by column chromatography using dichloromethane and hexane as a developing solvent to obtain Intermediate A1-4 (84 g, 87%).

### Preparation Example 1-2 (Preparation of Intermediate A1-3)

To a one-neck round bottom flask, Intermediate A1-4 (83.1 g, 230.04 mmol), 2-(4,4,5,5-tetramethyl-1,3,2-dioxaborolan-2-yl)aniline (42 g, 191.70 mmol), K₂CO₃ (79.49 g, 575.11 mmol), Pd(PPh₃)₄ (6.65 g, 5.75 mmol), toluene (600 ml), EtOH (120 ml) and H₂O (120 ml) were introduced, and stirred for 14 hours under reflux. After the reaction was finished, the result was extracted with methylene chloride (MC) and H₂O, and, after removing the solvent, purified by column chromatography using dichloromethane and hexane as a developing solvent to obtain Intermediate A1-3 (40 g, 56%).

### Preparation Example 1-3 (preparation of Intermediate A1-2)

After dissolving Intermediate A1-3 (40 g, 107.11 mmol) in methylene chloride (MC) (400 ml) in a one-neck round bottom flask, triethylamine (TEA) (32.52 g, 321.33 mmol) was introduced thereto. After lowering the temperature from room temperature to 0° C., 4-chlorobenzoyl chloride (20.62 g, 117.82 mmol) dissolved in methylene chloride (MC) was slowly added dropwise thereto. After the reaction was completed, the result was extracted with methylene chloride (MC) and distilled water. After drying the organic layer with anhydrous MgSO₄, the solvent was removed using a rotary evaporator, and the result was purified by column chromatography using dichloromethane and hexane as a developing solvent to obtain Intermediate A1-2 (40 g, 73%).

### Preparation Example 1-4 (Preparation of Intermediate A1-1)

To a one-neck round bottom flask, Intermediate A1-2 (40 g, 78.13 mmol), POCl₃ (13.18 g, 85.94 mmol) and nitrobenzene (400 ml) were introduced, and stirred for 6 hours under reflux. After the reaction was completed, the result was neutralized using an aqueous NaHCO₃ solution, and then extracted with methylene chloride (MC) and distilled water. After drying the organic layer with anhydrous MgSO₄, the solvent was removed using a rotary evaporator, and the result was purified by column chromatography using dichloromethane and hexane as a developing solvent to obtain Intermediate A1-1 (32 g, 83%).

### Preparation Example 1-5 (Preparation of Intermediate A1)

To a one-neck round bottom flask, Intermediate A1-1 (32 g, 64.78 mmol), bis(pinacolato)diboron (21.38 g, 84.21 mmol), KOAc (19.07 g, 194.34 mmol), Pd(dba)₂ (1.86 g, 3.24 mmol), Xphos (3.09 g, 6.48 mmol) and 1,4-dioxane (300 ml) were introduced, and stirred for 6 hours under reflux. After the reaction was finished, the result was extracted with methylene chloride (MC) and H₂O, and, after removing the solvent, purified by column chromatography using dichloromethane and hexane as a developing solvent to obtain Intermediate A1 (33 g, 87%).

Intermediates were synthesized in the same manner as in Preparation Example 1 except that Si of the following Table 1 was used instead of 3-bromobenzene-1,2-diamine, S2 of the following Table 1 was used instead of benzyl, and S3 of the following Table 1 was used instead of 4-chlorobenzoyl chloride.

TABLE 1

| Intermediate | S1 | S2 | S3 | compound | Yield |
|---|---|---|---|---|---|
| A2 | 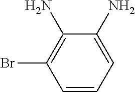 | 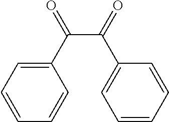 | 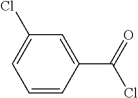 | 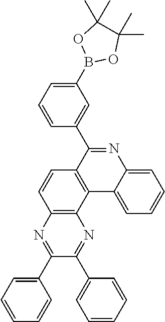 [SMILES: CC1(C)OB(c2cccc(-c3nc4ccccc4c4c3ccc3nc(-c5ccccc5)c(-c5ccccc5)nc34)c2)OC1(C)C] | 81% |
| A3 | 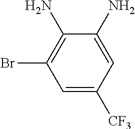 | 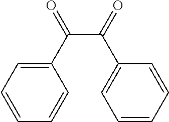 | 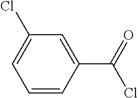 | 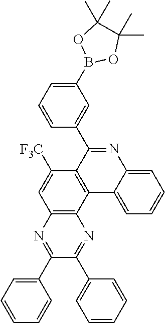 [SMILES: CC1(C)OB(c2cccc(-c3nc4ccccc4c4c3c(C(F)(F)F)cc3nc(-c5ccccc5)c(-c5ccccc5)nc34)c2)OC1(C)C] | 75% |
| A4 | 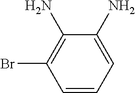 | 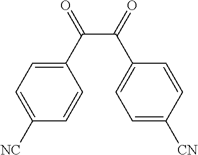 | 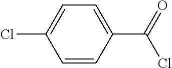 | 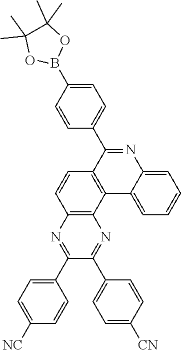 [SMILES: CC1(C)OB(c2ccc(-c3nc4ccccc4c4c3ccc3nc(-c5ccc(C#N)cc5)c(-c5ccc(C#N)cc5)nc34)cc2)OC1(C)C] | 85% |
| A5 | 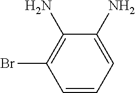 | 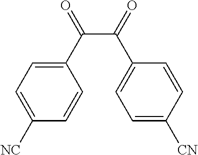 | 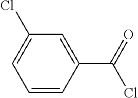 | 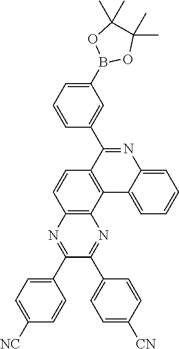 [SMILES: CC1(C)OB(c2cccc(-c3nc4ccccc4c4c3ccc3nc(-c5ccc(C#N)cc5)c(-c5ccc(C#N)cc5)nc34)c2)OC1(C)C] | 80% |
| A6 | 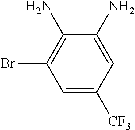 | 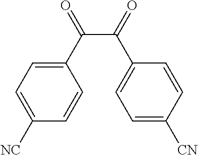 | 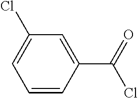 | 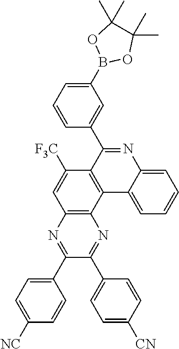 [SMILES: CC1(C)OB(c2cccc(-c3nc4ccccc4c4c3c(C(F)(F)F)cc3nc(-c5ccc(C#N)cc5)c(-c5ccc(C#N)cc5)nc34)c2)OC1(C)C] | 86% |
| A7 | 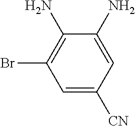 | 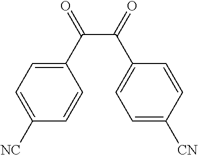 | 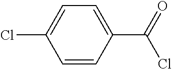 | 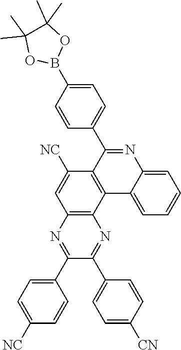 [SMILES: CC1(C)OB(c2ccc(-c3nc4ccccc4c4c3c(C#N)cc3nc(-c5ccc(C#N)cc5)c(-c5ccc(C#N)cc5)nc34)cc2)OC1(C)C] | 79% |

### [Preparation Example 2] Preparation of Intermediate B1

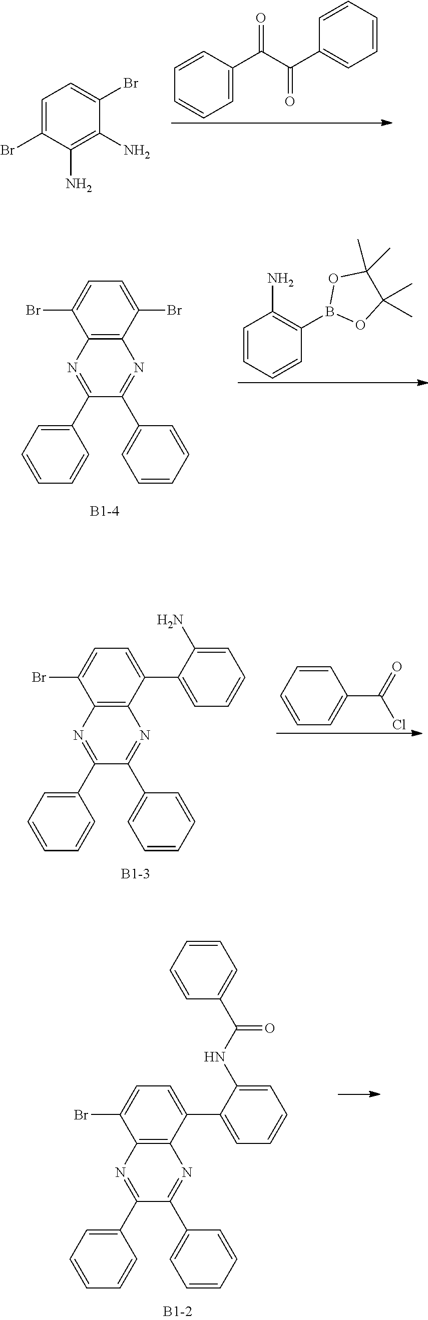

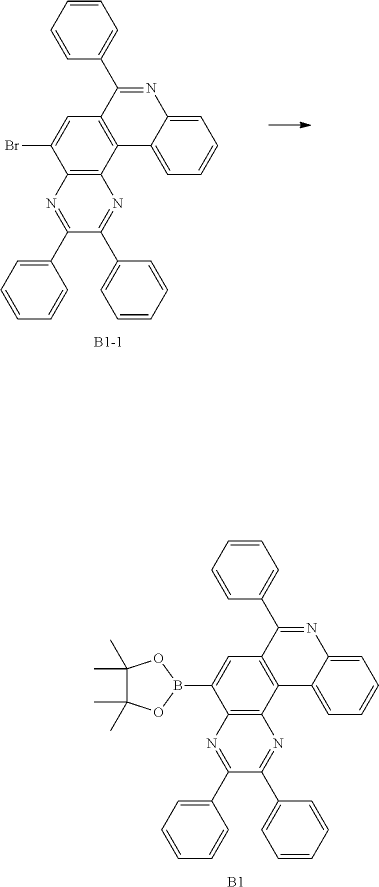

### Preparation of Intermediate B1-4

Intermediate B1-4 was synthesized in the same manner as in Preparation Example 1-1 except that 3,6-dibromobenzene-1,2-diamine was used instead of 3-bromobenzene-1,2-diamine.

### Preparation of Intermediate B1-3

Intermediate B1-3 was synthesized in the same manner as in Preparation Example 1-2 except that Intermediate B1-4 was used instead of Intermediate A1-4.

### Preparation of Intermediate B1-2

Intermediate B1-2 was synthesized in the same manner as in Preparation Example 1-3 except that Intermediate B1-3 was used instead of Intermediate A1-3, and benzoyl chloride was used instead of 4-chlorobenzoyl chloride.

### Preparation of Intermediate B1-1

Intermediate B1-1 was synthesized in the same manner as in Preparation Example 1-4 except that Intermediate B1-2 was used instead of Intermediate A1-2.

### Preparation of Intermediate B1

To a one-neck round bottom flask, Intermediate B1-1 (30 g, 55.72 mmol), bis(pinacolato)diboron (21.22 g, 83.57 mmol), KOAc (16.40 g, 167.15 mmol), Pd(dppf)Cl₂ (1.63 g, 2.23 mmol) and 1,4-dioxane (300 ml) were introduced, and stirred for 6 hours under reflux. After the reaction was finished, the result was extracted with methylene chloride (MC) and H₂O, and, after removing the solvent, purified by column chromatography using dichloromethane and hexane as a developing solvent to obtain Intermediate B1 (27 g, 83%).

Intermediates were synthesized in the same manner as in Preparation Example 2 except that S4 of the following Table 2 was used instead of 3-bromobenzene-1,2-diamine, S5 of the following Table 2 was used instead of benzyl, and S6 of the following Table 2 was used instead of benzoyl chloride.

TABLE 2

| Intermediate | S4 | S5 | S6 | compound | Yield |
|---|---|---|---|---|---|
| B2 | 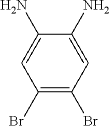 | 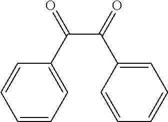 | 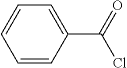 | 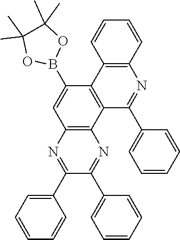 [SMILES: CC1(C)OB(c2cc3nc(-c4ccccc4)c(-c4ccccc4)nc3c3c(-c4ccccc4)nc4ccccc4c23)OC1(C)C] | 77% |
| B3 | 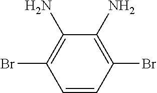 | 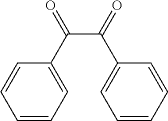 | 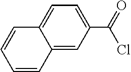 | 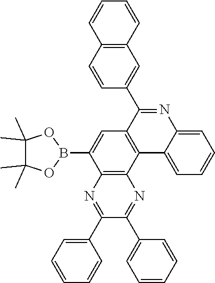 [SMILES: CC1(C)OB(c2cc3c(-c4ccc5ccccc5c4)nc4ccccc4c3c3nc(-c4ccccc4)c(-c4ccccc4)nc23)OC1(C)C] | 78% |
| B4 | 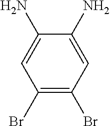 | 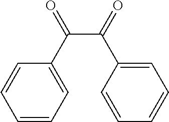 | 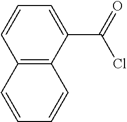 | 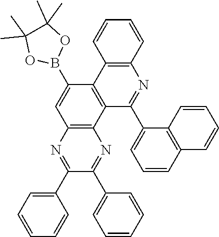 [SMILES: CC1(C)OB(c2cc3nc(-c4ccccc4)c(-c4ccccc4)nc3c3c(-c4cccc5ccccc45)nc4ccccc4c23)OC1(C)C] | 81% |
| B5 | 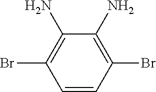 | 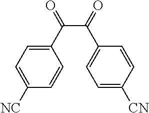 | 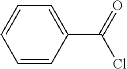 | 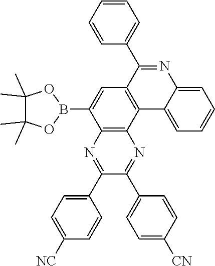 [SMILES: CC1(C)OB(c2cc3c(-c4ccccc4)nc4ccccc4c3c3nc(-c4ccc(C#N)cc4)c(-c4ccc(C#N)cc4)nc23)OC1(C)C] | 80% |

### [Preparation Example 3] Preparation of Intermediate C1

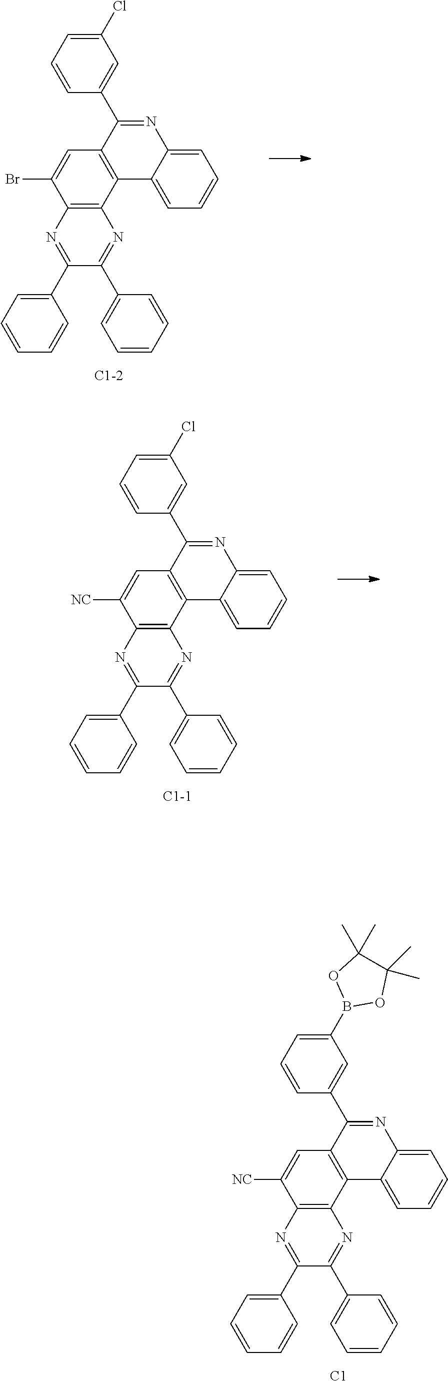

Intermediate C1-2 was synthesized in the same manner as in Preparation Example 2 except that 3-chlorobenzoyl chloride was used instead of benzoyl chloride in the preparations of Intermediates B1-4 to B1-1.

### Preparation of Intermediate C1-1

To a one-neck round bottom flask, Intermediate C1-2 (30 g, 52.37 mmol), zinc cyanide (3.07 g, 26.18 mmol), Pd(PPh₃)₄ (1.82 g, 1.57 mmol) and dimethylacetamide (300 ml) were introduced, and stirred for 3 hours under reflux. After the reaction was finished, water (300 ml) was introduced thereto, and white solids were filtered. The filtered white solids were washed twice each with ethanol and water to obtain Intermediate C1-1 (222 g, 81%).

### Preparation of Intermediate C1

Intermediate C1 was synthesized in the same manner as in Preparation Example 2 except that Intermediate C1-1 was used instead of Intermediate B1-1.

Intermediates were synthesized in the same manner as in Preparation Example 3 except that S7 of the following Table 3 was used instead of 3-chlorobenzoyl chloride.

TABLE 3

| Intermediate | S7 | compound | Yield |
|---|---|---|---|
| C2 | 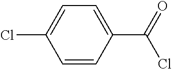 | 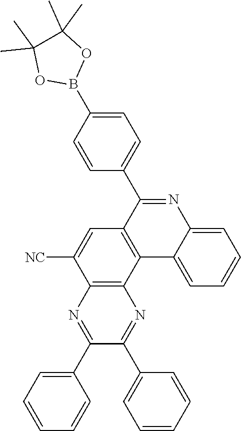 [SMILES: CC1(C)OB(c2ccc(-c3nc4ccccc4c4c3cc(C#N)c3nc(-c5ccccc5)c(-c5ccccc5)nc34)cc2)OC1(C)C] | 79% |

### [Preparation Example 4] Preparation of Intermediate D1

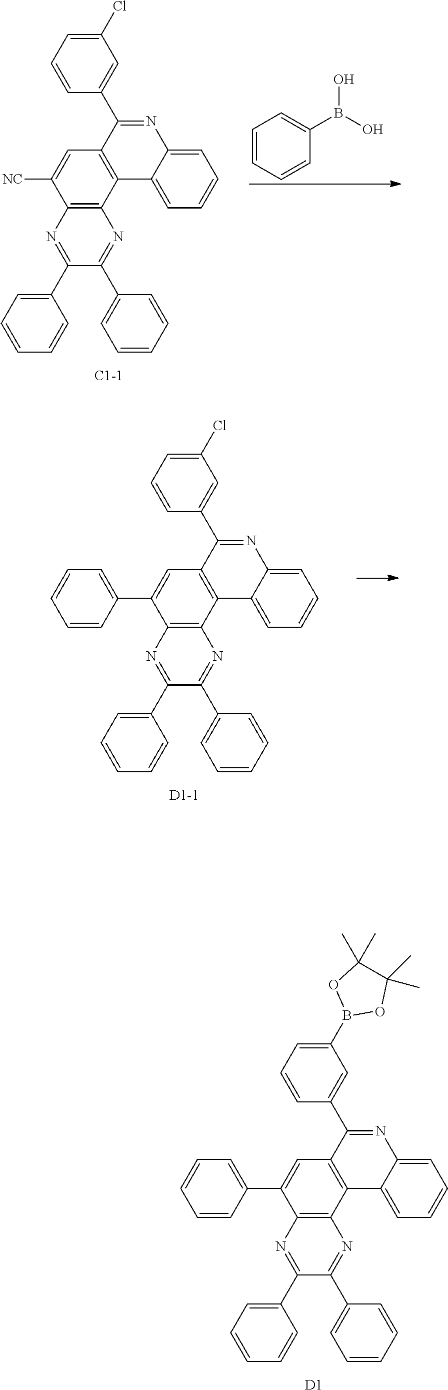

### Preparation of Intermediate D1-1

To a one-neck round bottom flask, Intermediate C1-1 (25 g, 43.64 mmol), phenylboronic acid (5.85 g, 48.00 mmol), K₂CO₃ (18.09 g, 130.92 mmol), Pd(PPh₃)₄ (1.51 g, 1.31 mmol), toluene (200 ml), EtOH (40 ml) and H₂O (40 ml) were introduced, and stirred for 14 hours under reflux. After the reaction was finished, the result was extracted with methylene chloride (MC) and H₂O, and, after removing the solvent, purified by column chromatography using dichloromethane and hexane as a developing solvent to obtain Intermediate D1-1 (20 g, 80%).

### Preparation of Intermediate D1

Intermediate D1 was synthesized in the same manner as in Preparation Example 2 except that Intermediate D1-1 was used instead of Intermediate B1-1.

### [Preparation Example 5] Preparation of Compound 001

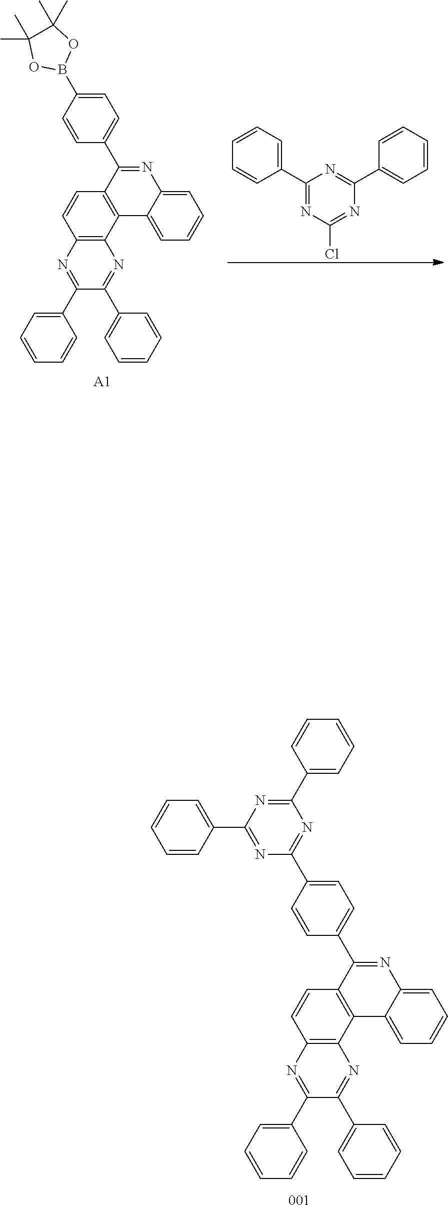

### Preparation of Compound 001

To a one-neck round bottom flask, Intermediate A1 (10 g, 17.08 mmol), 2-chloro-4,6-diphenyl-1,3,5-triazine (5.03 g, 18.79 mmol), K₂CO₃ (7.08 g, 51.24 mmol), Pd(PPh₃)₄ (0.59 g, 0.51 mmol), toluene (100 ml), EtOH (20 ml) and H₂O (20 ml) were introduced, and stirred for 8 hours under reflux. After the reaction was finished, the result was extracted with methylene chloride (MC) and H₂O, and, after removing the solvent, purified by column chromatography using dichloromethane and hexane as a developing solvent to obtain Compound 001 (9 g, 76%).

Final compounds were synthesized in the same manner as in Preparation Example 5 except that intermediates of the following Table 4 were used instead of Intermediate A1, and S8 of the following Table 4 was used instead of 2-chloro-4,6-diphenyl-1,3,5-triazine.

TABLE 4

| Compound | Intermediate | S8 | compound | Yield |
|---|---|---|---|---|
| 002 | 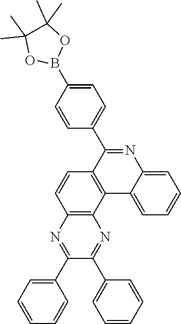 [SMILES: CC1(C)OB(c2ccc(-c3nc4ccccc4c4c3ccc3nc(-c5ccccc5)c(-c5ccccc5)nc34)cc2)OC1(C)C] | 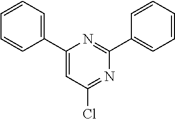 | 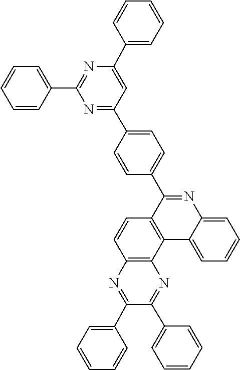 [SMILES: c1ccc(-c2cc(-c3ccc(-c4nc5ccccc5c5c4ccc4nc(-c6ccccc6)c(-c6ccccc6)nc45)cc3)nc(-c3ccccc3)n2)cc1] | 79% |
| 003 | 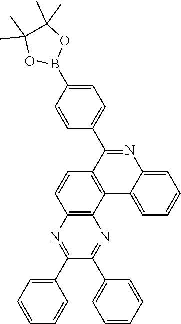 [SMILES: CC1(C)OB(c2ccc(-c3nc4ccccc4c4c3ccc3nc(-c5ccccc5)c(-c5ccccc5)nc34)cc2)OC1(C)C] | 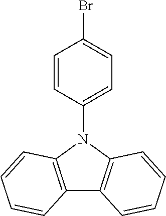 | 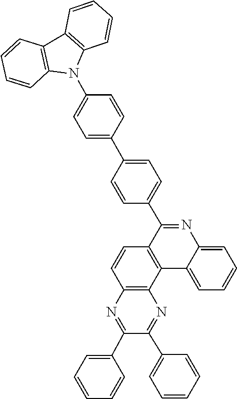 [SMILES: c1ccc(-c2nc3ccc4c(-c5ccc(-c6ccc(-n7c8ccccc8c8ccccc87)cc6)cc5)nc5ccccc5c4c3nc2-c2ccccc2)cc1] | 80% |
| 004 | 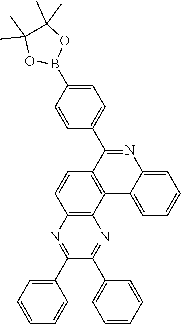 [SMILES: CC1(C)OB(c2ccc(-c3nc4ccccc4c4c3ccc3nc(-c5ccccc5)c(-c5ccccc5)nc34)cc2)OC1(C)C] | 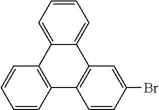 | 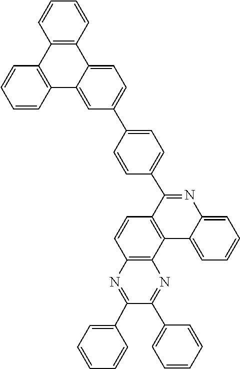 [SMILES: c1ccc(-c2nc3ccc4c(-c5ccc(-c6ccc7c8ccccc8c8ccccc8c7c6)cc5)nc5ccccc5c4c3nc2-c2ccccc2)cc1] | 77% |
| 007 | 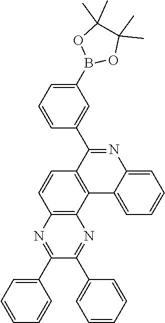 [SMILES: CC1(C)OB(c2cccc(-c3nc4ccccc4c4c3ccc3nc(-c5ccccc5)c(-c5ccccc5)nc34)c2)OC1(C)C] | 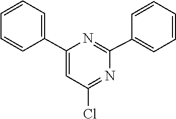 | 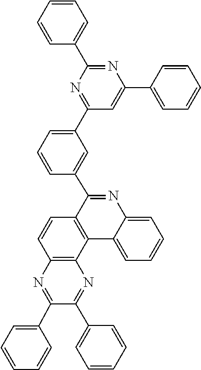 [SMILES: c1ccc(-c2cc(-c3cccc(-c4nc5ccccc5c5c4ccc4nc(-c6ccccc6)c(-c6ccccc6)nc45)c3)nc(-c3ccccc3)n2)cc1] | 71% |
| 009 | 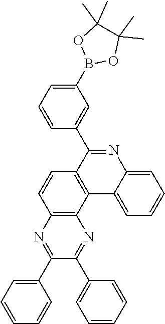 [SMILES: CC1(C)OB(c2cccc(-c3nc4ccccc4c4c3ccc3nc(-c5ccccc5)c(-c5ccccc5)nc34)c2)OC1(C)C] | 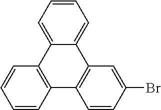 | 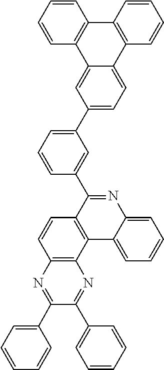 [SMILES: c1ccc(-c2nc3ccc4c(-c5cccc(-c6ccc7c8ccccc8c8ccccc8c7c6)c5)nc5ccccc5c4c3nc2-c2ccccc2)cc1] | 69% |
| 010 | 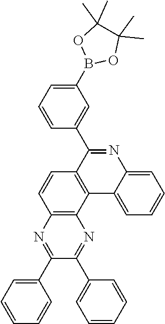 [SMILES: CC1(C)OB(c2cccc(-c3nc4ccccc4c4c3ccc3nc(-c5ccccc5)c(-c5ccccc5)nc34)c2)OC1(C)C] | 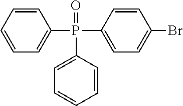 | 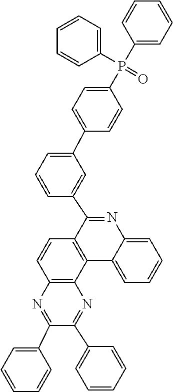 [SMILES: O=P(c1ccccc1)(c1ccccc1)c1ccc(-c2cccc(-c3nc4ccccc4c4c3ccc3nc(-c5ccccc5)c(-c5ccccc5)nc34)c2)cc1] | 82% |
| 031 | 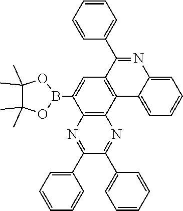 [SMILES: CC1(C)OB(c2cc3c(-c4ccccc4)nc4ccccc4c3c3nc(-c4ccccc4)c(-c4ccccc4)nc23)OC1(C)C] | 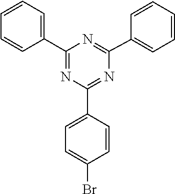 | 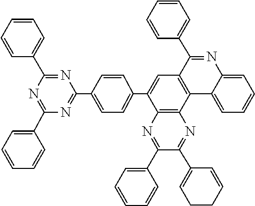 [SMILES: C1=CC(c2nc3c(nc2-c2ccccc2)c(-c2ccc(-c4nc(-c5ccccc5)nc(-c5ccccc5)n4)cc2)cc2c(-c4ccccc4)nc4ccccc4c23)=CCC1] | 79% |
| 032 | 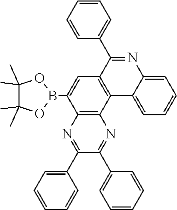 [SMILES: CC1(C)OB(c2cc3c(-c4ccccc4)nc4ccccc4c3c3nc(-c4ccccc4)c(-c4ccccc4)nc23)OC1(C)C] | 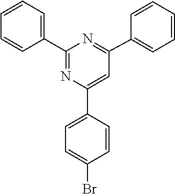 | 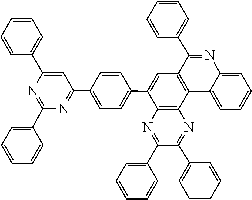 [SMILES: C1=CC(c2nc3c(nc2-c2ccccc2)c(-c2ccc(-c4cc(-c5ccccc5)nc(-c5ccccc5)n4)cc2)cc2c(-c4ccccc4)nc4ccccc4c23)=CCC1] | 80% |
| 033 | 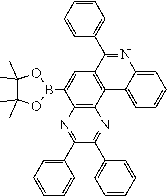 [SMILES: CC1(C)OB(c2cc3c(-c4ccccc4)nc4ccccc4c3c3nc(-c4ccccc4)c(-c4ccccc4)nc23)OC1(C)C] | 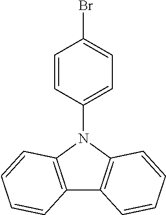 | 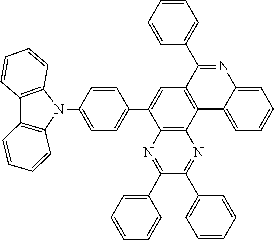 [SMILES: c1ccc(-c2nc3c(-c4ccc(-n5c6ccccc6c6ccccc65)cc4)cc4c(-c5ccccc5)nc5ccccc5c4c3nc2-c2ccccc2)cc1] | 82% |
| 056 | 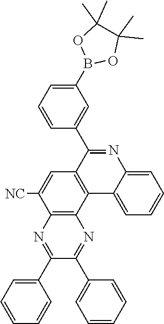 [SMILES: CC1(C)OB(c2cccc(-c3nc4ccccc4c4c3cc(C#N)c3nc(-c5ccccc5)c(-c5ccccc5)nc34)c2)OC1(C)C] | 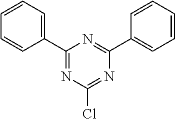 | 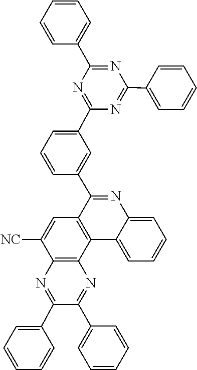 [SMILES: N#Cc1cc2c(-c3cccc(-c4nc(-c5ccccc5)nc(-c5ccccc5)n4)c3)nc3ccccc3c2c2nc(-c3ccccc3)c(-c3ccccc3)nc12] | 79% |
| 058 | 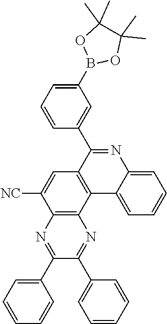 [SMILES: CC1(C)OB(c2cccc(-c3nc4ccccc4c4c3cc(C#N)c3nc(-c5ccccc5)c(-c5ccccc5)nc34)c2)OC1(C)C] | 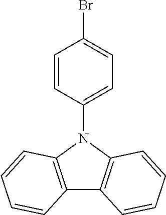 | 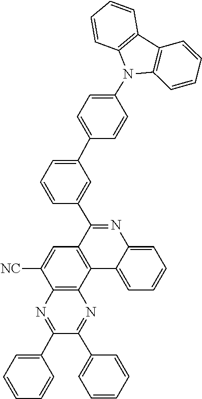 [SMILES: N#Cc1cc2c(-c3cccc(-c4ccc(-n5c6ccccc6c6ccccc65)cc4)c3)nc3ccccc3c2c2nc(-c3ccccc3)c(-c3ccccc3)nc12] | 74% |
| 086 | 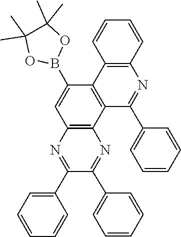 [SMILES: CC1(C)OB(c2cc3nc(-c4ccccc4)c(-c4ccccc4)nc3c3c(-c4ccccc4)nc4ccccc4c23)OC1(C)C] | 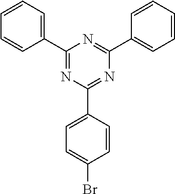 | 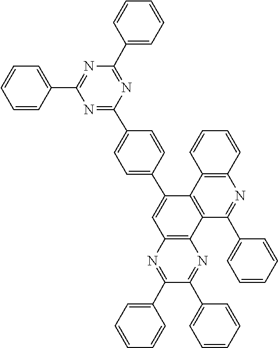 [SMILES: c1ccc(-c2nc(-c3ccccc3)nc(-c3ccc(-c4cc5nc(-c6ccccc6)c(-c6ccccc6)nc5c5c(-c6ccccc6)nc6ccccc6c45)cc3)n2)cc1] | 70% |
| 121 | 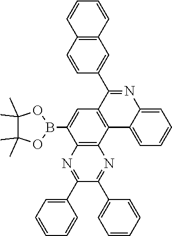 [SMILES: CC1(C)OB(c2cc3c(-c4ccc5ccccc5c4)nc4ccccc4c3c3nc(-c4ccccc4)c(-c4ccccc4)nc23)OC1(C)C] | 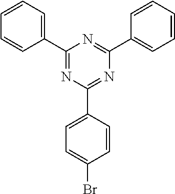 | 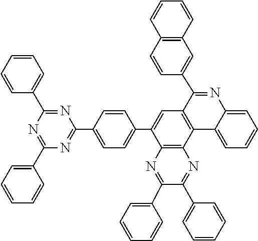 [SMILES: c1ccc(-c2nc(-c3ccccc3)nc(-c3ccc(-c4cc5c(-c6ccc7ccccc7c6)nc6ccccc6c5c5nc(-c6ccccc6)c(-c6ccccc6)nc45)cc3)n2)cc1] | 82% |
| 123 | 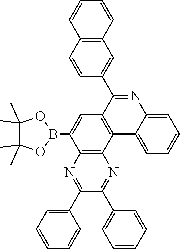 [SMILES: CC1(C)OB(c2cc3c(-c4ccc5ccccc5c4)nc4ccccc4c3c3nc(-c4ccccc4)c(-c4ccccc4)nc23)OC1(C)C] | 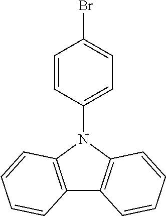 | 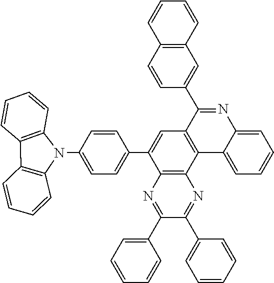 [SMILES: c1ccc(-c2nc3c(-c4ccc(-n5c6ccccc6c6ccccc65)cc4)cc4c(-c5ccc6ccccc6c5)nc5ccccc5c4c3nc2-c2ccccc2)cc1] | 80% |
| 124 | 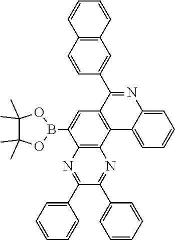 [SMILES: CC1(C)OB(c2cc3c(-c4ccc5ccccc5c4)nc4ccccc4c3c3nc(-c4ccccc4)c(-c4ccccc4)nc23)OC1(C)C] | 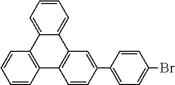 | 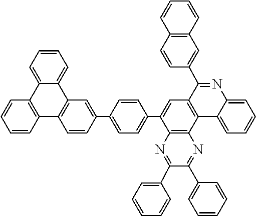 [SMILES: c1ccc(-c2nc3c(-c4ccc(-c5ccc6c7ccccc7c7ccccc7c6c5)cc4)cc4c(-c5ccc6ccccc6c5)nc5ccccc5c4c3nc2-c2ccccc2)cc1] | 79% |
| 136 | 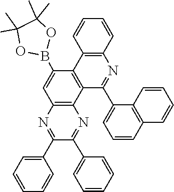 [SMILES: CC1(C)OB(c2cc3nc(-c4ccccc4)c(-c4ccccc4)nc3c3c(-c4cccc5ccccc45)nc4ccccc4c23)OC1(C)C] | 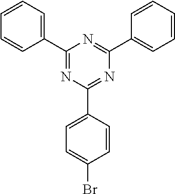 | 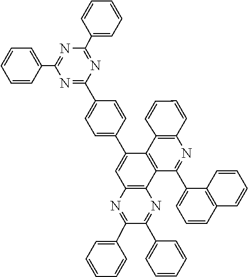 [SMILES: c1ccc(-c2nc(-c3ccccc3)nc(-c3ccc(-c4cc5nc(-c6ccccc6)c(-c6ccccc6)nc5c5c(-c6cccc7ccccc67)nc6ccccc6c45)cc3)n2)cc1] | 81% |
| 142 | 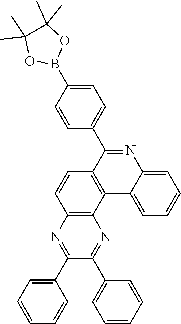 [SMILES: CC1(C)OB(c2ccc(-c3nc4ccccc4c4c3ccc3nc(-c5ccccc5)c(-c5ccccc5)nc34)cc2)OC1(C)C] | 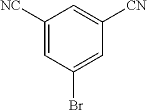 | 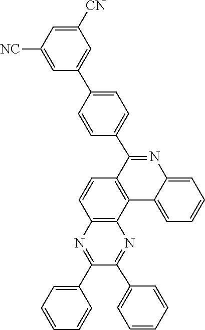 [SMILES: N#Cc1cc(C#N)cc(-c2ccc(-c3nc4ccccc4c4c3ccc3nc(-c5ccccc5)c(-c5ccccc5)nc34)cc2)c1] | 77% |
| 149 | 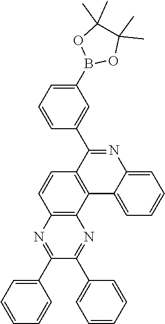 [SMILES: CC1(C)OB(c2cccc(-c3nc4ccccc4c4c3ccc3nc(-c5ccccc5)c(-c5ccccc5)nc34)c2)OC1(C)C] | 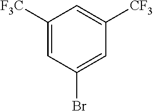 | 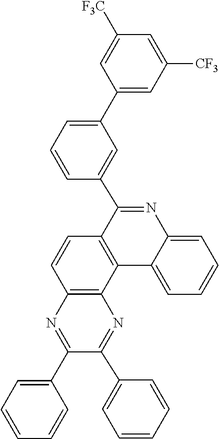 [SMILES: FC(F)(F)c1cc(-c2cccc(-c3nc4ccccc4c4c3ccc3nc(-c5ccccc5)c(-c5ccccc5)nc34)c2)cc(C(F)(F)F)c1] | 78% |
| 165 | 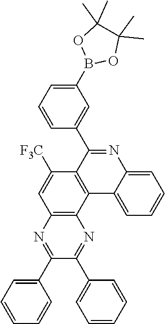 [SMILES: CC1(C)OB(c2cccc(-c3nc4ccccc4c4c3c(C(F)(F)F)cc3nc(-c5ccccc5)c(-c5ccccc5)nc34)c2)OC1(C)C] | 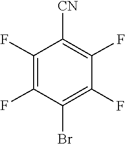 | 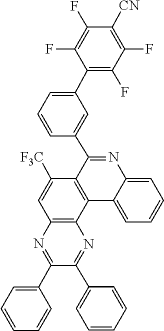 [SMILES: N#Cc1c(F)c(F)c(-c2cccc(-c3nc4ccccc4c4c3c(C(F)(F)F)cc3nc(-c5ccccc5)c(-c5ccccc5)nc34)c2)c(F)c1F] | 78% |
| 181 | 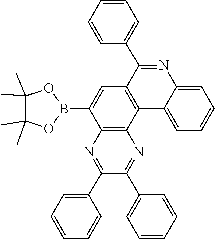 [SMILES: CC1(C)OB(c2cc3c(-c4ccccc4)nc4ccccc4c3c3nc(-c4ccccc4)c(-c4ccccc4)nc23)OC1(C)C] | 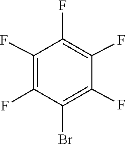 | 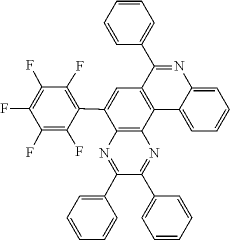 [SMILES: Fc1c(F)c(F)c(-c2cc3c(-c4ccccc4)nc4ccccc4c3c3nc(-c4ccccc4)c(-c4ccccc4)nc23)c(F)c1F] | 80% |
| 202 | 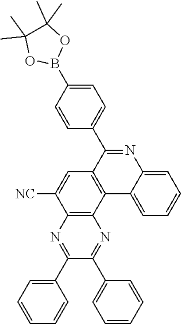 [SMILES: CC1(C)OB(c2ccc(-c3nc4ccccc4c4c3cc(C#N)c3nc(-c5ccccc5)c(-c5ccccc5)nc34)cc2)OC1(C)C] | 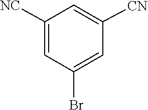 | 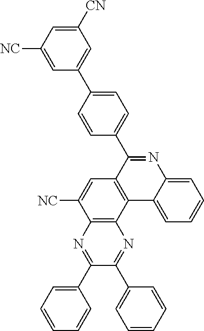 [SMILES: N#Cc1cc(C#N)cc(-c2ccc(-c3nc4ccccc4c4c3cc(C#N)c3nc(-c5ccccc5)c(-c5ccccc5)nc34)cc2)c1] | 69% |
| 203 | 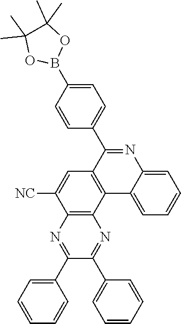 [SMILES: CC1(C)OB(c2ccc(-c3nc4ccccc4c4c3cc(C#N)c3nc(-c5ccccc5)c(-c5ccccc5)nc34)cc2)OC1(C)C] | 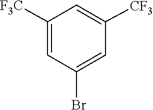 | 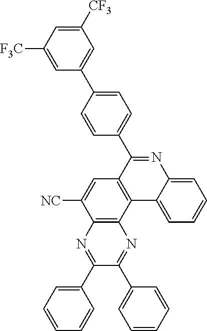 [SMILES: N#Cc1cc2c(-c3ccc(-c4cc(C(F)(F)F)cc(C(F)(F)F)c4)cc3)nc3ccccc3c2c2nc(-c3ccccc3)c(-c3ccccc3)nc12] | 72% |
| 208 | 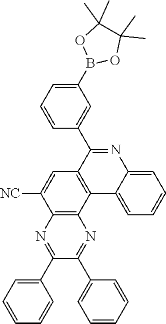 [SMILES: CC1(C)OB(c2cccc(-c3nc4ccccc4c4c3cc(C#N)c3nc(-c5ccccc5)c(-c5ccccc5)nc34)c2)OC1(C)C] | 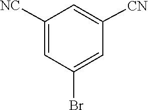 | 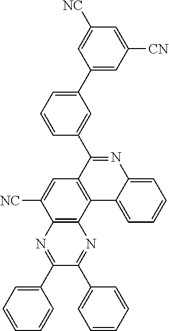 [SMILES: N#Cc1cc(C#N)cc(-c2cccc(-c3nc4ccccc4c4c3cc(C#N)c3nc(-c5ccccc5)c(-c5ccccc5)nc34)c2)c1] | 71% |
| 209 | 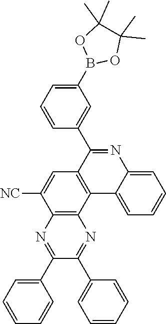 [SMILES: CC1(C)OB(c2cccc(-c3nc4ccccc4c4c3cc(C#N)c3nc(-c5ccccc5)c(-c5ccccc5)nc34)c2)OC1(C)C] | 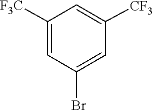 | 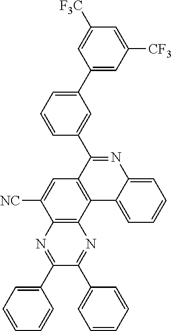 [SMILES: N#Cc1cc2c(-c3cccc(-c4cc(C(F)(F)F)cc(C(F)(F)F)c4)c3)nc3ccccc3c2c2nc(-c3ccccc3)c(-c3ccccc3)nc12] | 77% |
| 211 | 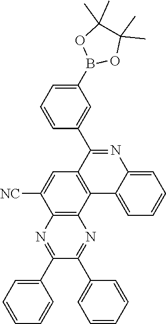 [SMILES: CC1(C)OB(c2cccc(-c3nc4ccccc4c4c3cc(C#N)c3nc(-c5ccccc5)c(-c5ccccc5)nc34)c2)OC1(C)C] | 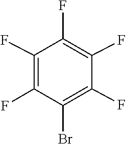 | 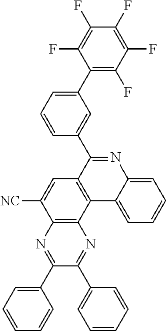 [SMILES: N#Cc1cc2c(-c3cccc(-c4c(F)c(F)c(F)c(F)c4F)c3)nc3ccccc3c2c2nc(-c3ccccc3)c(-c3ccccc3)nc12] | 79% |
| 243 | 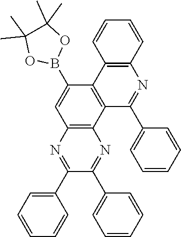 [SMILES: CC1(C)OB(c2cc3nc(-c4ccccc4)c(-c4ccccc4)nc3c3c(-c4ccccc4)nc4ccccc4c23)OC1(C)C] | 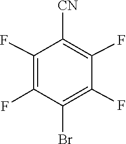 | 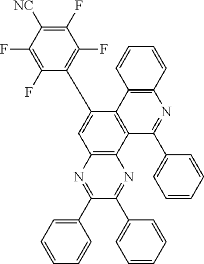 [SMILES: N#Cc1c(F)c(F)c(-c2cc3nc(-c4ccccc4)c(-c4ccccc4)nc3c3c(-c4ccccc4)nc4ccccc4c23)c(F)c1F] | 80% |
| 244 | 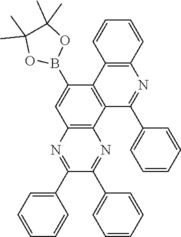 [SMILES: CC1(C)OB(c2cc3nc(-c4ccccc4)c(-c4ccccc4)nc3c3c(-c4ccccc4)nc4ccccc4c23)OC1(C)C] | 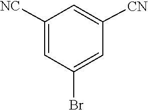 | 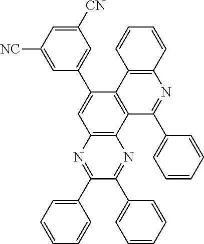 [SMILES: N#Cc1cc(C#N)cc(-c2cc3nc(-c4ccccc4)c(-c4ccccc4)nc3c3c(-c4ccccc4)nc4ccccc4c23)c1] | 80% |
| 286 | 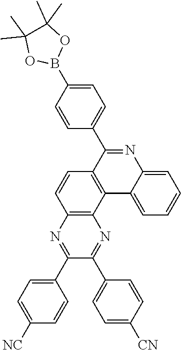 [SMILES: CC1(C)OB(c2ccc(-c3nc4ccccc4c4c3ccc3nc(-c5ccc(C#N)cc5)c(-c5ccc(C#N)cc5)nc34)cc2)OC1(C)C] | 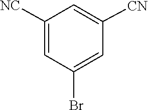 | 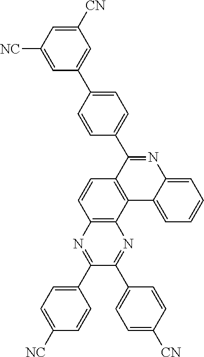 [SMILES: N#Cc1ccc(-c2nc3ccc4c(-c5ccc(-c6cc(C#N)cc(C#N)c6)cc5)nc5ccccc5c4c3nc2-c2ccc(C#N)cc2)cc1] | 79% |
| 287 | 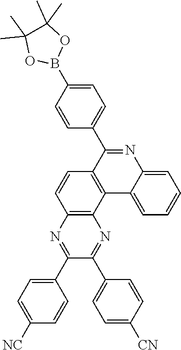 [SMILES: CC1(C)OB(c2ccc(-c3nc4ccccc4c4c3ccc3nc(-c5ccc(C#N)cc5)c(-c5ccc(C#N)cc5)nc34)cc2)OC1(C)C] | 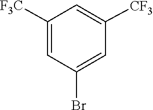 | 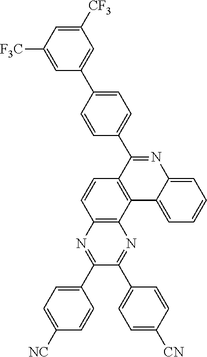 [SMILES: N#Cc1ccc(-c2nc3ccc4c(-c5ccc(-c6cc(C(F)(F)F)cc(C(F)(F)F)c6)cc5)nc5ccccc5c4c3nc2-c2ccc(C#N)cc2)cc1] | 77% |
| 292 | 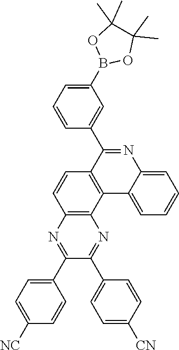 [SMILES: CC1(C)OB(c2cccc(-c3nc4ccccc4c4c3ccc3nc(-c5ccc(C#N)cc5)c(-c5ccc(C#N)cc5)nc34)c2)OC1(C)C] | 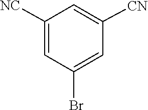 | 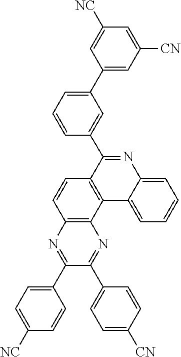 [SMILES: N#Cc1ccc(-c2nc3ccc4c(-c5cccc(-c6cc(C#N)cc(C#N)c6)c5)nc5ccccc5c4c3nc2-c2ccc(C#N)cc2)cc1] | 74% |
| 310 | 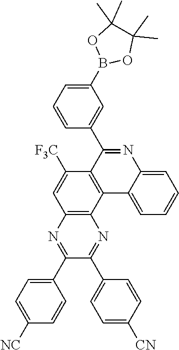 [SMILES: CC1(C)OB(c2cccc(-c3nc4ccccc4c4c3c(C(F)(F)F)cc3nc(-c5ccc(C#N)cc5)c(-c5ccc(C#N)cc5)nc34)c2)OC1(C)C] | 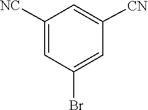 | 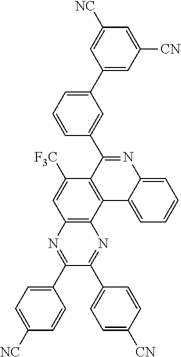 [SMILES: N#Cc1ccc(-c2nc3cc(C(F)(F)F)c4c(-c5cccc(-c6cc(C#N)cc(C#N)c6)c5)nc5ccccc5c4c3nc2-c2ccc(C#N)cc2)cc1] | 78% |
| 321 | 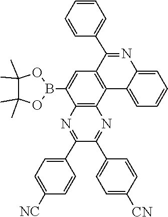 [SMILES: CC1(C)OB(c2cc3c(-c4ccccc4)nc4ccccc4c3c3nc(-c4ccc(C#N)cc4)c(-c4ccc(C#N)cc4)nc23)OC1(C)C] | 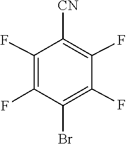 | 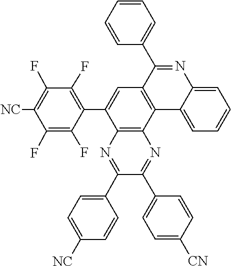 [SMILES: N#Cc1ccc(-c2nc3c(-c4c(F)c(F)c(C#N)c(F)c4F)cc4c(-c5ccccc5)nc5ccccc5c4c3nc2-c2ccc(C#N)cc2)cc1] | 71% |
| 334 | 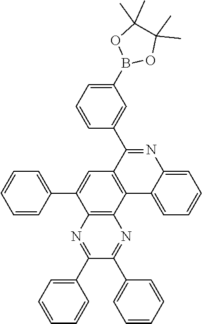 [SMILES: CC1(C)OB(c2cccc(-c3nc4ccccc4c4c3cc(-c3ccccc3)c3nc(-c5ccccc5)c(-c5ccccc5)nc34)c2)OC1(C)C] | 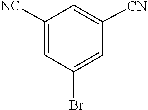 | 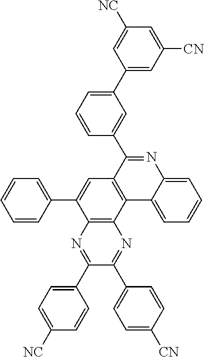 [SMILES: N#Cc1ccc(-c2nc3c(-c4ccccc4)cc4c(-c5cccc(-c6cc(C#N)cc(C#N)c6)c5)nc5ccccc5c4c3nc2-c2ccc(C#N)cc2)cc1] | 77% |
| 335 | 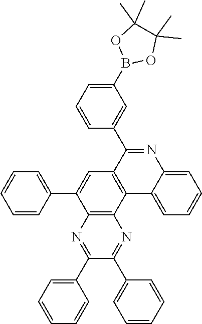 [SMILES: CC1(C)OB(c2cccc(-c3nc4ccccc4c4c3cc(-c3ccccc3)c3nc(-c5ccccc5)c(-c5ccccc5)nc34)c2)OC1(C)C] | 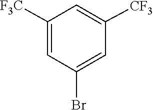 | 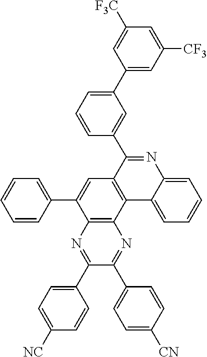 [SMILES: N#Cc1ccc(-c2nc3c(-c4ccccc4)cc4c(-c5cccc(-c6cc(C(F)(F)F)cc(C(F)(F)F)c6)c5)nc5ccccc5c4c3nc2-c2ccc(C#N)cc2)cc1] | 72% |
| 337 | 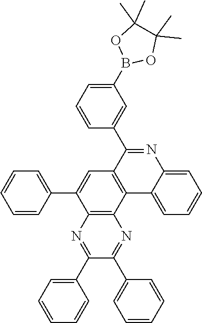 [SMILES: CC1(C)OB(c2cccc(-c3nc4ccccc4c4c3cc(-c3ccccc3)c3nc(-c5ccccc5)c(-c5ccccc5)nc34)c2)OC1(C)C] | 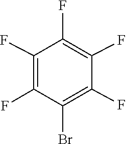 | 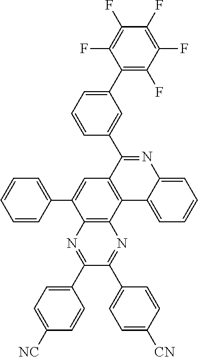 [SMILES: N#Cc1ccc(-c2nc3c(-c4ccccc4)cc4c(-c5cccc(-c6c(F)c(F)c(F)c(F)c6F)c5)nc5ccccc5c4c3nc2-c2ccc(C#N)cc2)cc1] | 78% |
| 370 | 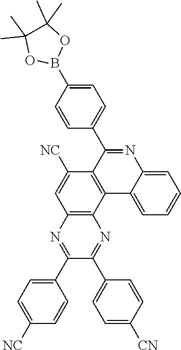 [SMILES: CC1(C)OB(c2ccc(-c3nc4ccccc4c4c3c(C#N)cc3nc(-c5ccc(C#N)cc5)c(-c5ccc(C#N)cc5)nc34)cc2)OC1(C)C] | 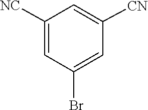 | 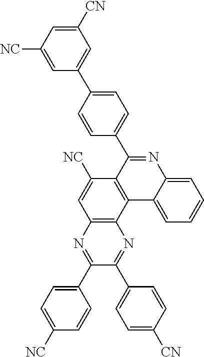 [SMILES: N#Cc1ccc(-c2nc3cc(C#N)c4c(-c5ccc(-c6cc(C#N)cc(C#N)c6)cc5)nc5ccccc5c4c3nc2-c2ccc(C#N)cc2)cc1] | 73% |
| 439 | 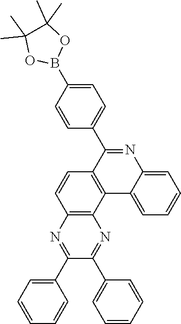 [SMILES: CC1(C)OB(c2ccc(-c3nc4ccccc4c4c3ccc3nc(-c5ccccc5)c(-c5ccccc5)nc34)cc2)OC1(C)C] | 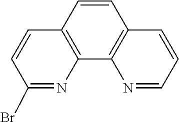 | 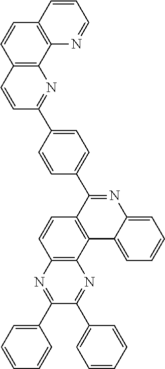 [SMILES: c1ccc(-c2nc3ccc4c(-c5ccc(-c6ccc7ccc8cccnc8c7n6)cc5)nc5ccccc5c4c3nc2-c2ccccc2)cc1] | 73% |
| 440 | 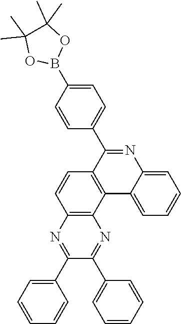 [SMILES: CC1(C)OB(c2ccc(-c3nc4ccccc4c4c3ccc3nc(-c5ccccc5)c(-c5ccccc5)nc34)cc2)OC1(C)C] | 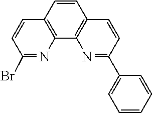 | 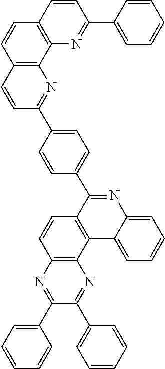 [SMILES: c1ccc(-c2ccc3ccc4ccc(-c5ccc(-c6nc7ccccc7c7c6ccc6nc(-c8ccccc8)c(-c8ccccc8)nc67)cc5)nc4c3n2)cc1] | 76% |
| 441 | 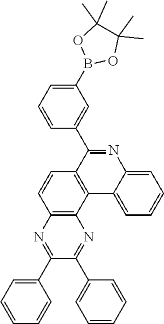 [SMILES: CC1(C)OB(c2cccc(-c3nc4ccccc4c4c3ccc3nc(-c5ccccc5)c(-c5ccccc5)nc34)c2)OC1(C)C] | 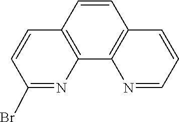 | 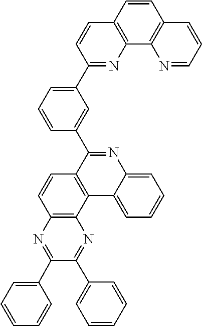 [SMILES: c1ccc(-c2nc3ccc4c(-c5cccc(-c6ccc7ccc8cccnc8c7n6)c5)nc5ccccc5c4c3nc2-c2ccccc2)cc1] | 75% |
| 442 | 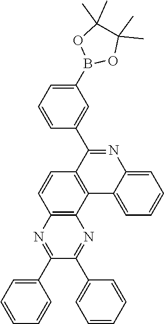 [SMILES: CC1(C)OB(c2cccc(-c3nc4ccccc4c4c3ccc3nc(-c5ccccc5)c(-c5ccccc5)nc34)c2)OC1(C)C] | 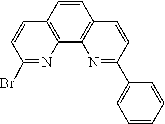 | 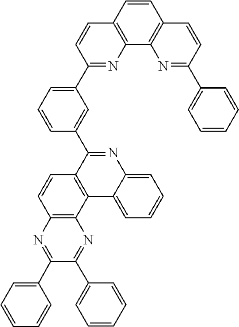 [SMILES: c1ccc(-c2ccc3ccc4ccc(-c5cccc(-c6nc7ccccc7c7c6ccc6nc(-c8ccccc8)c(-c8ccccc8)nc67)c5)nc4c3n2)cc1] | 72% |
| 443 | 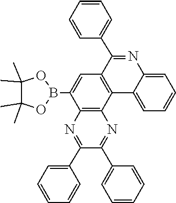 [SMILES: CC1(C)OB(c2cc3c(-c4ccccc4)nc4ccccc4c3c3nc(-c4ccccc4)c(-c4ccccc4)nc23)OC1(C)C] | 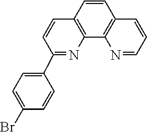 | 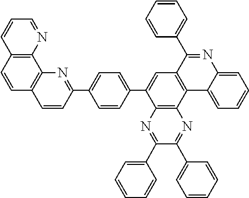 [SMILES: c1ccc(-c2nc3c(-c4ccc(-c5ccc6ccc7cccnc7c6n5)cc4)cc4c(-c5ccccc5)nc5ccccc5c4c3nc2-c2ccccc2)cc1] | 79% |
| 444 | 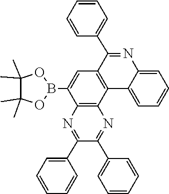 [SMILES: CC1(C)OB(c2cc3c(-c4ccccc4)nc4ccccc4c3c3nc(-c4ccccc4)c(-c4ccccc4)nc23)OC1(C)C] | 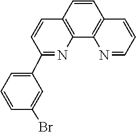 | 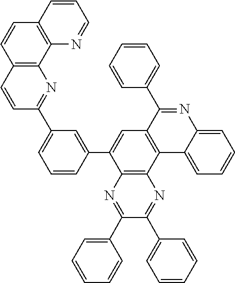 [SMILES: c1ccc(-c2nc3c(-c4cccc(-c5ccc6ccc7cccnc7c6n5)c4)cc4c(-c5ccccc5)nc5ccccc5c4c3nc2-c2ccccc2)cc1] | 80% |
| 445 | 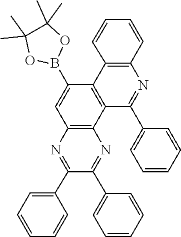 [SMILES: CC1(C)OB(c2cc3nc(-c4ccccc4)c(-c4ccccc4)nc3c3c(-c4ccccc4)nc4ccccc4c23)OC1(C)C] | 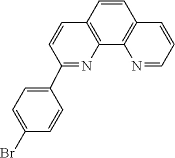 | 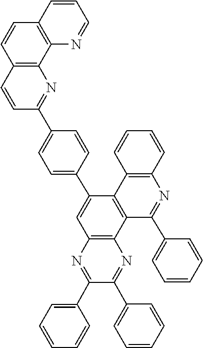 [SMILES: c1ccc(-c2nc3cc(-c4ccc(-c5ccc6ccc7cccnc7c6n5)cc4)c4c5ccccc5nc(-c5ccccc5)c4c3nc2-c2ccccc2)cc1] | 75% |
| 446 | 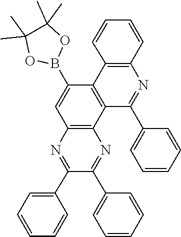 [SMILES: CC1(C)OB(c2cc3nc(-c4ccccc4)c(-c4ccccc4)nc3c3c(-c4ccccc4)nc4ccccc4c23)OC1(C)C] | 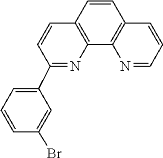 | 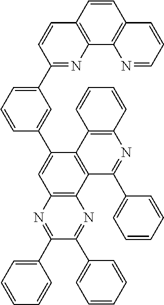 [SMILES: c1ccc(-c2nc3cc(-c4cccc(-c5ccc6ccc7cccnc7c6n5)c4)c4c5ccccc5nc(-c5ccccc5)c4c3nc2-c2ccccc2)cc1] | 71% |

Compounds other than the compounds described in Preparation Examples 1 to 5 and Tables 1 to 4 were also prepared in the same manner as the compounds described in Preparation Examples 1 to 5 and Tables 1 to 4, and the synthesis identification results are shown in the following Table 5 and Table 6.

Table 5 shows measurement values of 1H NMR (CDCl₃, 300 Mz), and Table 6 shows measurement values of FD-mass spectrometry (FD-MS: field desorption mass spectrometry).

TABLE 5

| NO | ¹H NMR (CDCl₃, 300 Mz) |
|---|---|
| 001 | 8.81 (2H, d), 8.28 (4H, d), 8.06 (1H, d), 7.98 (1H, d), |
| | 7.88 (2H, d), 7.78 (1H, t), 7.67-7.41 (19H, m) |
| 002 | 8.81 (2H, d), 8.33-8.23 (5H, m), 8.06 (1H, d), 7.98 (1H, |
| | d), 7.79-7.78 (3H, m), 7.67-7.41 (19H, m) |
| 003 | 8.81 (2H, d), 8.55 (1H, d), 8.12-8.06 (2H, m), 7.98-7.88 |
| | (4H, m), 7.79-7.78 (3H, m), 7.67-7.25 (20H, m) |
| 004 | 9.15 (1H, d), 8.93 (2H, d), 8.81 (2H, d), 8.18-7.98 (6H, |
| | m), 7.88-7.78 (7H, m), 7.67-7.41 (13H, m) |
| 007 | 8.28-8.23 (5H, m), 8.06 (1H, d), 7.98 (1H, d), 7.81-7.78 |
| | (4H, m), 7.67-7.41 (20H, m) |
| 009 | 9.15 (1H, s), 8.93 (2H, d), 8.26-7.98 (8H, m), 7.88-7.78 |
| | (5H, m), 7.67-7.41 (15H, m) |
| 010 | 8.26-8.21 (2H, m), 8.06 (1H, d), 7.98 (1H, d), 7.83-7.77 |
| | (9H, m), 7.67-7.41 (21H, m) |
| 031 | 8.30-8.28 (6H, m), 8.06 (1H, d), 7.98 (1H, d), 7.89-7.78 |
| | (4H, m), 7.60-7.41 (20H, m), 7.25 (2H, d) |
| 032 | 8.30-8.23 (7H, m), 8.06 (1H, d), 7.98 (1H, d), 7.89 (1H, |
| | s), 7.79-7.78 (3H, m), 7.60-7.41 (20H, m), 7.25 (2H, d) |
| 033 | 8.55 (1H, d), 8.30 (2H, d), 8.12-8.06 (2H, m), 7.98-7.89 |
| | (3H, m), 7.79-7.78 (3H, m), 7.68-7.25 (21H, m) |
| 056 | 8.30-8.21 (7H, m), 8.06 (1H, d), 7.98 (1H, d), 7.92 (1H, |
| | s), 7.78 (1H, t), 7.60-7.41 (18H, m) |
| 058 | 8.56 (1H, d), 8.26-8.21 (2H, m), 8.06 (1H, d), 7.98-7.92 |
| | (3H, m), 7.79-7.78 (3H, m), 7.68-7.25 (20H, m) |
| 086 | 8.30-8.28 (7H, m), 8.06 (1H, d), 7.98 (1H, d), 7.85-7.78 |
| | (3H, m), 7.60-7.41 (20H, m), 7.25 (2H, d) |
| 121 | 8.85 (1H, s), 8.38 (1H, d), 8.28 (4H, d), 8.06-7.95 (5H, |
| | m), 7.89-7.78 (4H, m), 7.60-7.41 (19H, m) |
| 123 | 8.85 (1H, s), 8.55 (1H, d), 8.38 (1H, d), 8.12-7.89 (8H, |
| | m), 7.79-7.78 (3H, m), 7.68-7.25 (20H, m) |
| 124 | 8.99-8.39 (3H, m), 8.85 (1H, s), 8.38-8.34 (2H, m), 8.12- |
| | 7.82 (14H, m), 7.60-7.41 (13H, m), 7.25 (4H, d) |
| 136 | 8.55 (1H, d), 8.46 (1H, d), 8.28 (5H, d), 8.10-8.06 (3H, |
| | m), 7.98 (1H, d), 7.85-7.78 (3H, m), 7.60-7.41 (20H, m), |
| | 7.25 (2H, d) |
| 142 | 8.81 (2H, d), 8.06-7.98 (3H, m), 7.88 (2H, d), 7.78 (1H, |
| | t), 7.67-7.41 (14H, m) |
| 149 | 8.33 (1H, s), 8.26-8.18 (4H, m), 8.06 (1H, d), 7.98 (1H, |
| | d), 7.78 (1H, t), 7.67-7.41 (15H, m) |
| 165 | 8.28-8.21 (3H, m), 8.06 (1H, d), 7.98 (1H, d), 7.78 (1H, |
| | t), 7.60-7.41 (13H, m) |
| 181 | 8.30 (2H, d), 8.06 (1H, d), 7.98 (1H, d), 7.89 (1H, s), |
| | 7.78 (1H, t), 7.60-7.41 (14H, m) |
| 202 | 8.81 (2H, d), 8.06-7.88 (7H, m), 7.78 (1H, t), 7.60-7.41 |
| | (12H, m) |
| 203 | 8.81 (2H, d), 8.33 (1H, s), 8.18 (2H, s), 8.06 (1H, d), |
| | 7.96-7.88 (4H, m), 7.78 (1H, t), 7.60-7.41 (11H, m) |
| 208 | 8.26-8.21 (2H, m), 8.06-7.98 (4H, m), 7.92 (1H, s), 7.78 |
| | (1H, t), 7.60-7.41 (14H, m) |
| 209 | 8.33 (1H, s), 8.26-8.18 (4H, m), 8.06 (1H, d), 7.98 (1H, |
| | d), 7.92 (1H, s), 7.78 (1H, t), 7.60-7.41 (13H, m) |
| 211 | 8.26-8.21 (2H, m), 8.06 (1H, d), 7.98 (1H, d), 7.92 (1H, |
| | s), 7.78 (1H, t), 7.60-7.41 (13H, m) |
| 243 | 8.30-8.28 (3H, m), 8.06 (1H, d), 7.98 (1H, d), 7.78 (1H, |
| | t), 7.60-7.41 (14H, m) |
| 244 | 8.30-8.28 (3H, m), 8.01-7.98 (4H, m), 7.78 (1H, t), 7.60- |
| | 7.41 (15H, m) |
| 286 | 8.81 (2H, d), 8.06-7.78 (15H, m), 7.67-7.60 (3H, m), 7.47 |
| | (1H, s) |
| 287 | 8.81 (2H, d), 8.33 (1H, s), 8.18 (2H, s), 8.06 (13H, m), |
| | 7.67-7.60 (3H, m) |
| 292 | 8.26-8.21 (2H, m), 8.06-7.97 (8H, m), 7.82-7.78 (5H, m), |
| | 7.67-7.54 (5H, m), 7.41 (1H, s) |
| 310 | 8.28-8.21 (3H, m), 8.06-7.97 (8H, m), 7.82-7.78 (5H, m), |
| | 7.60-7.54 (3H, m), 7.47 (1H, s) |
| 321 | 8.30 (2H, d), 8.06-7.97 (6H, m), 7.89-7.78 (6H, m), 7.60- |
| | 7.47 (4H, m) |
| 334 | 8.26-8.21 (2H, m), 8.06-7.97 (8H, m), 7.89-7.78 (6H, m), |
| | 7.60-7.41 (7H, m), 7.19 (2H, d) |
| 335 | 8.33 (1H, s), 8.26-8.18 (4H, m), 8.06-7.97 (6H, m), 7.89- |
| | 7.78 (6H, m), 7.60-7.51 (5H, m), 7.41 (1H, t), 7.19 (2H, |
| | d) |
| 337 | 8.26-8.21 (2H, m), 8.06 (1H, d), 7.98-7.97 (5H, m), 7.89- |
| | 7.78 (6H, m), 7.60-7.51 (5H, m), 7.41 (1H, t), 7.19 (2H, |
| | d) |
| 370 | 8.81 (2H, d), 8.31 (1H, s), 8.06-7.78 (15H, m), 7.60 (1H, |
| | t), 7.47 (1H, s) |
| 439 | 8.84-8.83 (5H, m), 8.38 (1H, d), 8.10-8.06 (3H, m), 7.98 |
| | (1H, d), 7.81-7.78 (2H, m), 7.67-7.35 (15H, m) |
| 440 | 8.84 (4H, d), 8.30 (2H, d), 8.10-8.06 (4H, m), 7.98 (1H, |
| | d), 7.81-7.78 (2H, m), 7.67-7.35 (18H, m) |
| 441 | 8.83 (1H, d), 8.72 (1H, s), 8.38-8.32 (3H, m), 8.10-8.06 |
| | (3H, m), 7.98 (1H, d), 7.81-7.78 (2H, m), 7.67-7.35 (16H, |
| | m) |
| 442 | 8.72 (1H, s), 8.32-8.30 (4H, m), 8.10-8.06 (4H, m), 7.98 |
| | (1H, d), 7.81-7.78 (2H, m), 7.67-7.35 (19H, m) |
| 443 | 8.83-8.81 (3H, m), 8.38 (1H, d), 8.30 (2H, d), 8.10-8.06 |
| | (3H, m), 7.98 (1H, d), 7.89 (1H, s), 7.81-7.78 (2H, m), |
| | 7.60-7.28 (18H, m) |
| 444 | 8.83 (1H, d), 8.38 (1H, d), 8.30-8.21 (4H, m), 8.10-8.06 |
| | (3H, m), 7.98 (1H, d), 7.89 (1H, s), 7.81-7.78 (2H, m), |
| | 7.60-7.35 (18H, m) |
| 445 | 8.83-8.81 (3H, m), 8.38 (1H, d), 8.30-8.28 (3H, m), 8.10- |
| | 8.06 (3H, m), 7.98 (1H, d), 7.81-7.78 (2H, m), 7.60-7.35 |
| | (18H, m) |
| 446 | 8.83 (1H, d), 8.38 (1H, d), 8.30-8.26 (5H, m), 8.10-8.06 |
| | (3H, m), 7.98 (1H, d), 7.78-7.71 (2H, m), 7.60-7.35 (18H, |
| | m) |

TABLE 6

| Com- | | Com- | |
|---|---|---|---|
| pound | FD-MS | pound | FD-MS |
| 001 | m/z = 690.79 | 002 | m/z = 689.80 |
| | (C₄₈H₃₀N₆ = 690.25) | | (C₄₉H₃₁N₅ = 689.26) |
| 003 | m/z = 700.83 | 004 | m/z = 685.81 |
| | (C₅₁H₃₂N₄ = 700.26) | | (C₅₁H₃₁N₃ = 685.25) |
| 007 | m/z = 689.80 | 009 | m/z = 685.81 |
| | (C₄₉H₃₁N₅ = 689.26) | | (C₅₁H₃₁N₃ = 685.25) |
| 010 | m/z = 735.81 | 031 | m/z = 766.89 |
| | (C₅₁H₃₄N₃OP = 735.24) | | (C₅₄H₃₄N₆ = 766.28) |
| 032 | m/z = 765.90 | 033 | m/z = 700.83 |
| | (C₅₅H₃₅N₅ = 765.29) | | (C₅₁H₃₂N₄ = 700.26) |
| 056 | m/z = 715.80 | 058 | m/z = 725.84 |
| | (C₄₉H₂₉N₇ = 715.25) | | (C₅₂H₃₁N₅ = 725.26) |
| 086 | m/z = 766.89 | 121 | m/z = 816.95 |
| | (C₅₄H₃₄N₆ = 766.28) | | (C₅₈H₃₆N₆ = 816.30) |
| 123 | m/z = 750.89 | 124 | m/z = 811.97 |
| | (C₅₅H₃₄N₄ = 750.28) | | (C₆₁H₃₇N₃ = 811.30) |
| 136 | m/z = 816.95 | 142 | m/z = 585.65 |
| | (C₅₈H₃₆N₆ = 816.30) | | (C₄₁H₂₃N₅ = 585.20) |
| 149 | m/z = 671.63 | 165 | m/z = 700.61 |
| | (C₄₁H₂₃F₆N₃ = 671.18) | | (C₄₁H₁₉F₇N₄ = 700.15) |
| 181 | m/z = 625.59 | 202 | m/z = 610.66 |
| | (C₃₉H₂₀F₅N₃ = 625.16) | | (C₄₂H₂₂N₆ = 610.19) |
| 203 | m/z = 696.64 | 208 | m/z = 610.66 |
| | (C₄₂H₂₂F₆N₄ = 696.17) | | (C₄₂H₂₂N₆ = 610.19) |
| 209 | m/z = 696.64 | 211 | m/z = 650.60 |
| | (C₄₂H₂₂F₆N₄ = 696.17) | | (C₄₀H₁₉F₅N₄ = 650.15) |
| 243 | m/z = 632.61 | 244 | m/z = 585.65 |
| | (C₄₀H₂₀F₄N₄ = 632.16) | | (C₄₁H₂₃N₅ = 585.20) |
| 286 | m/z = 635.67 | 287 | m/z = 721.65 |
| | (C₄₃H₂₁N₇ = 635.19) | | (C₄₃H₂₁F₆N₅ = 721.17) |
| 292 | m/z = 635.67 | 310 | m/z = 703.67 |
| | (C₄₃H₂₁N₇ = 635.19) | | (C₄₄H₂₀F₃N₇ = 703.17) |
| 321 | m/z = 682.63 | 334 | m/z = 711.77 |
| | (C₄₂H₁₈F₄N₆ = 682.15) | | (C₄₉H₂₅N₇ = 711.22) |
| 335 | m/z = 797.75 | 337 | m/z = 751.70 |
| | (C₄₉H₂₅F₆N₅ = 797.20) | | (C₄₇H₂₂F₅N₅ = 751.18) |
| 370 | m/z = 660.68 | 439 | m/z = 637.73 |
| | (C₄₄H₂₀N₈ = 660.18) | | (C₄₅H₂₇N₅ = 637.23) |
| 440 | m/z = 713.83 | 441 | m/z = 637.73 |
| | (C₅₁H₃₁N₅ = 713.26) | | (C₄₅H₂₇N₅ = 637.23) |
| 442 | m/z = 713.83 | 443 | m/z = 713.83 |
| | (C₅₁H₃₁N₅ = 713.26) | | (C₅₁H₃₁N₅ = 713.26) |
| 444 | m/z = 713.83 | 445 | m/z = 713.83 |
| | (C₅₁H₃₁N₅ = 713.26) | | (C₅₁H₃₁N₅ = 713.26) |

| | |
|---|---|
| 446 | m/z = 713.83 |
| | (C₅₁H₃₁N₅ = 713.26) |

### EXPERIMENTAL EXAMPLE

### Experimental Example 1

1) Manufacture of Organic Light Emitting Device

A transparent indium tin oxide (ITO) electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, the ITO substrate was installed in a substrate folder of a vacuum deposition apparatus, and the following dipyrazino[2,3-f:2′,3′-h]quinoxaline-2,3,6,7,10,11-hexacarbonitrile (HAT) was introduced to a cell in the vacuum deposition apparatus.

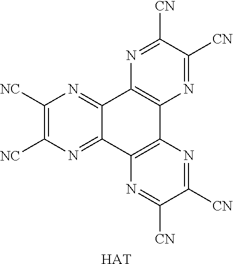 [SMILES: N#Cc1nc2c3nc(C#N)c(C#N)nc3c3nc(C#N)c(C#N)nc3c2nc1C#N ; "HAT"]

Subsequently, the chamber was evacuated until the degree of vacuum therein reached 10⁻⁶ torr, and then HAT was evaporated by applying a current to the cell to deposit a hole injection layer having a thickness of 600 Å on the ITO substrate.

To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

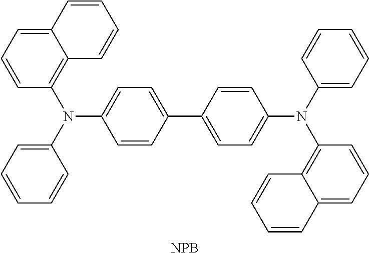 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

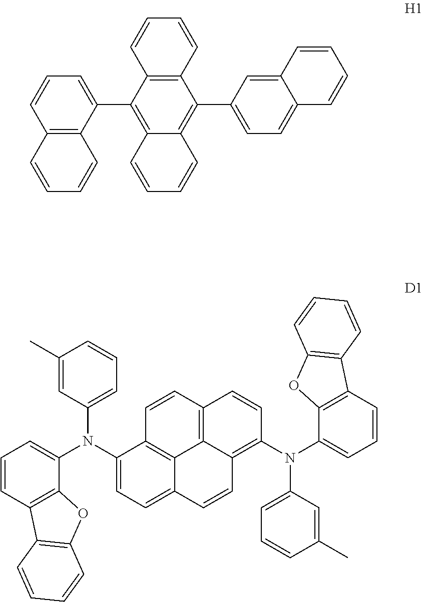

Subsequently, a compound shown in the following Table 7 was deposited to a thickness of 300 Å as an electron transfer layer.

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁸ torr to 10⁻⁶ torr by each material to be used in the OLED manufacture.

Results of measuring driving voltage, light emission efficiency, color coordinate (CIE) and lifetime of the blue organic light emitting devices manufactured according to the present disclosure are as shown in the following Table 7.

TABLE 7

| | | | Light | | |
|---|---|---|---|---|---|
| | | Driving | Emission | | |
| | | Voltage | Efficiency | | Lifetime |
| | Compound | (V) | (cd/A) | CIE (x, y) | (T95) |
| Comparative | E1 | 4.84 | 6.43 | (0.134, 0.100) | 255 |
| Example 1-1 | | | | | |
| Comparative | E2 | 4.96 | 6.12 | (0.134, 0.101) | 252 |
| Example 1-2 | | | | | |
| Comparative | E3 | 4.98 | 6.14 | (0.134, 0.102) | 245 |
| Example 1-3 | | | | | |
| Example 1 | 001 | 4.63 | 6.65 | (0.134, 0.101) | 321 |
| Example 2 | 002 | 4.54 | 6.84 | (0.134, 0.102) | 307 |
| Example 3 | 003 | 4.78 | 6.40 | (0.134, 0.101) | 268 |
| Example 4 | 004 | 4.92 | 6.45 | (0.134, 0.103) | 271 |
| Example 5 | 007 | 4.73 | 6.43 | (0.134, 0.102) | 283 |
| Example 6 | 009 | 4.83 | 6.59 | (0.134, 0.101) | 273 |
| Example 7 | 010 | 4.74 | 6.52 | (0.134, 0.102) | 269 |
| Example 8 | 031 | 4.59 | 6.71 | (0.134, 0.101) | 318 |
| Example 9 | 032 | 4.86 | 6.59 | (0.134, 0.101) | 267 |
| Example 10 | 033 | 4.78 | 6.58 | (0.134, 0.100) | 269 |
| Example 11 | 056 | 4.79 | 6.45 | (0.134, 0.101) | 265 |
| Example 12 | 058 | 4.81 | 6.51 | (0.134, 0.100) | 277 |
| Example 13 | 086 | 4.82 | 6.59 | (0.134, 0.100) | 285 |
| Example 14 | 121 | 4.61 | 6.68 | (0.134, 0.100) | 334 |
| Example 15 | 123 | 4.80 | 6.47 | (0.134, 0.100) | 272 |
| Example 16 | 124 | 4.81 | 6.48 | (0.134, 0.100) | 288 |
| Example 17 | 136 | 4.74 | 6.57 | (0.134, 0.102) | 282 |

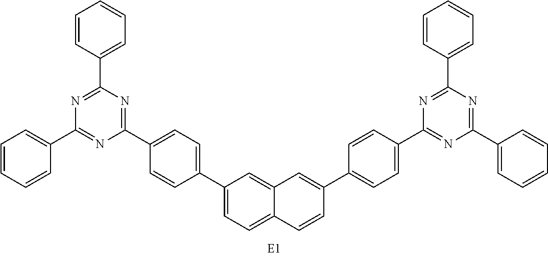 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

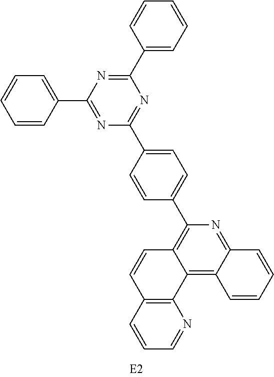 [SMILES: c1ccc(-c2nc(-c3ccccc3)nc(-c3ccc(-c4nc5ccccc5c5c4ccc4cccnc45)cc3)n2)cc1]

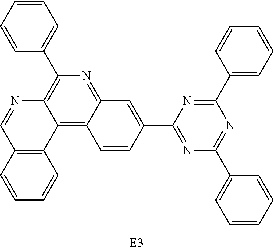 [SMILES: c1ccc(-c2nc(-c3ccccc3)nc(-c3ccc4c(c3)nc(-c3ccccc3)c3ncc5ccccc5c34)n2)cc1]

As seen from the results of Table 7, the organic light emitting device using the electron transfer layer material of the blue organic light emitting device of the present disclosure had lower driving voltage, and improved light emission efficiency and lifetime compared to Comparative Example 1-1 to Comparative Example 1-3. Particularly, it was identified that Compounds 001, 002, 031 and 121 were superior in all aspects of driving, efficiency and lifetime.

Such a result is considered to be due to the fact that, when using the disclosed compound having proper length and strength, and flatness as the electron transfer layer, a compound in an excited state is made by receiving electrons under a specific condition, and particularly when an excited state is formed in the hetero-skeleton site of the compound, excited energy moves to a stable state before the excited hetero-skeleton site goes through other reactions, and as a result, the relatively stabilized compound is capable of efficiently transferring electrons without the compound being decomposed or destroyed.

For reference, those that are stable when excited are aryl or acene-based compounds or polycyclic hetero-compounds. When compared with Comparative Examples 1-2 and 1-3, the compound of the present disclosure has a form in which quinoxaline and quinoline are fused, whereas Compounds E2 and E3 have a form in which quinoline and quinoline are fused. In was identified that electron mobility was enhanced by fusing quinoxaline instead of quinoline in the present disclosure, which resultantly improved all of driving, lifetime and efficiency by enhancing a charge balance in the light emitting layer. In conclusion, it is considered that the compound of the present disclosure brings superiority in all aspects of driving, efficiency and lifetime by enhancing enhanced electron-transfer properties or improved stability.

### <Experimental Example 2>—Comparative Example 2

1) Manufacture of Organic Light Emitting Device

A transparent indium tin oxide (ITO) electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, the ITO substrate was installed in a substrate folder of a vacuum deposition apparatus, and the following dipyrazino[2,3-f:2′,3′-h]quinoxaline-2,3,6,7,10,11-hexacarbonitrile (HAT) was introduced to a cell in the vacuum deposition apparatus.

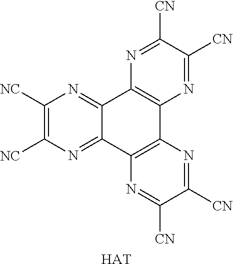 [SMILES: N#Cc1nc2c3nc(C#N)c(C#N)nc3c3nc(C#N)c(C#N)nc3c2nc1C#N ; "HAT"]

Subsequently, the chamber was evacuated until the degree of vacuum therein reached 10⁻⁶ torr, and then HAT was evaporated by applying a current to the cell to deposit a hole injection layer having a thickness of 600 Å on the ITO substrate.

To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

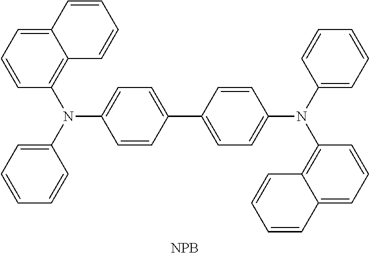 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

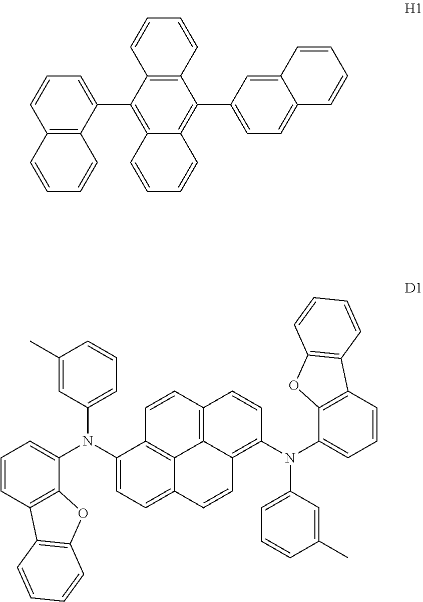

Subsequently, a compound of the following Structural Formula E1 was deposited to a thickness of 300 Å as an electron transfer layer.

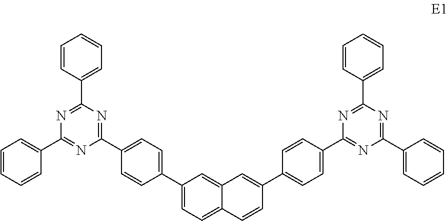 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁸ torr to 10⁻⁶ torr by each material to be used in the OLED manufacture.

Organic electroluminescent devices were manufactured in the same manner as in Experimental Example 2 (Comparative Example 2) except that, instead of E1 used as the electron transfer layer to 300 Å, a hole blocking layer was formed to a thickness of 50 Å using a compound shown in Table 8 and then an electron transfer layer was formed on the hole blocking layer to a thickness of 250 Å using E1.

Results of measuring driving voltage, light emission efficiency, color coordinate (CIE) and lifetime of the blue organic light emitting devices manufactured according to the present disclosure are as shown in the following Table 8.

TABLE 8

| | | | Light | | |
|---|---|---|---|---|---|
| | | Driving | Emission | | Life- |
| | | Voltage | Efficiency | | time |
| | Compound | (V) | (cd/A) | CIE (x, y) | (T95) |
| Comparative | — | 4.82 | 6.45 | (0.134, 0.100) | 252 |
| Example 2 | | | | | |
| Example 18 | 003 | 4.72 | 6.98 | (0.134, 0.101) | 285 |
| Example 19 | 004 | 4.62 | 7.02 | (0.134, 0.102) | 291 |
| Example 20 | 009 | 4.76 | 6.89 | (0.134, 0.101) | 301 |
| Example 21 | 033 | 4.56 | 6.72 | (0.134, 0.103) | 296 |
| Example 22 | 058 | 4.62 | 6.89 | (0.134, 0.101) | 292 |
| Example 23 | 123 | 4.61 | 6.91 | (0.134, 0.102) | 305 |
| Example 24 | 124 | 4.69 | 6.88 | (0.134, 0.101) | 298 |

As seen from the results of Table 8, it was identified that the organic light emitting device using the hole blocking layer material of blue organic electroluminescent device of the present disclosure had lower driving voltage, and significantly improved light emission efficiency and lifetime compared to Comparative Example 2.

### Experimental Example 3

1) Manufacture of Organic Light Emitting Device

A transparent indium tin oxide (ITO) electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, the ITO substrate was installed in a substrate folder of a vacuum deposition apparatus, and a compound of the following Table 9 was introduced to a cell in the vacuum deposition apparatus.

Subsequently, the chamber was evacuated until the degree of vacuum therein reached 10⁻⁶ torr, and then the compound of the following Table 9 was evaporated by applying a current to the cell to deposit a hole injection layer having a thickness of 600 Å on the ITO substrate.

To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

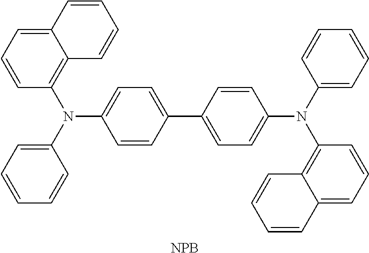 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

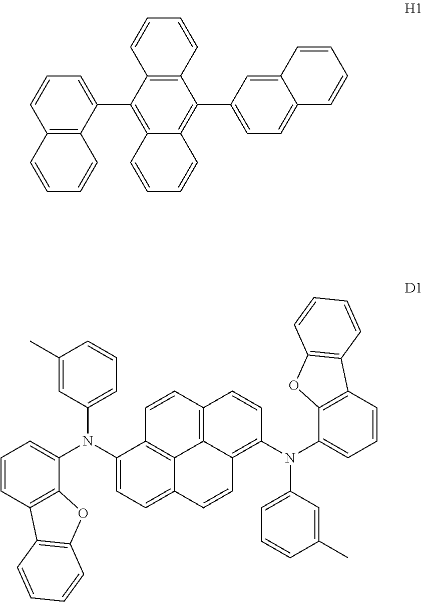

Subsequently, E1 was deposited to a thickness of 300 Å as an electron transfer layer.

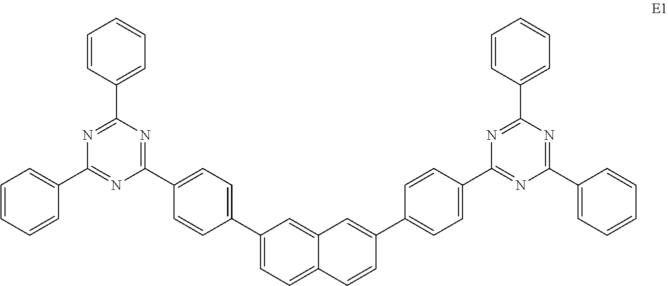 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁸ torr to 10⁻⁶ torr by each material to be used in the OLED manufacture. Results of measuring driving voltage, light emission efficiency, color coordinate (CIE) and lifetime of the blue organic light emitting devices manufactured according to the present disclosure are as shown in the following Table 9.

TABLE 9

| | | | Light | | |
|---|---|---|---|---|---|
| | | Driving | Emission | | |
| | | Voltage | Efficiency | | Lifetime |
| | Compound | (V) | (cd/A) | CIE (x, y) | (T95) |
| Comparative | HAT | 4.80 | 6.36 | (0.134, 0.100) | 255 |
| Example 3-1 | | | | | |
| Comparative | HI-1 | 4.82 | 6.38 | (0.134, 0.101) | 252 |
| Example 3-2 | | | | | |
| Example 25 | 142 | 4.77 | 6.65 | (0.134, 0.101) | 280 |
| Example 26 | 149 | 4.67 | 6.54 | (0.134, 0.102) | 287 |
| Example 27 | 165 | 4.68 | 6.60 | (0.134, 0.101) | 268 |
| Example 28 | 181 | 4.72 | 6.66 | (0.134, 0.103) | 275 |
| Example 29 | 202 | 4.75 | 6.53 | (0.134, 0.102) | 281 |
| Example 30 | 203 | 4.66 | 6.59 | (0.134, 0.101) | 277 |
| Example 31 | 208 | 4.71 | 6.59 | (0.134, 0.102) | 287 |
| Example 32 | 209 | 4.70 | 6.73 | (0.134, 0.101) | 297 |
| Example 33 | 211 | 4.69 | 6.64 | (0.134, 0.101) | 279 |
| Example 34 | 243 | 4.72 | 6.58 | (0.134, 0.100) | 302 |
| Example 35 | 244 | 4.74 | 6.57 | (0.134, 0.101) | 295 |
| Example 36 | 286 | 4.67 | 6.53 | (0.134, 0.100) | 277 |
| Example 37 | 287 | 4.68 | 6.59 | (0.134, 0.100) | 282 |
| Example 38 | 292 | 4.70 | 6.68 | (0.134, 0.100) | 304 |
| Example 39 | 310 | 4.75 | 6.57 | (0.134, 0.100) | 291 |
| Example 40 | 321 | 4.72 | 6.62 | (0.134, 0.100) | 288 |
| Example 41 | 334 | 4.65 | 6.98 | (0.134, 0.102) | 281 |
| Example 42 | 335 | 4.64 | 6.89 | (0.134, 0.100) | 285 |
| Example 43 | 337 | 4.74 | 6.68 | (0.134, 0.100) | 299 |
| Example 44 | 370 | 4.63 | 6.92 | (0.134, 0.100) | 278 |

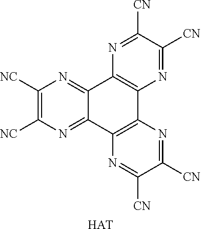 [SMILES: N#Cc1nc2c3nc(C#N)c(C#N)nc3c3nc(C#N)c(C#N)nc3c2nc1C#N ; "HAT"]

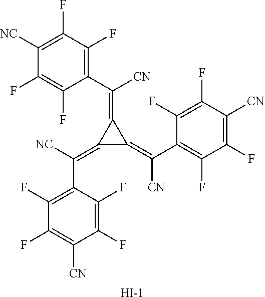 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]]

As seen from the results of Table 9, it was identified that the organic electroluminescent device using the hole injection layer material of the blue organic electroluminescent device of the present disclosure had lower driving voltage, and improved light emission efficiency and lifetime compared to Comparative Example 3-1 and Comparative Example 3-2.

### Experimental Example 4

1) Manufacture of Organic Light Emitting Device

A transparent indium tin oxide (ITO) electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

On the transparent ITO electrode (anode), organic materials were formed in a 2-stack white organic light emitting device (WOLED) structure. As for the first stack, TAPC was thermal vacuum deposited first to a thickness of 300 Å to form a hole transfer layer. After forming the hole transfer layer, a light emitting layer was thermal vacuum deposited thereon as follows. The light emitting layer was deposited to 300 Å by doping TCz1, a host, with FIrpic, a blue phosphorescent dopant, by 8%. After forming an electron transfer layer to 400 Å using TmPyPB, a compound described in the following Table 10 was doped with Cs₂CO₃ by 20% to form a charge generation layer to 100 Å.

As for the second stack, MoO₃ was thermal vacuum deposited first to a thickness of 50 Å to form a hole injection layer. A hole transfer layer that is a common layer was formed to 100 Å by doping MoO₃ to TAPC by 20%, and then depositing TAPC to 300 Å. A light emitting layer was deposited to 300 Å thereon by doping TCz1, a host, with Ir(ppy)₃, a green phosphorescent dopant, by 8%, and an electron transfer layer was formed to 600 Å using TmPyPB. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device was manufactured.

Meanwhile, all the organic compounds required to manufacture the OLED were vacuum sublimation purified under 10⁻⁸ torr to 10⁻⁶ torr for each material to be used in the OLED manufacture.

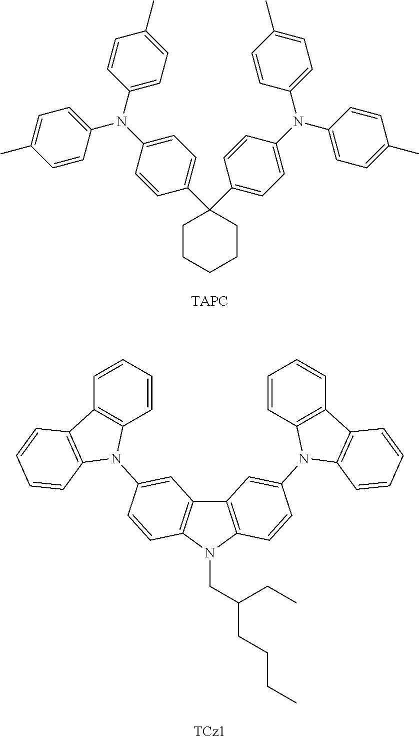

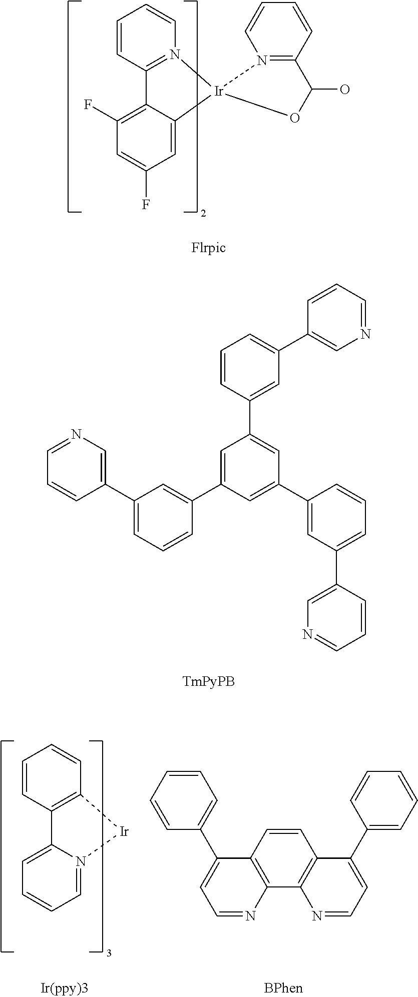

Results of measuring driving voltage, light emission efficiency, color coordinate (CIE) and lifetime of the white organic light emitting devices manufactured according to the present disclosure are as shown in the following Table 10.

TABLE 10

| | | | Light | | |
|---|---|---|---|---|---|
| | | Driving | Emission | | Life- |
| | | Voltage | Efficiency | | time |
| | Compound | (V) | (cd/A) | CIE (x, y) | (T95) |
| Comparative | BPhen | 7.24 | 56.15 | (0.213, 0.430) | 102 |
| Example 4 | | | | | |
| Example 45 | 439 | 6.74 | 62.47 | (0.212, 0.421) | 110 |
| Example 46 | 440 | 6.49 | 64.35 | (0.211, 0.433) | 133 |
| Example 47 | 441 | 6.65 | 66.62 | (0.214, 0.439) | 124 |
| Example 48 | 442 | 6.46 | 64.91 | (0.212, 0.424) | 141 |
| Example 49 | 443 | 6.72 | 64.22 | (0.214, 0.437) | 129 |
| Example 50 | 444 | 6.71 | 65.10 | (0.212, 0.426) | 117 |
| Example 51 | 445 | 6.63 | 63.15 | (0.214, 0.437) | 127 |
| Example 52 | 446 | 6.44 | 65.62 | (0.213, 0.424) | 133 |

As seen from the results of Table 10, it was identified that the organic electroluminescent device using the charge generation layer material of the 2-stack white organic electroluminescent device of the present disclosure had lower driving voltage and improved light emission efficiency compared to Comparative Example 4. Such a result is considered to be due to the fact that the compound of the present disclosure used as an N-type charge generation layer formed with the disclosed skeleton having proper length and strength, and flatness and a proper hetero-compound capable of binding to metals forms a gap state in the N-type charge generation layer by doping an alkali metal or an alkaline earth metal thereto, and electrons produced from a P-type charge generation layer are readily injected into the electron transfer layer through the gap state produced in the N-type charge generation layer. Accordingly, it is considered that the P-type charge generation layer may favorably inject and transfer electrons to the N-type charge generation layer, and as a result, driving voltage was lowered, and efficiency and lifetime were improved in the organic light emitting device.

The compound represented by Chemical Formula 1 of the present application has a structure having substituents linked to the quinoxaline group, a core structure, with two phenyl groups, and by having two phenyl groups, thermal stability is excellent, and as a result, significantly superior lifetime properties are obtained.

In other words, when there is no substituent linked to the quinoxaline group with a phenyl group or when there is only one such substituent, reactivity of carbon next to nitrogen of the quinoxaline group is high causing a problem in thermal stability, which results in a phenomenon of significantly reduced lifetime compared to a structure having two phenyl group-linked substituents as in Chemical Formula 1 of the present application.

In addition, Chemical Formula 1 of the present application has a core structure in which a quinoxaline group is fused to a quinoline group, and when another structure such as an imidazole group is fused to the quinoline group, electron distribution is concentrated in the skeleton structure, a core structure, compared to when a quinoxaline group is fused resulting in decreased electron mobility, and it was seen that driving voltage increased and efficiency decreased in this case compared to the compound of Chemical Formula 1 of the present application.

1. A heterocyclic compound represented by the following Chemical Formula 1: 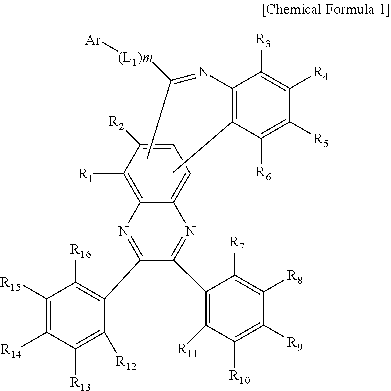
wherein, in Chemical Formula 1,
R₁ to R₆ are the same as or different from each other, and each independently selected from the group consisting of hydrogen; deuterium; a halogen group; —CN; a substituted or unsubstituted C1 to C60 alkyl group; a substituted or unsubstituted C6 to C60 aryl group; a substituted or unsubstituted C2 to C60 heteroaryl group; —SiRR′R″; —P(═O)RR′; and an amine group unsubstituted or substituted with a substituted or unsubstituted C1 to C60 alkyl group, a substituted or unsubstituted C6 to C60 aryl group or a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring;
L₁ is a substituted or unsubstituted C6 to C60 arylene group; or a substituted or unsubstituted C2 to C60 heteroarylene group;
Ar is hydrogen; a halogen group; —CN; a substituted or unsubstituted C1 to C60 alkyl group; a substituted or unsubstituted C6 to C60 aryl group; a substituted or unsubstituted C2 to C60 heteroaryl group; —SiRR′R″; or —P(═O)RR′;
R₇ to R₁₆ are the same as or different from each other, and each independently hydrogen; a halogen group; —CN; a substituted or unsubstituted C6 to C60 aryl group; or a substituted or unsubstituted C2 to C60 heteroaryl group;
R, R′ and R″ are the same as or different from each other, and each independently hydrogen; a substituted or unsubstituted C1 to C40 alkyl group; or a substituted or unsubstituted C6 to C40 aryl group; and
m is an integer of 1 to 4, and when m is 2 or greater, the two or more Lis are the same as or different from each other.

2. The heterocyclic compound of claim 1, wherein the “substituted or unsubstituted” means being substituted with one or more substituents selected from the group consisting of C1 to C60 haloalkyl; C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted; and
R, R′ and R″ have the same definitions as in Chemical Formula 1.

3. The heterocyclic compound of claim 1, wherein Chemical Formula 1 is the following Chemical Formula 2 or Chemical Formula 3: 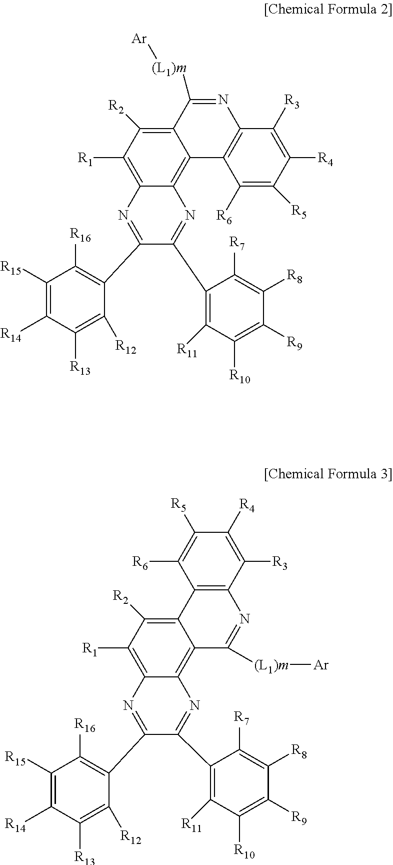
in Chemical Formulae 2 and 3,
R₁ to R₁₆, Ar, m and L₁ have the same definitions as in Chemical Formula 1.

4. The heterocyclic compound of claim 1, wherein Chemical Formula 1 is represented by any one of the following Chemical Formula 4 to Chemical Formula 8: 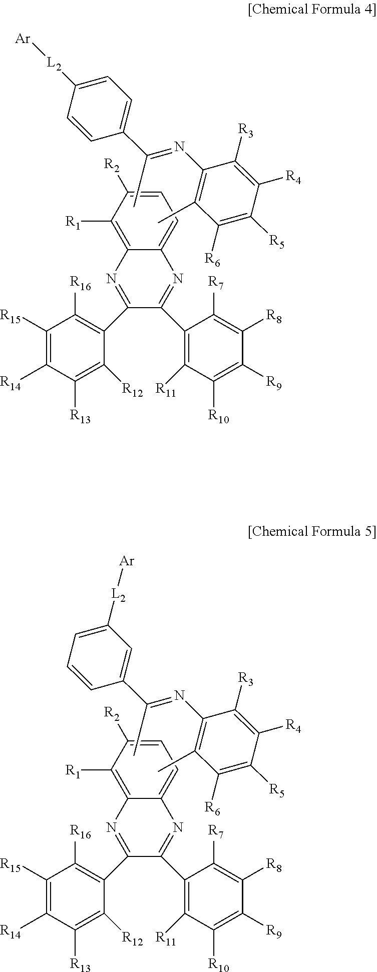 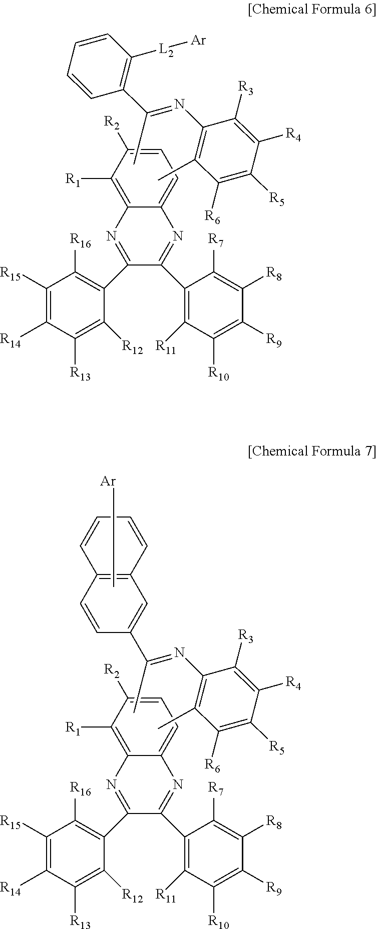 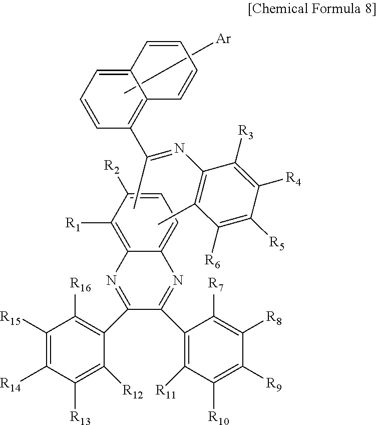
in Chemical Formulae 4 to 8,
R₁ to R₁₆ and Ar have the same definitions as in Chemical Formula 1; and
L₂ is a direct bond; a substituted or unsubstituted C6 to C60 arylene group; or a substituted or unsubstituted C2 to C60 heteroarylene group.

5. The heterocyclic compound of claim 1, wherein R₇ to R₁₆ are the same as or different from each other, and each independently hydrogen; or —CN.

6. The heterocyclic compound of claim 1, wherein, when Ar of Chemical Formula 1 is hydrogen,
at least one of R₁ to R₆ is represented by -(L₃)p-(Z₁)q, and the rest are hydrogen; or a substituted or unsubstituted C6 to C60 aryl group;
L₃ is a direct bond; a substituted or unsubstituted C6 to C60 arylene group; or a substituted or unsubstituted C2 to C60 heteroarylene group;
Z₁ is a halogen group; —CN; a substituted or unsubstituted C1 to C60 alkyl group; a substituted or unsubstituted C6 to C60 aryl group; a substituted or unsubstituted C2 to C60 heteroaryl group; —SiRR′R″; or —P(═O)RR′;
R, R′ and R″ have the same definitions as in Chemical Formula 1;
p is an integer of 0 to 3; and
q is an integer of 1 to 3.

7. The heterocyclic compound of claim 1, wherein Ar is hydrogen; a halogen group; —CN; a C1 to C40 alkyl group unsubstituted or substituted with a halogen group; a C6 to C40 aryl group unsubstituted or substituted with one or more substituents selected from the group consisting of a halogen group, —CN and —CF₃; a C2 to C40 heteroaryl group unsubstituted or substituted with a C6 to C40 aryl group; or —P(═O)RR′; and
R, R′ and R″ have the same definitions as in Chemical Formula 1.

8. The heterocyclic compound of claim 1, wherein Chemical Formula 1 is represented by any one of the following compounds:

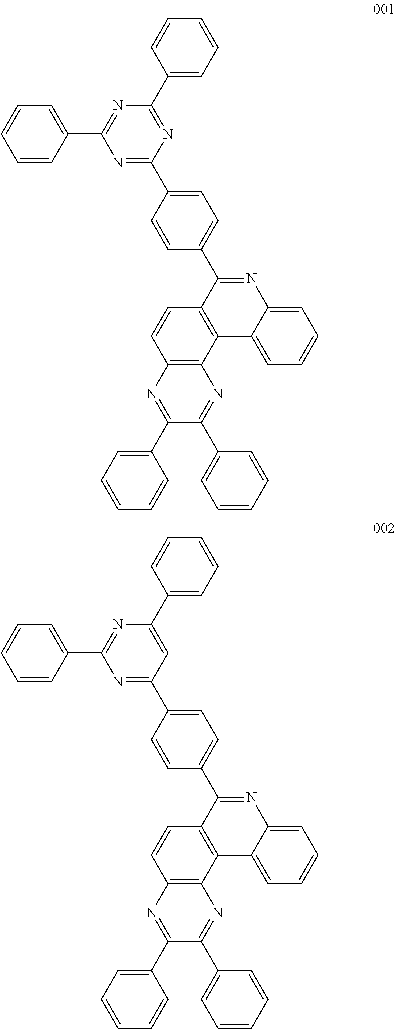

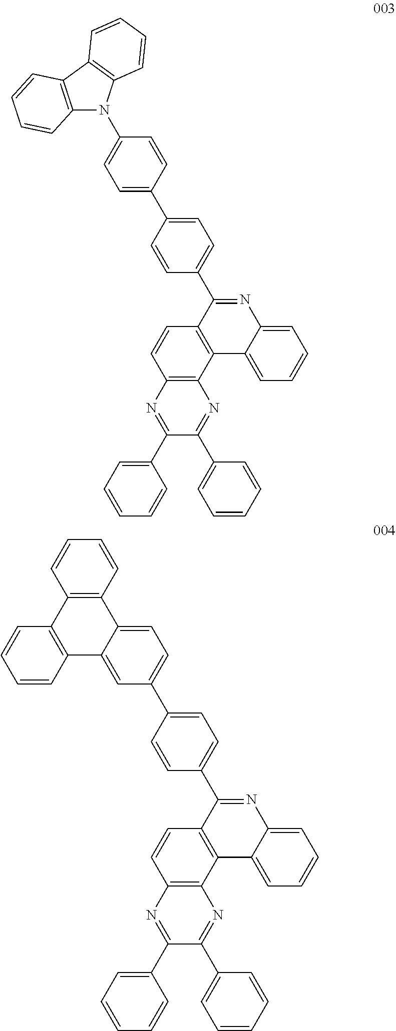

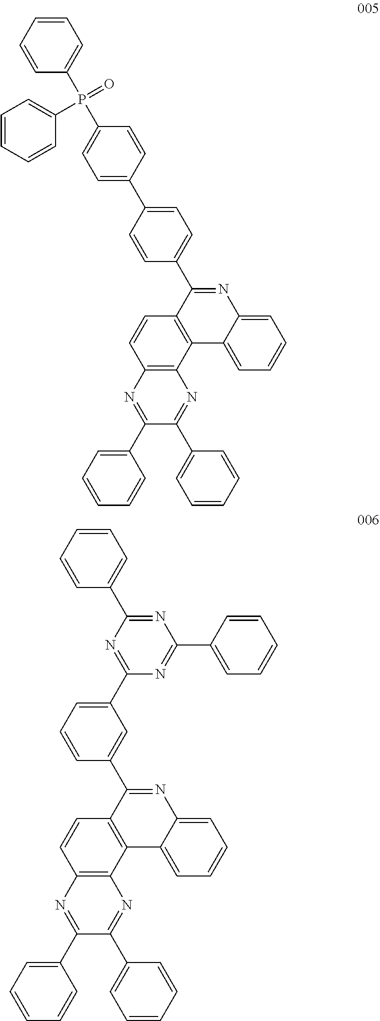

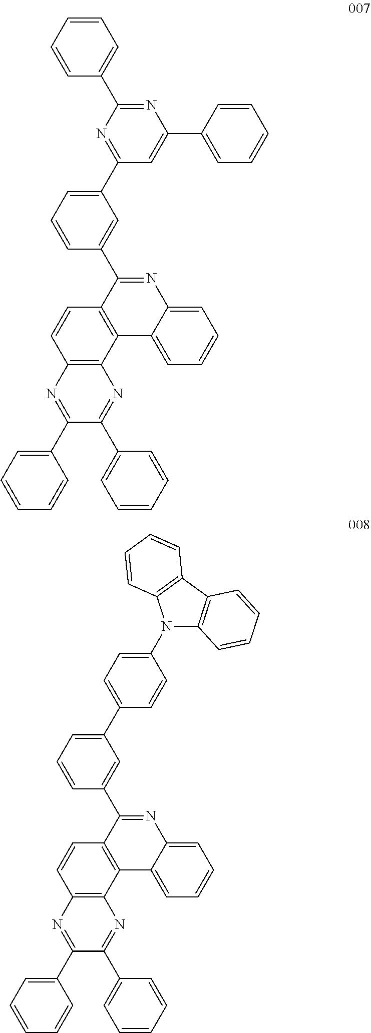

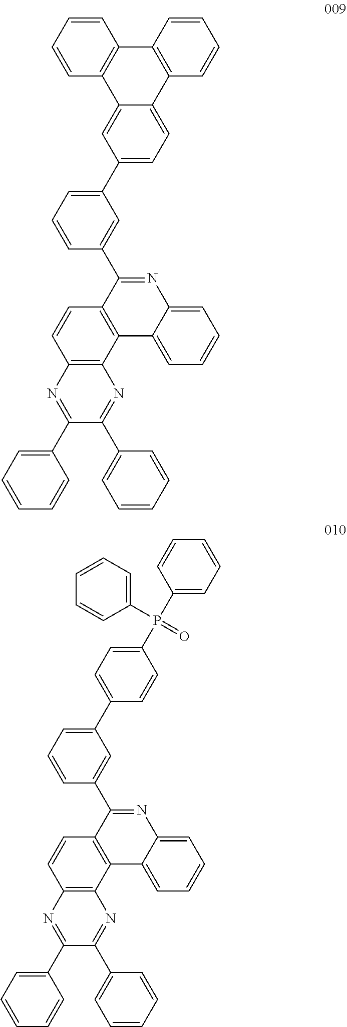

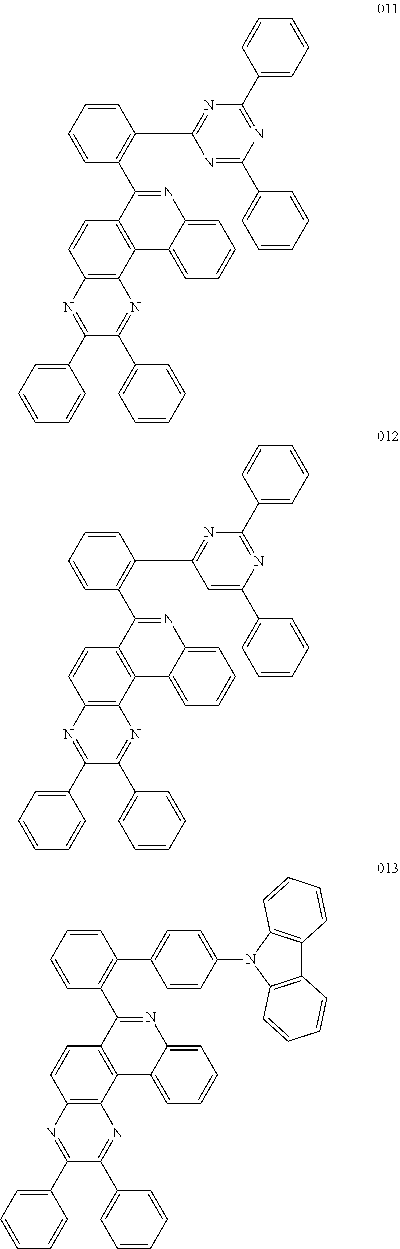 [SMILES: c1ccc(-c2nc(-c3ccccc3)nc(-c3ccccc3-c3nc4ccccc4c4c3ccc3nc(-c5ccccc5)c(-c5ccccc5)nc34)n2)cc1 ; c1ccc(-c2cc(-c3ccccc3-c3nc4ccccc4c4c3ccc3nc(-c5ccccc5)c(-c5ccccc5)nc34)nc(-c3ccccc3)n2)cc1 ; c1ccc(-c2nc3ccc4c(-c5ccccc5-c5ccc(-n6c7ccccc7c7ccccc76)cc5)nc5ccccc5c4c3nc2-c2ccccc2)cc1]

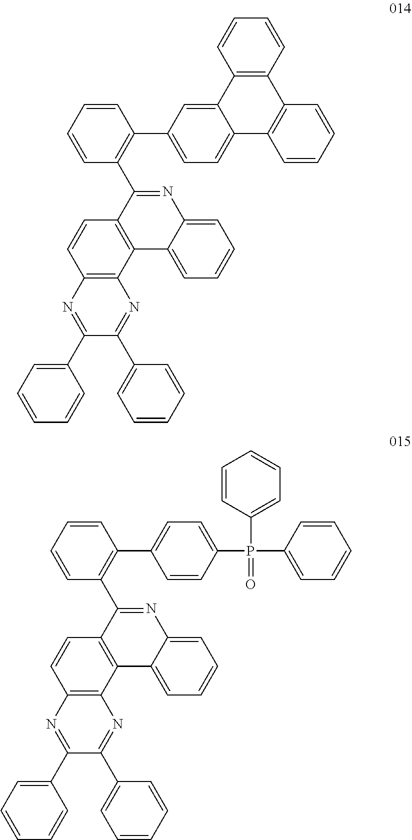 [SMILES: c1ccc(-c2nc3ccc4c(-c5ccccc5-c5ccc6c7ccccc7c7ccccc7c6c5)nc5ccccc5c4c3nc2-c2ccccc2)cc1 ; O=P(c1ccccc1)(c1ccccc1)c1ccc(-c2ccccc2-c2nc3ccccc3c3c2ccc2nc(-c4ccccc4)c(-c4ccccc4)nc23)cc1]

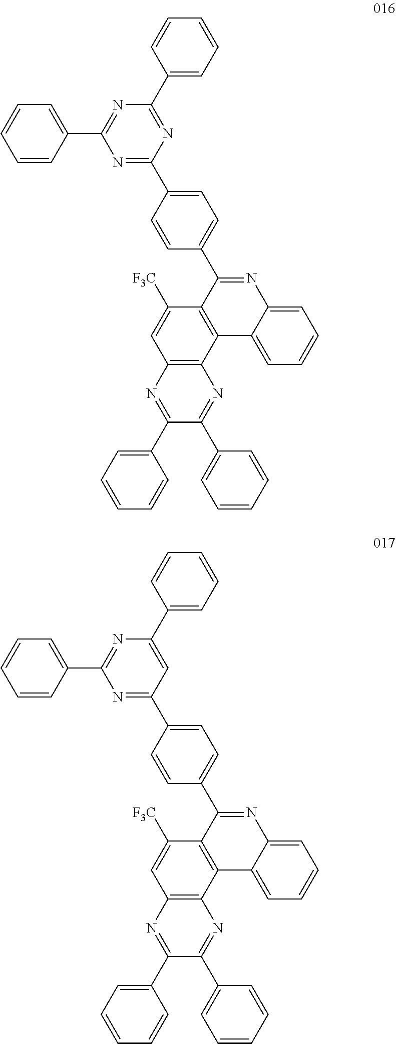 [SMILES: FC(F)(F)c1cc2nc(-c3ccccc3)c(-c3ccccc3)nc2c2c1c(-c1ccc(-c3nc(-c4ccccc4)nc(-c4ccccc4)n3)cc1)nc1ccccc12 ; FC(F)(F)c1cc2nc(-c3ccccc3)c(-c3ccccc3)nc2c2c1c(-c1ccc(-c3cc(-c4ccccc4)nc(-c4ccccc4)n3)cc1)nc1ccccc12]

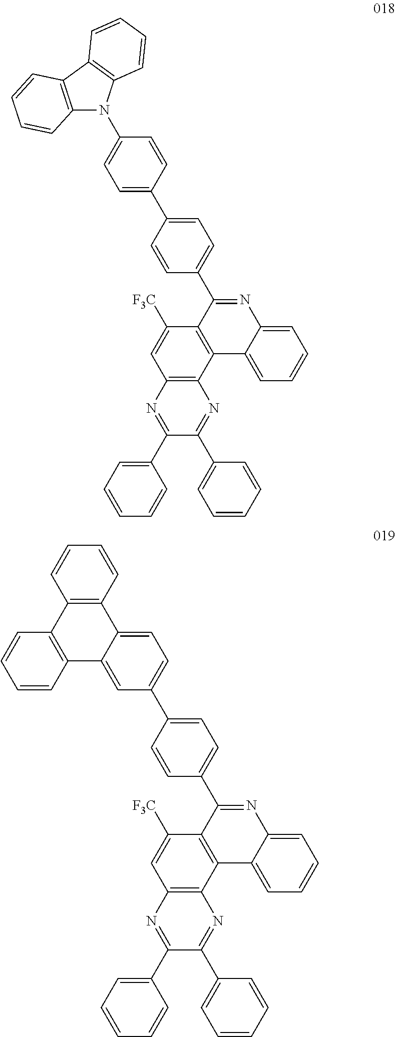

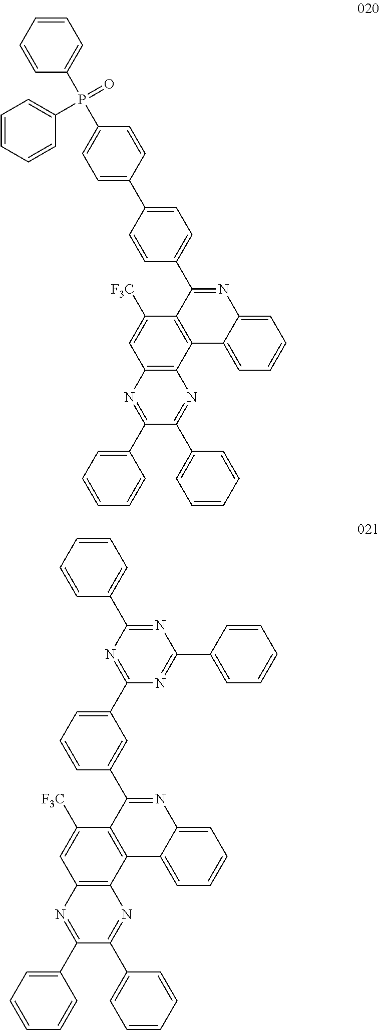

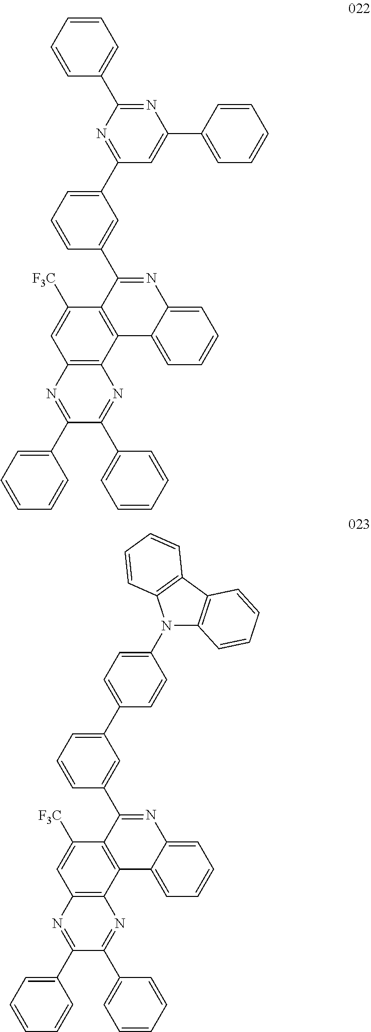

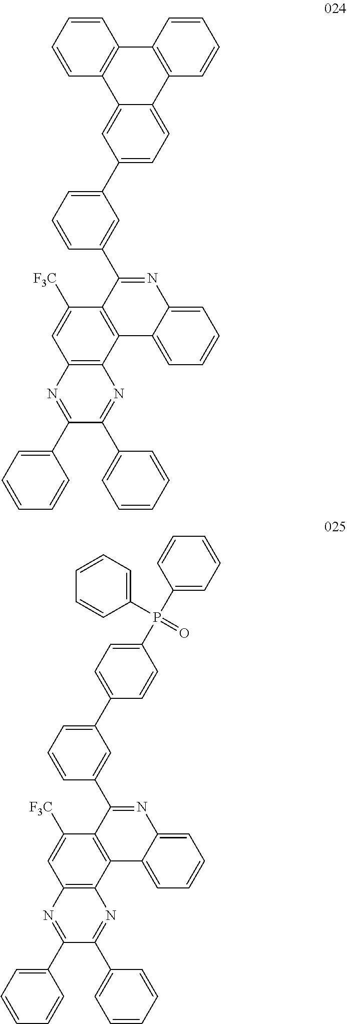

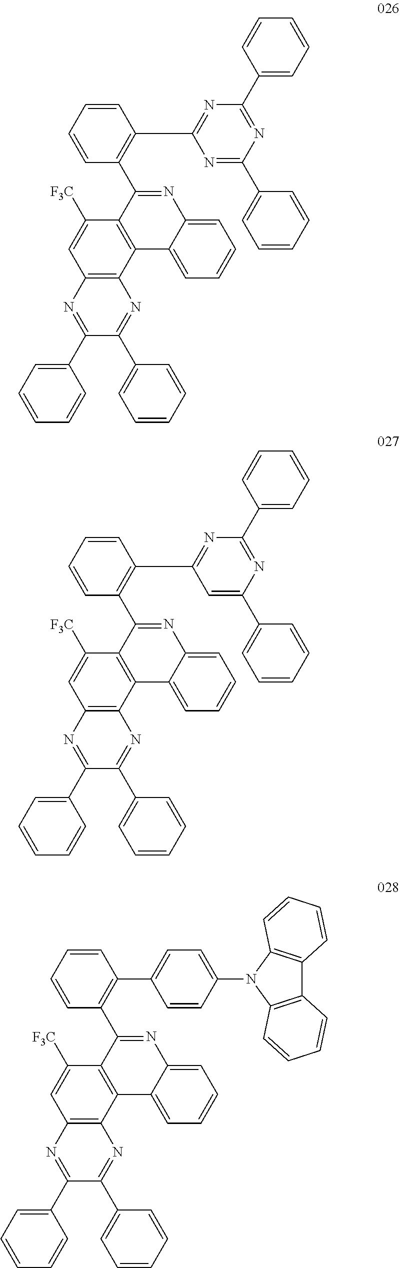 [SMILES: FC(F)(F)c1cc2nc(-c3ccccc3)c(-c3ccccc3)nc2c2c1c(-c1ccccc1-c1nc(-c3ccccc3)nc(-c3ccccc3)n1)nc1ccccc12 ; FC(F)(F)c1cc2nc(-c3ccccc3)c(-c3ccccc3)nc2c2c1c(-c1ccccc1-c1cc(-c3ccccc3)nc(-c3ccccc3)n1)nc1ccccc12 ; FC(F)(F)c1cc2nc(-c3ccccc3)c(-c3ccccc3)nc2c2c1c(-c1ccccc1-c1ccc(-n3c4ccccc4c4ccccc43)cc1)nc1ccccc12]

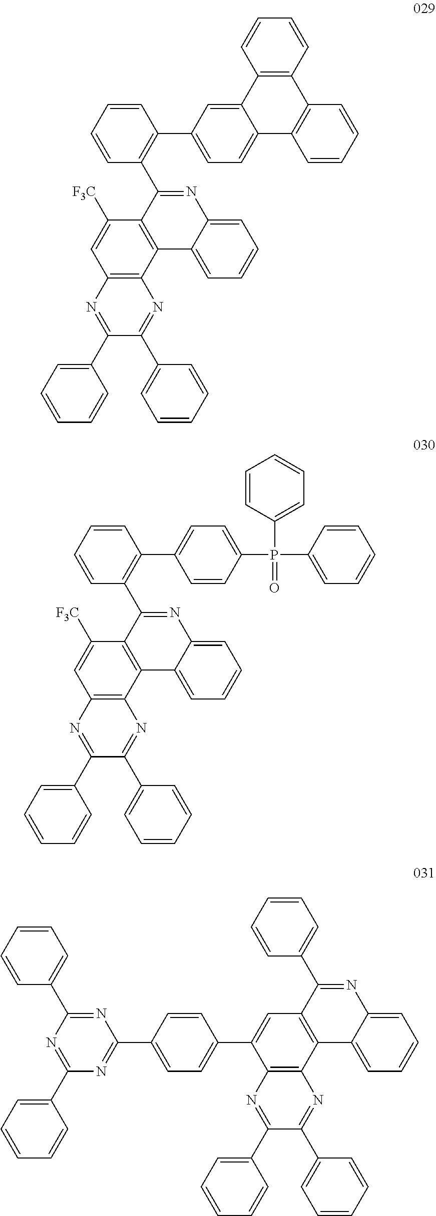 [SMILES: FC(F)(F)c1cc2nc(-c3ccccc3)c(-c3ccccc3)nc2c2c1c(-c1ccccc1-c1ccc3c4ccccc4c4ccccc4c3c1)nc1ccccc12 ; O=P(c1ccccc1)(c1ccccc1)c1ccc(-c2ccccc2-c2nc3ccccc3c3c2c(C(F)(F)F)cc2nc(-c4ccccc4)c(-c4ccccc4)nc23)cc1 ; c1ccc(-c2nc(-c3ccccc3)nc(-c3ccc(-c4cc5c(-c6ccccc6)nc6ccccc6c5c5nc(-c6ccccc6)c(-c6ccccc6)nc45)cc3)n2)cc1]

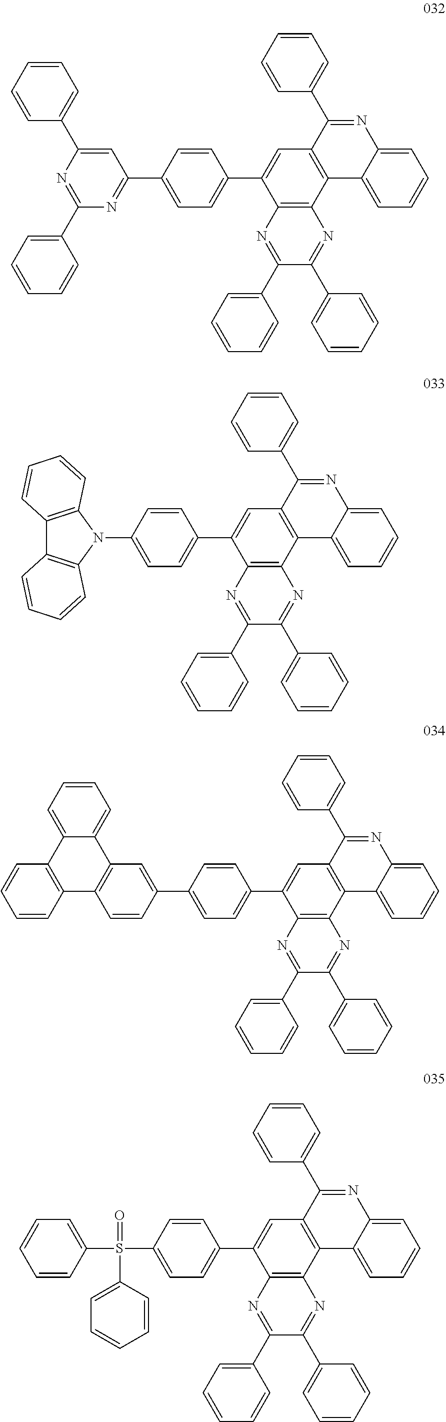

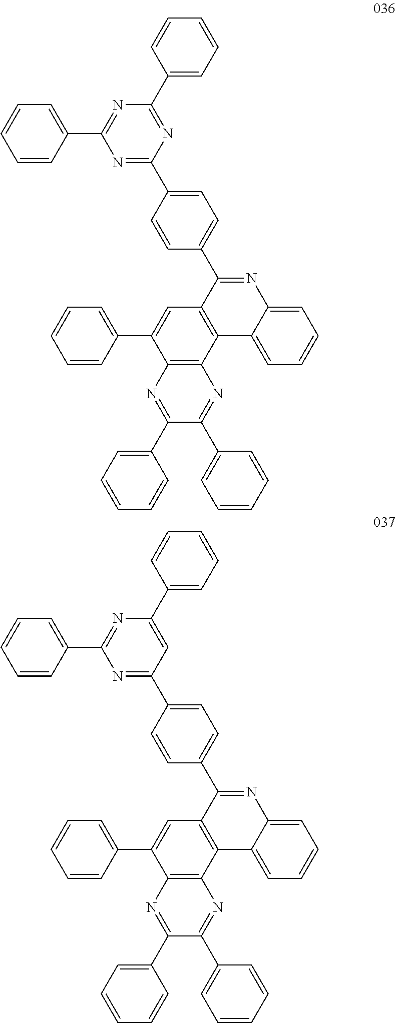

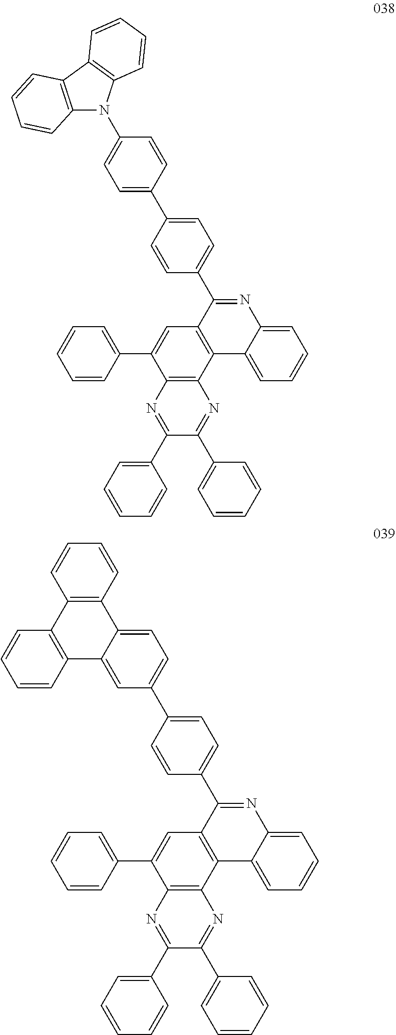

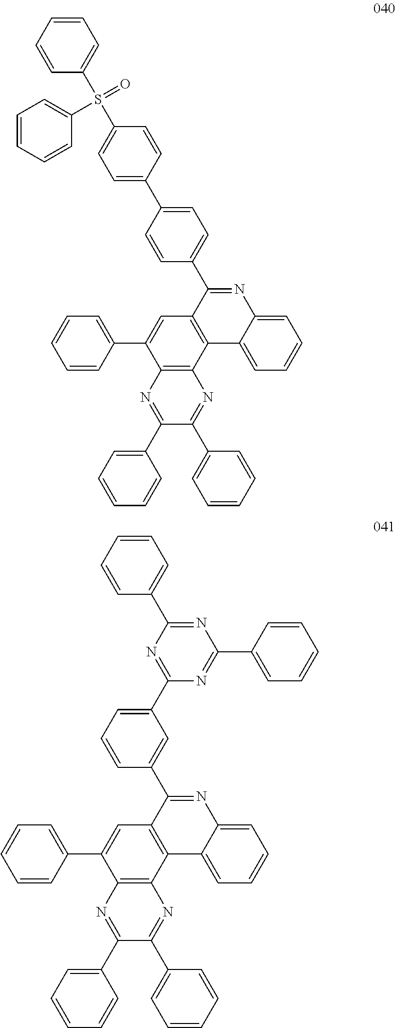

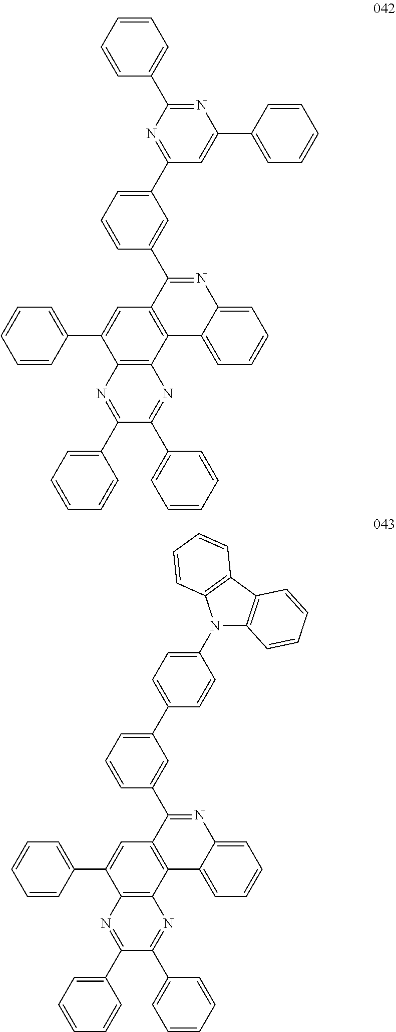

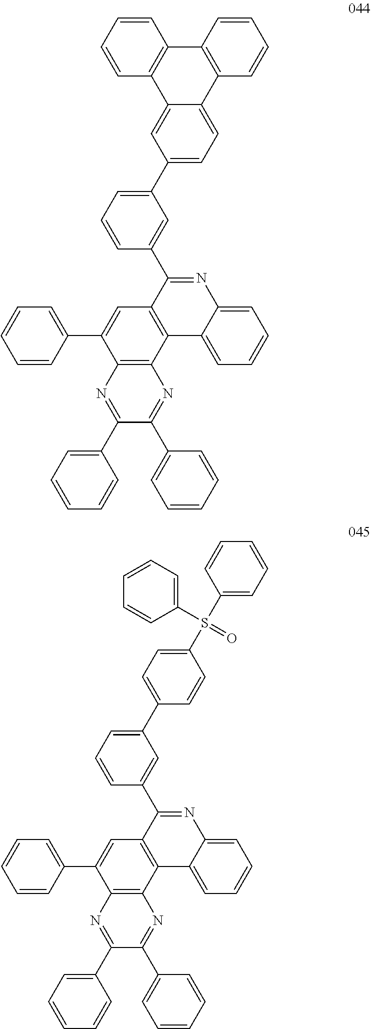

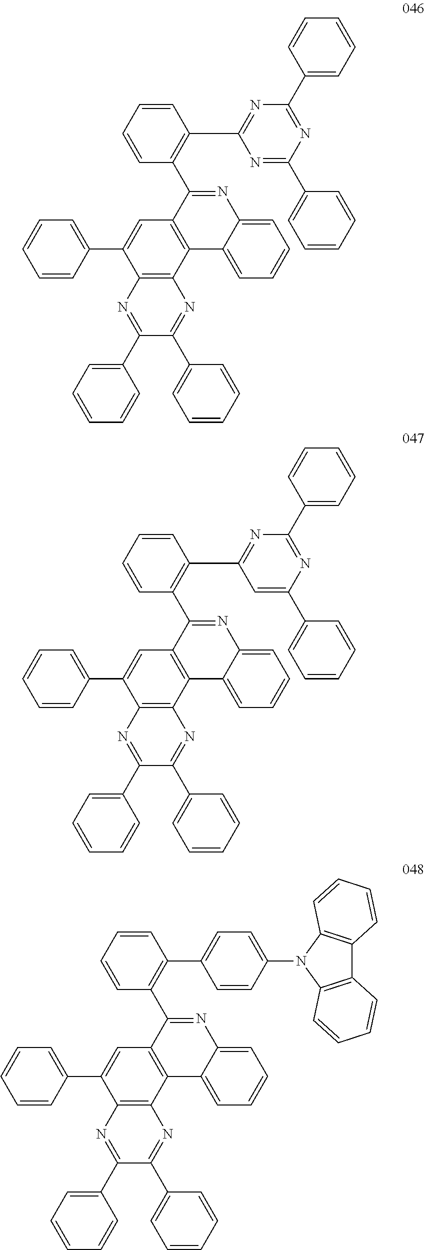 [SMILES: c1ccc(-c2nc(-c3ccccc3)nc(-c3ccccc3-c3nc4ccccc4c4c3cc(-c3ccccc3)c3nc(-c5ccccc5)c(-c5ccccc5)nc34)n2)cc1 ; c1ccc(-c2cc(-c3ccccc3-c3nc4ccccc4c4c3cc(-c3ccccc3)c3nc(-c5ccccc5)c(-c5ccccc5)nc34)nc(-c3ccccc3)n2)cc1 ; c1ccc(-c2nc3c(-c4ccccc4)cc4c(-c5ccccc5-c5ccc(-n6c7ccccc7c7ccccc76)cc5)nc5ccccc5c4c3nc2-c2ccccc2)cc1]

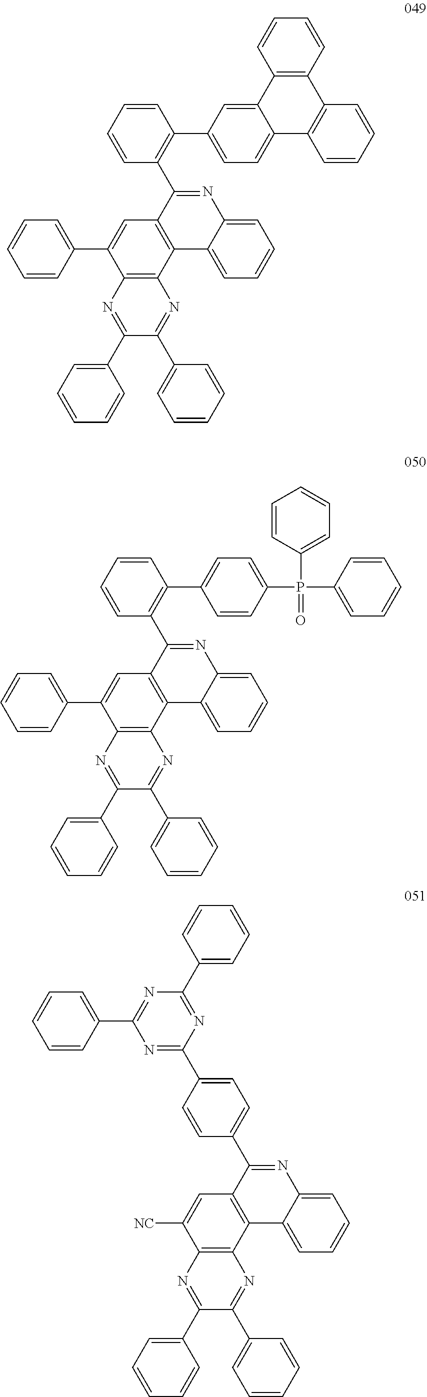

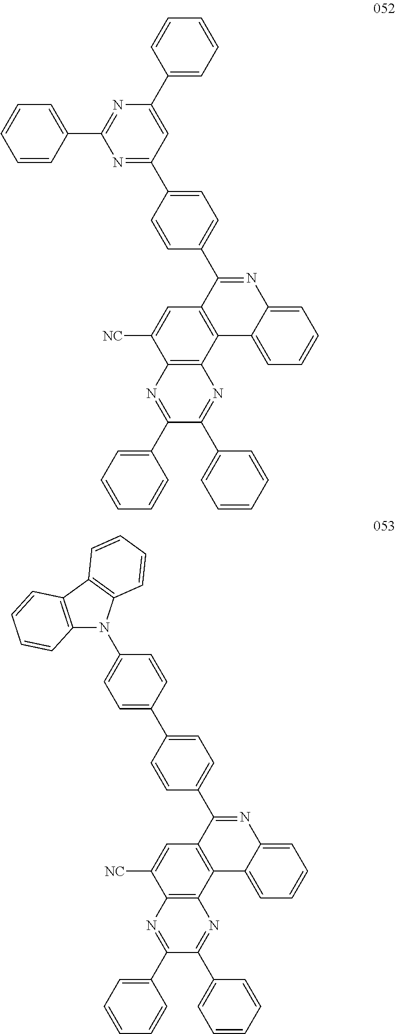

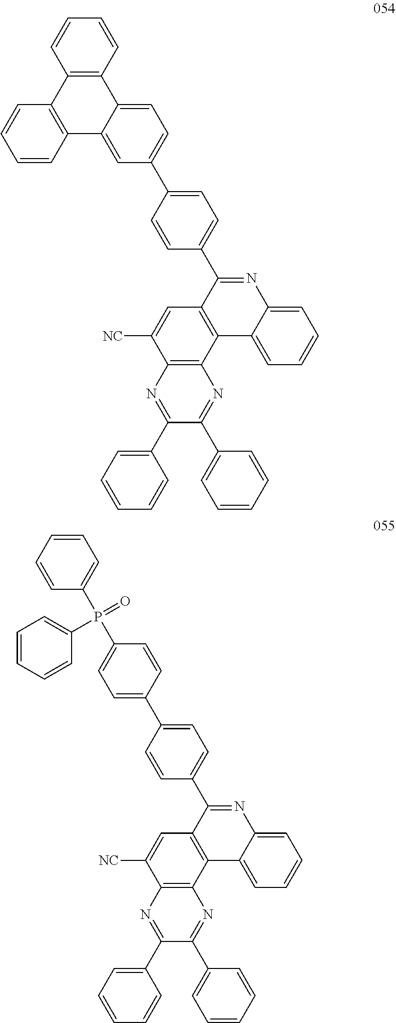

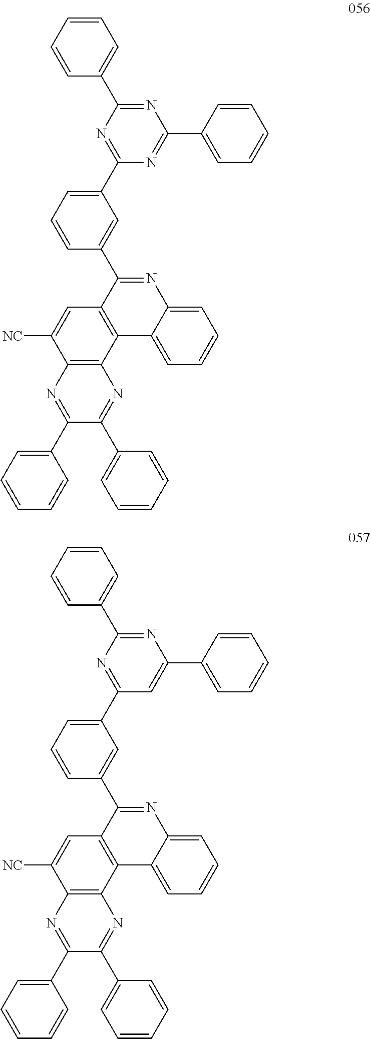

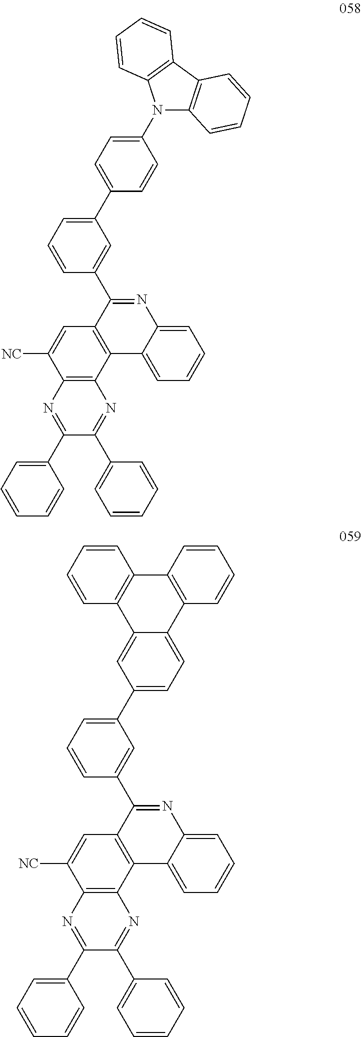

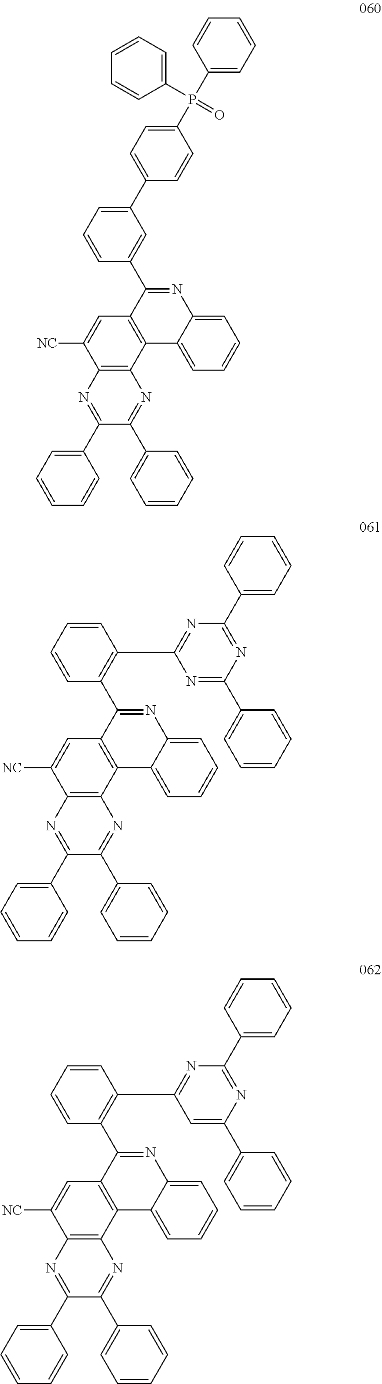 [SMILES: N#Cc1cc2c(-c3cccc(-c4ccc(P(=O)(c5ccccc5)c5ccccc5)cc4)c3)nc3ccccc3c2c2nc(-c3ccccc3)c(-c3ccccc3)nc12 ; N#Cc1cc2c(-c3ccccc3-c3nc(-c4ccccc4)nc(-c4ccccc4)n3)nc3ccccc3c2c2nc(-c3ccccc3)c(-c3ccccc3)nc12 ; N#Cc1cc2c(-c3ccccc3-c3cc(-c4ccccc4)nc(-c4ccccc4)n3)nc3ccccc3c2c2nc(-c3ccccc3)c(-c3ccccc3)nc12]

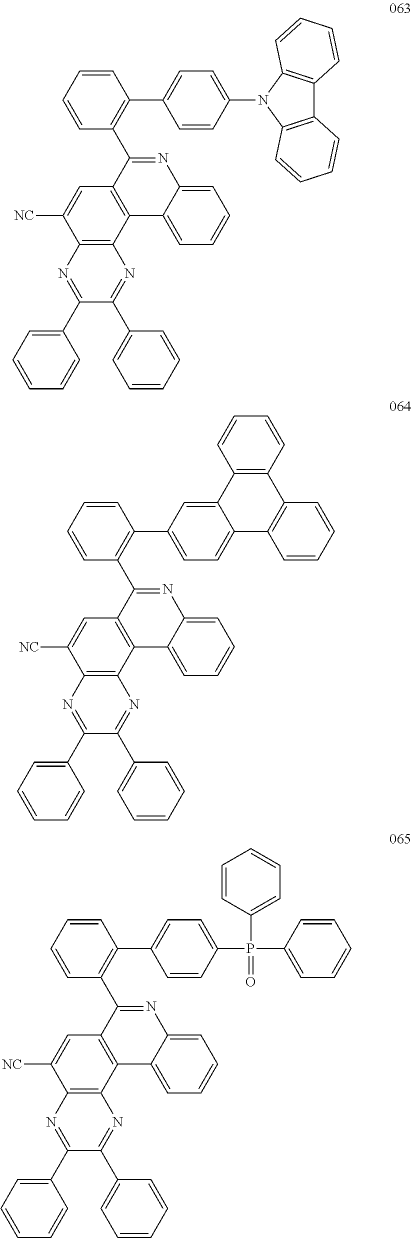 [SMILES: N#Cc1cc2c(-c3ccccc3-c3ccc(-n4c5ccccc5c5ccccc54)cc3)nc3ccccc3c2c2nc(-c3ccccc3)c(-c3ccccc3)nc12 ; N#Cc1cc2c(-c3ccccc3-c3ccc4c5ccccc5c5ccccc5c4c3)nc3ccccc3c2c2nc(-c3ccccc3)c(-c3ccccc3)nc12 ; N#Cc1cc2c(-c3ccccc3-c3ccc(P(=O)(c4ccccc4)c4ccccc4)cc3)nc3ccccc3c2c2nc(-c3ccccc3)c(-c3ccccc3)nc12]

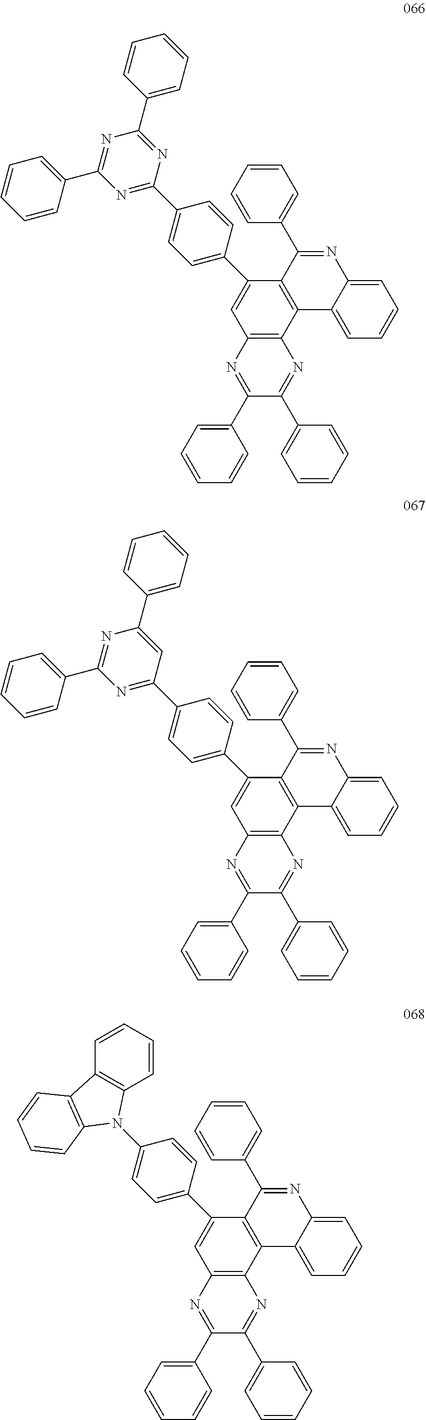 [SMILES: c1ccc(-c2nc(-c3ccccc3)nc(-c3ccc(-c4cc5nc(-c6ccccc6)c(-c6ccccc6)nc5c5c4c(-c4ccccc4)nc4ccccc45)cc3)n2)cc1 ; c1ccc(-c2cc(-c3ccc(-c4cc5nc(-c6ccccc6)c(-c6ccccc6)nc5c5c4c(-c4ccccc4)nc4ccccc45)cc3)nc(-c3ccccc3)n2)cc1 ; c1ccc(-c2nc3cc(-c4ccc(-n5c6ccccc6c6ccccc65)cc4)c4c(-c5ccccc5)nc5ccccc5c4c3nc2-c2ccccc2)cc1]

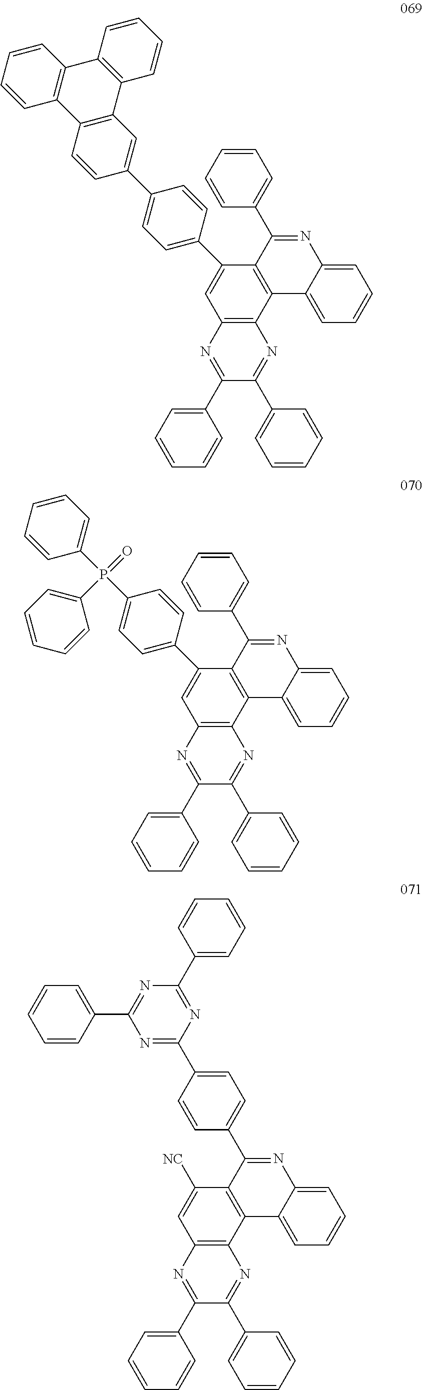

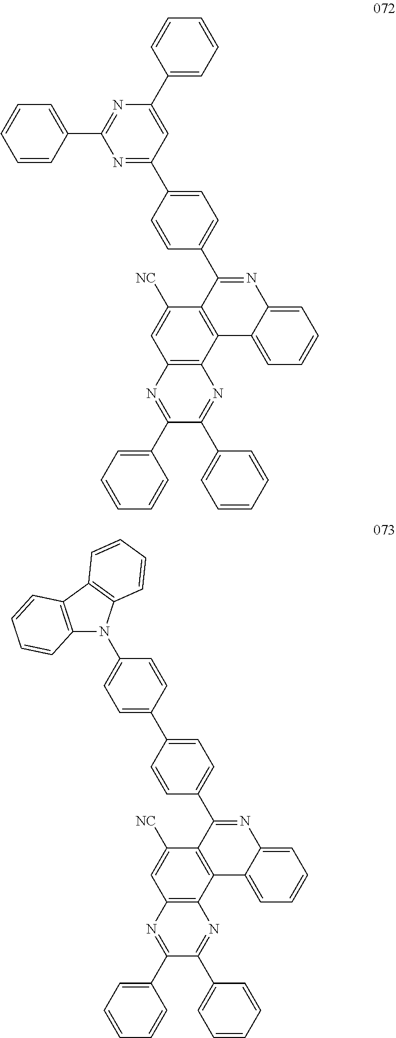

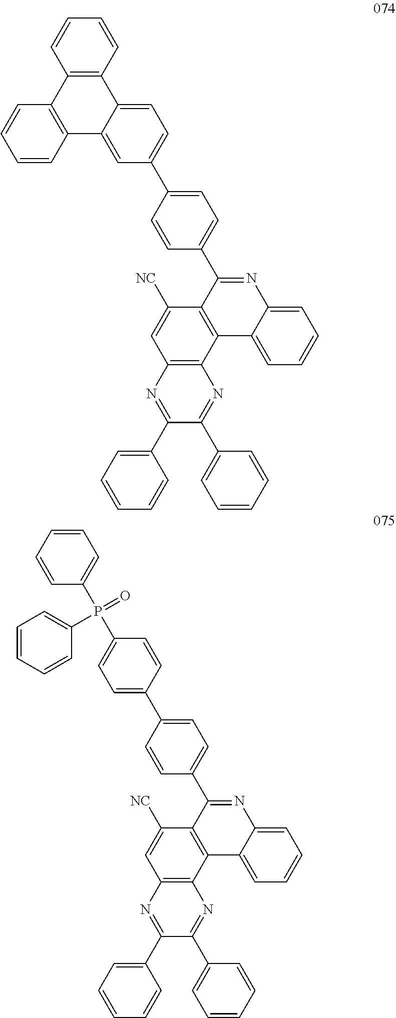

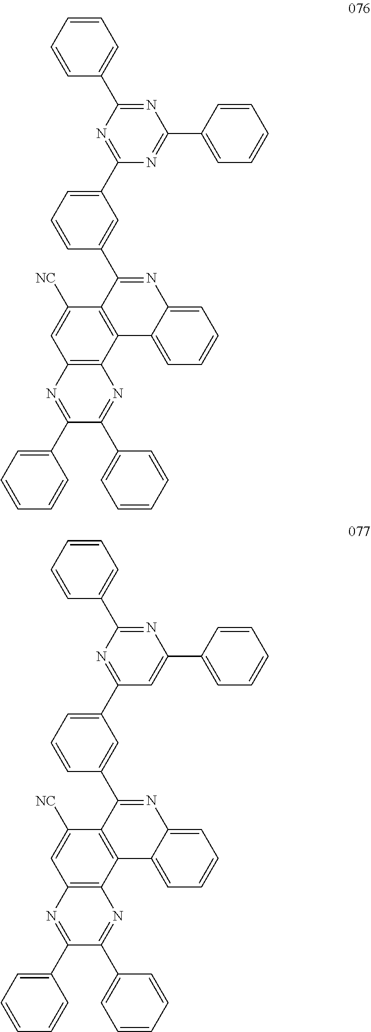

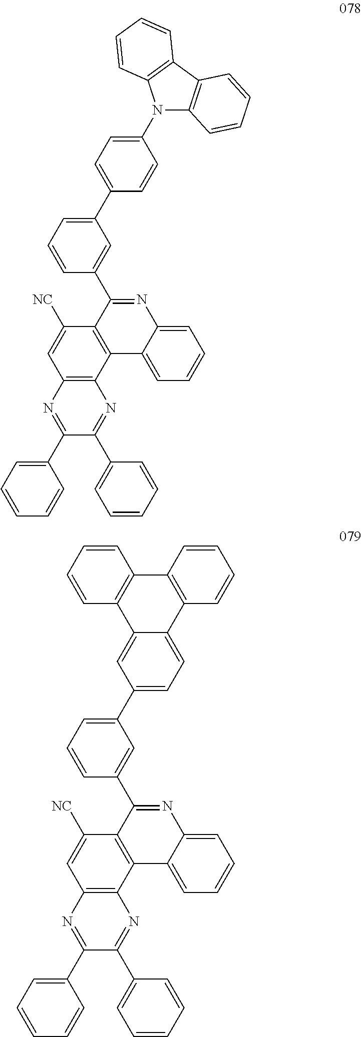

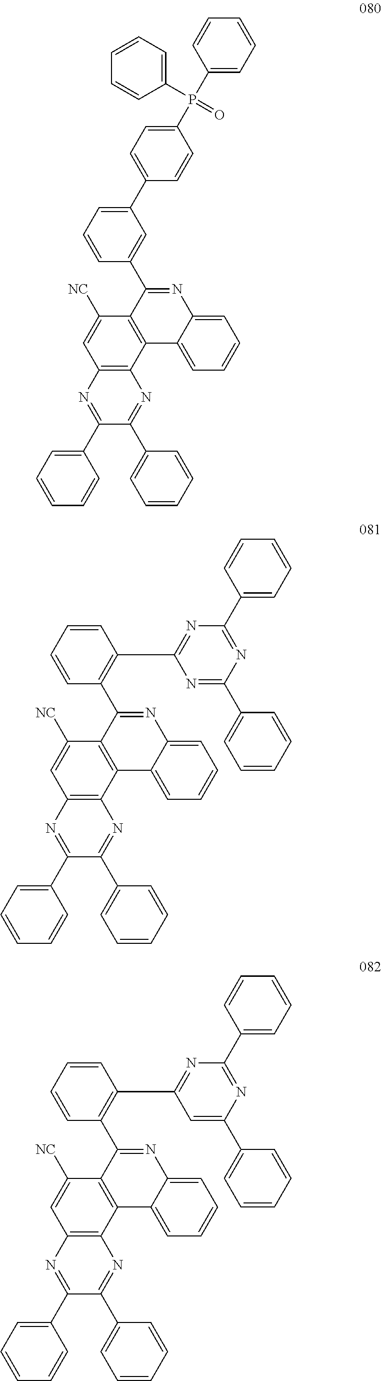 [SMILES: N#Cc1cc2nc(-c3ccccc3)c(-c3ccccc3)nc2c2c1c(-c1cccc(-c3ccc(P(=O)(c4ccccc4)c4ccccc4)cc3)c1)nc1ccccc12 ; N#Cc1cc2nc(-c3ccccc3)c(-c3ccccc3)nc2c2c1c(-c1ccccc1-c1nc(-c3ccccc3)nc(-c3ccccc3)n1)nc1ccccc12 ; N#Cc1cc2nc(-c3ccccc3)c(-c3ccccc3)nc2c2c1c(-c1ccccc1-c1cc(-c3ccccc3)nc(-c3ccccc3)n1)nc1ccccc12]

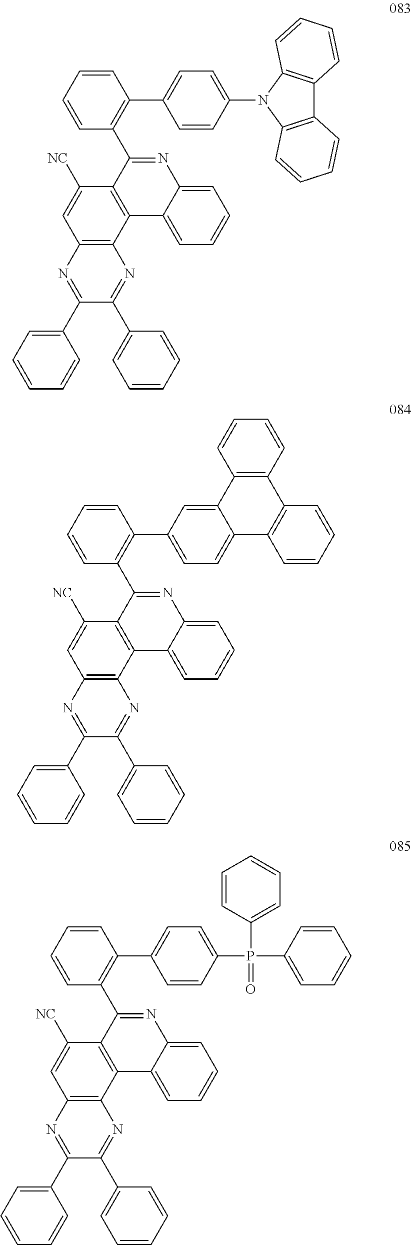 [SMILES: N#Cc1cc2nc(-c3ccccc3)c(-c3ccccc3)nc2c2c1c(-c1ccccc1-c1ccc(-n3c4ccccc4c4ccccc43)cc1)nc1ccccc12 ; N#Cc1cc2nc(-c3ccccc3)c(-c3ccccc3)nc2c2c1c(-c1ccccc1-c1ccc3c4ccccc4c4ccccc4c3c1)nc1ccccc12 ; N#Cc1cc2nc(-c3ccccc3)c(-c3ccccc3)nc2c2c1c(-c1ccccc1-c1ccc(P(=O)(c3ccccc3)c3ccccc3)cc1)nc1ccccc12]

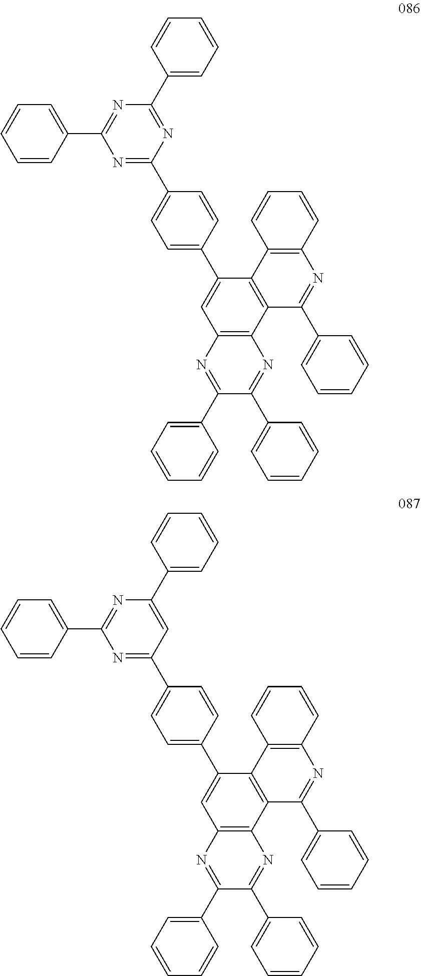

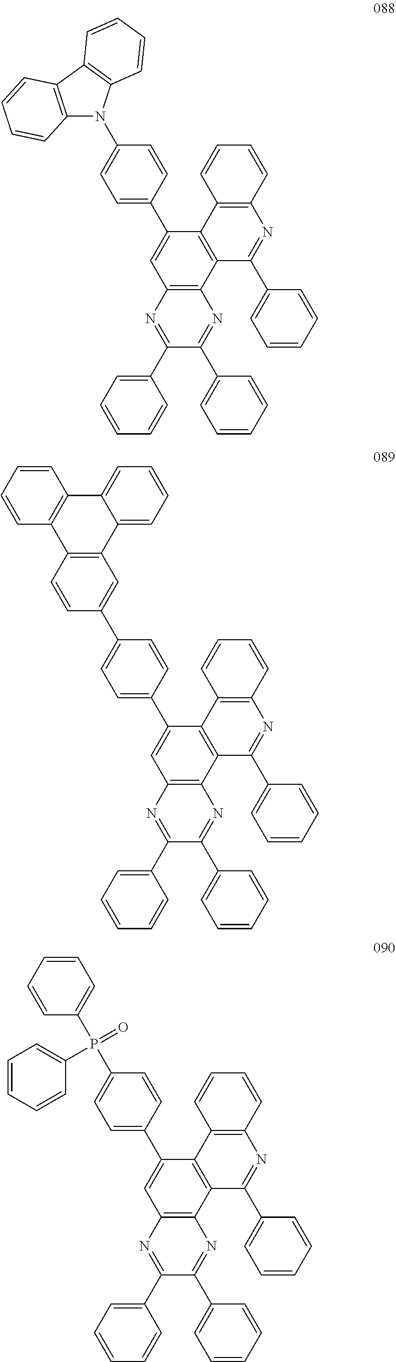

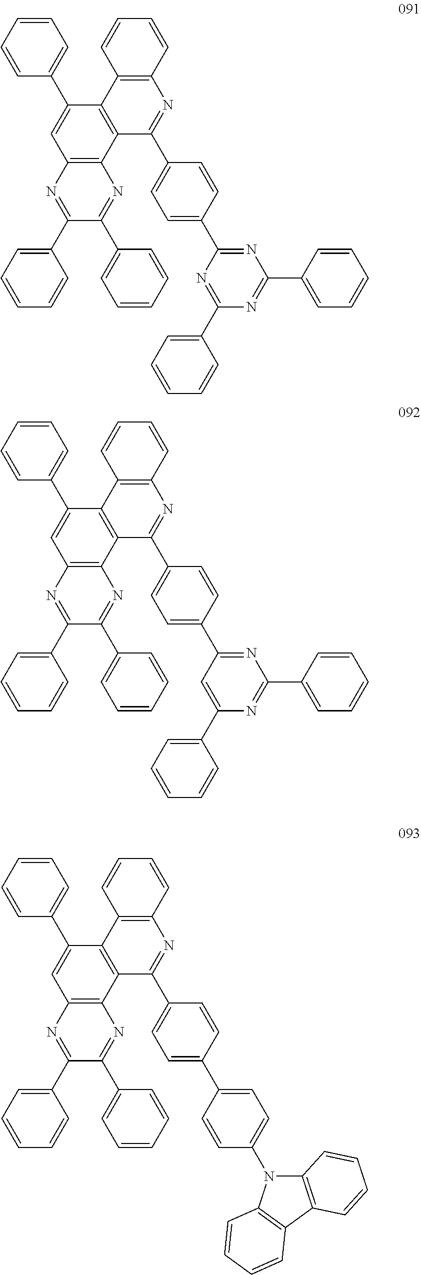

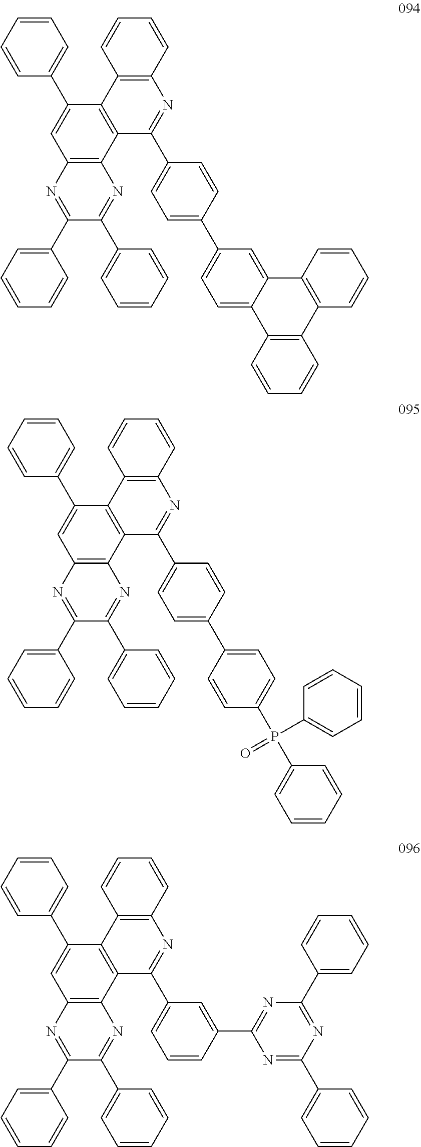 [SMILES: c1ccc(-c2nc3cc(-c4ccccc4)c4c5ccccc5nc(-c5ccc(-c6ccc7c8ccccc8c8ccccc8c7c6)cc5)c4c3nc2-c2ccccc2)cc1 ; O=P(c1ccccc1)(c1ccccc1)c1ccc(-c2ccc(-c3nc4ccccc4c4c(-c5ccccc5)cc5nc(-c6ccccc6)c(-c6ccccc6)nc5c34)cc2)cc1 ; c1ccc(-c2nc(-c3ccccc3)nc(-c3cccc(-c4nc5ccccc5c5c(-c6ccccc6)cc6nc(-c7ccccc7)c(-c7ccccc7)nc6c45)c3)n2)cc1]

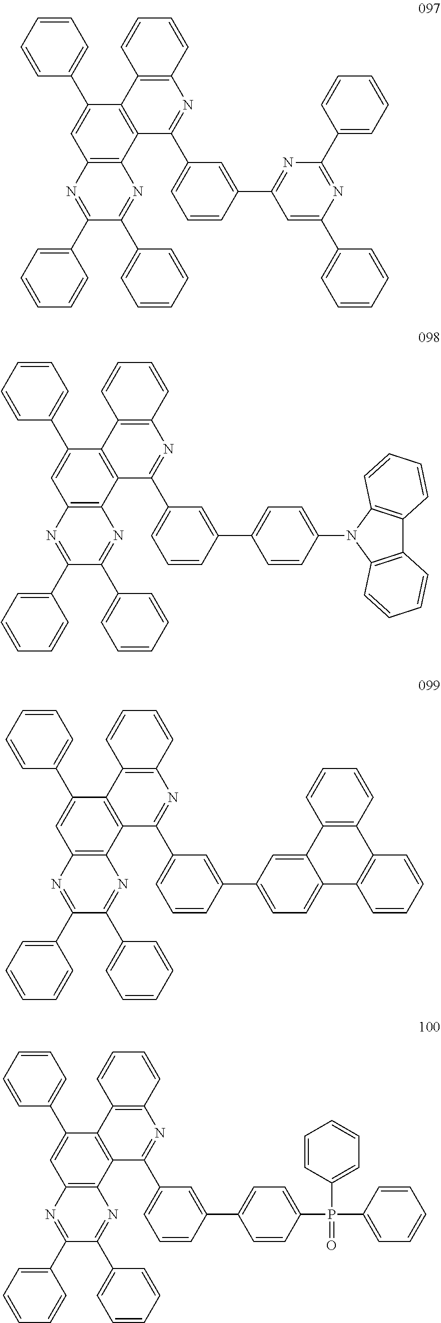 [SMILES: c1ccc(-c2cc(-c3cccc(-c4nc5ccccc5c5c(-c6ccccc6)cc6nc(-c7ccccc7)c(-c7ccccc7)nc6c45)c3)nc(-c3ccccc3)n2)cc1 ; c1ccc(-c2nc3cc(-c4ccccc4)c4c5ccccc5nc(-c5cccc(-c6ccc(-n7c8ccccc8c8ccccc87)cc6)c5)c4c3nc2-c2ccccc2)cc1 ; c1ccc(-c2nc3cc(-c4ccccc4)c4c5ccccc5nc(-c5cccc(-c6ccc7c8ccccc8c8ccccc8c7c6)c5)c4c3nc2-c2ccccc2)cc1 ; O=P(c1ccccc1)(c1ccccc1)c1ccc(-c2cccc(-c3nc4ccccc4c4c(-c5ccccc5)cc5nc(-c6ccccc6)c(-c6ccccc6)nc5c34)c2)cc1]

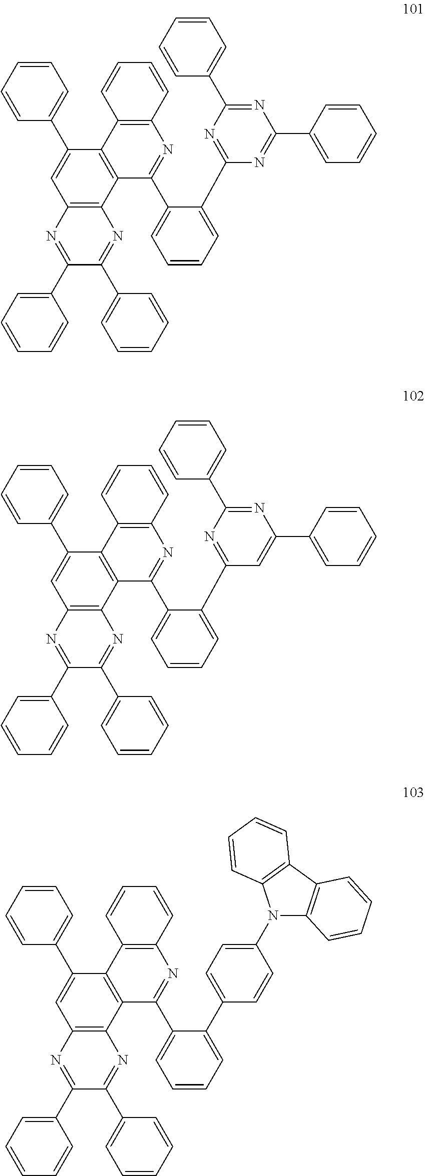 [SMILES: c1ccc(-c2nc(-c3ccccc3)nc(-c3ccccc3-c3nc4ccccc4c4c(-c5ccccc5)cc5nc(-c6ccccc6)c(-c6ccccc6)nc5c34)n2)cc1 ; c1ccc(-c2cc(-c3ccccc3-c3nc4ccccc4c4c(-c5ccccc5)cc5nc(-c6ccccc6)c(-c6ccccc6)nc5c34)nc(-c3ccccc3)n2)cc1 ; c1ccc(-c2nc3cc(-c4ccccc4)c4c5ccccc5nc(-c5ccccc5-c5ccc(-n6c7ccccc7c7ccccc76)cc5)c4c3nc2-c2ccccc2)cc1]

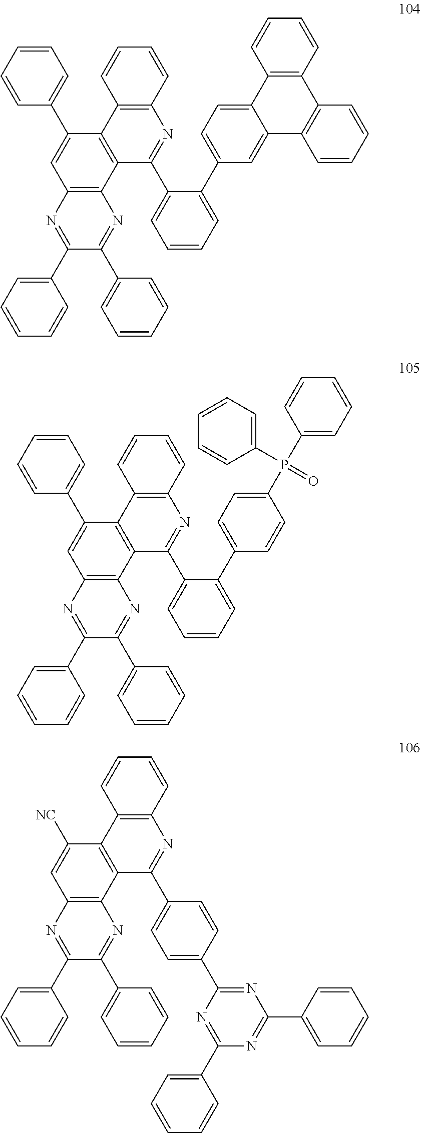

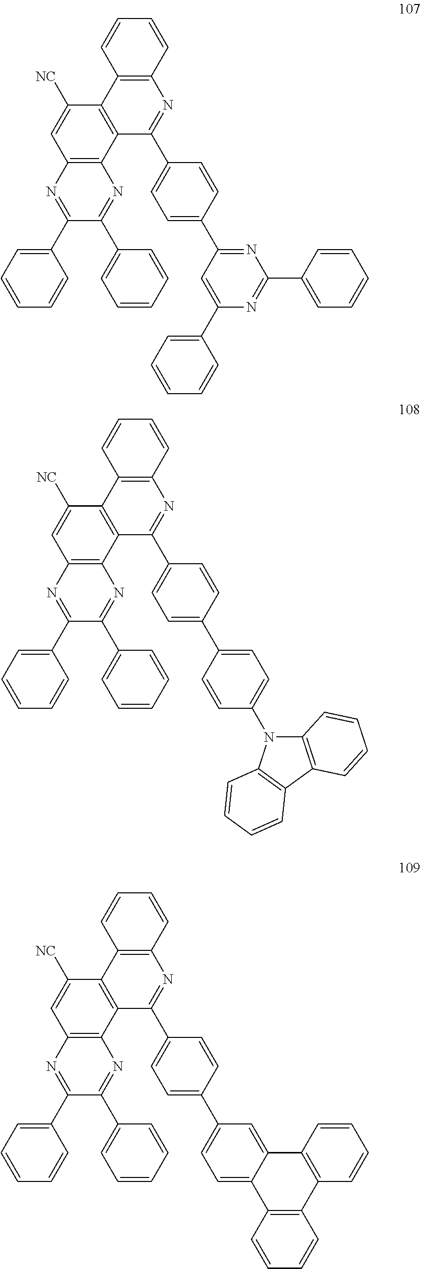

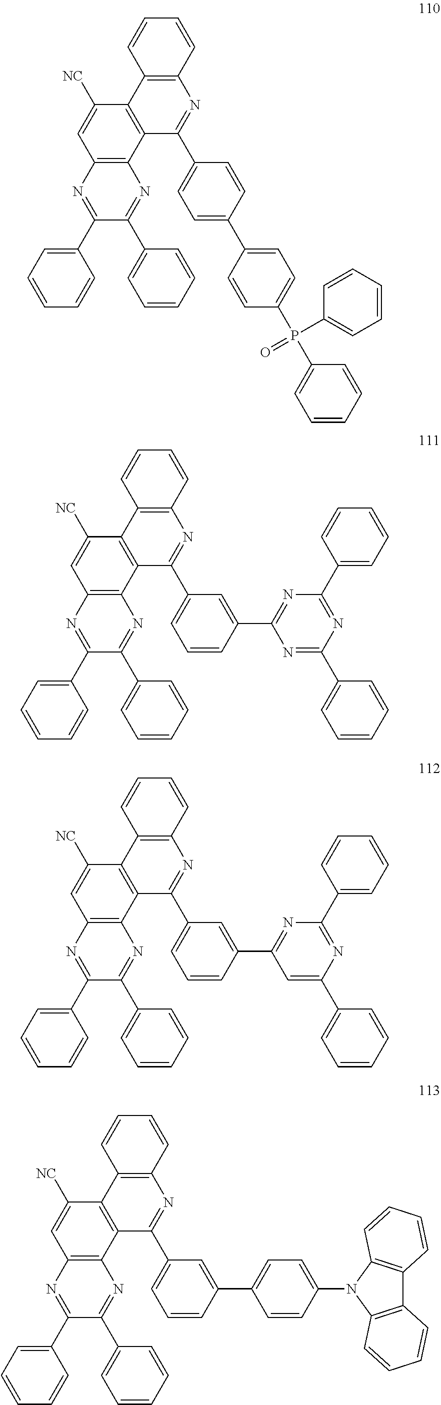

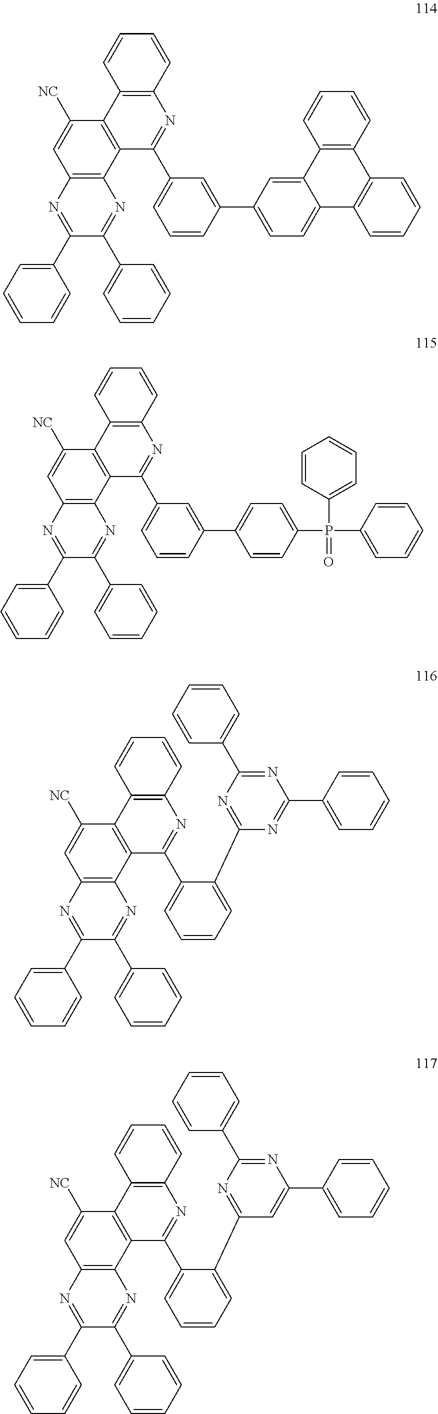 [SMILES: N#Cc1cc2nc(-c3ccccc3)c(-c3ccccc3)nc2c2c(-c3cccc(-c4ccc5c6ccccc6c6ccccc6c5c4)c3)nc3ccccc3c12 ; N#Cc1cc2nc(-c3ccccc3)c(-c3ccccc3)nc2c2c(-c3cccc(-c4ccc(P(=O)(c5ccccc5)c5ccccc5)cc4)c3)nc3ccccc3c12 ; N#Cc1cc2nc(-c3ccccc3)c(-c3ccccc3)nc2c2c(-c3ccccc3-c3nc(-c4ccccc4)nc(-c4ccccc4)n3)nc3ccccc3c12 ; N#Cc1cc2nc(-c3ccccc3)c(-c3ccccc3)nc2c2c(-c3ccccc3-c3cc(-c4ccccc4)nc(-c4ccccc4)n3)nc3ccccc3c12]

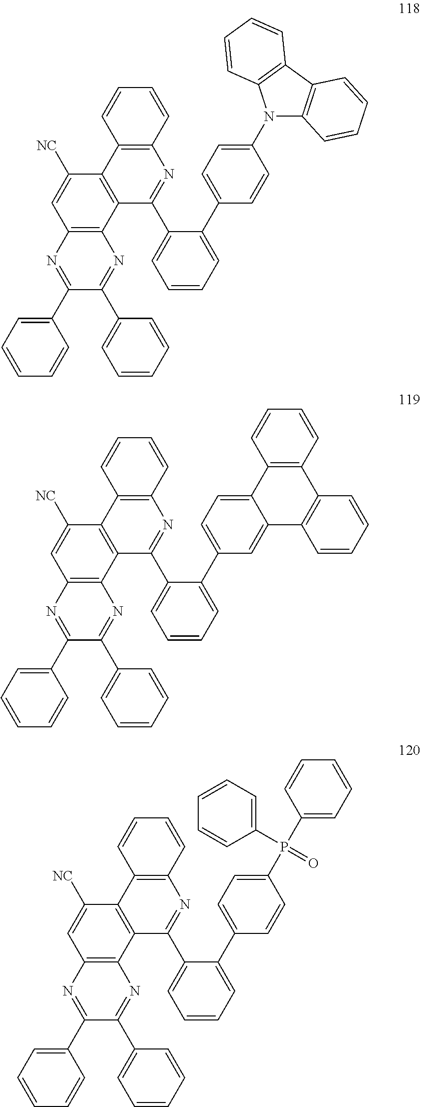 [SMILES: N#Cc1cc2nc(-c3ccccc3)c(-c3ccccc3)nc2c2c(-c3ccccc3-c3ccc(-n4c5ccccc5c5ccccc54)cc3)nc3ccccc3c12 ; N#Cc1cc2nc(-c3ccccc3)c(-c3ccccc3)nc2c2c(-c3ccccc3-c3ccc4c5ccccc5c5ccccc5c4c3)nc3ccccc3c12 ; N#Cc1cc2nc(-c3ccccc3)c(-c3ccccc3)nc2c2c(-c3ccccc3-c3ccc(P(=O)(c4ccccc4)c4ccccc4)cc3)nc3ccccc3c12]

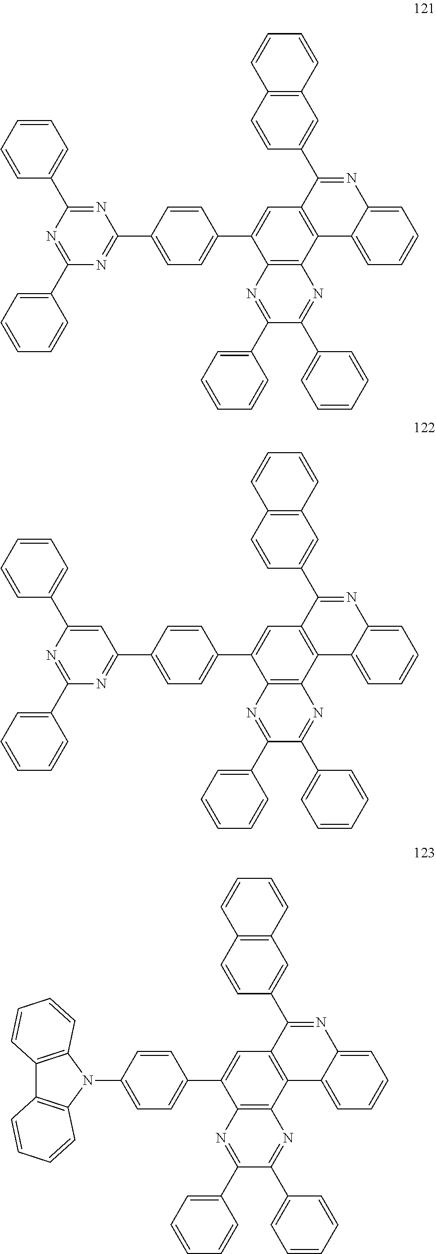

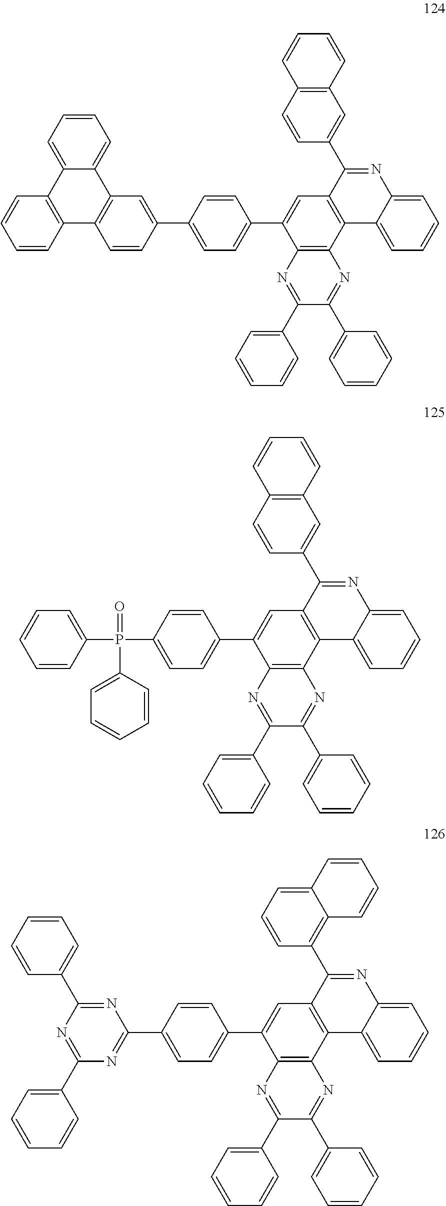 [SMILES: c1ccc(-c2nc3c(-c4ccc(-c5ccc6c7ccccc7c7ccccc7c6c5)cc4)cc4c(-c5ccc6ccccc6c5)nc5ccccc5c4c3nc2-c2ccccc2)cc1 ; O=P(c1ccccc1)(c1ccccc1)c1ccc(-c2cc3c(-c4ccc5ccccc5c4)nc4ccccc4c3c3nc(-c4ccccc4)c(-c4ccccc4)nc23)cc1 ; c1ccc(-c2nc(-c3ccccc3)nc(-c3ccc(-c4cc5c(-c6cccc7ccccc67)nc6ccccc6c5c5nc(-c6ccccc6)c(-c6ccccc6)nc45)cc3)n2)cc1]

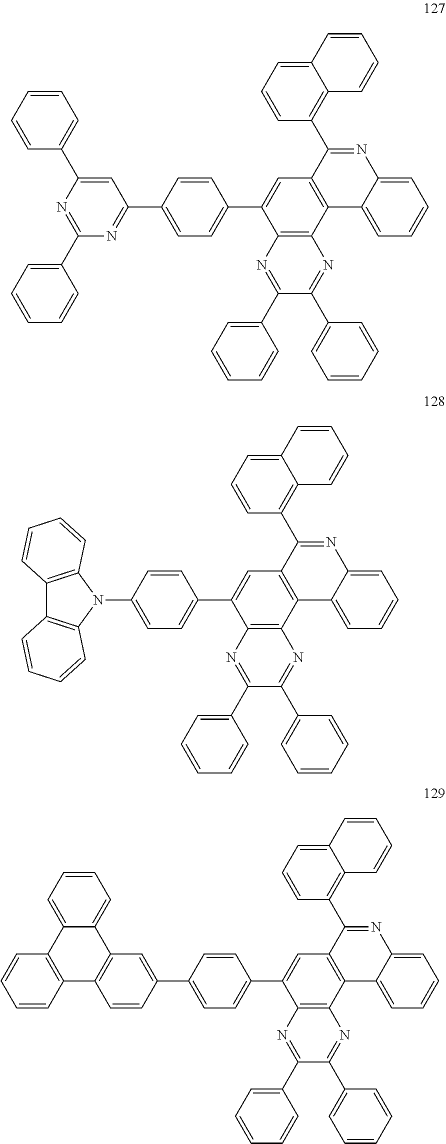

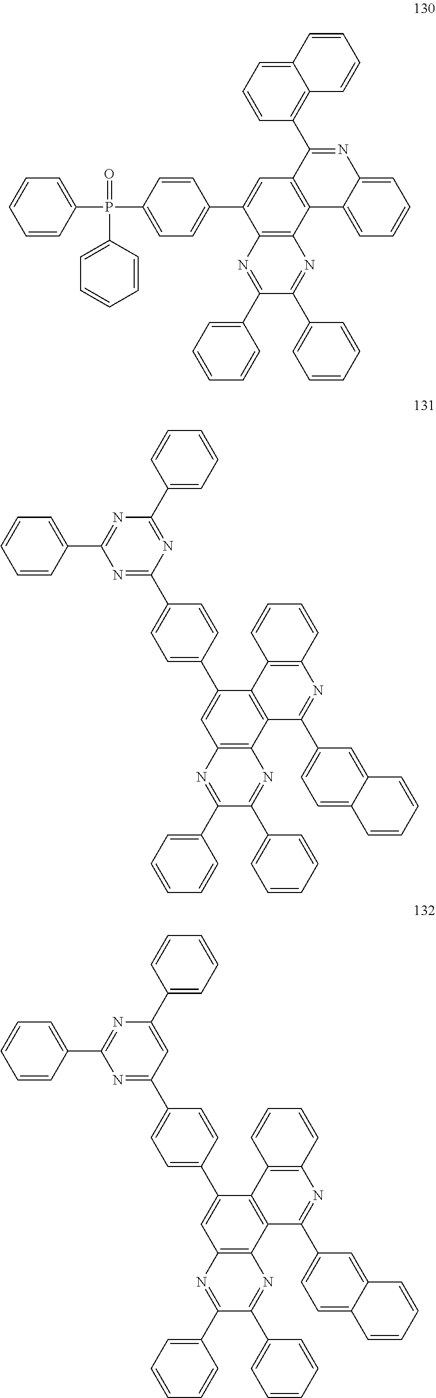

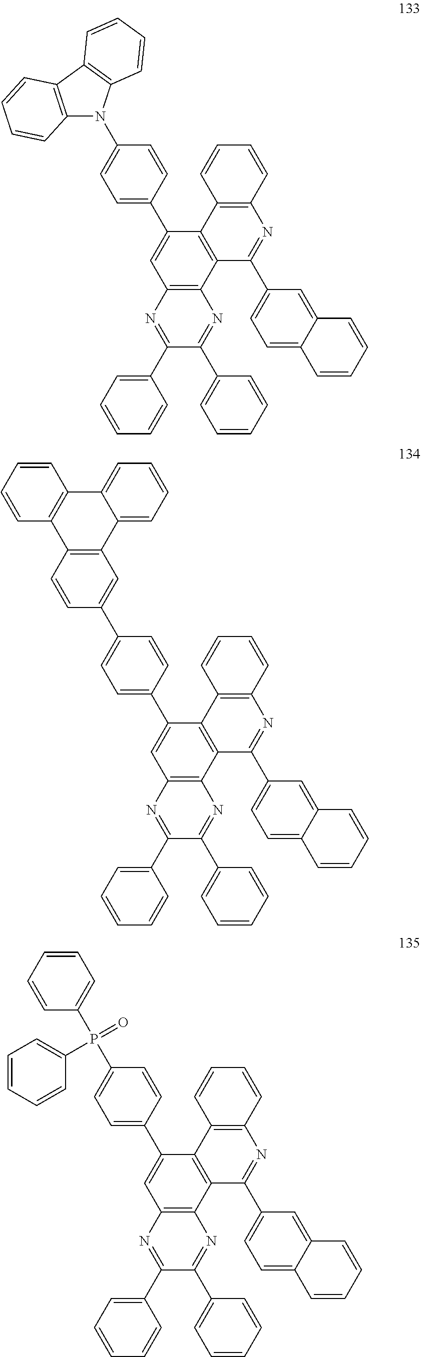

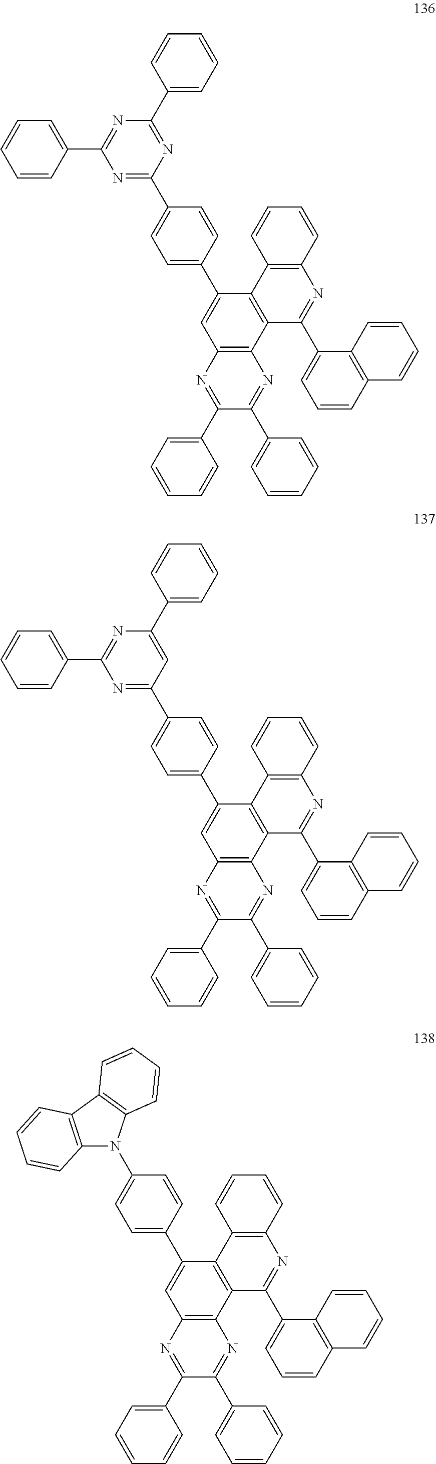 [SMILES: c1ccc(-c2nc(-c3ccccc3)nc(-c3ccc(-c4cc5nc(-c6ccccc6)c(-c6ccccc6)nc5c5c(-c6cccc7ccccc67)nc6ccccc6c45)cc3)n2)cc1 ; c1ccc(-c2cc(-c3ccc(-c4cc5nc(-c6ccccc6)c(-c6ccccc6)nc5c5c(-c6cccc7ccccc67)nc6ccccc6c45)cc3)nc(-c3ccccc3)n2)cc1 ; c1ccc(-c2nc3cc(-c4ccc(-n5c6ccccc6c6ccccc65)cc4)c4c5ccccc5nc(-c5cccc6ccccc56)c4c3nc2-c2ccccc2)cc1]

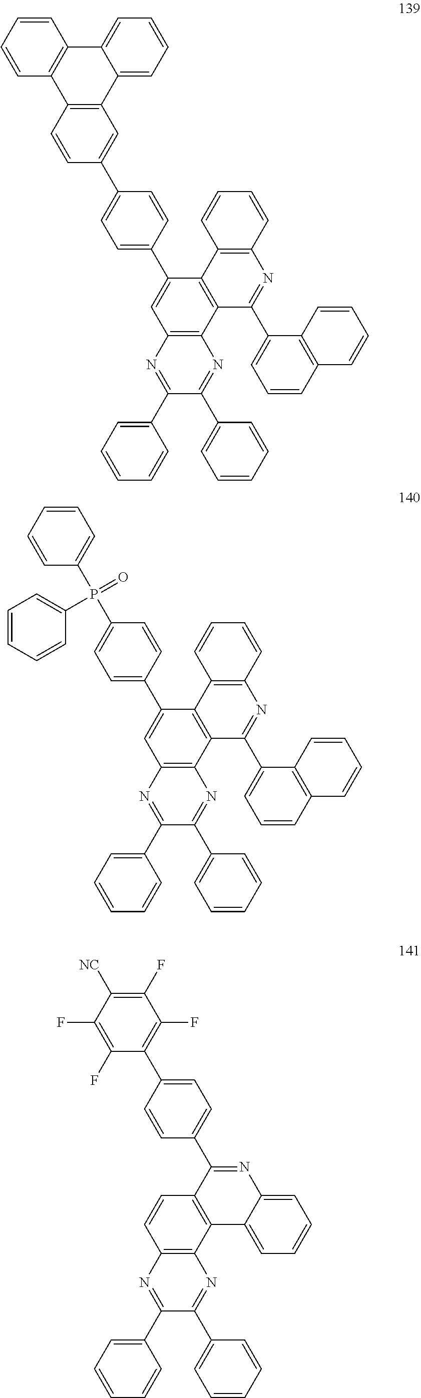 [SMILES: c1ccc(-c2nc3cc(-c4ccc(-c5ccc6c7ccccc7c7ccccc7c6c5)cc4)c4c5ccccc5nc(-c5cccc6ccccc56)c4c3nc2-c2ccccc2)cc1 ; O=P(c1ccccc1)(c1ccccc1)c1ccc(-c2cc3nc(-c4ccccc4)c(-c4ccccc4)nc3c3c(-c4cccc5ccccc45)nc4ccccc4c23)cc1 ; N#Cc1c(F)c(F)c(-c2ccc(-c3nc4ccccc4c4c3ccc3nc(-c5ccccc5)c(-c5ccccc5)nc34)cc2)c(F)c1F]

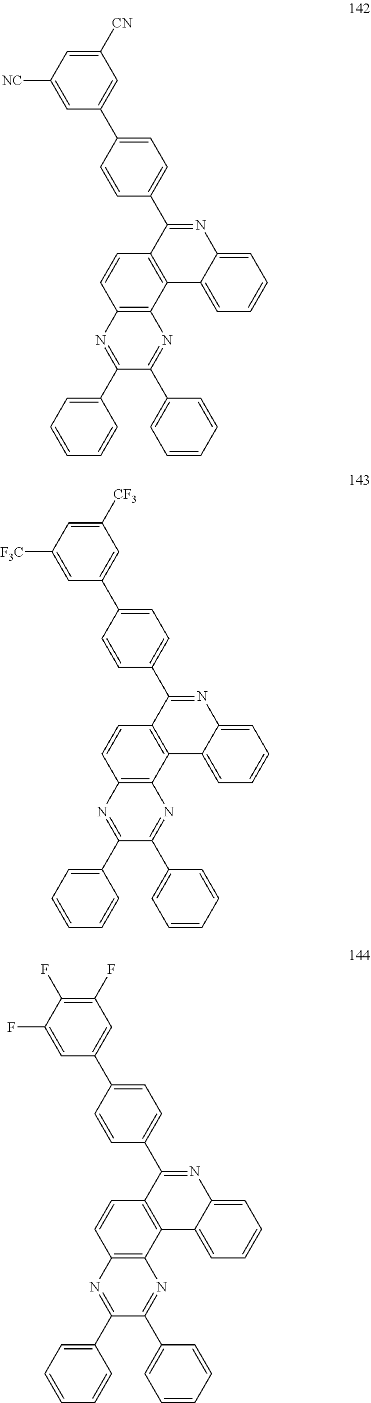

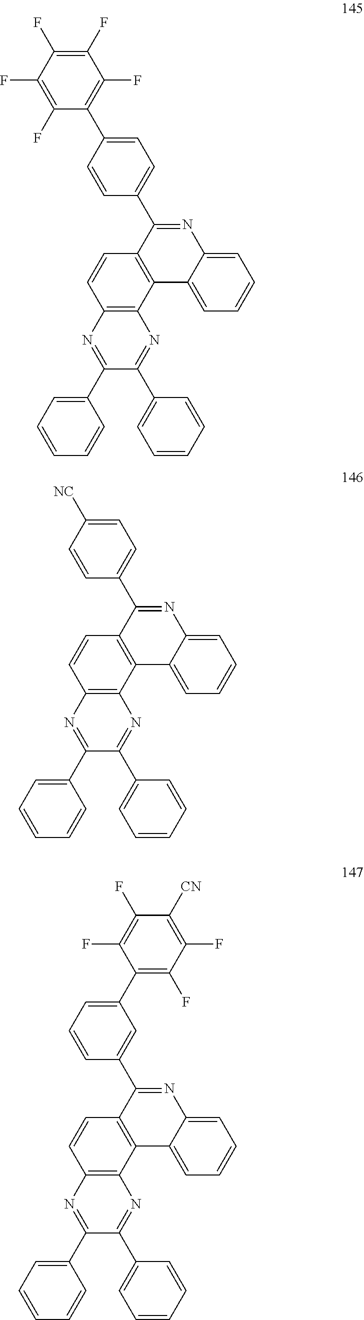

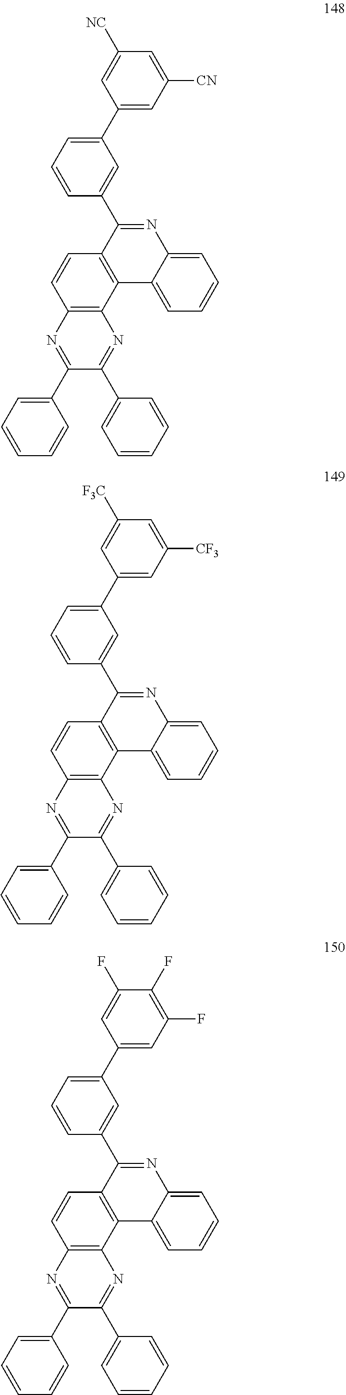

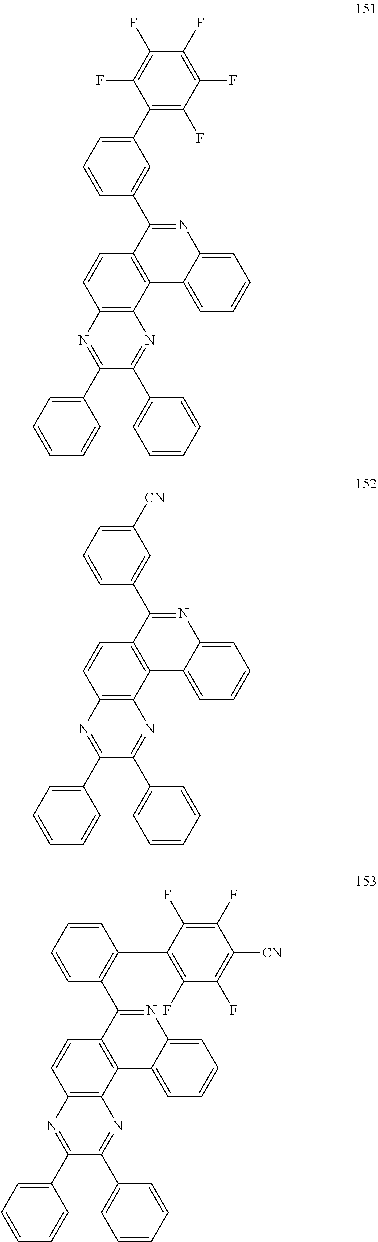

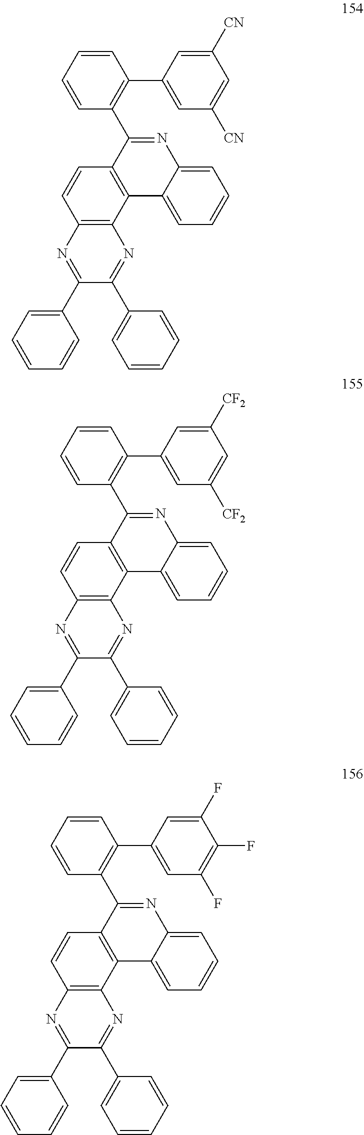

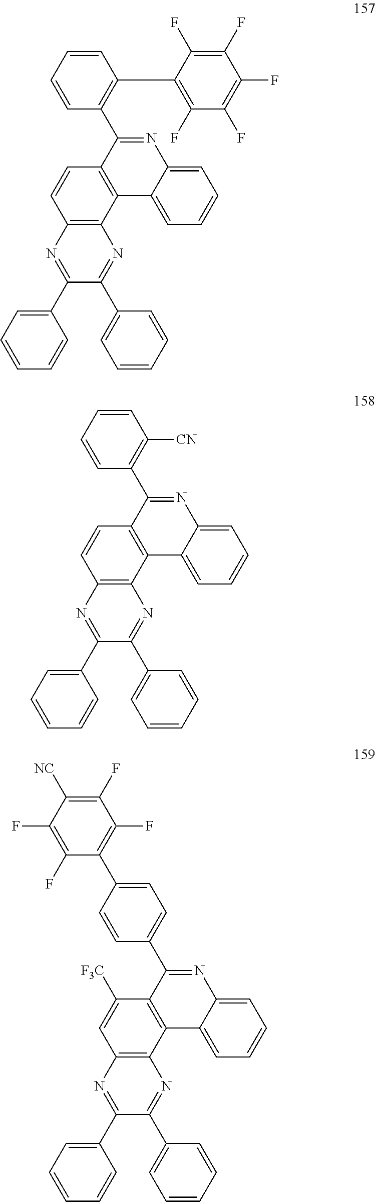

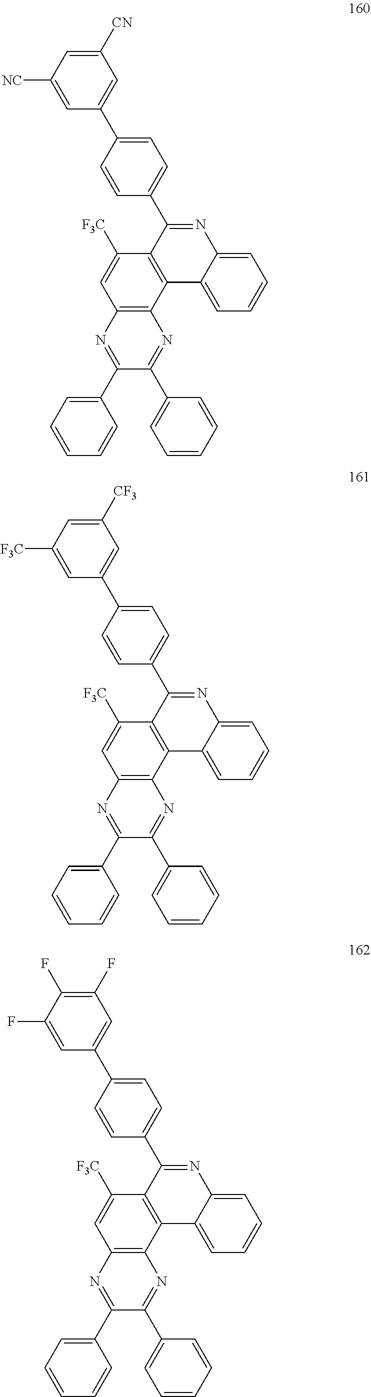

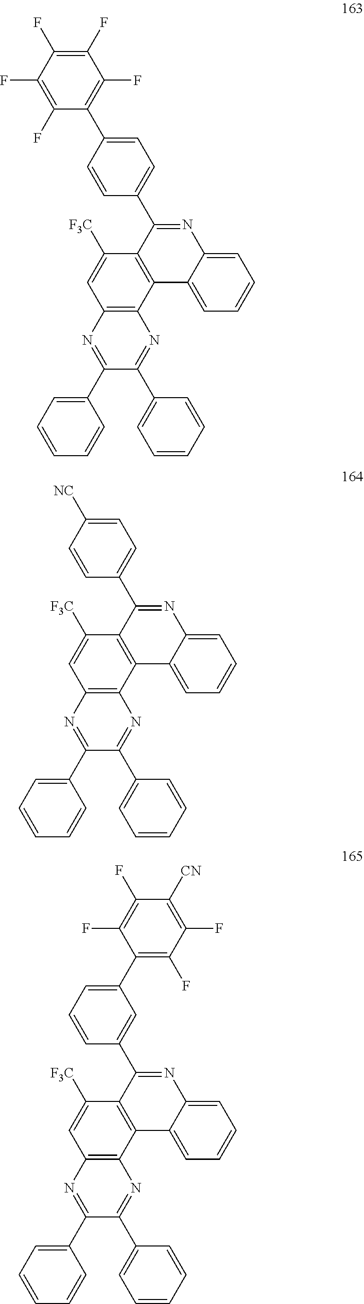

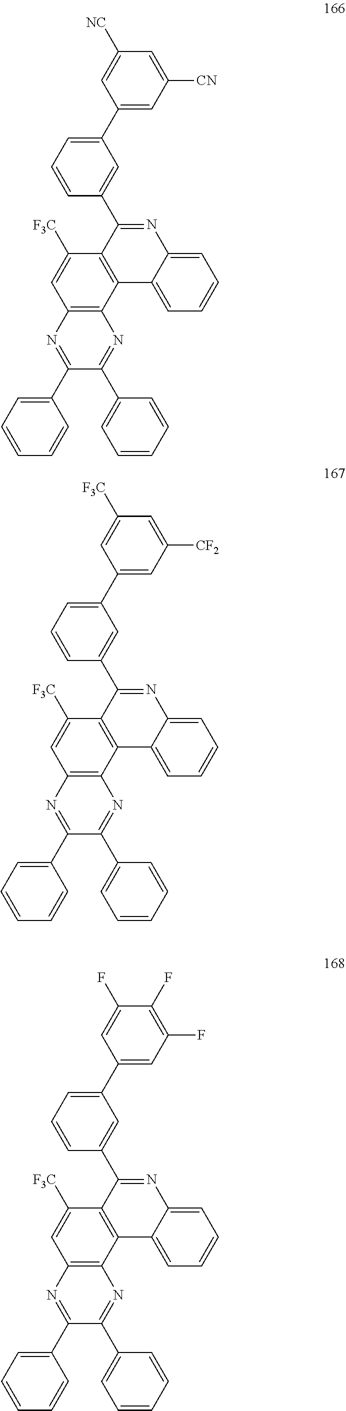

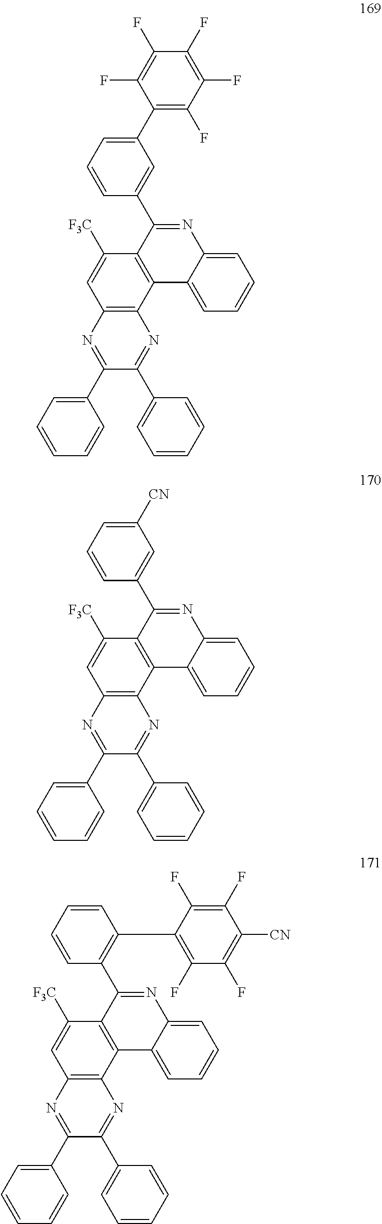

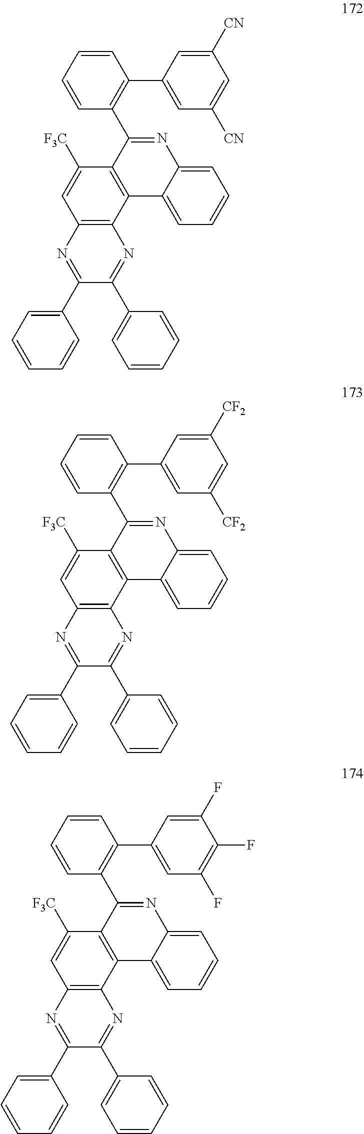

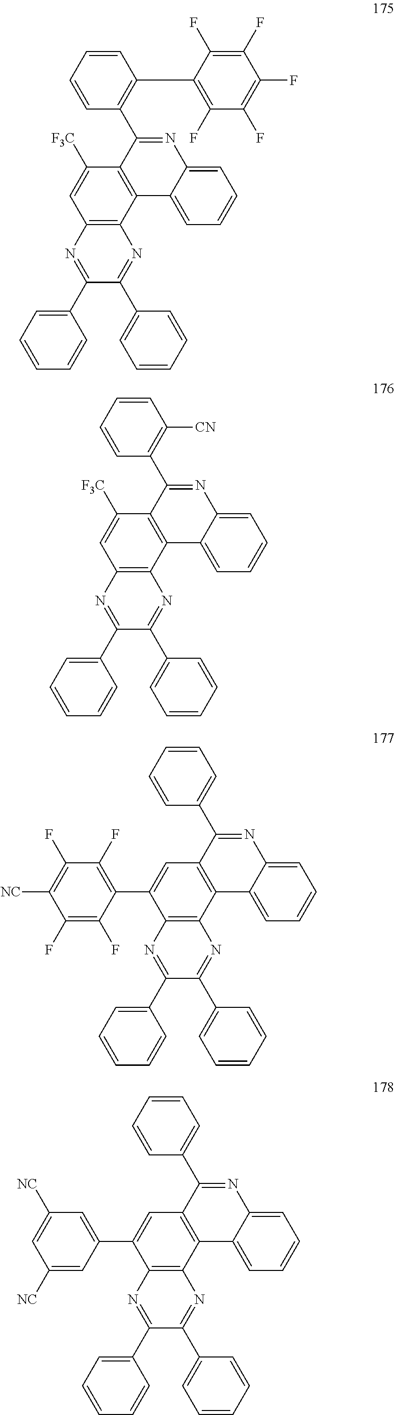

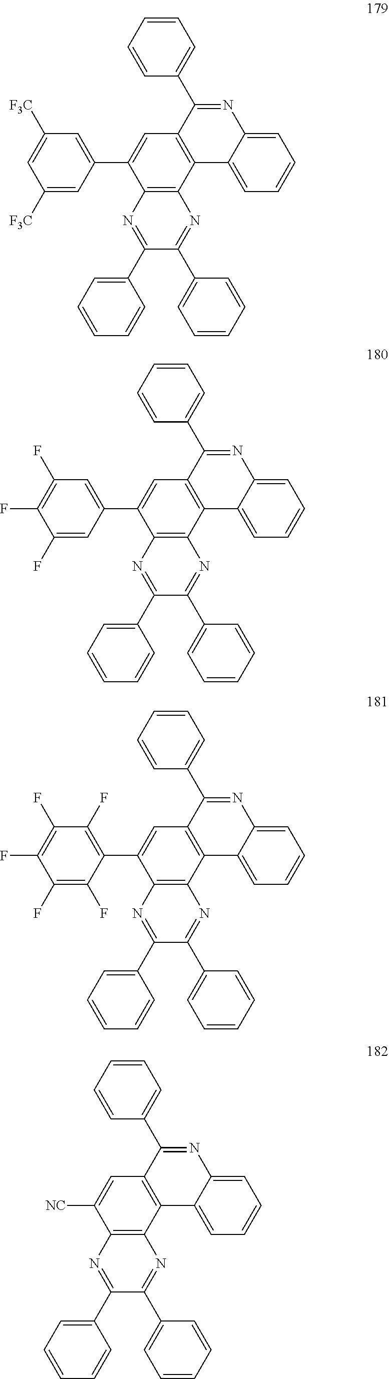

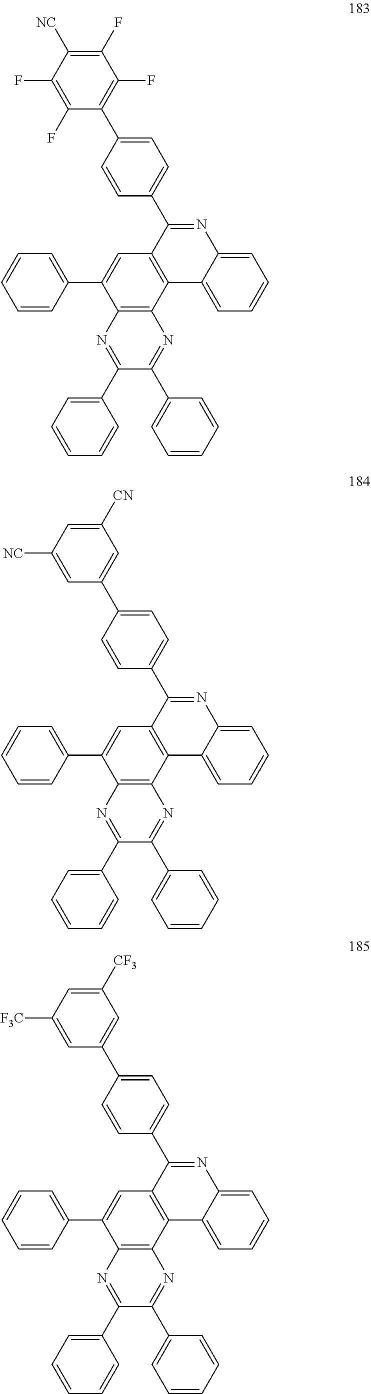

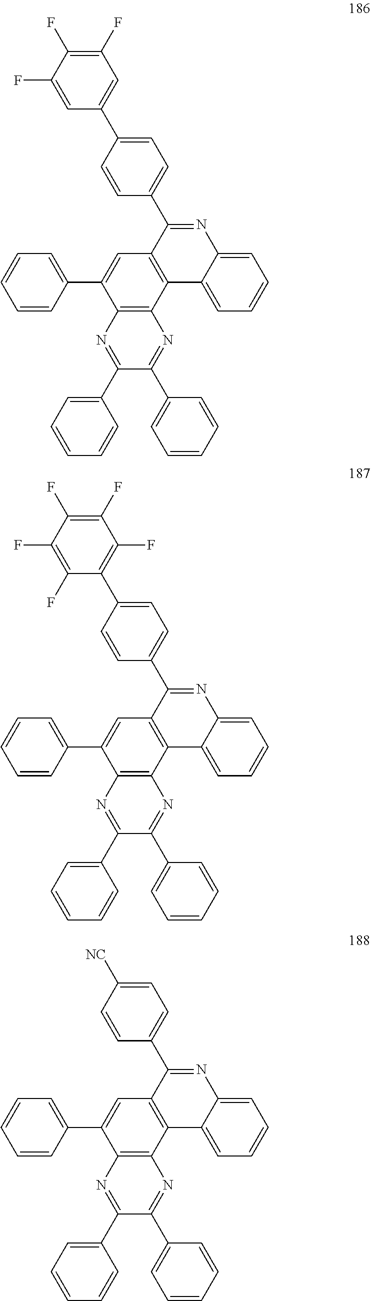

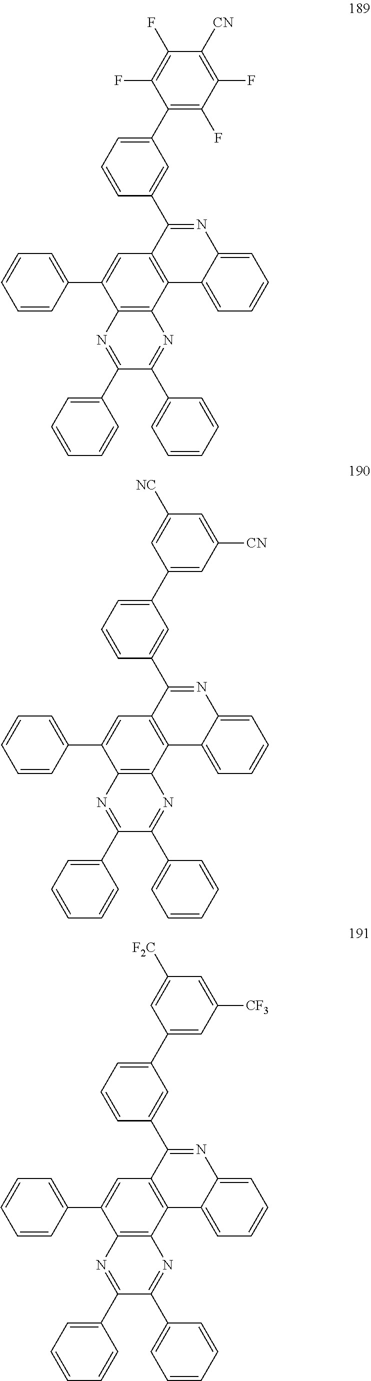

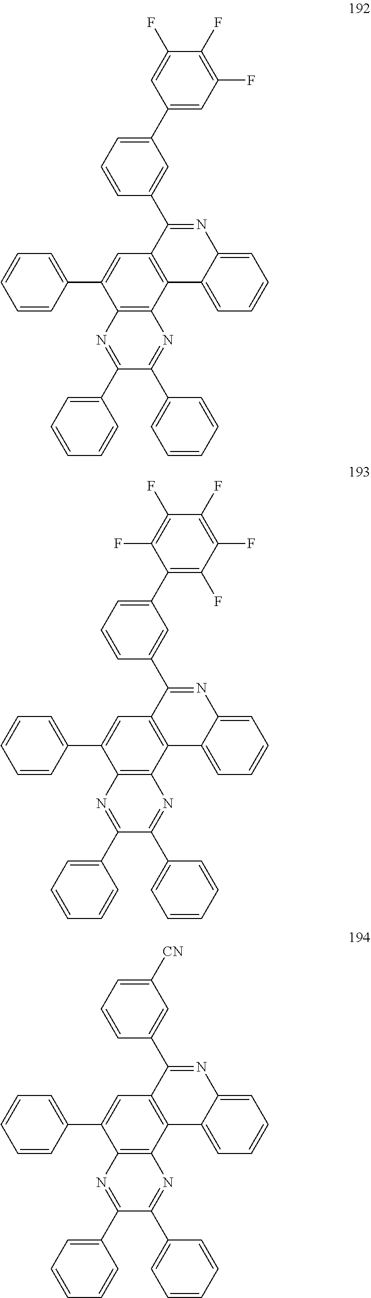

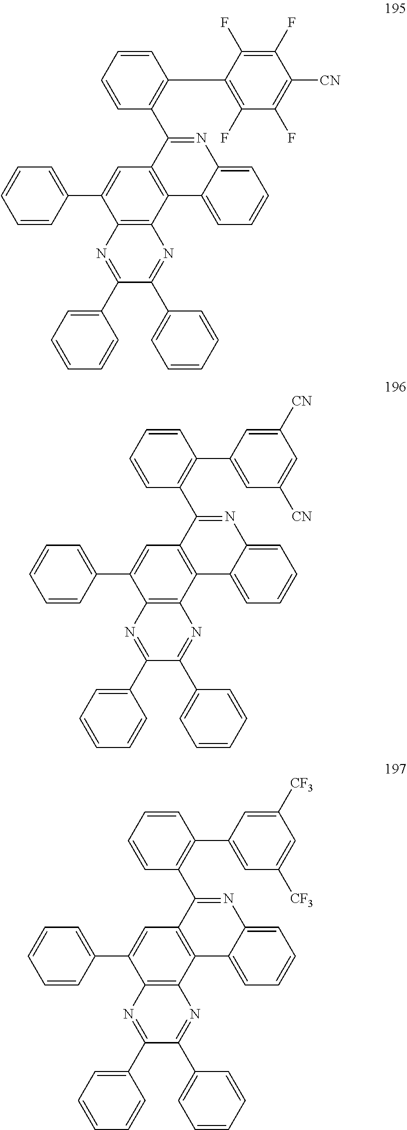 [SMILES: N#Cc1c(F)c(F)c(-c2ccccc2-c2nc3ccccc3c3c2cc(-c2ccccc2)c2nc(-c4ccccc4)c(-c4ccccc4)nc23)c(F)c1F ; N#Cc1cc(C#N)cc(-c2ccccc2-c2nc3ccccc3c3c2cc(-c2ccccc2)c2nc(-c4ccccc4)c(-c4ccccc4)nc23)c1 ; FC(F)(F)c1cc(-c2ccccc2-c2nc3ccccc3c3c2cc(-c2ccccc2)c2nc(-c4ccccc4)c(-c4ccccc4)nc23)cc(C(F)(F)F)c1]

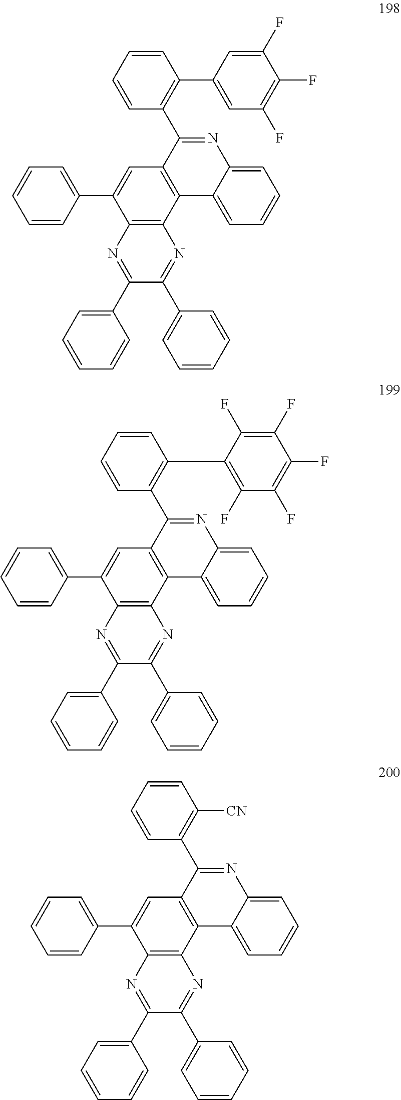

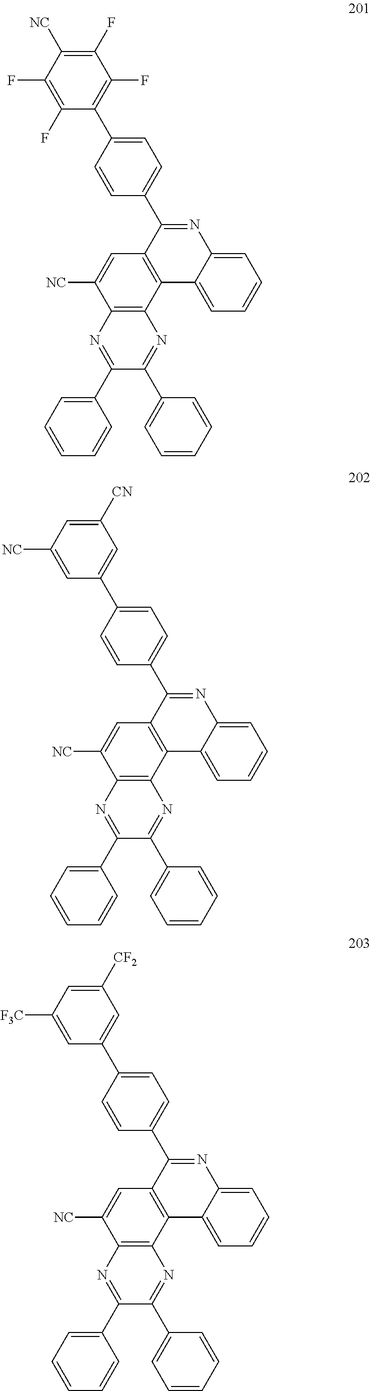

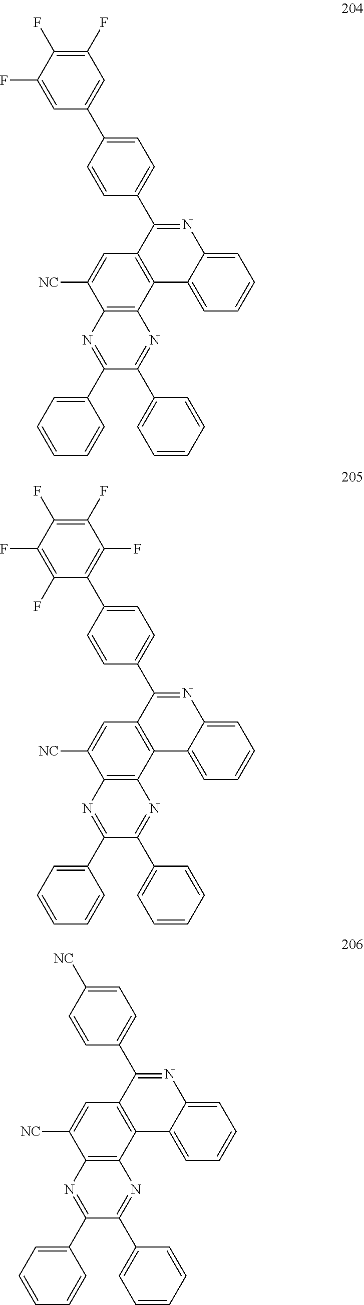

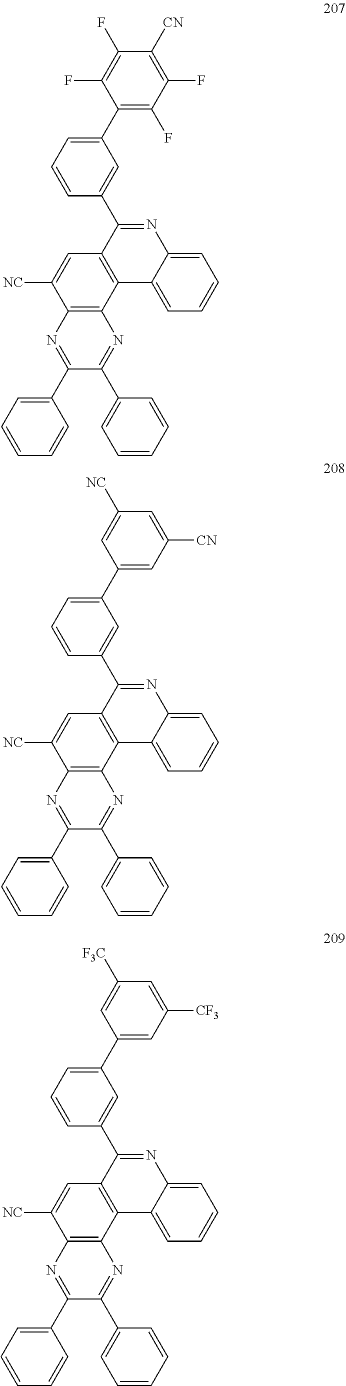

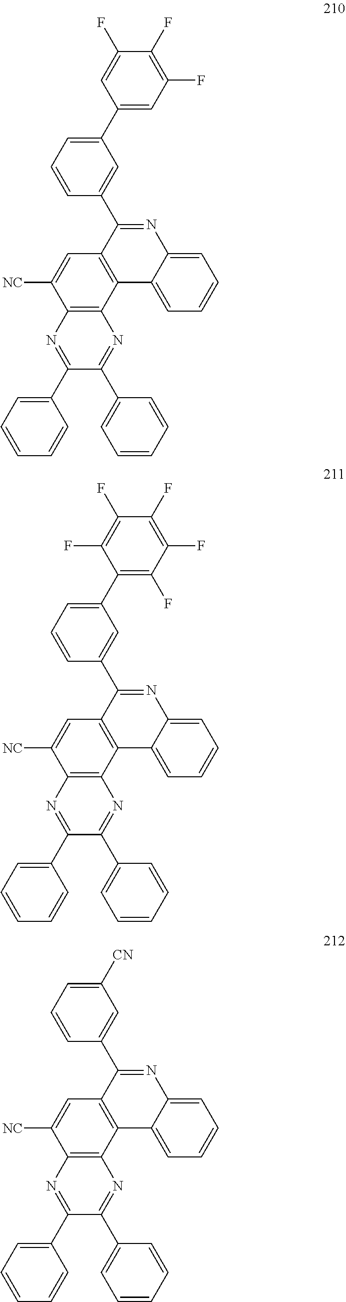

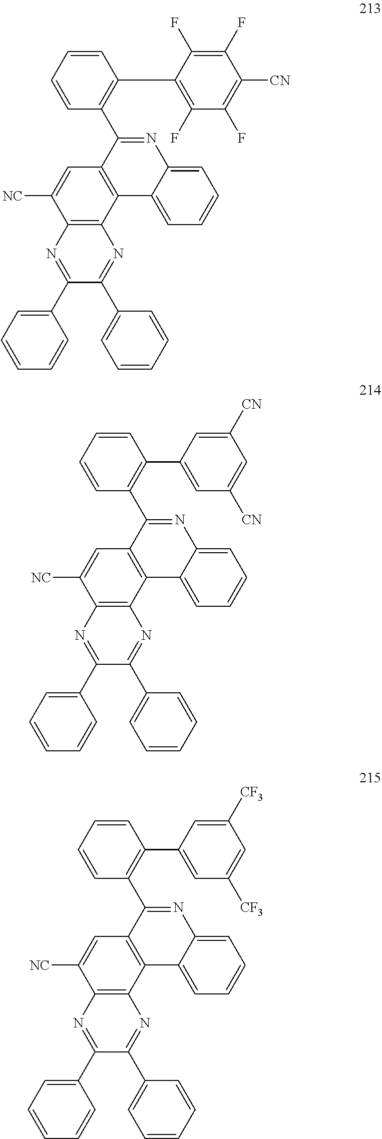 [SMILES: N#Cc1c(F)c(F)c(-c2ccccc2-c2nc3ccccc3c3c2cc(C#N)c2nc(-c4ccccc4)c(-c4ccccc4)nc23)c(F)c1F ; N#Cc1cc(C#N)cc(-c2ccccc2-c2nc3ccccc3c3c2cc(C#N)c2nc(-c4ccccc4)c(-c4ccccc4)nc23)c1 ; N#Cc1cc2c(-c3ccccc3-c3cc(C(F)(F)F)cc(C(F)(F)F)c3)nc3ccccc3c2c2nc(-c3ccccc3)c(-c3ccccc3)nc12]

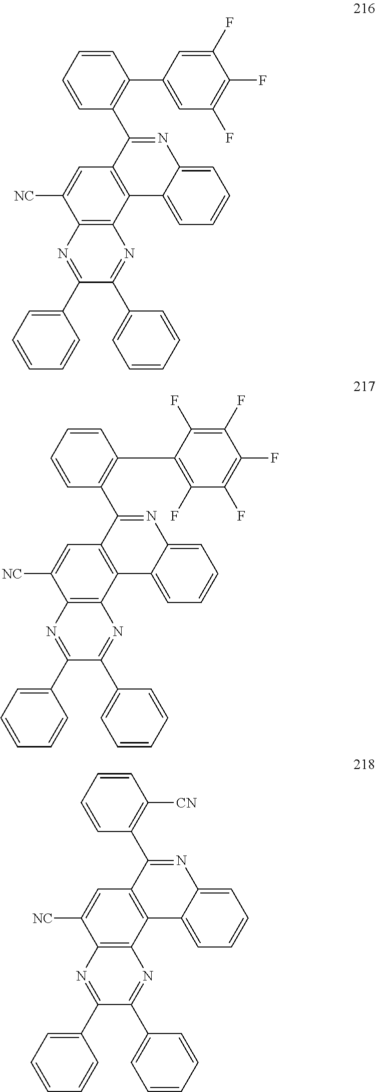

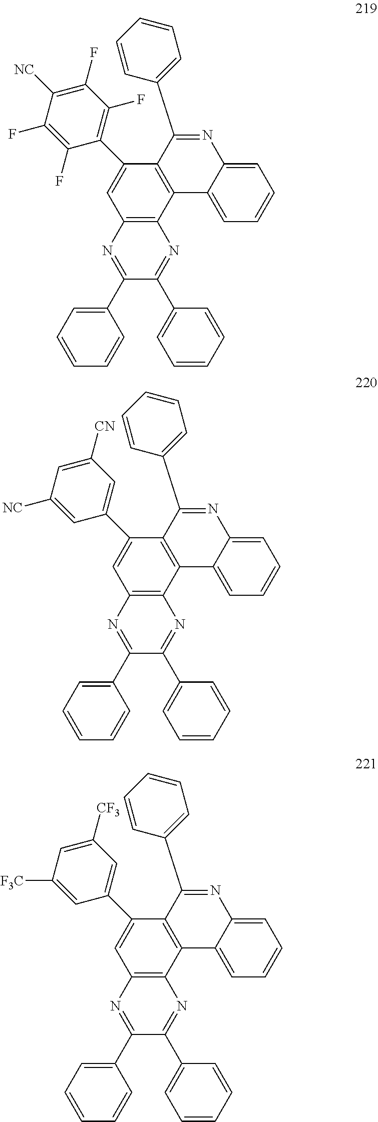

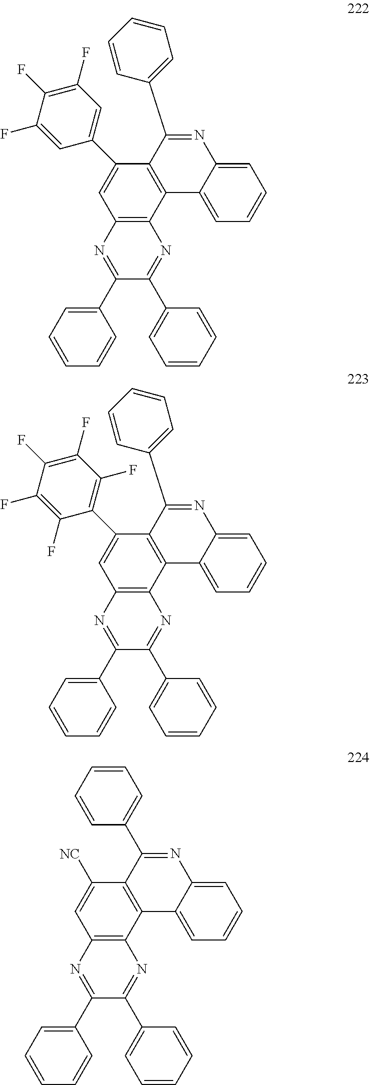

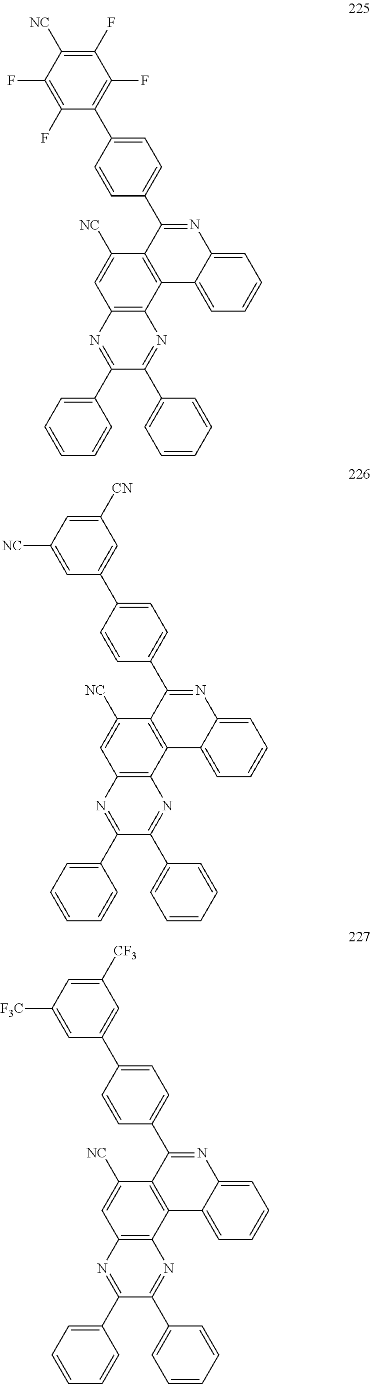

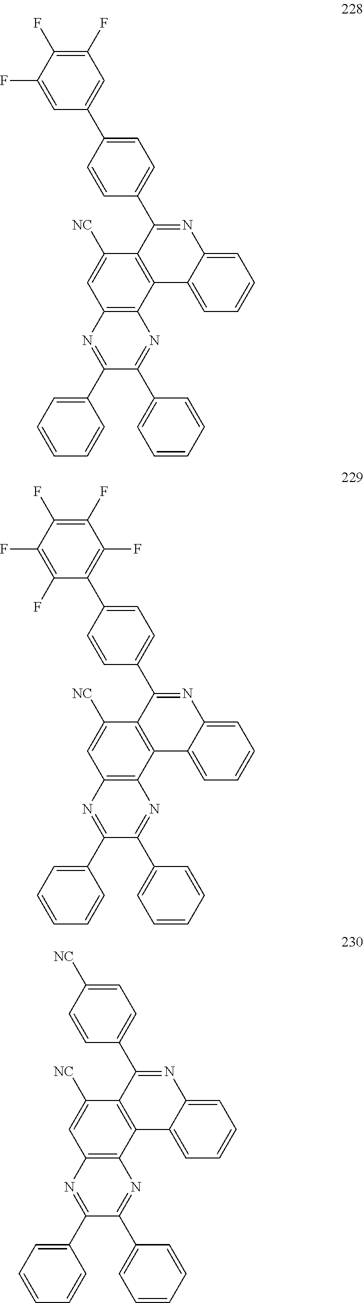

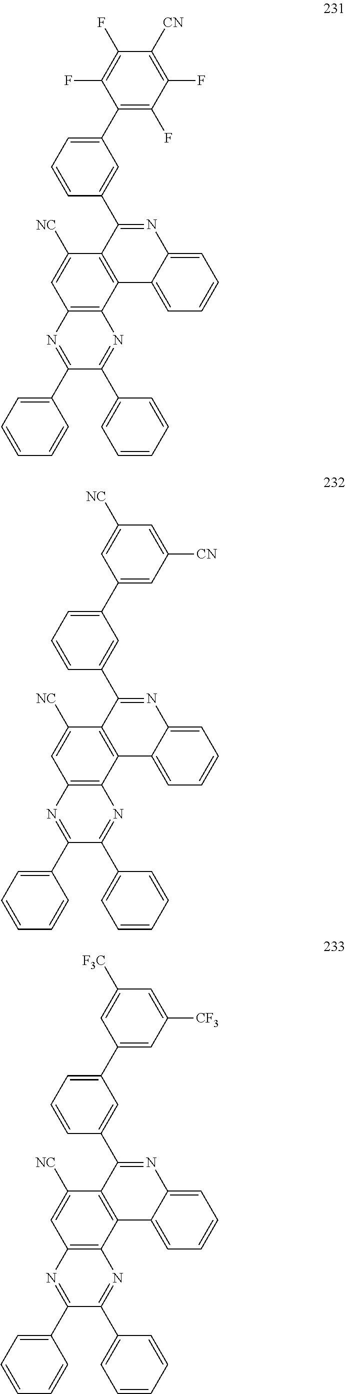

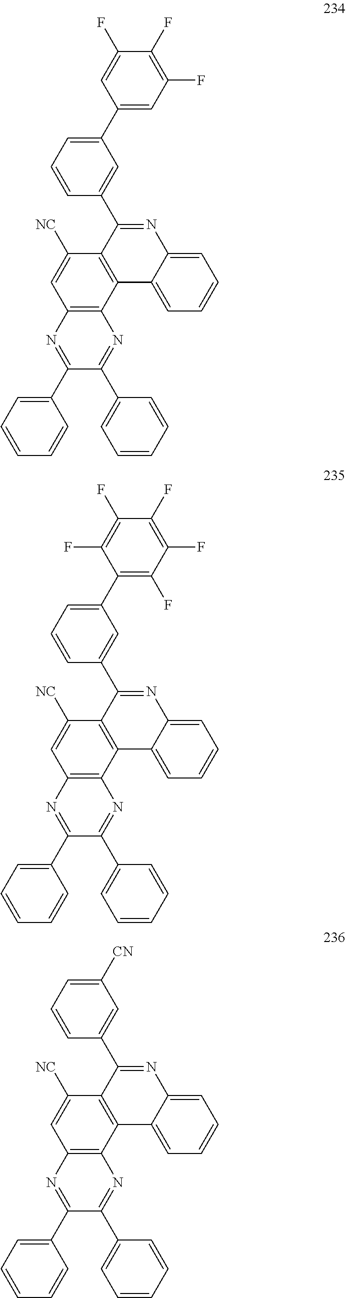

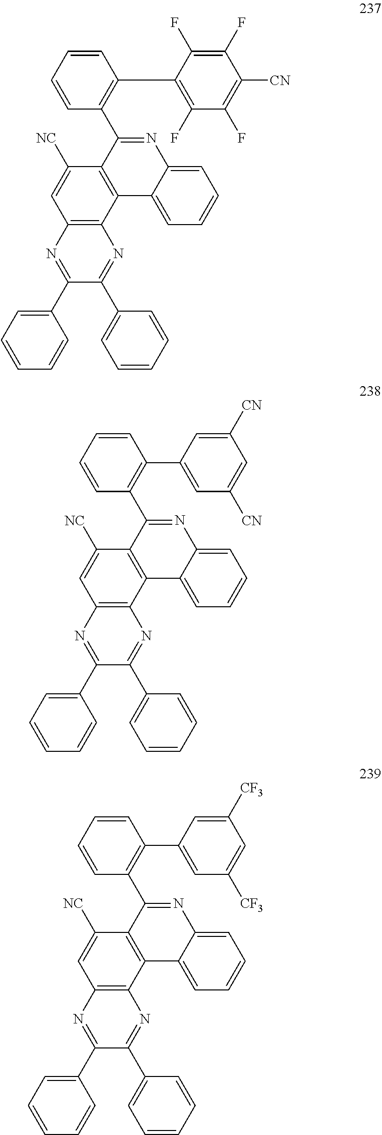 [SMILES: N#Cc1c(F)c(F)c(-c2ccccc2-c2nc3ccccc3c3c2c(C#N)cc2nc(-c4ccccc4)c(-c4ccccc4)nc23)c(F)c1F ; N#Cc1cc(C#N)cc(-c2ccccc2-c2nc3ccccc3c3c2c(C#N)cc2nc(-c4ccccc4)c(-c4ccccc4)nc23)c1 ; N#Cc1cc2nc(-c3ccccc3)c(-c3ccccc3)nc2c2c1c(-c1ccccc1-c1cc(C(F)(F)F)cc(C(F)(F)F)c1)nc1ccccc12]

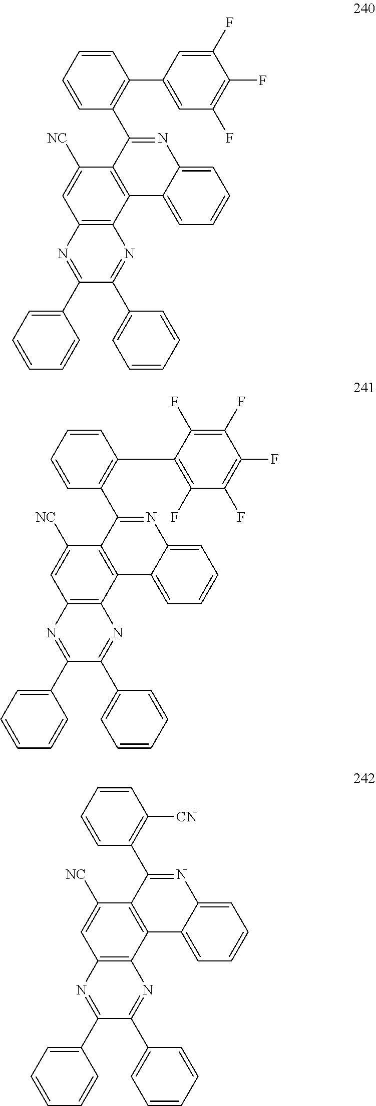

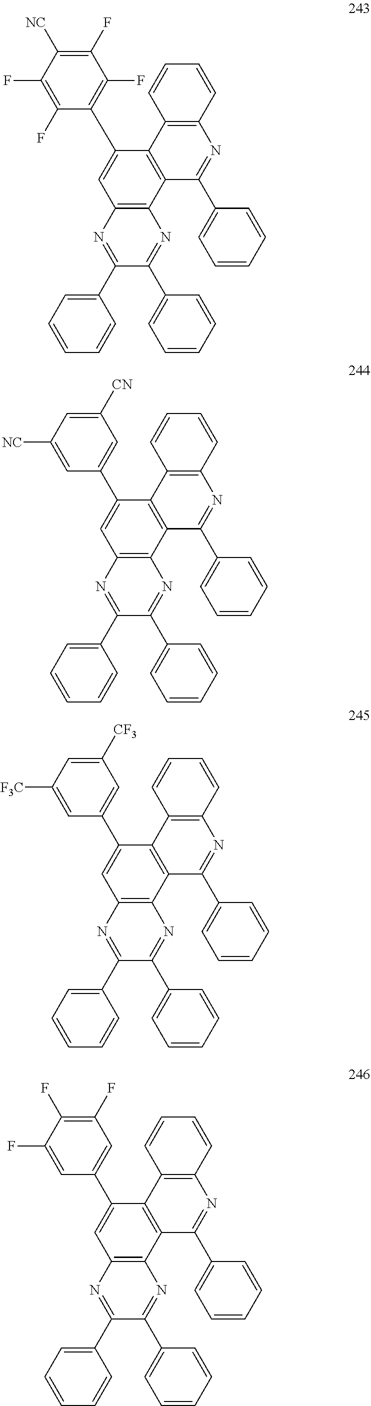

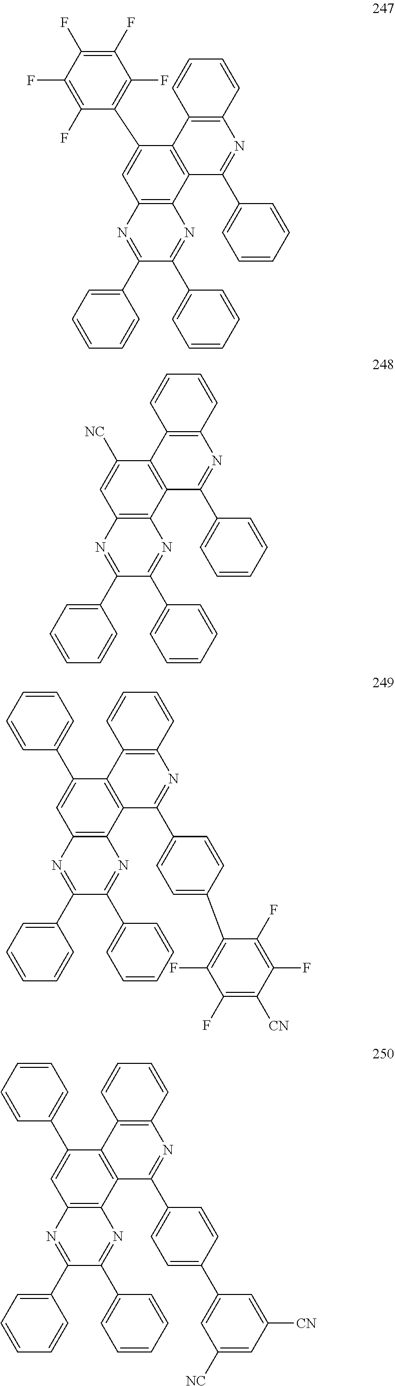

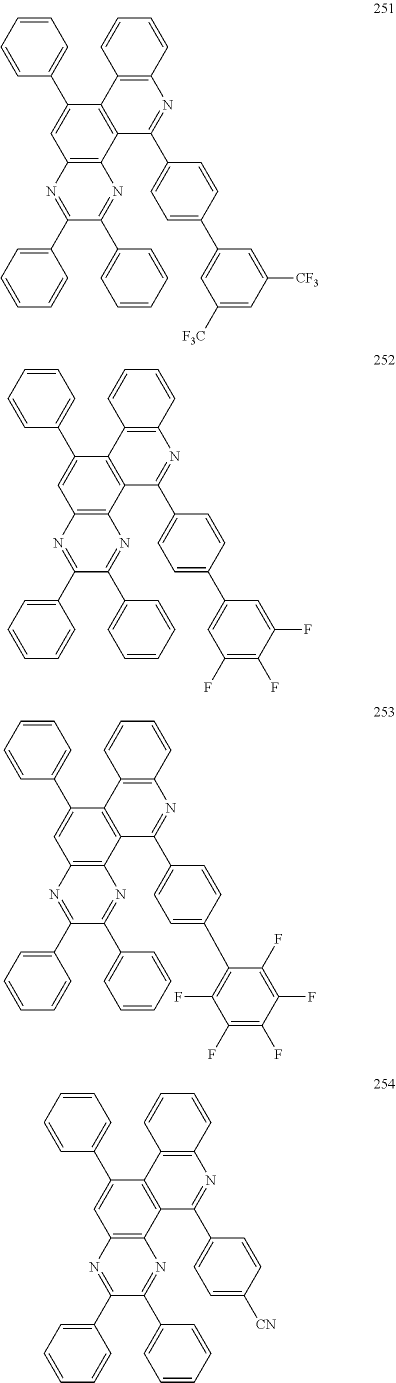

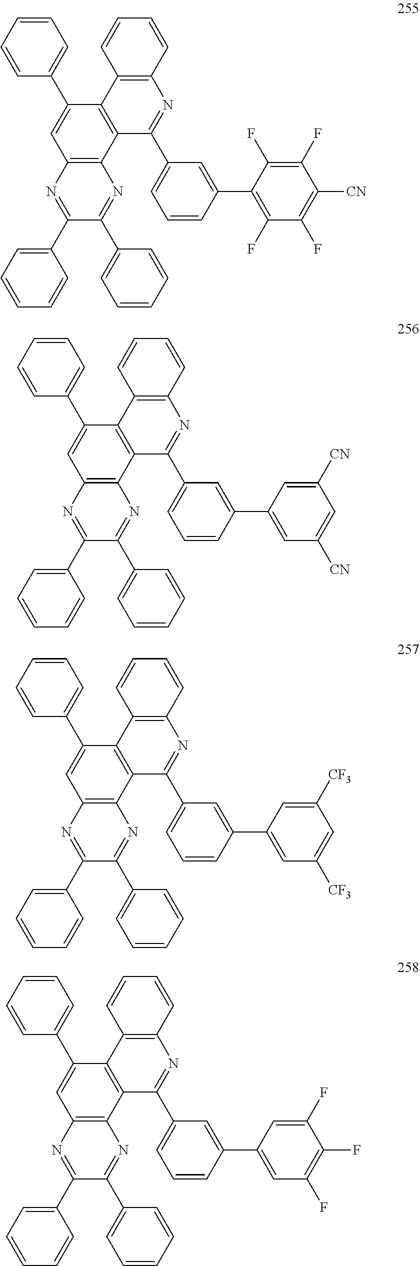

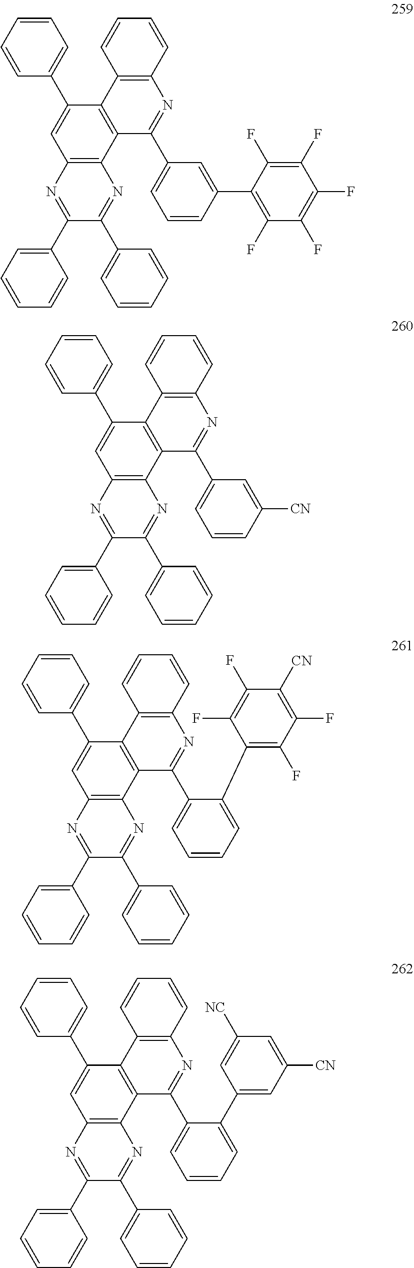

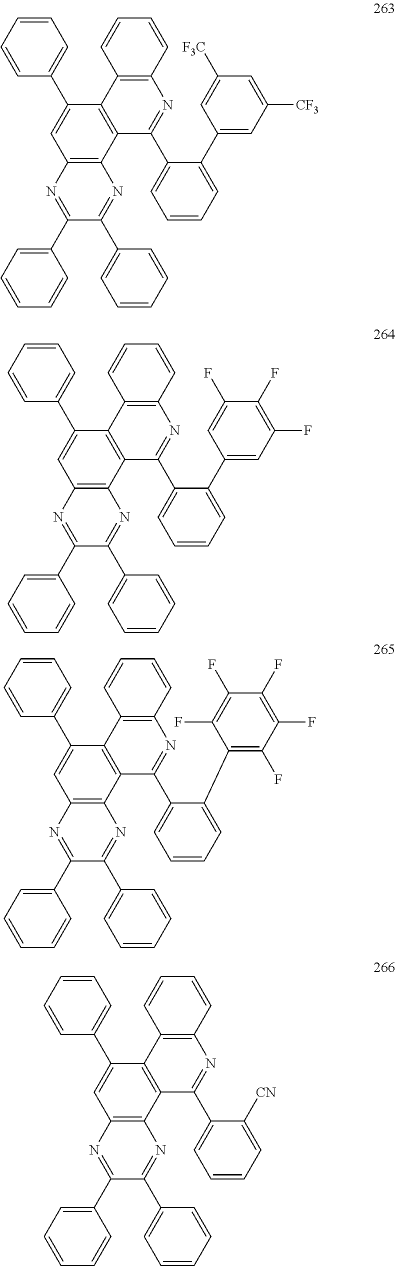

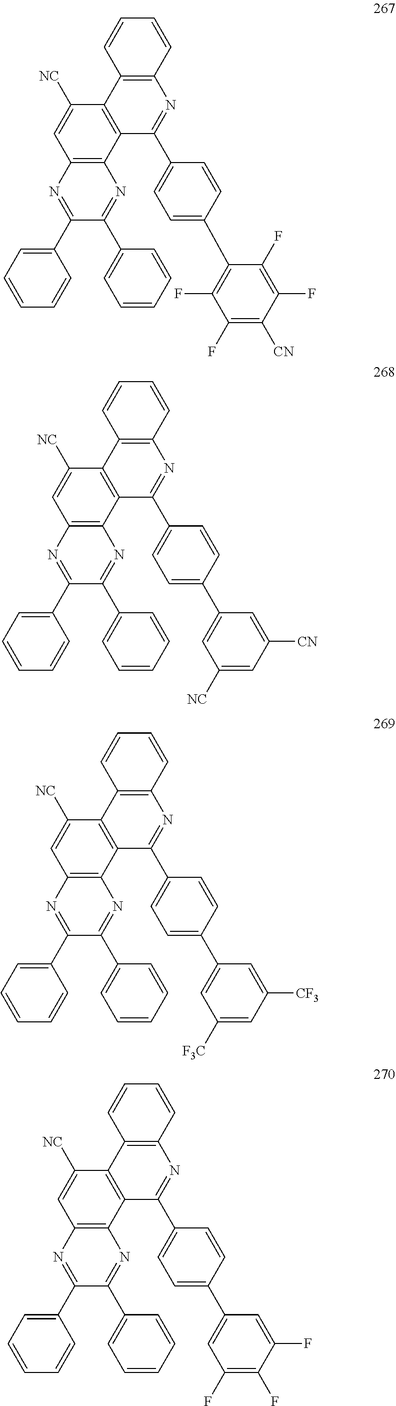 [SMILES: N#Cc1c(F)c(F)c(-c2ccc(-c3nc4ccccc4c4c(C#N)cc5nc(-c6ccccc6)c(-c6ccccc6)nc5c34)cc2)c(F)c1F ; N#Cc1cc(C#N)cc(-c2ccc(-c3nc4ccccc4c4c(C#N)cc5nc(-c6ccccc6)c(-c6ccccc6)nc5c34)cc2)c1 ; N#Cc1cc2nc(-c3ccccc3)c(-c3ccccc3)nc2c2c(-c3ccc(-c4cc(C(F)(F)F)cc(C(F)(F)F)c4)cc3)nc3ccccc3c12 ; N#Cc1cc2nc(-c3ccccc3)c(-c3ccccc3)nc2c2c(-c3ccc(-c4cc(F)c(F)c(F)c4)cc3)nc3ccccc3c12]

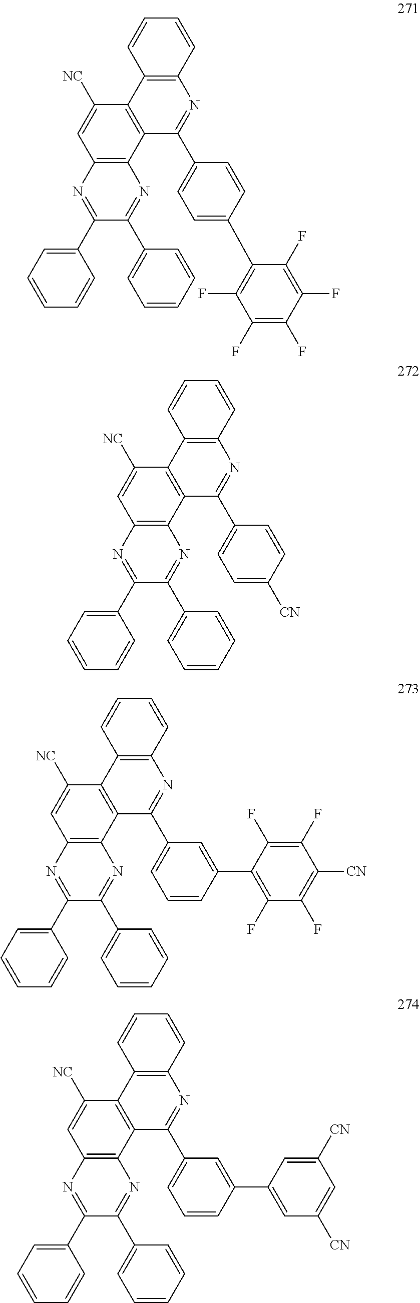

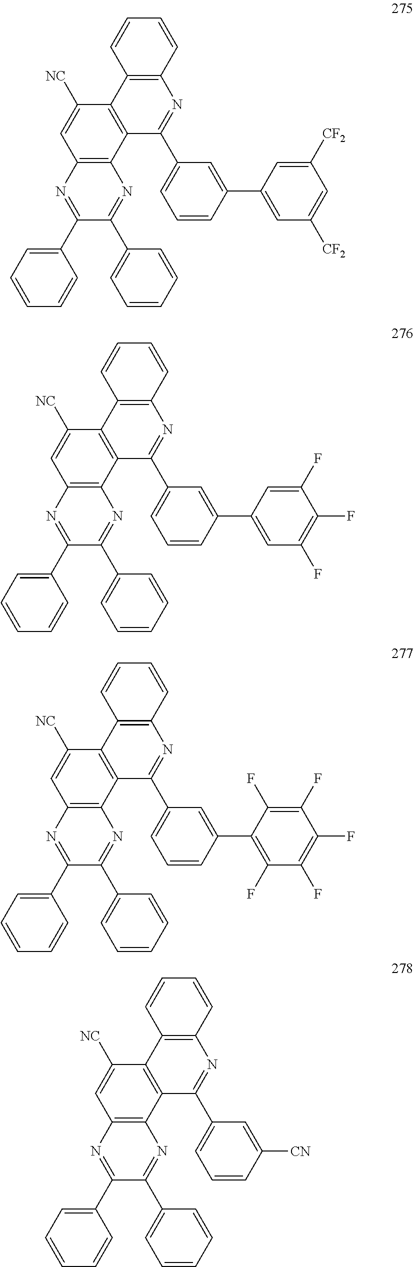

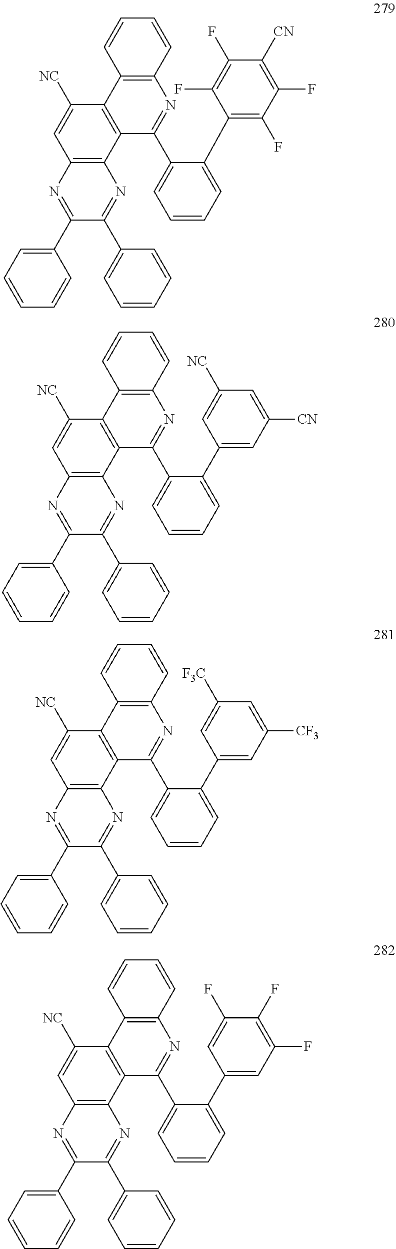

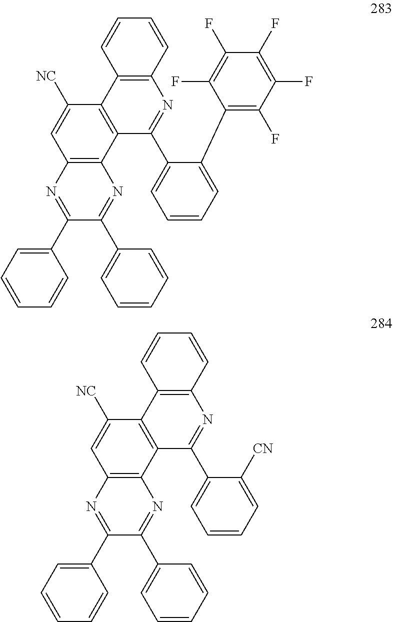

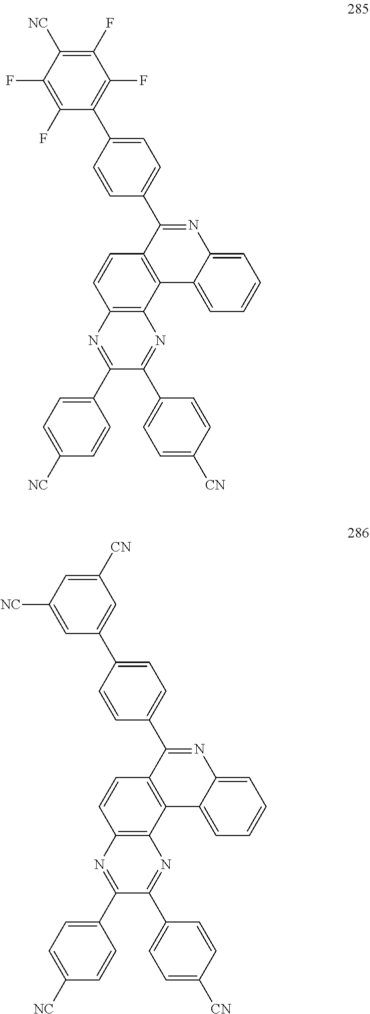 [SMILES: N#Cc1ccc(-c2nc3ccc4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3ccc4c(-c5ccc(-c6cc(C#N)cc(C#N)c6)cc5)nc5ccccc5c4c3nc2-c2ccc(C#N)cc2)cc1]

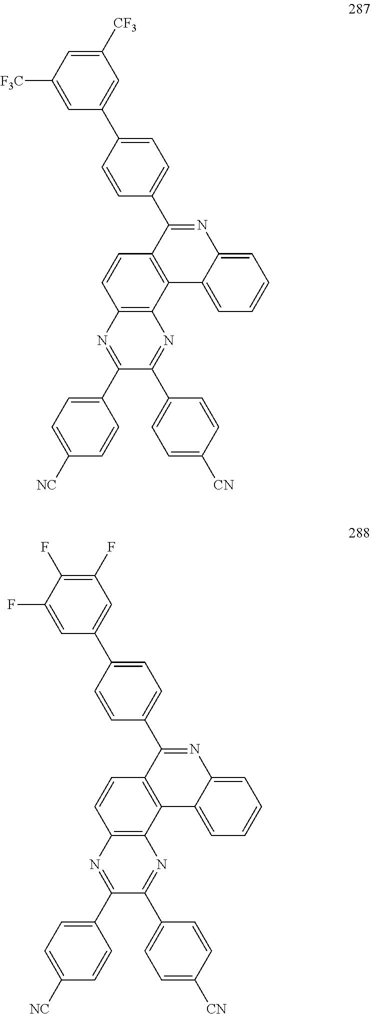 [SMILES: N#Cc1ccc(-c2nc3ccc4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3ccc4c(-c5ccc(-c6cc(F)c(F)c(F)c6)cc5)nc5ccccc5c4c3nc2-c2ccc(C#N)cc2)cc1]

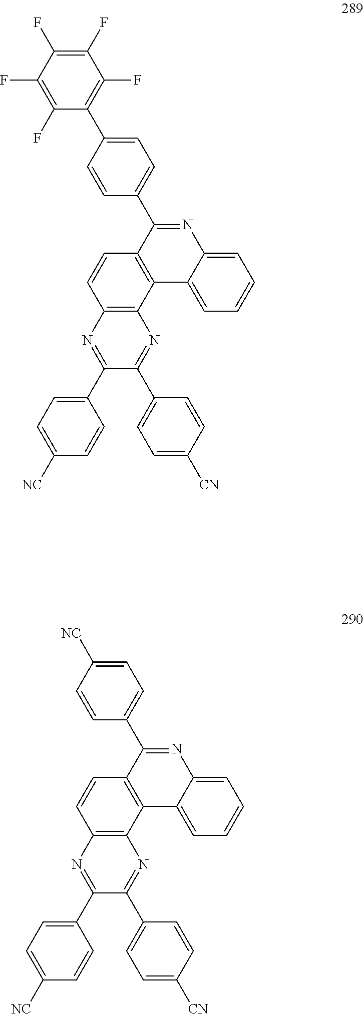 [SMILES: N#Cc1ccc(-c2nc3ccc4c(-c5ccc(-c6c(F)c(F)c(F)c(F)c6F)cc5)nc5ccccc5c4c3nc2-c2ccc(C#N)cc2)cc1 ; N#Cc1ccc(-c2nc3ccc4c(-c5ccc(C#N)cc5)nc5ccccc5c4c3nc2-c2ccc(C#N)cc2)cc1]

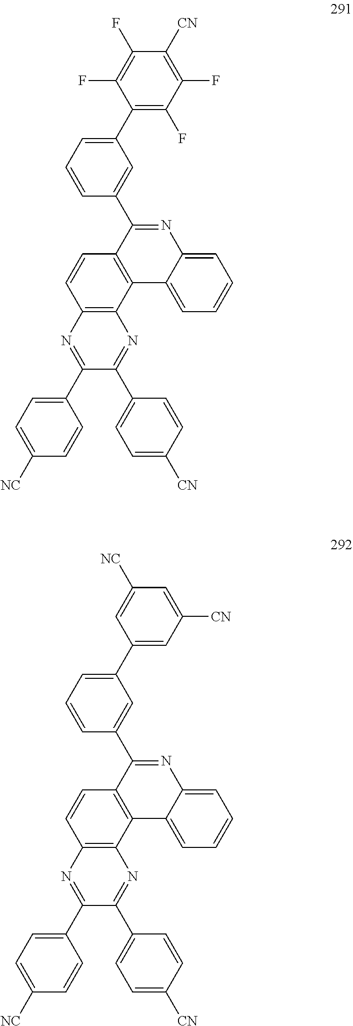 [SMILES: N#Cc1ccc(-c2nc3ccc4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3ccc4c(-c5cccc(-c6cc(C#N)cc(C#N)c6)c5)nc5ccccc5c4c3nc2-c2ccc(C#N)cc2)cc1]

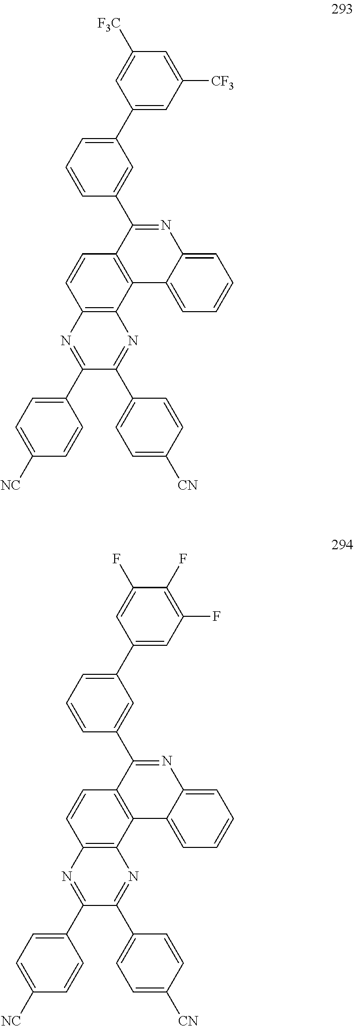 [SMILES: N#Cc1ccc(-c2nc3ccc4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3ccc4c(-c5cccc(-c6cc(F)c(F)c(F)c6)c5)nc5ccccc5c4c3nc2-c2ccc(C#N)cc2)cc1]

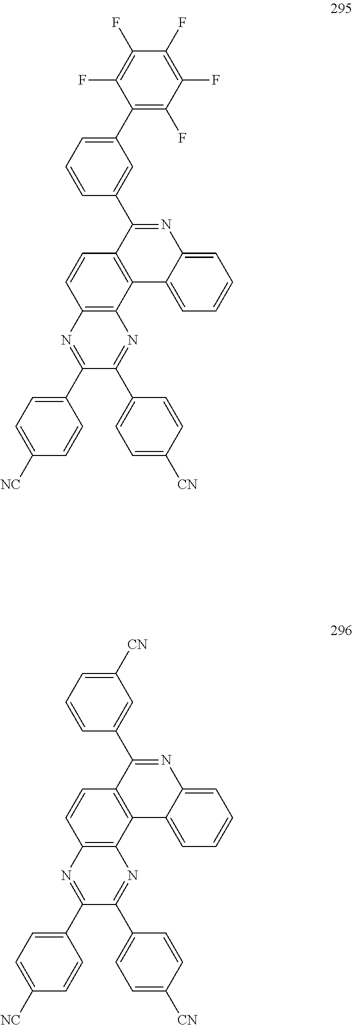 [SMILES: N#Cc1ccc(-c2nc3ccc4c(-c5cccc(-c6c(F)c(F)c(F)c(F)c6F)c5)nc5ccccc5c4c3nc2-c2ccc(C#N)cc2)cc1 ; N#Cc1ccc(-c2nc3ccc4c(-c5cccc(C#N)c5)nc5ccccc5c4c3nc2-c2ccc(C#N)cc2)cc1]

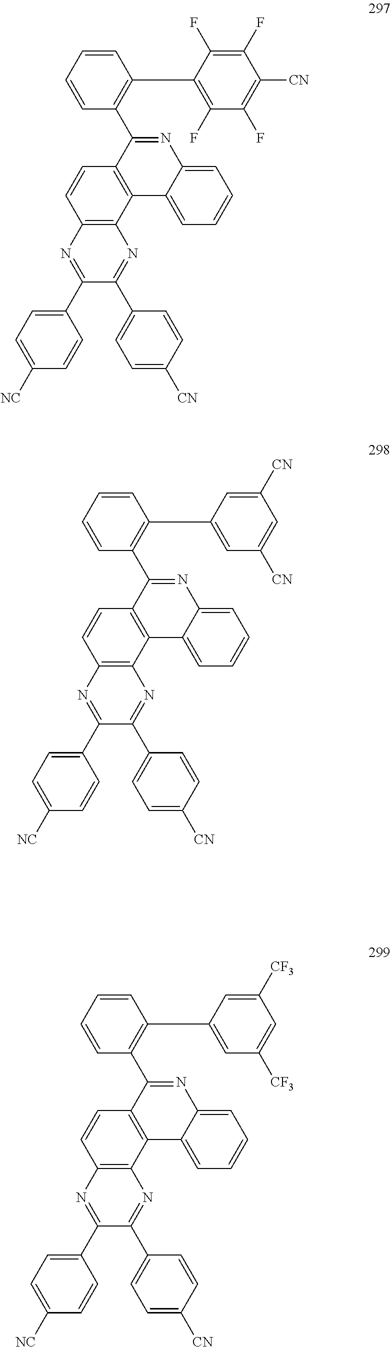 [SMILES: N#Cc1ccc(-c2nc3ccc4c(-c5ccccc5-c5c(F)c(F)c(C#N)c(F)c5F)nc5ccccc5c4c3nc2-c2ccc(C#N)cc2)cc1 ; N#Cc1ccc(-c2nc3ccc4c(-c5ccccc5-c5cc(C#N)cc(C#N)c5)nc5ccccc5c4c3nc2-c2ccc(C#N)cc2)cc1 ; N#Cc1ccc(-c2nc3ccc4c(-c5ccccc5-c5cc(C(F)(F)F)cc(C(F)(F)F)c5)nc5ccccc5c4c3nc2-c2ccc(C#N)cc2)cc1]

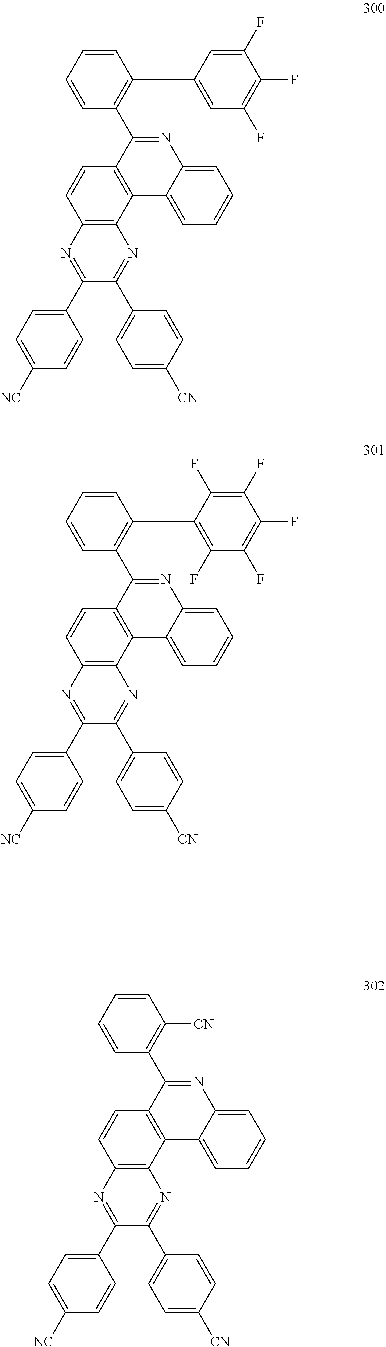 [SMILES: N#Cc1ccc(-c2nc3ccc4c(-c5ccccc5-c5cc(F)c(F)c(F)c5)nc5ccccc5c4c3nc2-c2ccc(C#N)cc2)cc1 ; N#Cc1ccc(-c2nc3ccc4c(-c5ccccc5-c5c(F)c(F)c(F)c(F)c5F)nc5ccccc5c4c3nc2-c2ccc(C#N)cc2)cc1 ; N#Cc1ccc(-c2nc3ccc4c(-c5ccccc5C#N)nc5ccccc5c4c3nc2-c2ccc(C#N)cc2)cc1]

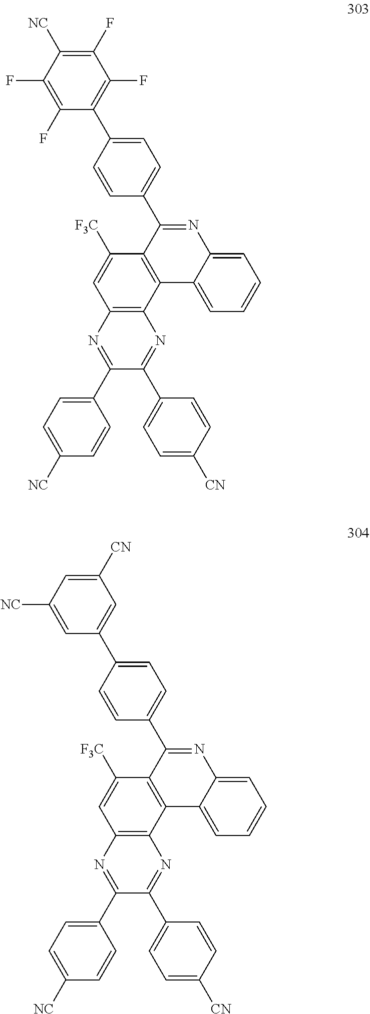 [SMILES: N#Cc1ccc(-c2nc3cc(C(F)(F)F)c4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3cc(C(F)(F)F)c4c(-c5ccc(-c6cc(C#N)cc(C#N)c6)cc5)nc5ccccc5c4c3nc2-c2ccc(C#N)cc2)cc1]

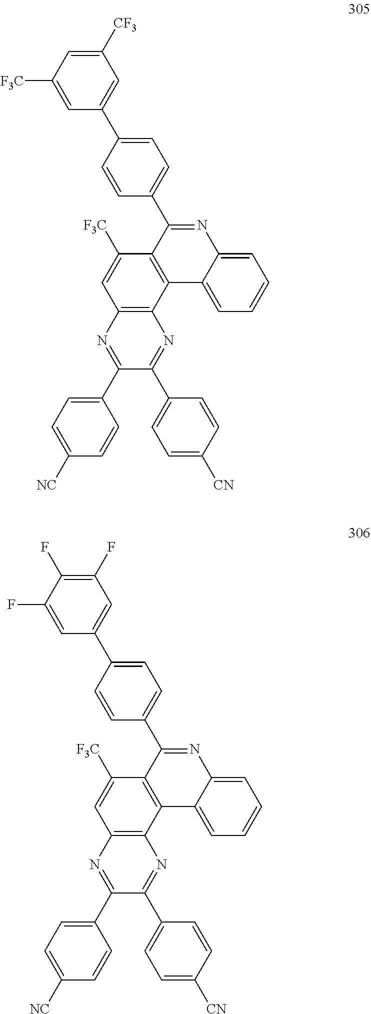 [SMILES: N#Cc1ccc(-c2nc3cc(C(F)(F)F)c4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3cc(C(F)(F)F)c4c(-c5ccc(-c6cc(F)c(F)c(F)c6)cc5)nc5ccccc5c4c3nc2-c2ccc(C#N)cc2)cc1]

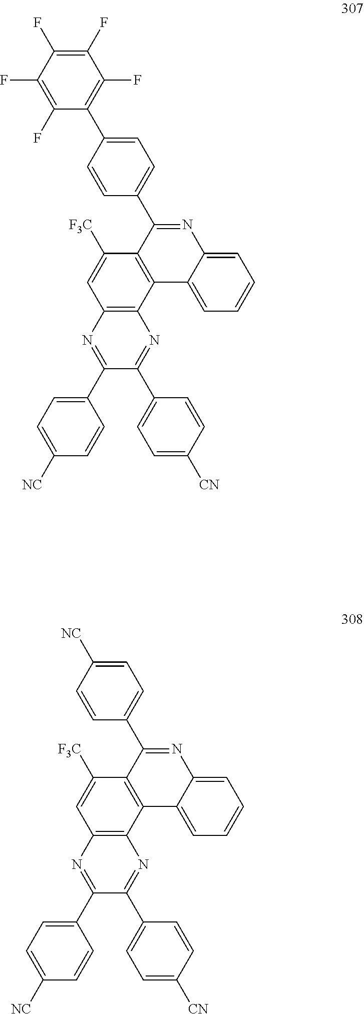 [SMILES: N#Cc1ccc(-c2nc3cc(C(F)(F)F)c4c(-c5ccc(-c6c(F)c(F)c(F)c(F)c6F)cc5)nc5ccccc5c4c3nc2-c2ccc(C#N)cc2)cc1 ; N#Cc1ccc(-c2nc3cc(C(F)(F)F)c4c(-c5ccc(C#N)cc5)nc5ccccc5c4c3nc2-c2ccc(C#N)cc2)cc1]

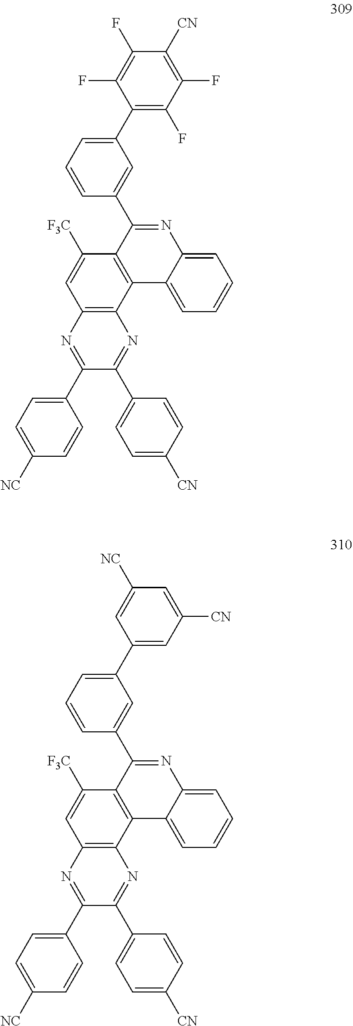 [SMILES: N#Cc1ccc(-c2nc3cc(C(F)(F)F)c4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3cc(C(F)(F)F)c4c(-c5cccc(-c6cc(C#N)cc(C#N)c6)c5)nc5ccccc5c4c3nc2-c2ccc(C#N)cc2)cc1]

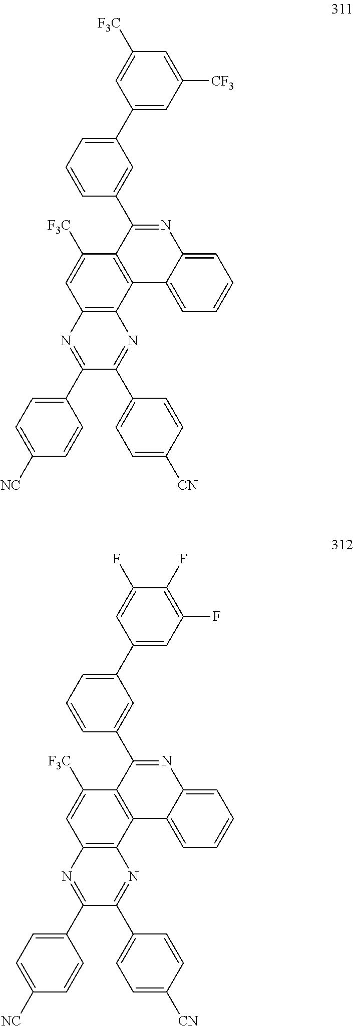 [SMILES: N#Cc1ccc(-c2nc3cc(C(F)(F)F)c4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3cc(C(F)(F)F)c4c(-c5cccc(-c6cc(F)c(F)c(F)c6)c5)nc5ccccc5c4c3nc2-c2ccc(C#N)cc2)cc1]

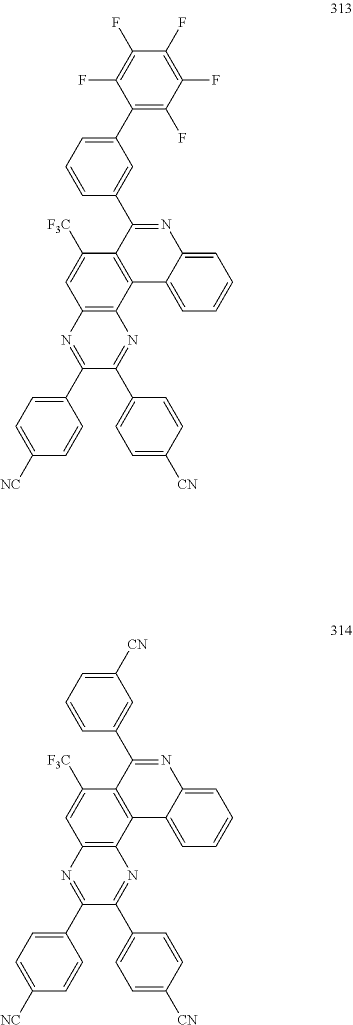 [SMILES: N#Cc1ccc(-c2nc3cc(C(F)(F)F)c4c(-c5cccc(-c6c(F)c(F)c(F)c(F)c6F)c5)nc5ccccc5c4c3nc2-c2ccc(C#N)cc2)cc1 ; N#Cc1ccc(-c2nc3cc(C(F)(F)F)c4c(-c5cccc(C#N)c5)nc5ccccc5c4c3nc2-c2ccc(C#N)cc2)cc1]

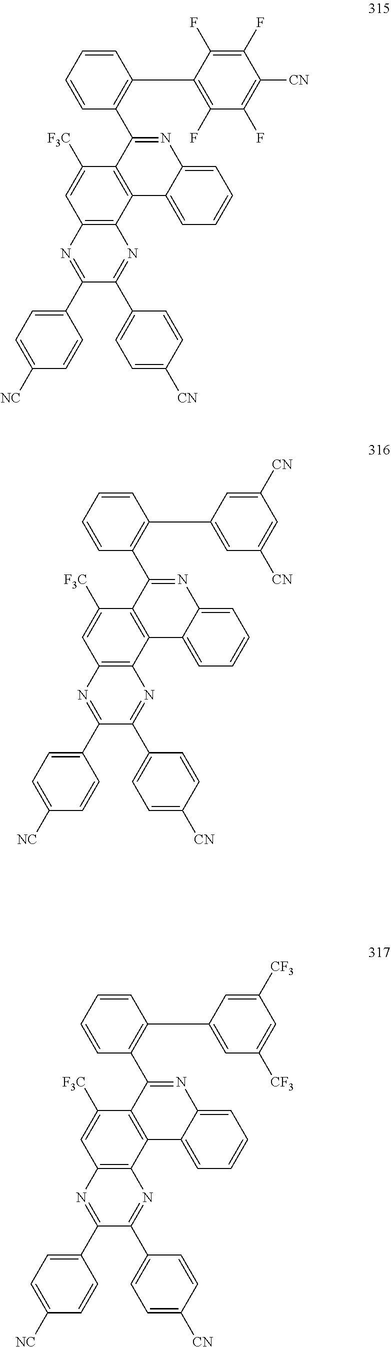 [SMILES: N#Cc1ccc(-c2nc3cc(C(F)(F)F)c4c(-c5ccccc5-c5c(F)c(F)c(C#N)c(F)c5F)nc5ccccc5c4c3nc2-c2ccc(C#N)cc2)cc1 ; N#Cc1ccc(-c2nc3cc(C(F)(F)F)c4c(-c5ccccc5-c5cc(C#N)cc(C#N)c5)nc5ccccc5c4c3nc2-c2ccc(C#N)cc2)cc1 ; N#Cc1ccc(-c2nc3cc(C(F)(F)F)c4c(-c5ccccc5-c5cc(C(F)(F)F)cc(C(F)(F)F)c5)nc5ccccc5c4c3nc2-c2ccc(C#N)cc2)cc1]

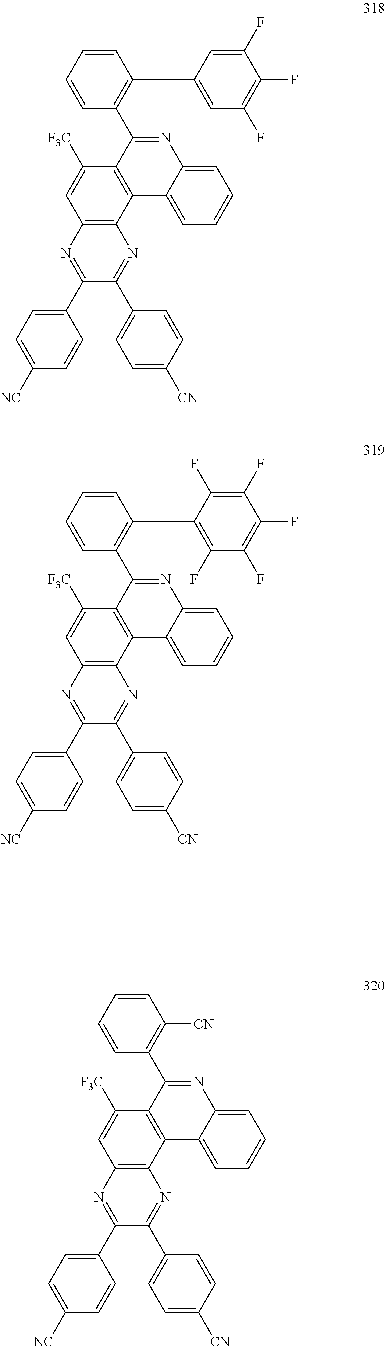 [SMILES: N#Cc1ccc(-c2nc3cc(C(F)(F)F)c4c(-c5ccccc5-c5cc(F)c(F)c(F)c5)nc5ccccc5c4c3nc2-c2ccc(C#N)cc2)cc1 ; N#Cc1ccc(-c2nc3cc(C(F)(F)F)c4c(-c5ccccc5-c5c(F)c(F)c(F)c(F)c5F)nc5ccccc5c4c3nc2-c2ccc(C#N)cc2)cc1 ; N#Cc1ccc(-c2nc3cc(C(F)(F)F)c4c(-c5ccccc5C#N)nc5ccccc5c4c3nc2-c2ccc(C#N)cc2)cc1]

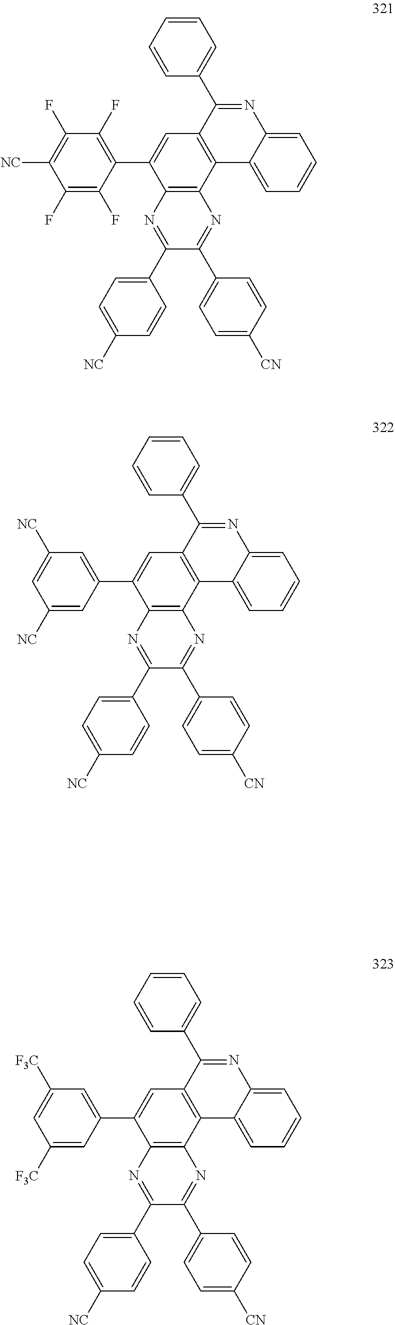 [SMILES: N#Cc1ccc(-c2nc3c(-c4c(F)c(F)c(C#N)c(F)c4F)cc4c(-c5ccccc5)nc5ccccc5c4c3nc2-c2ccc(C#N)cc2)cc1 ; N#Cc1ccc(-c2nc3c(-c4cc(C#N)cc(C#N)c4)cc4c(-c5ccccc5)nc5ccccc5c4c3nc2-c2ccc(C#N)cc2)cc1 ; N#Cc1ccc(-c2nc3c(-c4cc(C(F)(F)F)cc(C(F)(F)F)c4)cc4c(-c5ccccc5)nc5ccccc5c4c3nc2-c2ccc(C#N)cc2)cc1]

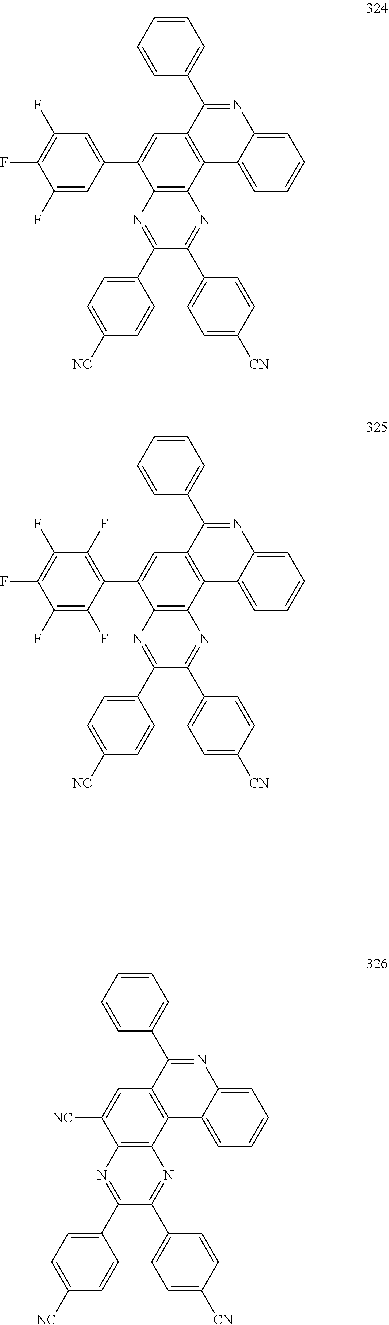 [SMILES: N#Cc1ccc(-c2nc3c(-c4cc(F)c(F)c(F)c4)cc4c(-c5ccccc5)nc5ccccc5c4c3nc2-c2ccc(C#N)cc2)cc1 ; N#Cc1ccc(-c2nc3c(-c4c(F)c(F)c(F)c(F)c4F)cc4c(-c5ccccc5)nc5ccccc5c4c3nc2-c2ccc(C#N)cc2)cc1 ; N#Cc1ccc(-c2nc3c(C#N)cc4c(-c5ccccc5)nc5ccccc5c4c3nc2-c2ccc(C#N)cc2)cc1]

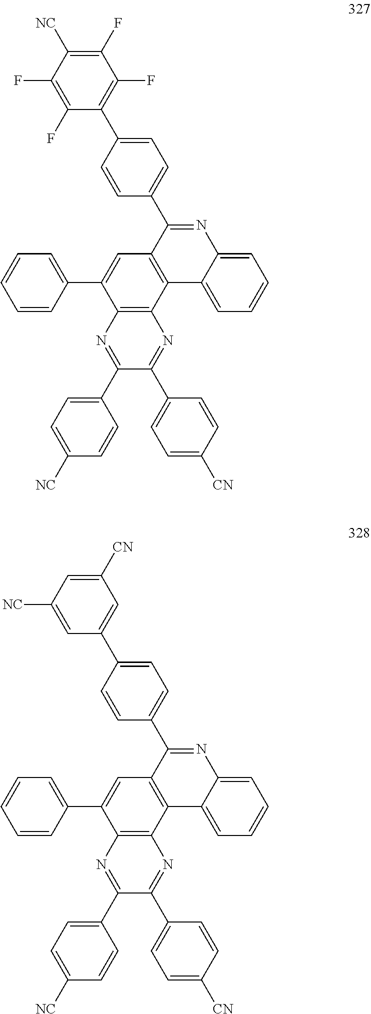 [SMILES: N#Cc1ccc(-c2nc3c(-c4ccccc4)cc4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3c(-c4ccccc4)cc4c(-c5ccc(-c6cc(C#N)cc(C#N)c6)cc5)nc5ccccc5c4c3nc2-c2ccc(C#N)cc2)cc1]

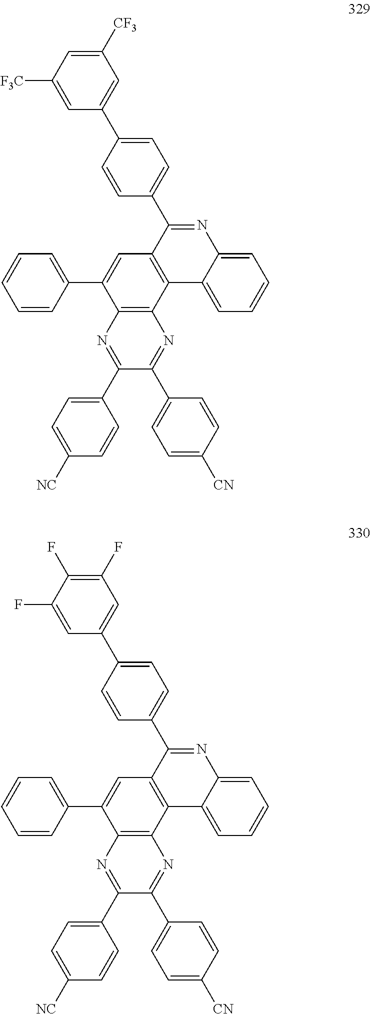 [SMILES: N#Cc1ccc(-c2nc3c(-c4ccccc4)cc4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3c(-c4ccccc4)cc4c(-c5ccc(-c6cc(F)c(F)c(F)c6)cc5)nc5ccccc5c4c3nc2-c2ccc(C#N)cc2)cc1]

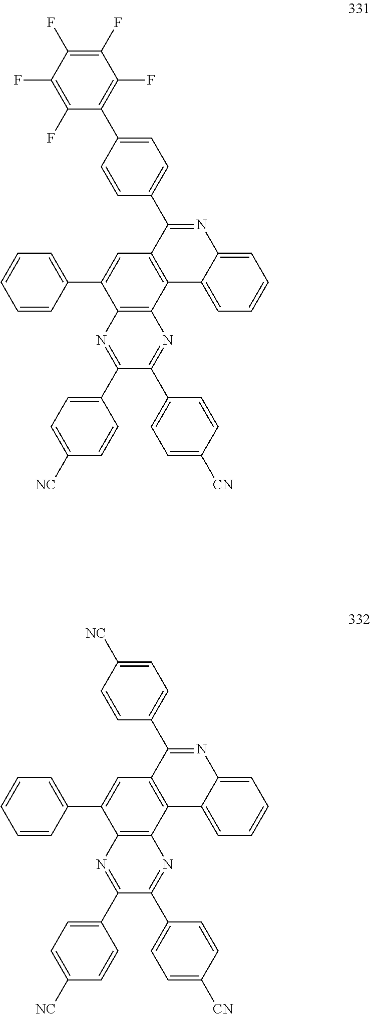 [SMILES: N#Cc1ccc(-c2nc3c(-c4ccccc4)cc4c(-c5ccc(-c6c(F)c(F)c(F)c(F)c6F)cc5)nc5ccccc5c4c3nc2-c2ccc(C#N)cc2)cc1 ; N#Cc1ccc(-c2nc3c(-c4ccccc4)cc4c(-c5ccc(C#N)cc5)nc5ccccc5c4c3nc2-c2ccc(C#N)cc2)cc1]

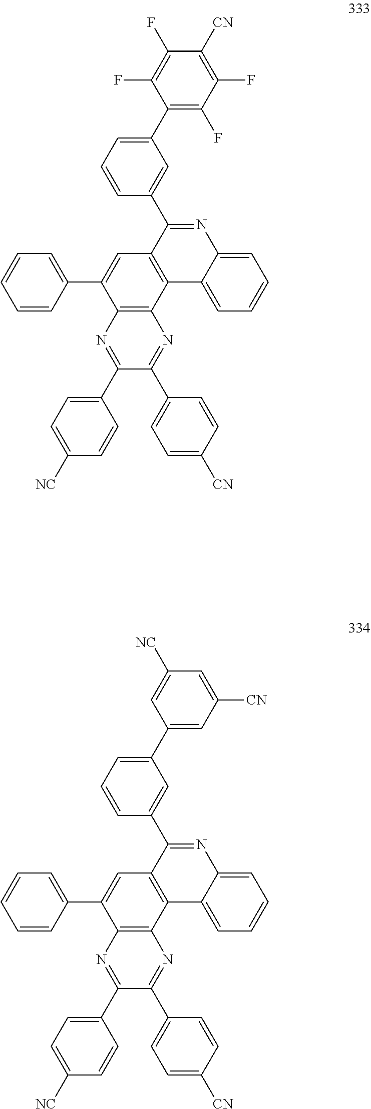 [SMILES: N#Cc1ccc(-c2nc3c(-c4ccccc4)cc4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3c(-c4ccccc4)cc4c(-c5cccc(-c6cc(C#N)cc(C#N)c6)c5)nc5ccccc5c4c3nc2-c2ccc(C#N)cc2)cc1]

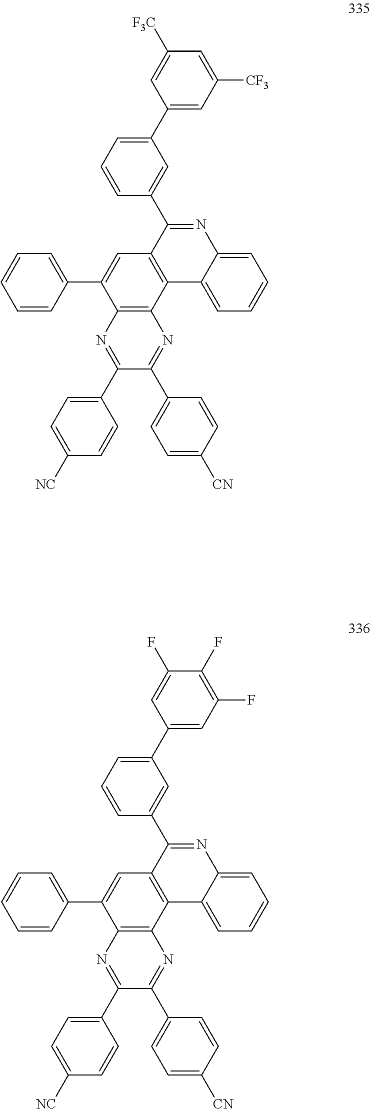 [SMILES: N#Cc1ccc(-c2nc3c(-c4ccccc4)cc4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3c(-c4ccccc4)cc4c(-c5cccc(-c6cc(F)c(F)c(F)c6)c5)nc5ccccc5c4c3nc2-c2ccc(C#N)cc2)cc1]

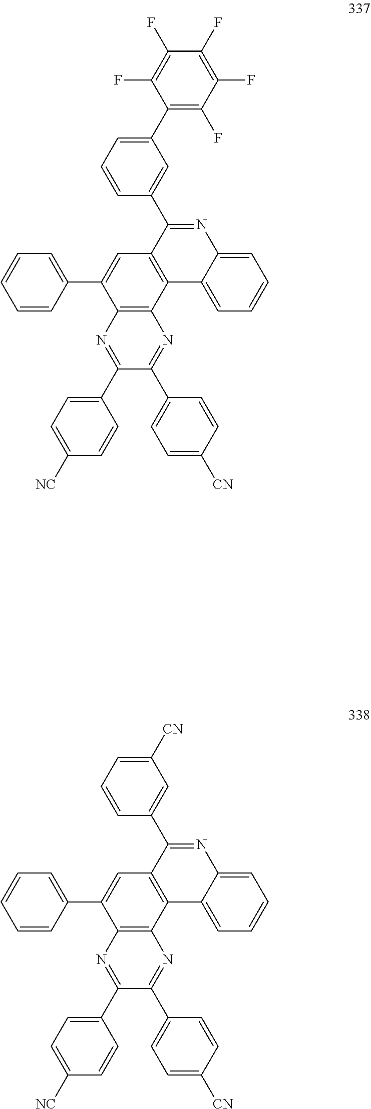 [SMILES: N#Cc1ccc(-c2nc3c(-c4ccccc4)cc4c(-c5cccc(-c6c(F)c(F)c(F)c(F)c6F)c5)nc5ccccc5c4c3nc2-c2ccc(C#N)cc2)cc1 ; N#Cc1ccc(-c2nc3c(-c4ccccc4)cc4c(-c5cccc(C#N)c5)nc5ccccc5c4c3nc2-c2ccc(C#N)cc2)cc1]

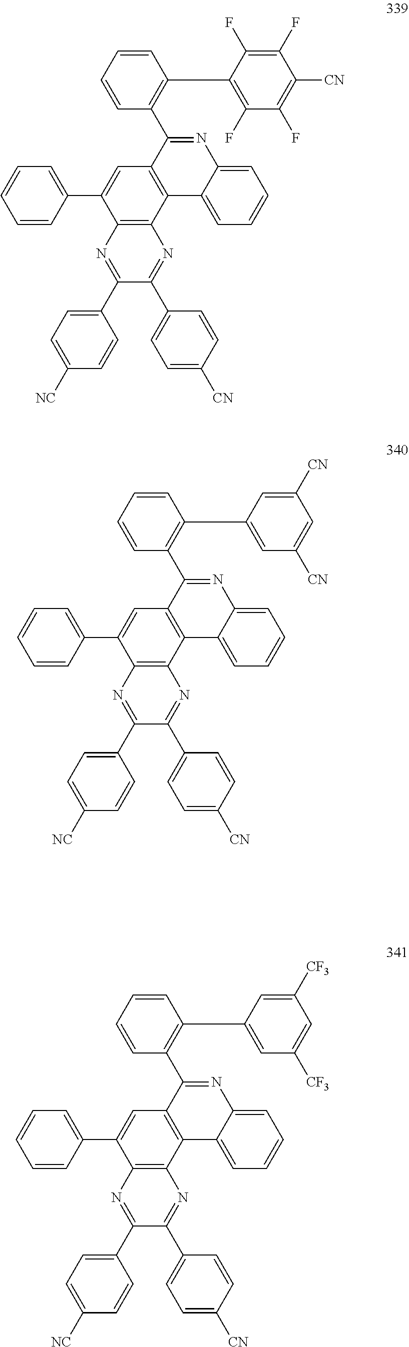 [SMILES: N#Cc1ccc(-c2nc3c(-c4ccccc4)cc4c(-c5ccccc5-c5c(F)c(F)c(C#N)c(F)c5F)nc5ccccc5c4c3nc2-c2ccc(C#N)cc2)cc1 ; N#Cc1ccc(-c2nc3c(-c4ccccc4)cc4c(-c5ccccc5-c5cc(C#N)cc(C#N)c5)nc5ccccc5c4c3nc2-c2ccc(C#N)cc2)cc1 ; N#Cc1ccc(-c2nc3c(-c4ccccc4)cc4c(-c5ccccc5-c5cc(C(F)(F)F)cc(C(F)(F)F)c5)nc5ccccc5c4c3nc2-c2ccc(C#N)cc2)cc1]

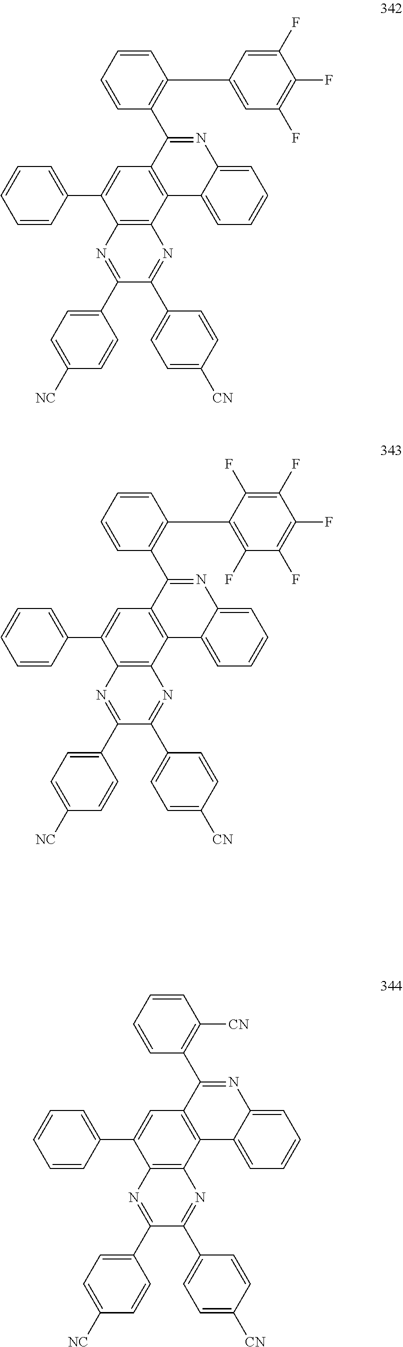 [SMILES: N#Cc1ccc(-c2nc3c(-c4ccccc4)cc4c(-c5ccccc5-c5cc(F)c(F)c(F)c5)nc5ccccc5c4c3nc2-c2ccc(C#N)cc2)cc1 ; N#Cc1ccc(-c2nc3c(-c4ccccc4)cc4c(-c5ccccc5-c5c(F)c(F)c(F)c(F)c5F)nc5ccccc5c4c3nc2-c2ccc(C#N)cc2)cc1 ; N#Cc1ccc(-c2nc3c(-c4ccccc4)cc4c(-c5ccccc5C#N)nc5ccccc5c4c3nc2-c2ccc(C#N)cc2)cc1]

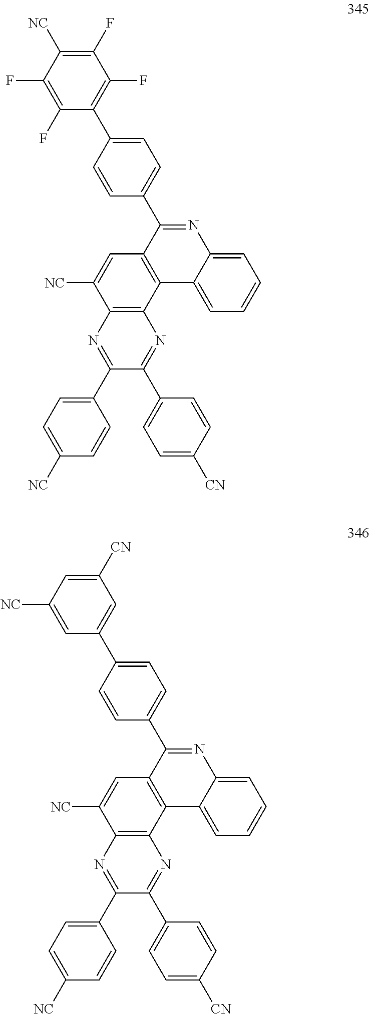 [SMILES: N#Cc1ccc(-c2nc3c(C#N)cc4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3c(C#N)cc4c(-c5ccc(-c6cc(C#N)cc(C#N)c6)cc5)nc5ccccc5c4c3nc2-c2ccc(C#N)cc2)cc1]

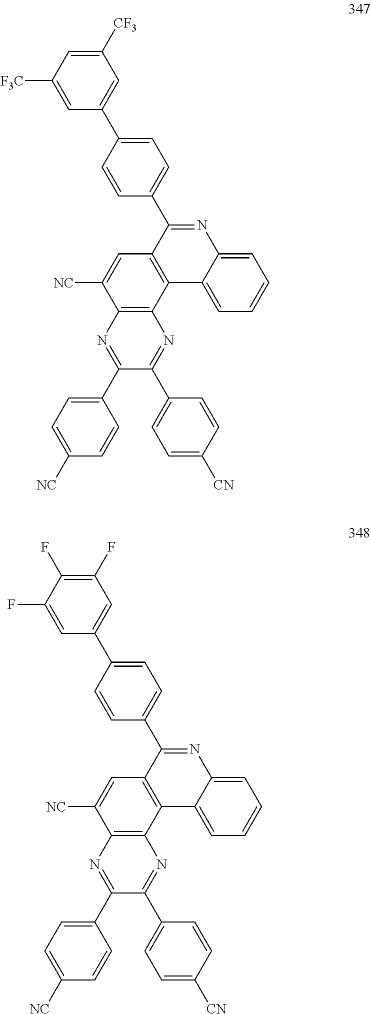 [SMILES: N#Cc1ccc(-c2nc3c(C#N)cc4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3c(C#N)cc4c(-c5ccc(-c6cc(F)c(F)c(F)c6)cc5)nc5ccccc5c4c3nc2-c2ccc(C#N)cc2)cc1]

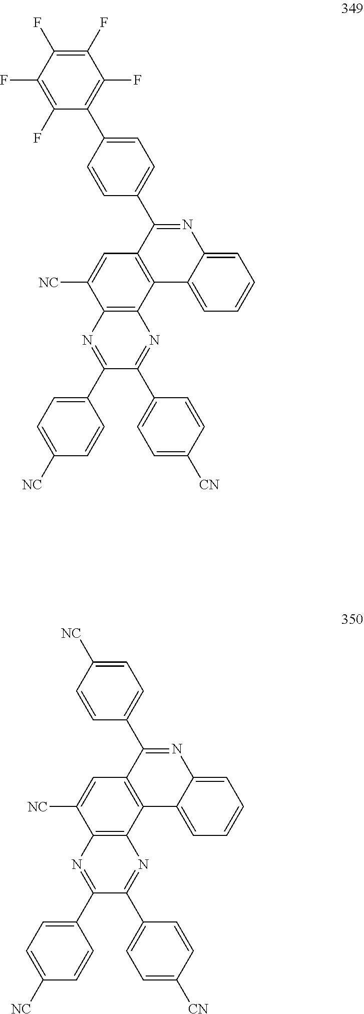 [SMILES: N#Cc1ccc(-c2nc3c(C#N)cc4c(-c5ccc(-c6c(F)c(F)c(F)c(F)c6F)cc5)nc5ccccc5c4c3nc2-c2ccc(C#N)cc2)cc1 ; N#Cc1ccc(-c2nc3c(C#N)cc4c(-c5ccc(C#N)cc5)nc5ccccc5c4c3nc2-c2ccc(C#N)cc2)cc1]

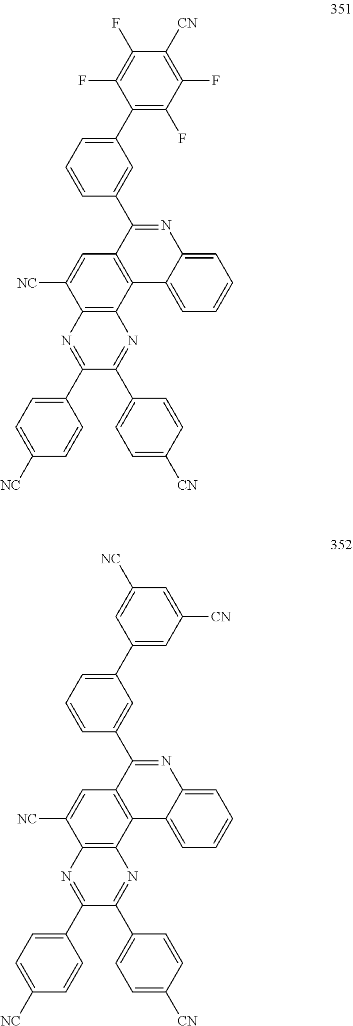 [SMILES: N#Cc1ccc(-c2nc3c(C#N)cc4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3c(C#N)cc4c(-c5cccc(-c6cc(C#N)cc(C#N)c6)c5)nc5ccccc5c4c3nc2-c2ccc(C#N)cc2)cc1]

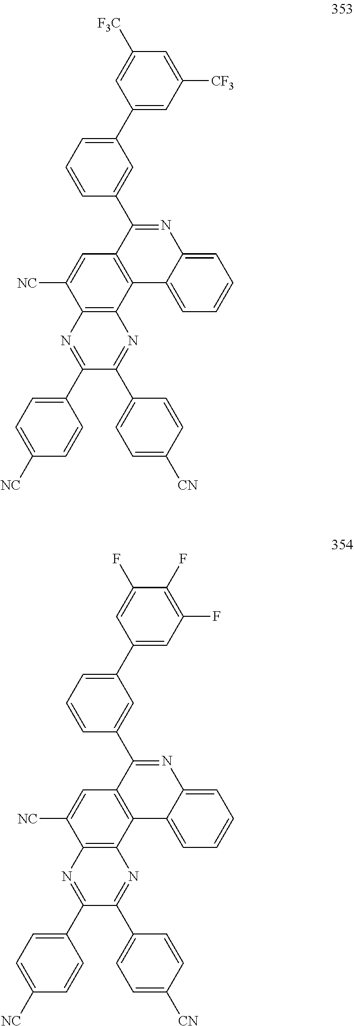 [SMILES: N#Cc1ccc(-c2nc3c(C#N)cc4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3c(C#N)cc4c(-c5cccc(-c6cc(F)c(F)c(F)c6)c5)nc5ccccc5c4c3nc2-c2ccc(C#N)cc2)cc1]

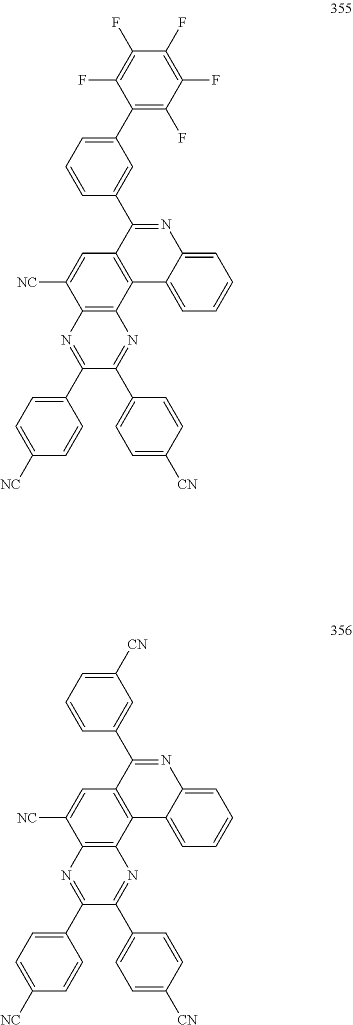 [SMILES: N#Cc1ccc(-c2nc3c(C#N)cc4c(-c5cccc(-c6c(F)c(F)c(F)c(F)c6F)c5)nc5ccccc5c4c3nc2-c2ccc(C#N)cc2)cc1 ; N#Cc1ccc(-c2nc3c(C#N)cc4c(-c5cccc(C#N)c5)nc5ccccc5c4c3nc2-c2ccc(C#N)cc2)cc1]

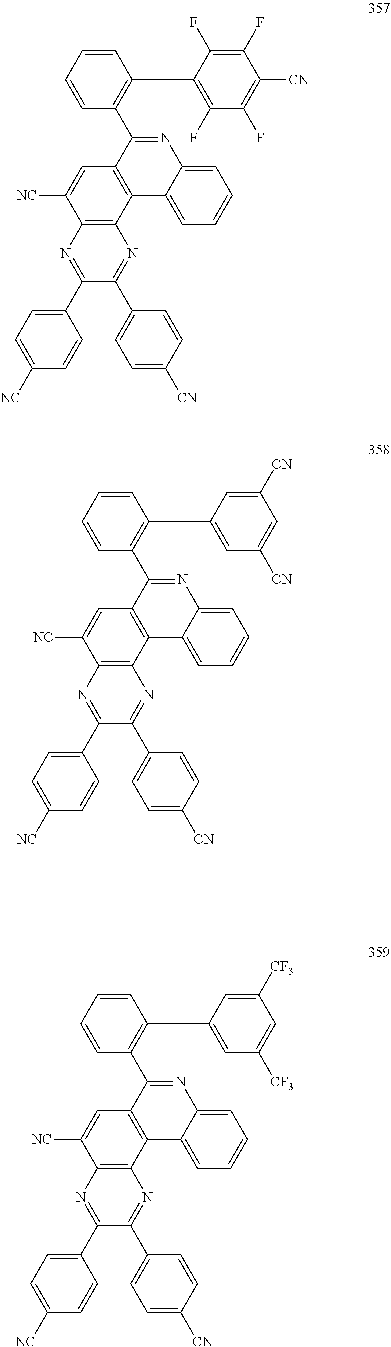 [SMILES: N#Cc1ccc(-c2nc3c(C#N)cc4c(-c5ccccc5-c5c(F)c(F)c(C#N)c(F)c5F)nc5ccccc5c4c3nc2-c2ccc(C#N)cc2)cc1 ; N#Cc1ccc(-c2nc3c(C#N)cc4c(-c5ccccc5-c5cc(C#N)cc(C#N)c5)nc5ccccc5c4c3nc2-c2ccc(C#N)cc2)cc1 ; N#Cc1ccc(-c2nc3c(C#N)cc4c(-c5ccccc5-c5cc(C(F)(F)F)cc(C(F)(F)F)c5)nc5ccccc5c4c3nc2-c2ccc(C#N)cc2)cc1]

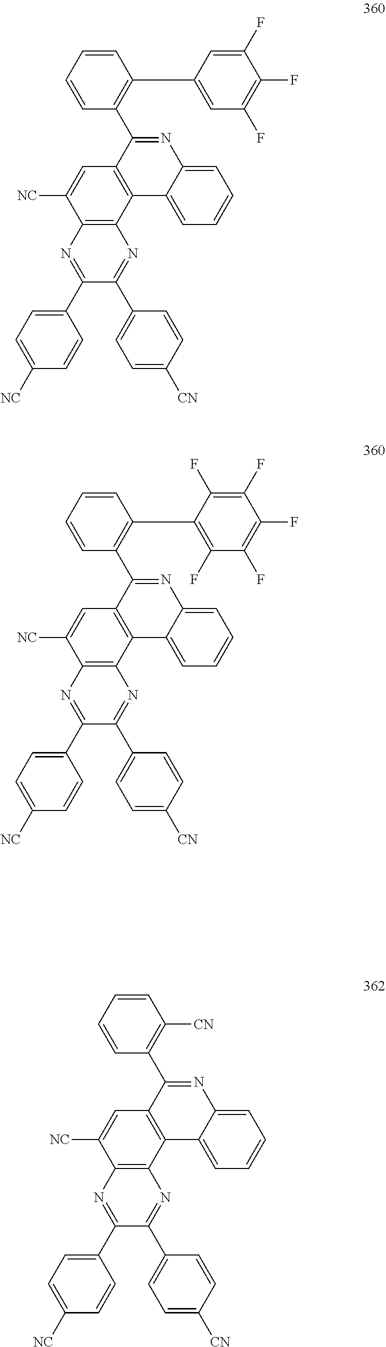 [SMILES: N#Cc1ccc(-c2nc3c(C#N)cc4c(-c5ccccc5-c5cc(F)c(F)c(F)c5)nc5ccccc5c4c3nc2-c2ccc(C#N)cc2)cc1 ; N#Cc1ccc(-c2nc3c(C#N)cc4c(-c5ccccc5-c5c(F)c(F)c(F)c(F)c5F)nc5ccccc5c4c3nc2-c2ccc(C#N)cc2)cc1 ; N#Cc1ccc(-c2nc3c(C#N)cc4c(-c5ccccc5C#N)nc5ccccc5c4c3nc2-c2ccc(C#N)cc2)cc1]

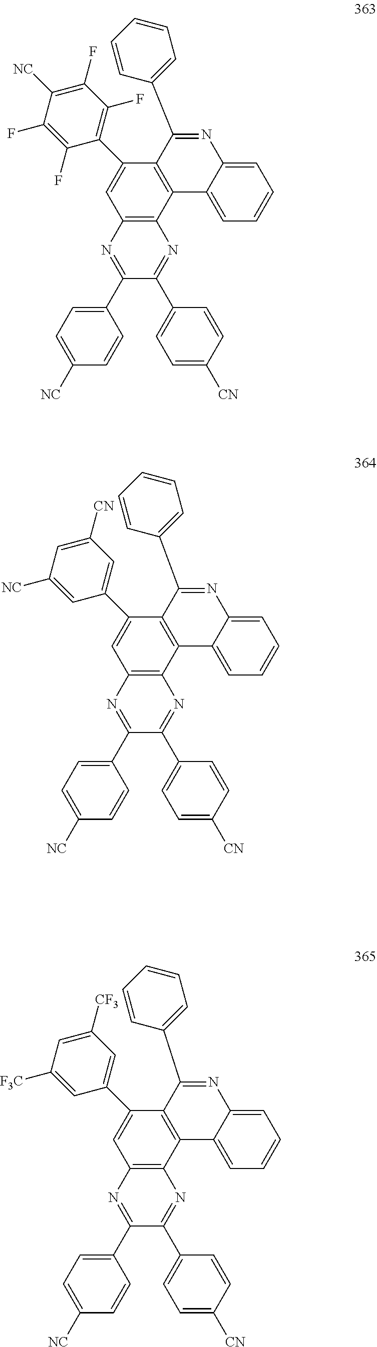 [SMILES: N#Cc1ccc(-c2nc3cc(-c4c(F)c(F)c(C#N)c(F)c4F)c4c(-c5ccccc5)nc5ccccc5c4c3nc2-c2ccc(C#N)cc2)cc1 ; N#Cc1ccc(-c2nc3cc(-c4cc(C#N)cc(C#N)c4)c4c(-c5ccccc5)nc5ccccc5c4c3nc2-c2ccc(C#N)cc2)cc1 ; N#Cc1ccc(-c2nc3cc(-c4cc(C(F)(F)F)cc(C(F)(F)F)c4)c4c(-c5ccccc5)nc5ccccc5c4c3nc2-c2ccc(C#N)cc2)cc1]

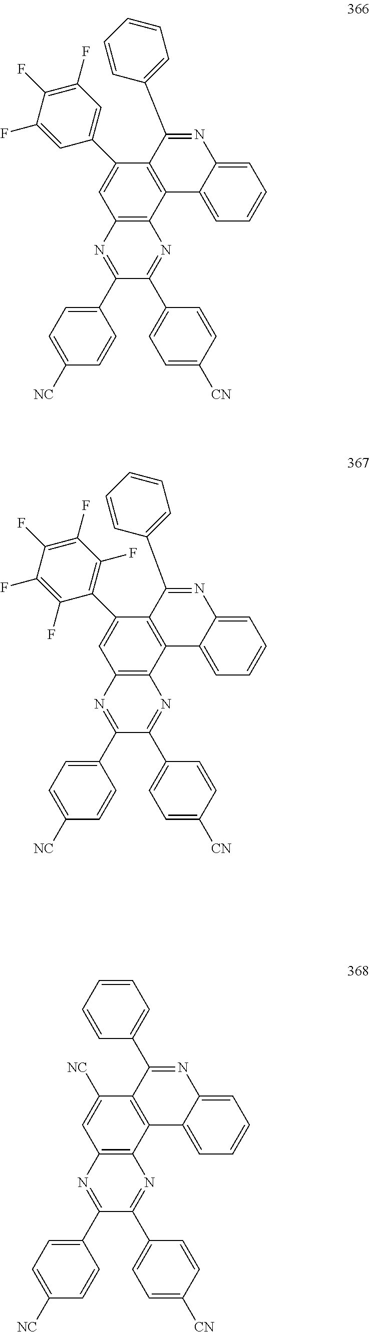 [SMILES: N#Cc1ccc(-c2nc3cc(-c4cc(F)c(F)c(F)c4)c4c(-c5ccccc5)nc5ccccc5c4c3nc2-c2ccc(C#N)cc2)cc1 ; N#Cc1ccc(-c2nc3cc(-c4c(F)c(F)c(F)c(F)c4F)c4c(-c5ccccc5)nc5ccccc5c4c3nc2-c2ccc(C#N)cc2)cc1 ; N#Cc1ccc(-c2nc3cc(C#N)c4c(-c5ccccc5)nc5ccccc5c4c3nc2-c2ccc(C#N)cc2)cc1]

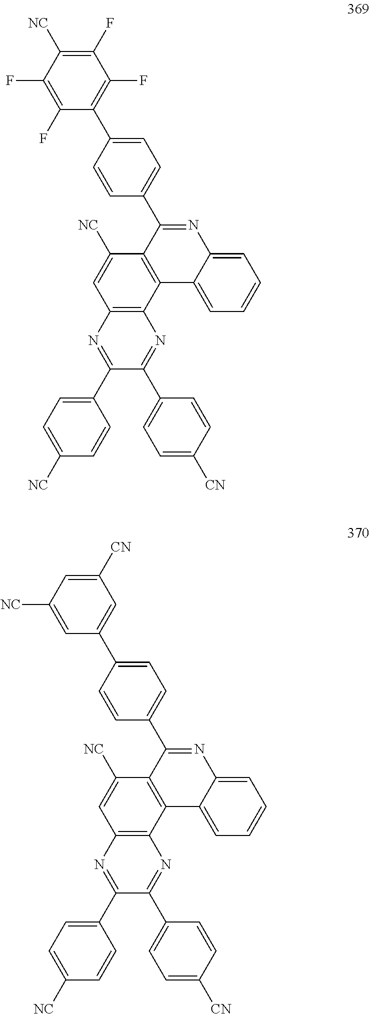 [SMILES: N#Cc1ccc(-c2nc3cc(C#N)c4c(-c5ccc(-c6c(F)c(F)c(C#N)c(F)c6F)cc5)nc5ccccc5c4c3nc2-c2ccc(C#N)cc2)cc1 ; N#Cc1ccc(-c2nc3cc(C#N)c4c(-c5ccc(-c6cc(C#N)cc(C#N)c6)cc5)nc5ccccc5c4c3nc2-c2ccc(C#N)cc2)cc1]

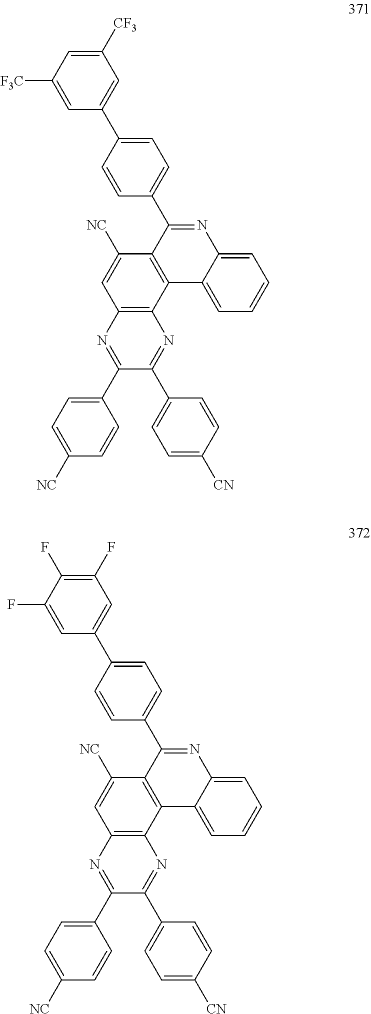 [SMILES: N#Cc1ccc(-c2nc3cc(C#N)c4c(-c5ccc(-c6cc(C(F)(F)F)cc(C(F)(F)F)c6)cc5)nc5ccccc5c4c3nc2-c2ccc(C#N)cc2)cc1 ; N#Cc1ccc(-c2nc3cc(C#N)c4c(-c5ccc(-c6cc(F)c(F)c(F)c6)cc5)nc5ccccc5c4c3nc2-c2ccc(C#N)cc2)cc1]

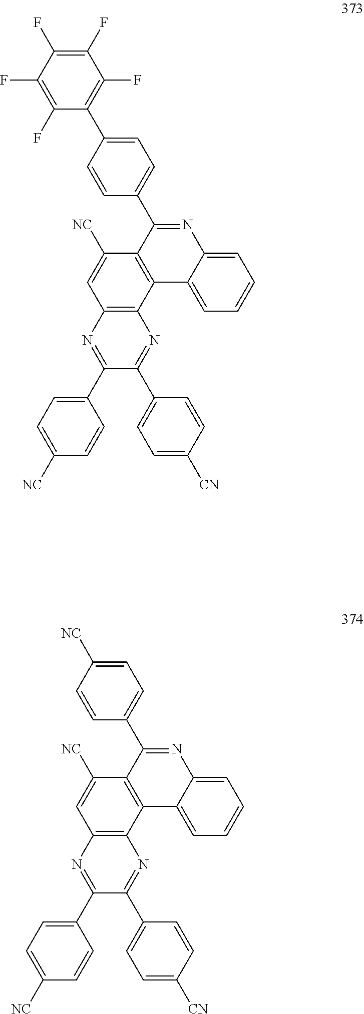 [SMILES: N#Cc1ccc(-c2nc3cc(C#N)c4c(-c5ccc(-c6c(F)c(F)c(F)c(F)c6F)cc5)nc5ccccc5c4c3nc2-c2ccc(C#N)cc2)cc1 ; N#Cc1ccc(-c2nc3cc(C#N)c4c(-c5ccc(C#N)cc5)nc5ccccc5c4c3nc2-c2ccc(C#N)cc2)cc1]

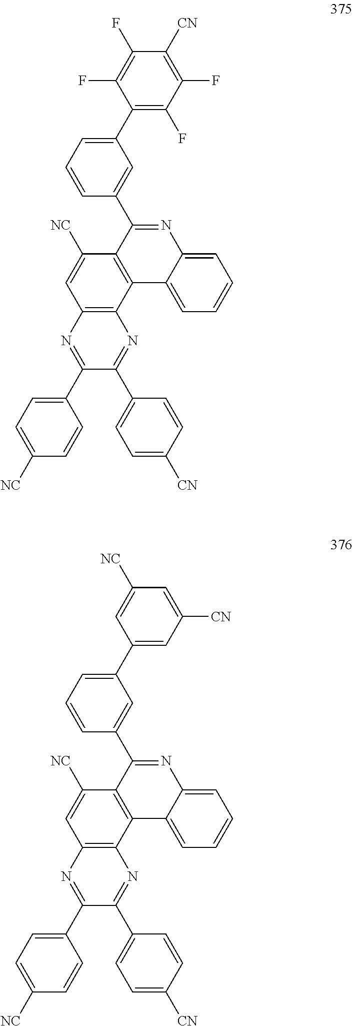 [SMILES: N#Cc1ccc(-c2nc3cc(C#N)c4c(-c5cccc(-c6c(F)c(F)c(C#N)c(F)c6F)c5)nc5ccccc5c4c3nc2-c2ccc(C#N)cc2)cc1 ; N#Cc1ccc(-c2nc3cc(C#N)c4c(-c5cccc(-c6cc(C#N)cc(C#N)c6)c5)nc5ccccc5c4c3nc2-c2ccc(C#N)cc2)cc1]

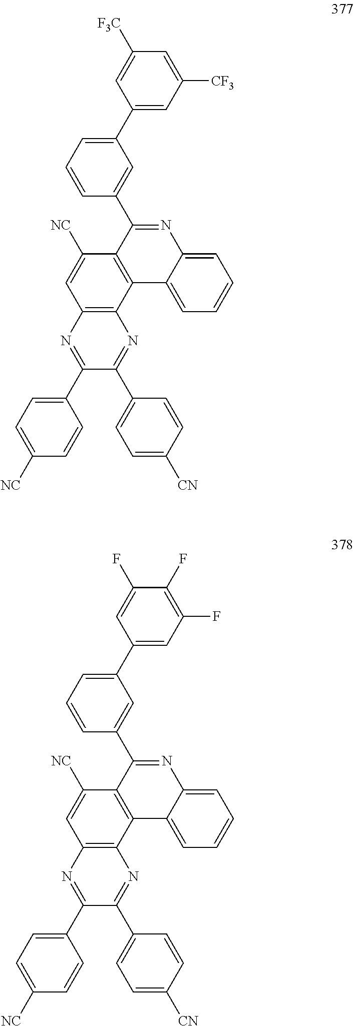 [SMILES: N#Cc1ccc(-c2nc3cc(C#N)c4c(-c5cccc(-c6cc(C(F)(F)F)cc(C(F)(F)F)c6)c5)nc5ccccc5c4c3nc2-c2ccc(C#N)cc2)cc1 ; N#Cc1ccc(-c2nc3cc(C#N)c4c(-c5cccc(-c6cc(F)c(F)c(F)c6)c5)nc5ccccc5c4c3nc2-c2ccc(C#N)cc2)cc1]

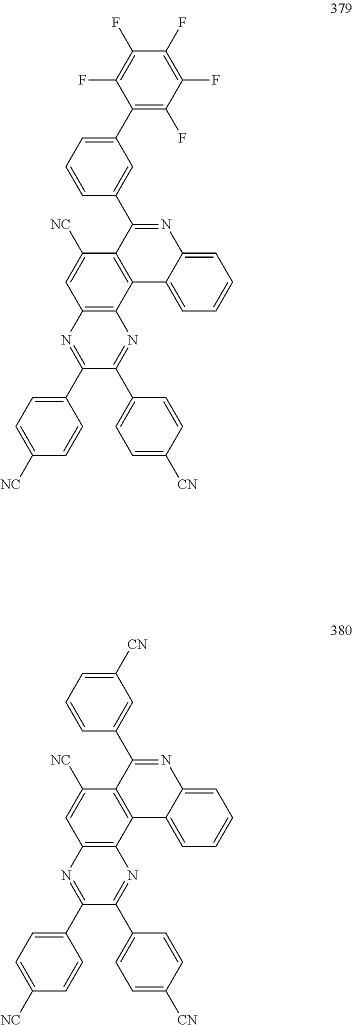 [SMILES: N#Cc1ccc(-c2nc3cc(C#N)c4c(-c5cccc(-c6c(F)c(F)c(F)c(F)c6F)c5)nc5ccccc5c4c3nc2-c2ccc(C#N)cc2)cc1 ; N#Cc1ccc(-c2nc3cc(C#N)c4c(-c5cccc(C#N)c5)nc5ccccc5c4c3nc2-c2ccc(C#N)cc2)cc1]

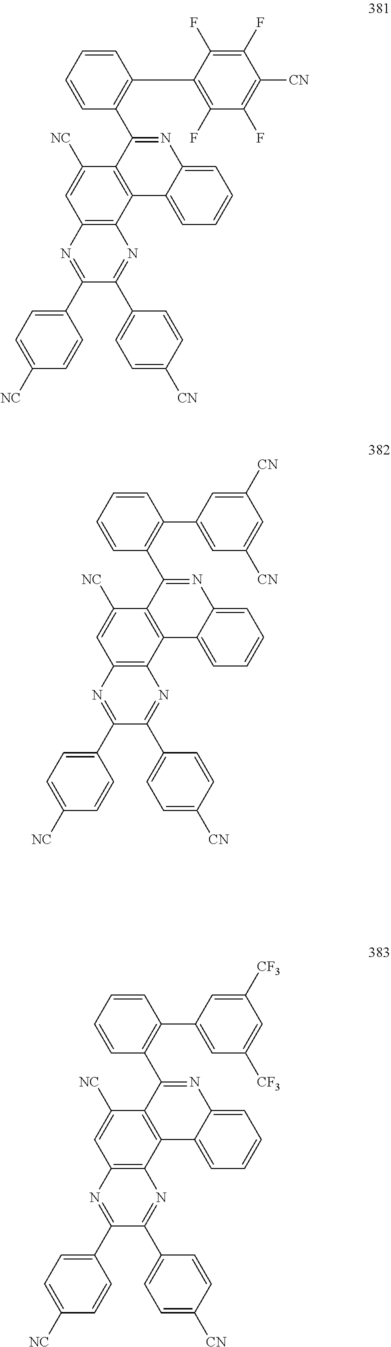 [SMILES: N#Cc1ccc(-c2nc3cc(C#N)c4c(-c5ccccc5-c5c(F)c(F)c(C#N)c(F)c5F)nc5ccccc5c4c3nc2-c2ccc(C#N)cc2)cc1 ; N#Cc1ccc(-c2nc3cc(C#N)c4c(-c5ccccc5-c5cc(C#N)cc(C#N)c5)nc5ccccc5c4c3nc2-c2ccc(C#N)cc2)cc1 ; N#Cc1ccc(-c2nc3cc(C#N)c4c(-c5ccccc5-c5cc(C(F)(F)F)cc(C(F)(F)F)c5)nc5ccccc5c4c3nc2-c2ccc(C#N)cc2)cc1]

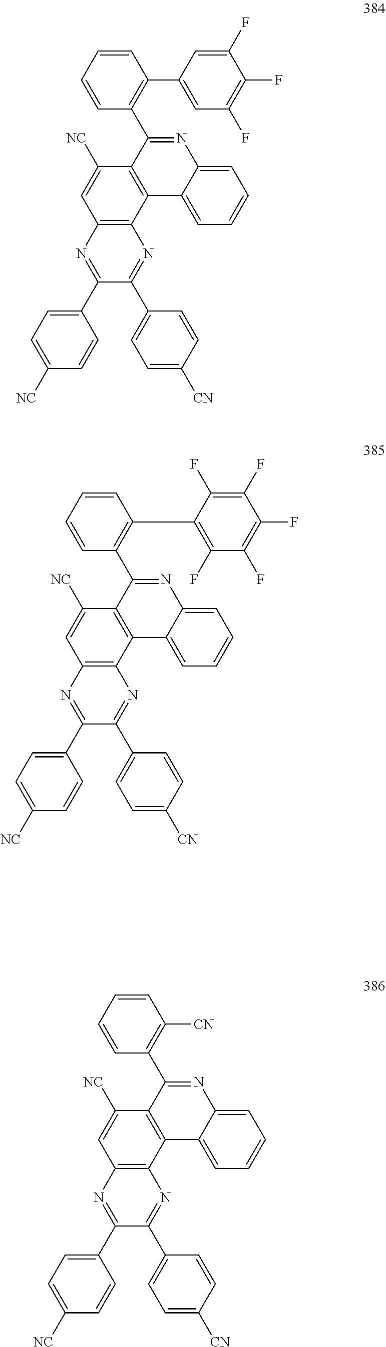 [SMILES: N#Cc1ccc(-c2nc3cc(C#N)c4c(-c5ccccc5-c5cc(F)c(F)c(F)c5)nc5ccccc5c4c3nc2-c2ccc(C#N)cc2)cc1 ; N#Cc1ccc(-c2nc3cc(C#N)c4c(-c5ccccc5-c5c(F)c(F)c(F)c(F)c5F)nc5ccccc5c4c3nc2-c2ccc(C#N)cc2)cc1 ; N#Cc1ccc(-c2nc3cc(C#N)c4c(-c5ccccc5C#N)nc5ccccc5c4c3nc2-c2ccc(C#N)cc2)cc1]

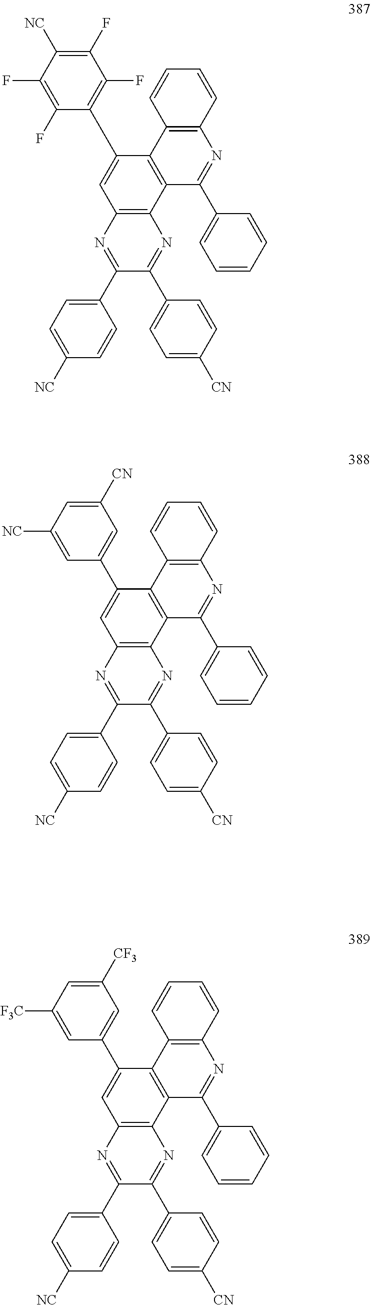 [SMILES: N#Cc1ccc(-c2nc3cc(-c4c(F)c(F)c(C#N)c(F)c4F)c4c5ccccc5nc(-c5ccccc5)c4c3nc2-c2ccc(C#N)cc2)cc1 ; N#Cc1ccc(-c2nc3cc(-c4cc(C#N)cc(C#N)c4)c4c5ccccc5nc(-c5ccccc5)c4c3nc2-c2ccc(C#N)cc2)cc1 ; N#Cc1ccc(-c2nc3cc(-c4cc(C(F)(F)F)cc(C(F)(F)F)c4)c4c5ccccc5nc(-c5ccccc5)c4c3nc2-c2ccc(C#N)cc2)cc1]

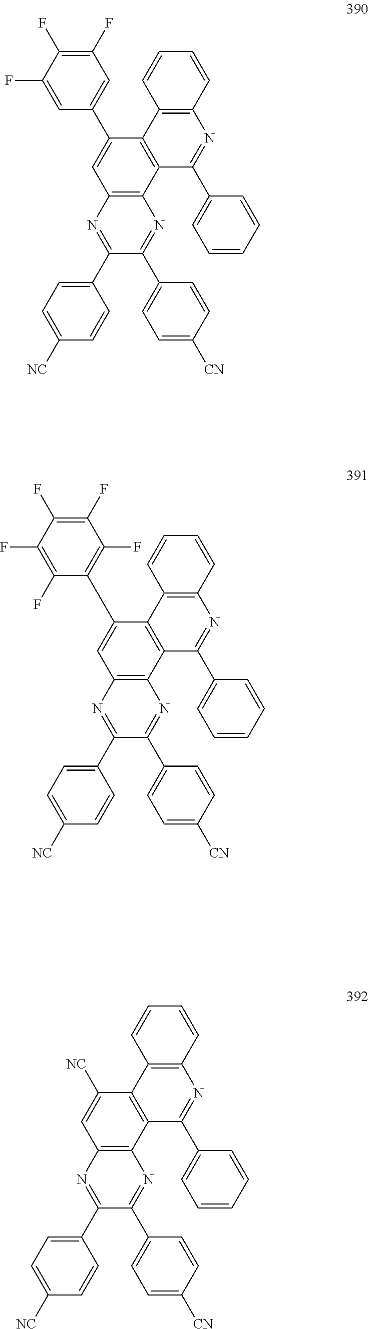 [SMILES: N#Cc1ccc(-c2nc3cc(-c4cc(F)c(F)c(F)c4)c4c5ccccc5nc(-c5ccccc5)c4c3nc2-c2ccc(C#N)cc2)cc1 ; N#Cc1ccc(-c2nc3cc(-c4c(F)c(F)c(F)c(F)c4F)c4c5ccccc5nc(-c5ccccc5)c4c3nc2-c2ccc(C#N)cc2)cc1 ; N#Cc1ccc(-c2nc3cc(C#N)c4c5ccccc5nc(-c5ccccc5)c4c3nc2-c2ccc(C#N)cc2)cc1]

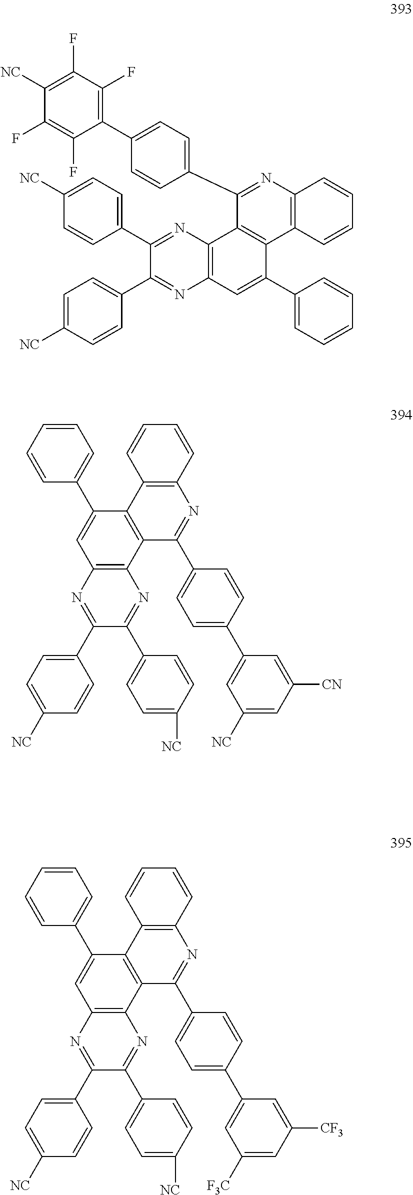 [SMILES: N#Cc1ccc(-c2nc3cc(-c4ccccc4)c4c5ccccc5nc(-c5ccc(-c6c(F)c(F)c(C#N)c(F)c6F)cc5)c4c3nc2-c2ccc(C#N)cc2)cc1 ; N#Cc1ccc(-c2nc3cc(-c4ccccc4)c4c5ccccc5nc(-c5ccc(-c6cc(C#N)cc(C#N)c6)cc5)c4c3nc2-c2ccc(C#N)cc2)cc1 ; N#Cc1ccc(-c2nc3cc(-c4ccccc4)c4c5ccccc5nc(-c5ccc(-c6cc(C(F)(F)F)cc(C(F)(F)F)c6)cc5)c4c3nc2-c2ccc(C#N)cc2)cc1]

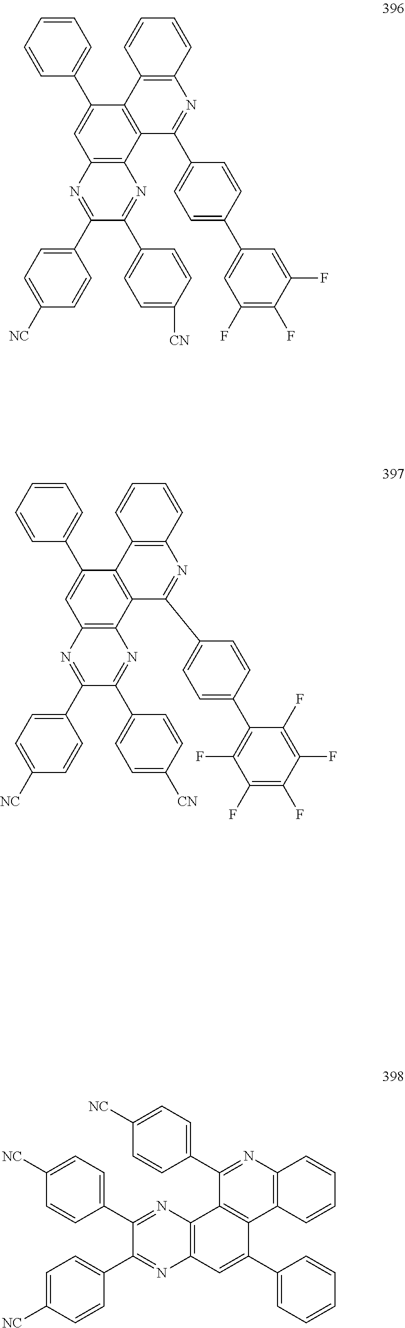 [SMILES: N#Cc1ccc(-c2nc3cc(-c4ccccc4)c4c5ccccc5nc(-c5ccc(-c6cc(F)c(F)c(F)c6)cc5)c4c3nc2-c2ccc(C#N)cc2)cc1 ; N#Cc1ccc(-c2nc3cc(-c4ccccc4)c4c5ccccc5nc(-c5ccc(-c6c(F)c(F)c(F)c(F)c6F)cc5)c4c3nc2-c2ccc(C#N)cc2)cc1 ; N#Cc1ccc(-c2nc3cc(-c4ccccc4)c4c5ccccc5nc(-c5ccc(C#N)cc5)c4c3nc2-c2ccc(C#N)cc2)cc1]

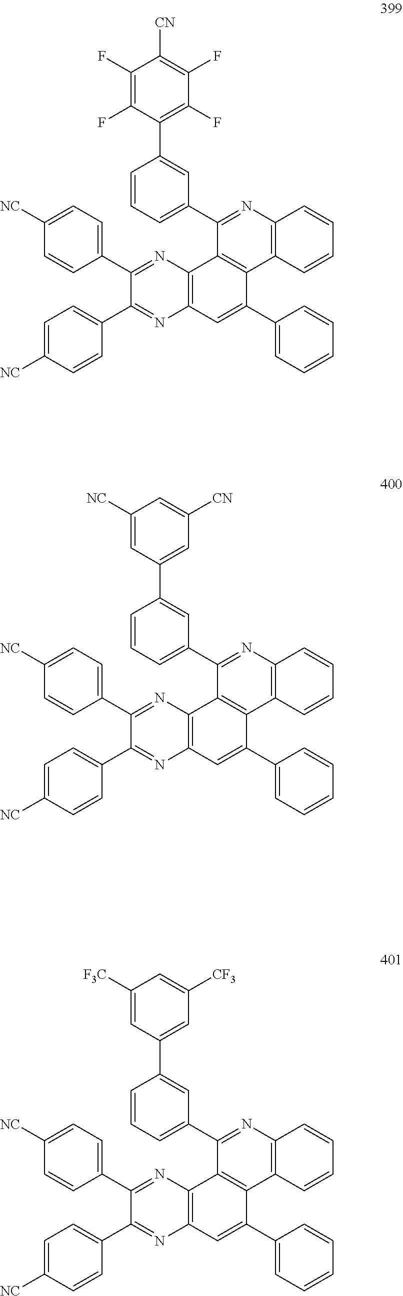 [SMILES: N#Cc1ccc(-c2nc3cc(-c4ccccc4)c4c5ccccc5nc(-c5cccc(-c6c(F)c(F)c(C#N)c(F)c6F)c5)c4c3nc2-c2ccc(C#N)cc2)cc1 ; N#Cc1ccc(-c2nc3cc(-c4ccccc4)c4c5ccccc5nc(-c5cccc(-c6cc(C#N)cc(C#N)c6)c5)c4c3nc2-c2ccc(C#N)cc2)cc1 ; N#Cc1ccc(-c2nc3cc(-c4ccccc4)c4c5ccccc5nc(-c5cccc(-c6cc(C(F)(F)F)cc(C(F)(F)F)c6)c5)c4c3nc2-c2ccc(C#N)cc2)cc1]

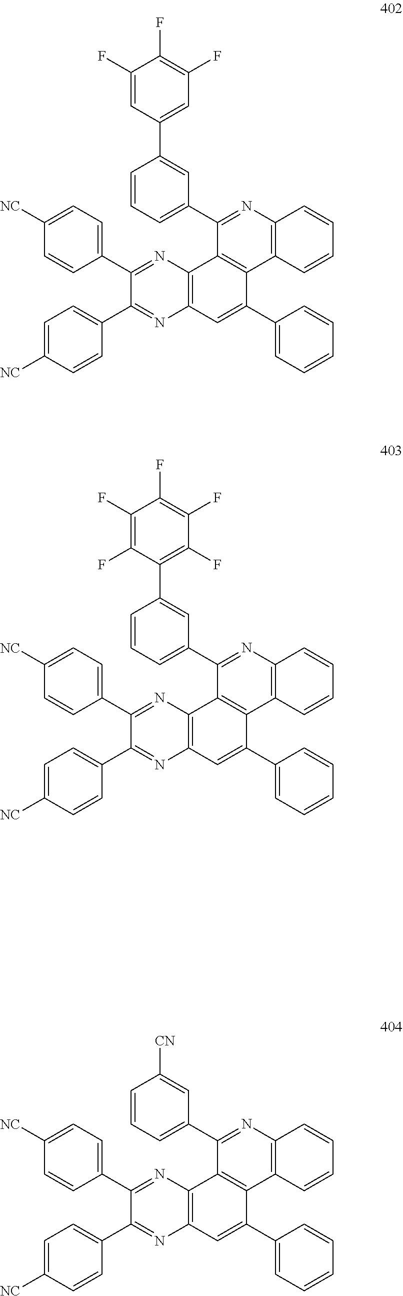 [SMILES: N#Cc1ccc(-c2nc3cc(-c4ccccc4)c4c5ccccc5nc(-c5cccc(-c6cc(F)c(F)c(F)c6)c5)c4c3nc2-c2ccc(C#N)cc2)cc1 ; N#Cc1ccc(-c2nc3cc(-c4ccccc4)c4c5ccccc5nc(-c5cccc(-c6c(F)c(F)c(F)c(F)c6F)c5)c4c3nc2-c2ccc(C#N)cc2)cc1 ; N#Cc1ccc(-c2nc3cc(-c4ccccc4)c4c5ccccc5nc(-c5cccc(C#N)c5)c4c3nc2-c2ccc(C#N)cc2)cc1]

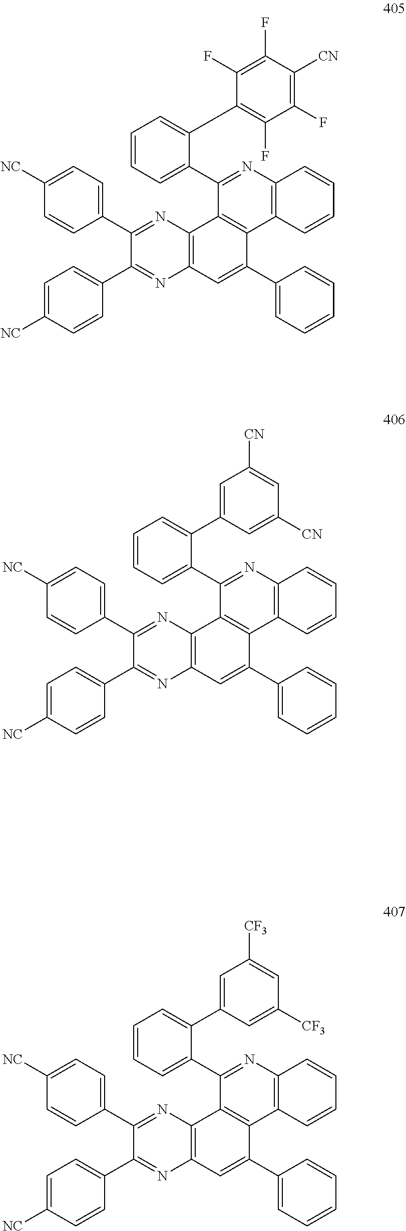 [SMILES: N#Cc1ccc(-c2nc3cc(-c4ccccc4)c4c5ccccc5nc(-c5ccccc5-c5c(F)c(F)c(C#N)c(F)c5F)c4c3nc2-c2ccc(C#N)cc2)cc1 ; N#Cc1ccc(-c2nc3cc(-c4ccccc4)c4c5ccccc5nc(-c5ccccc5-c5cc(C#N)cc(C#N)c5)c4c3nc2-c2ccc(C#N)cc2)cc1 ; N#Cc1ccc(-c2nc3cc(-c4ccccc4)c4c5ccccc5nc(-c5ccccc5-c5cc(C(F)(F)F)cc(C(F)(F)F)c5)c4c3nc2-c2ccc(C#N)cc2)cc1]

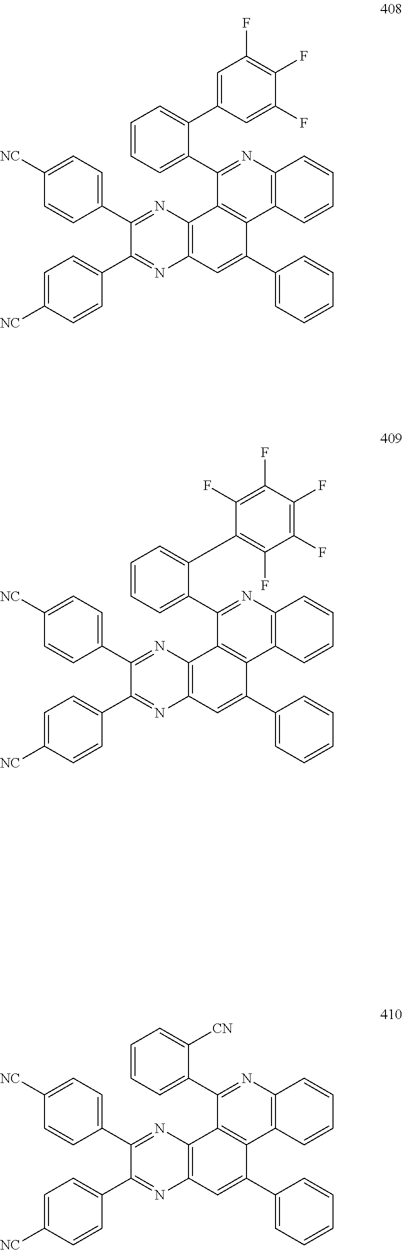 [SMILES: N#Cc1ccc(-c2nc3cc(-c4ccccc4)c4c5ccccc5nc(-c5ccccc5-c5cc(F)c(F)c(F)c5)c4c3nc2-c2ccc(C#N)cc2)cc1 ; N#Cc1ccc(-c2nc3cc(-c4ccccc4)c4c5ccccc5nc(-c5ccccc5-c5c(F)c(F)c(F)c(F)c5F)c4c3nc2-c2ccc(C#N)cc2)cc1 ; N#Cc1ccc(-c2nc3cc(-c4ccccc4)c4c5ccccc5nc(-c5ccccc5C#N)c4c3nc2-c2ccc(C#N)cc2)cc1]

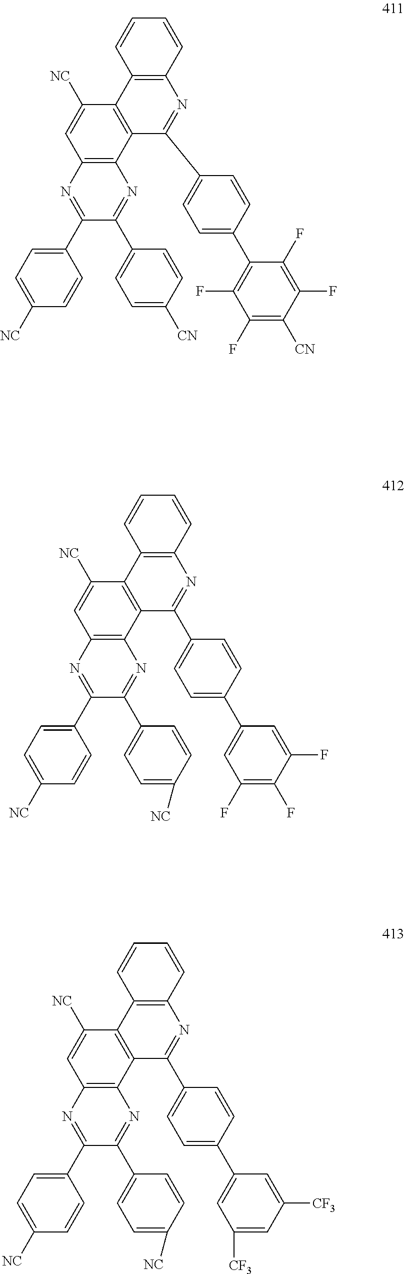 [SMILES: N#Cc1ccc(-c2nc3cc(C#N)c4c5ccccc5nc(-c5ccc(-c6c(F)c(F)c(C#N)c(F)c6F)cc5)c4c3nc2-c2ccc(C#N)cc2)cc1 ; N#Cc1ccc(-c2nc3cc(C#N)c4c5ccccc5nc(-c5ccc(-c6cc(F)c(F)c(F)c6)cc5)c4c3nc2-c2ccc(C#N)cc2)cc1 ; N#Cc1ccc(-c2nc3cc(C#N)c4c5ccccc5nc(-c5ccc(-c6cc(C(F)(F)F)cc(C(F)(F)F)c6)cc5)c4c3nc2-c2ccc(C#N)cc2)cc1]

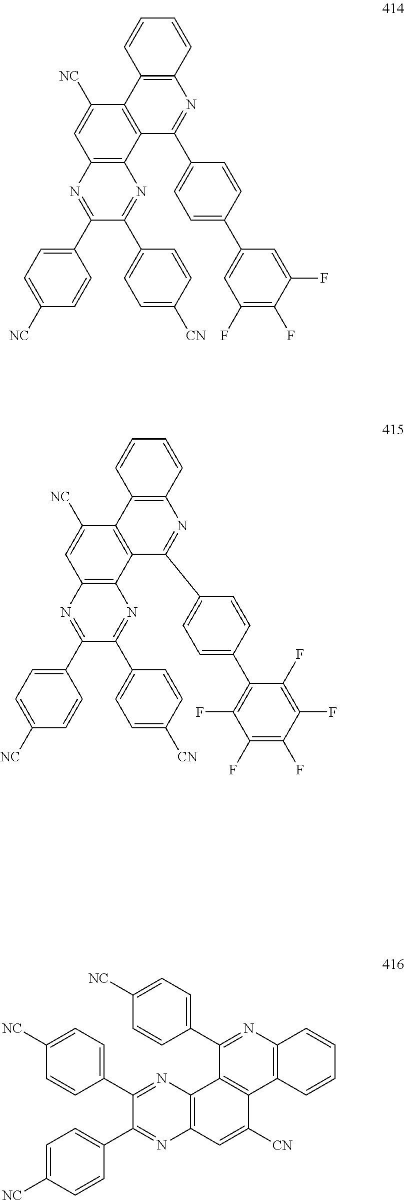 [SMILES: N#Cc1ccc(-c2nc3cc(C#N)c4c5ccccc5nc(-c5ccc(-c6cc(F)c(F)c(F)c6)cc5)c4c3nc2-c2ccc(C#N)cc2)cc1 ; N#Cc1ccc(-c2nc3cc(C#N)c4c5ccccc5nc(-c5ccc(-c6c(F)c(F)c(F)c(F)c6F)cc5)c4c3nc2-c2ccc(C#N)cc2)cc1 ; N#Cc1ccc(-c2nc3cc(C#N)c4c5ccccc5nc(-c5ccc(C#N)cc5)c4c3nc2-c2ccc(C#N)cc2)cc1]

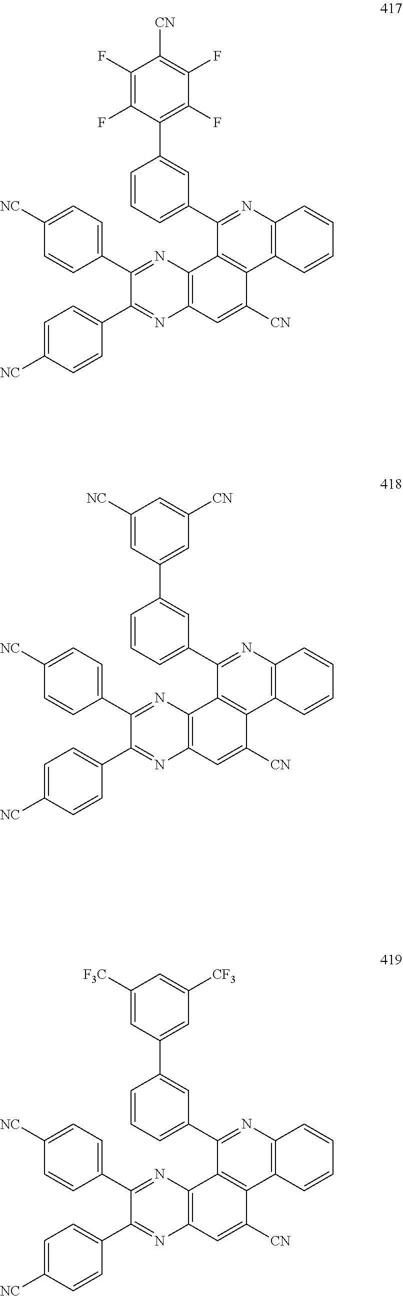 [SMILES: N#Cc1ccc(-c2nc3cc(C#N)c4c5ccccc5nc(-c5cccc(-c6c(F)c(F)c(C#N)c(F)c6F)c5)c4c3nc2-c2ccc(C#N)cc2)cc1 ; N#Cc1ccc(-c2nc3cc(C#N)c4c5ccccc5nc(-c5cccc(-c6cc(C#N)cc(C#N)c6)c5)c4c3nc2-c2ccc(C#N)cc2)cc1 ; N#Cc1ccc(-c2nc3cc(C#N)c4c5ccccc5nc(-c5cccc(-c6cc(C(F)(F)F)cc(C(F)(F)F)c6)c5)c4c3nc2-c2ccc(C#N)cc2)cc1]

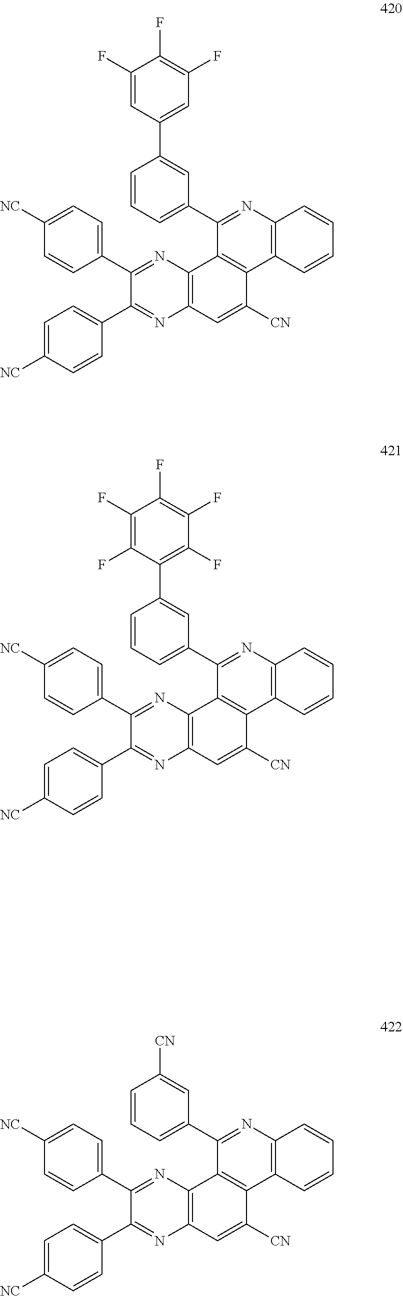 [SMILES: N#Cc1ccc(-c2nc3cc(C#N)c4c5ccccc5nc(-c5cccc(-c6cc(F)c(F)c(F)c6)c5)c4c3nc2-c2ccc(C#N)cc2)cc1 ; N#Cc1ccc(-c2nc3cc(C#N)c4c5ccccc5nc(-c5cccc(-c6c(F)c(F)c(F)c(F)c6F)c5)c4c3nc2-c2ccc(C#N)cc2)cc1 ; N#Cc1ccc(-c2nc3cc(C#N)c4c5ccccc5nc(-c5cccc(C#N)c5)c4c3nc2-c2ccc(C#N)cc2)cc1]

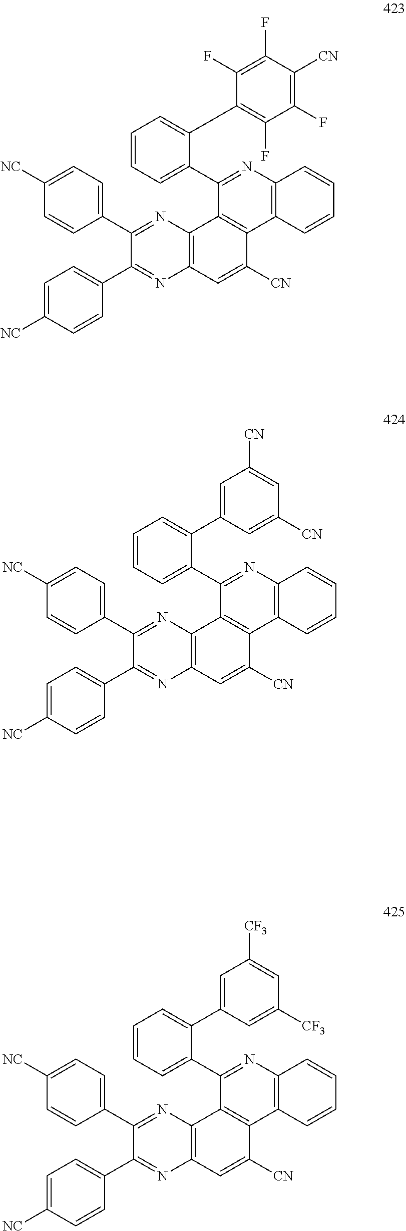 [SMILES: N#Cc1ccc(-c2nc3cc(C#N)c4c5ccccc5nc(-c5ccccc5-c5c(F)c(F)c(C#N)c(F)c5F)c4c3nc2-c2ccc(C#N)cc2)cc1 ; N#Cc1ccc(-c2nc3cc(C#N)c4c5ccccc5nc(-c5ccccc5-c5cc(C#N)cc(C#N)c5)c4c3nc2-c2ccc(C#N)cc2)cc1 ; N#Cc1ccc(-c2nc3cc(C#N)c4c5ccccc5nc(-c5ccccc5-c5cc(C(F)(F)F)cc(C(F)(F)F)c5)c4c3nc2-c2ccc(C#N)cc2)cc1]

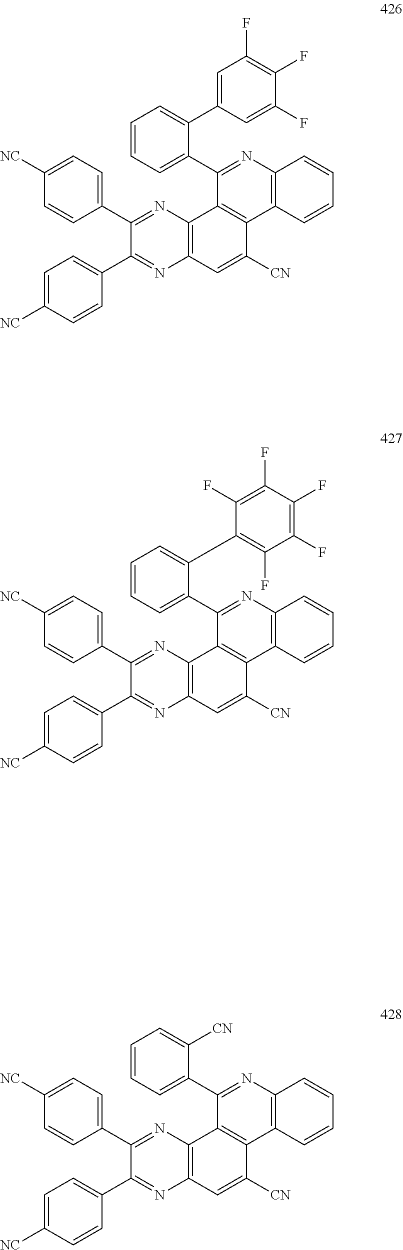 [SMILES: N#Cc1ccc(-c2nc3cc(C#N)c4c5ccccc5nc(-c5ccccc5-c5cc(F)c(F)c(F)c5)c4c3nc2-c2ccc(C#N)cc2)cc1 ; N#Cc1ccc(-c2nc3cc(C#N)c4c5ccccc5nc(-c5ccccc5-c5c(F)c(F)c(F)c(F)c5F)c4c3nc2-c2ccc(C#N)cc2)cc1 ; N#Cc1ccc(-c2nc3cc(C#N)c4c5ccccc5nc(-c5ccccc5C#N)c4c3nc2-c2ccc(C#N)cc2)cc1]

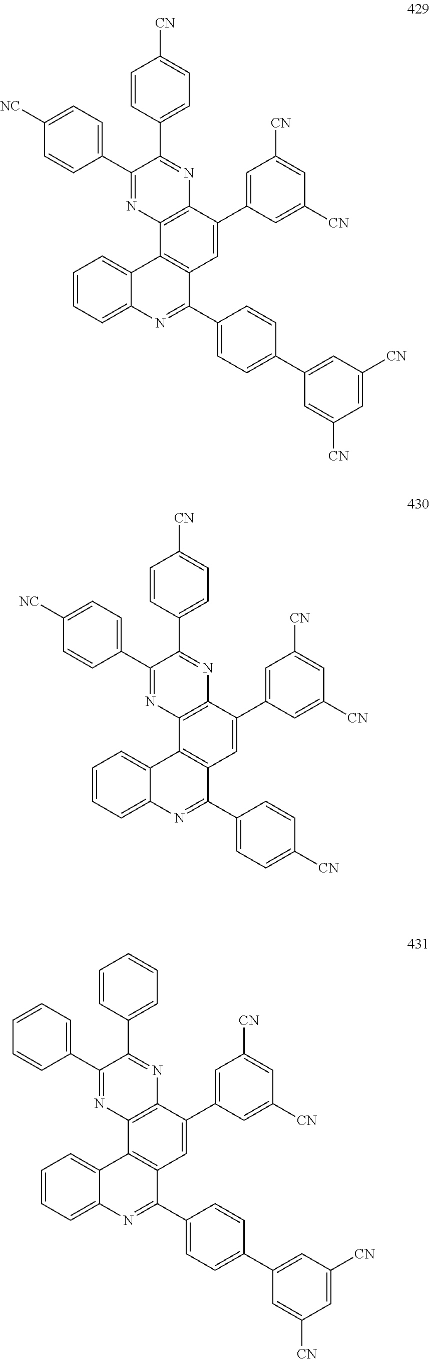 [SMILES: N#Cc1ccc(-c2nc3c(-c4cc(C#N)cc(C#N)c4)cc4c(-c5ccc(-c6cc(C#N)cc(C#N)c6)cc5)nc5ccccc5c4c3nc2-c2ccc(C#N)cc2)cc1 ; N#Cc1ccc(-c2nc3c(-c4cc(C#N)cc(C#N)c4)cc4c(-c5ccc(C#N)cc5)nc5ccccc5c4c3nc2-c2ccc(C#N)cc2)cc1 ; N#Cc1cc(C#N)cc(-c2ccc(-c3nc4ccccc4c4c3cc(-c3cc(C#N)cc(C#N)c3)c3nc(-c5ccccc5)c(-c5ccccc5)nc34)cc2)c1]

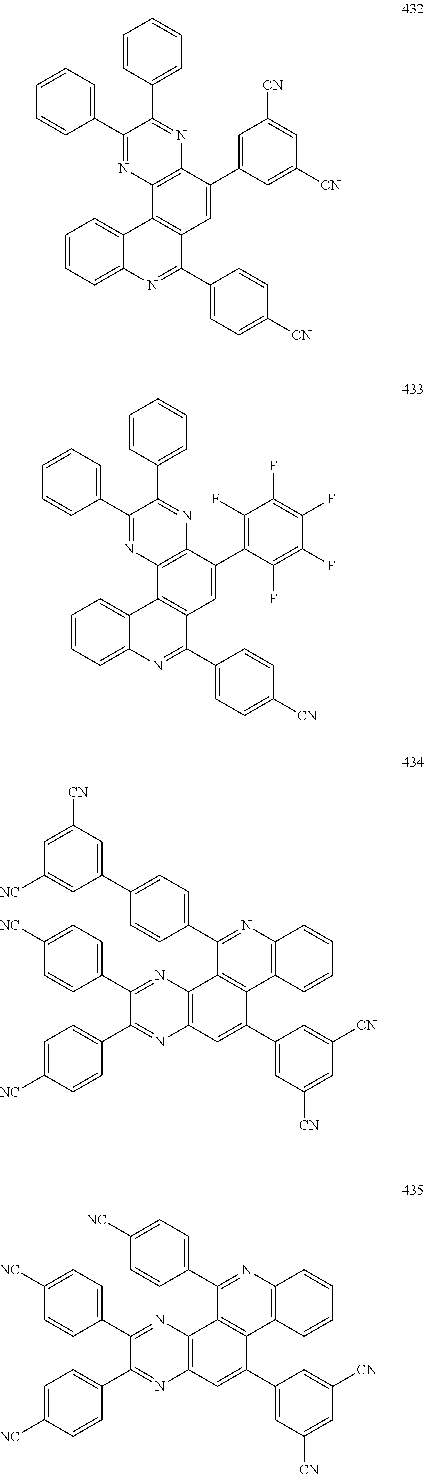 [SMILES: N#Cc1ccc(-c2nc3ccccc3c3c2cc(-c2cc(C#N)cc(C#N)c2)c2nc(-c4ccccc4)c(-c4ccccc4)nc23)cc1 ; N#Cc1ccc(-c2nc3ccccc3c3c2cc(-c2c(F)c(F)c(F)c(F)c2F)c2nc(-c4ccccc4)c(-c4ccccc4)nc23)cc1 ; N#Cc1ccc(-c2nc3cc(-c4cc(C#N)cc(C#N)c4)c4c5ccccc5nc(-c5ccc(-c6cc(C#N)cc(C#N)c6)cc5)c4c3nc2-c2ccc(C#N)cc2)cc1 ; N#Cc1ccc(-c2nc3cc(-c4cc(C#N)cc(C#N)c4)c4c5ccccc5nc(-c5ccc(C#N)cc5)c4c3nc2-c2ccc(C#N)cc2)cc1]

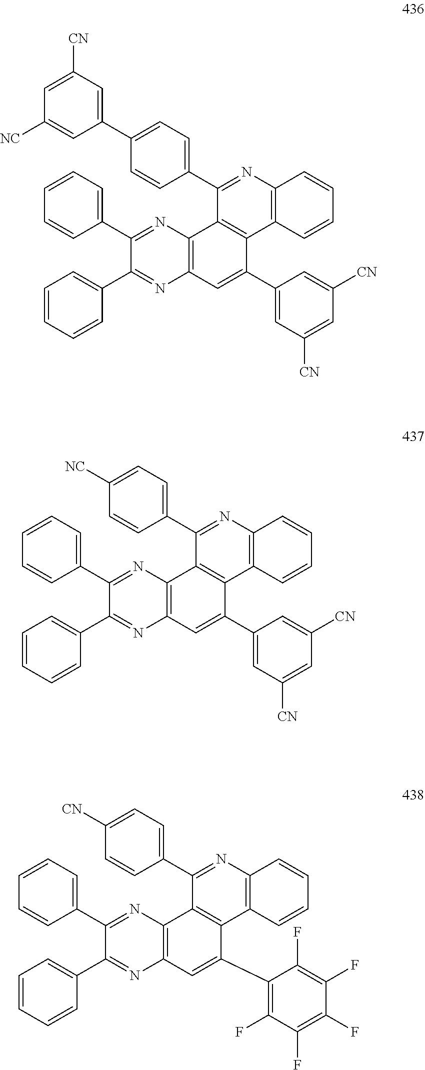

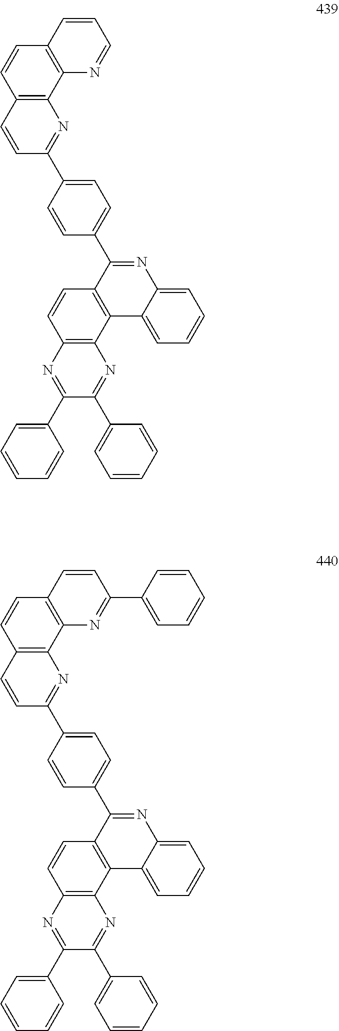 [SMILES: c1ccc(-c2nc3ccc4c(-c5ccc(-c6ccc7ccc8cccnc8c7n6)cc5)nc5ccccc5c4c3nc2-c2ccccc2)cc1 ; c1ccc(-c2ccc3ccc4ccc(-c5ccc(-c6nc7ccccc7c7c6ccc6nc(-c8ccccc8)c(-c8ccccc8)nc67)cc5)nc4c3n2)cc1]

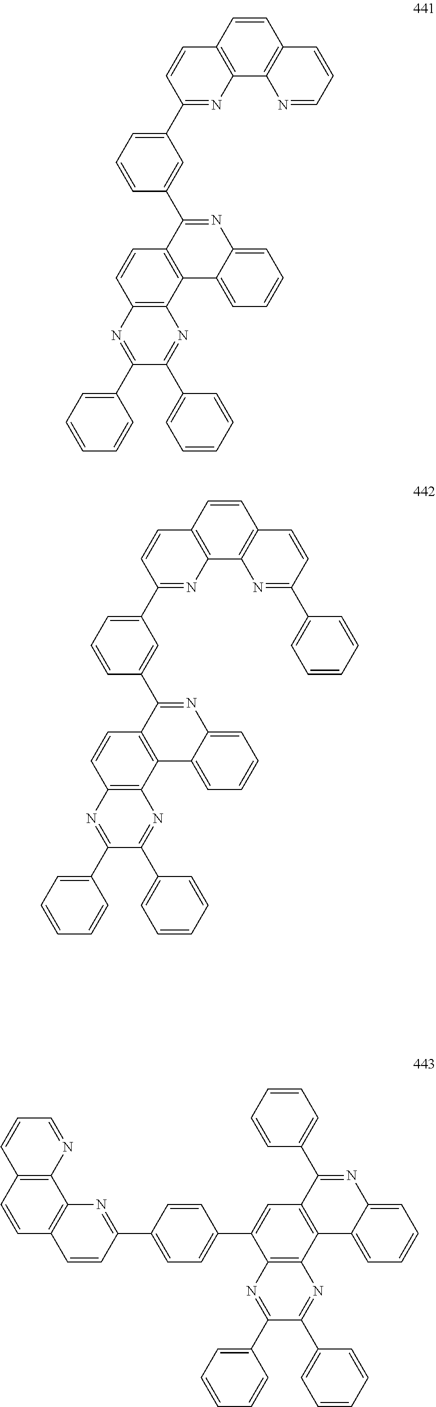 [SMILES: c1ccc(-c2nc3ccc4c(-c5cccc(-c6ccc7ccc8cccnc8c7n6)c5)nc5ccccc5c4c3nc2-c2ccccc2)cc1 ; c1ccc(-c2ccc3ccc4ccc(-c5cccc(-c6nc7ccccc7c7c6ccc6nc(-c8ccccc8)c(-c8ccccc8)nc67)c5)nc4c3n2)cc1 ; c1ccc(-c2nc3c(-c4ccc(-c5ccc6ccc7cccnc7c6n5)cc4)cc4c(-c5ccccc5)nc5ccccc5c4c3nc2-c2ccccc2)cc1]

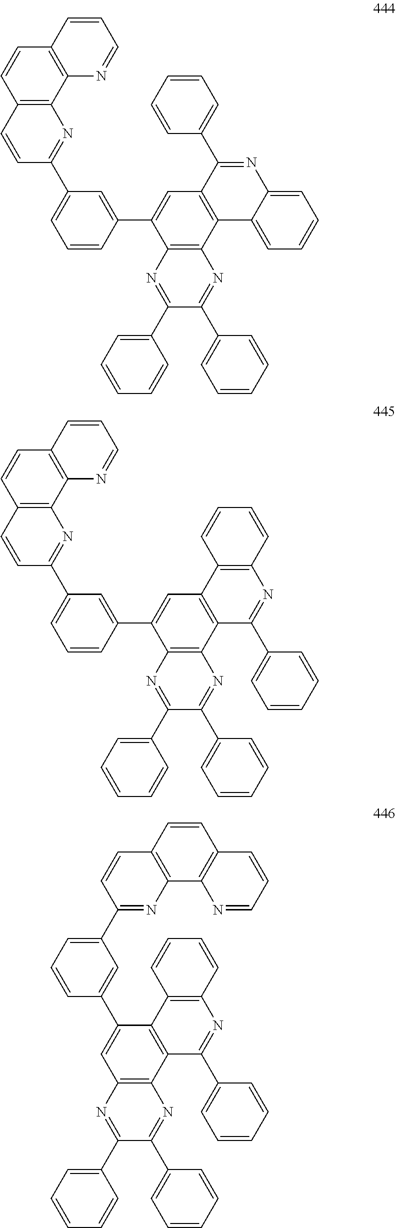

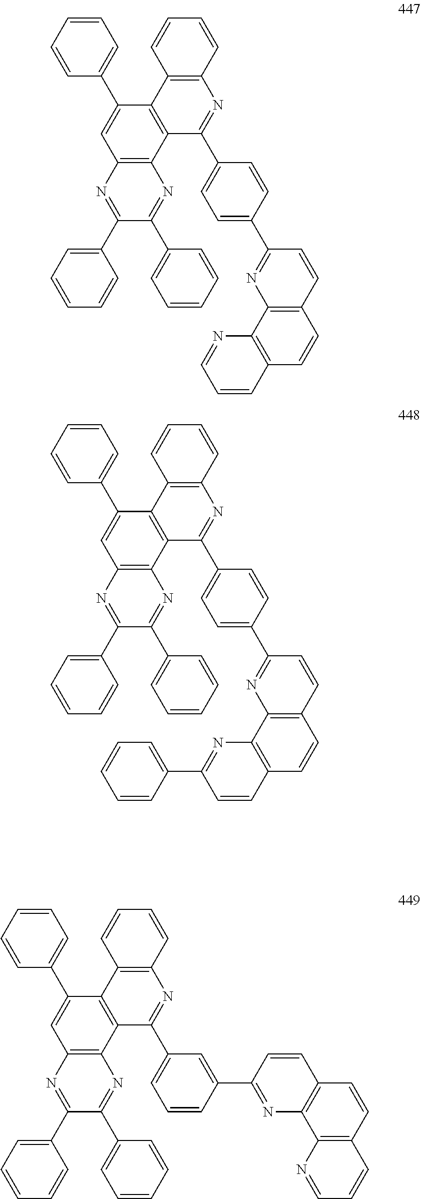

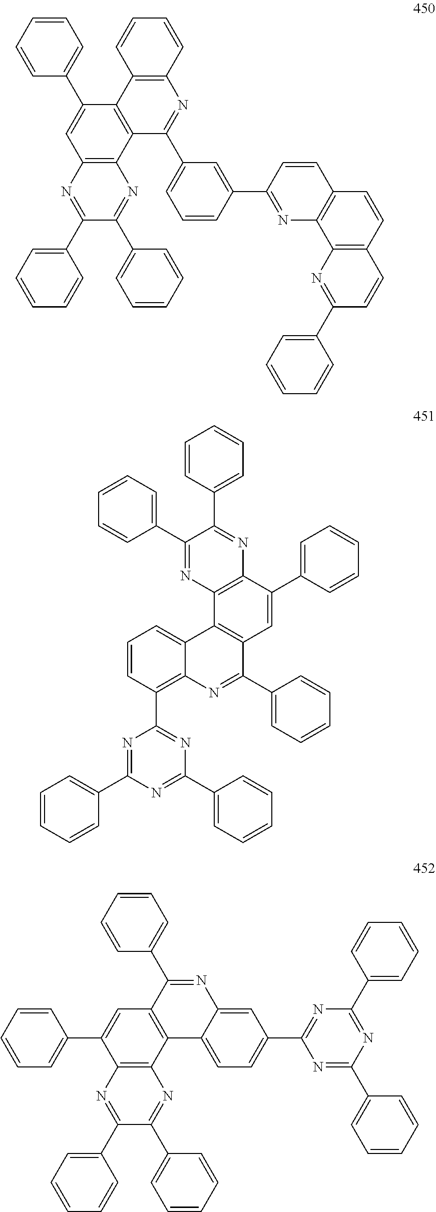 [SMILES: c1ccc(-c2ccc3ccc4ccc(-c5cccc(-c6nc7ccccc7c7c(-c8ccccc8)cc8nc(-c9ccccc9)c(-c9ccccc9)nc8c67)c5)nc4c3n2)cc1 ; c1ccc(-c2nc(-c3ccccc3)nc(-c3cccc4c3nc(-c3ccccc3)c3cc(-c5ccccc5)c5nc(-c6ccccc6)c(-c6ccccc6)nc5c34)n2)cc1 ; c1ccc(-c2nc(-c3ccccc3)nc(-c3ccc4c(c3)nc(-c3ccccc3)c3cc(-c5ccccc5)c5nc(-c6ccccc6)c(-c6ccccc6)nc5c34)n2)cc1]

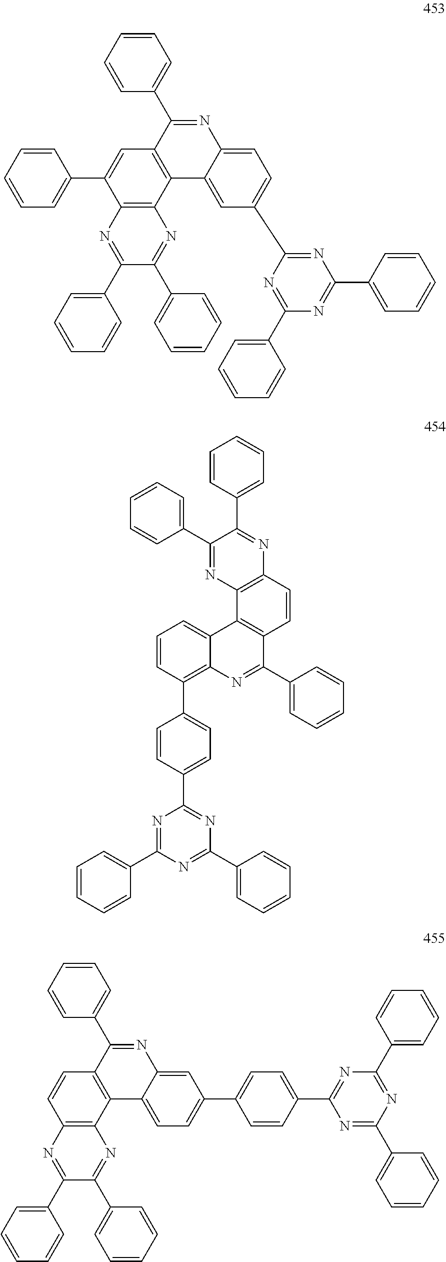

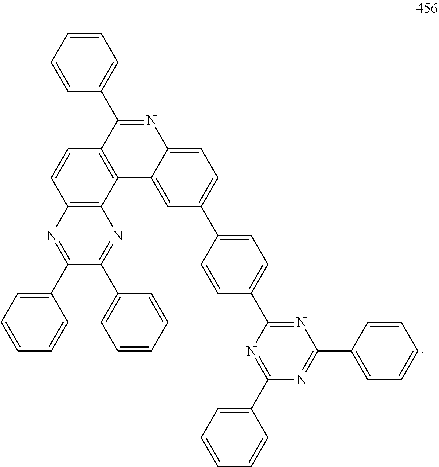 [SMILES: c1ccc(-c2nc(-c3ccccc3)nc(-c3ccc(-c4ccc5nc(-c6ccccc6)c6ccc7nc(-c8ccccc8)c(-c8ccccc8)nc7c6c5c4)cc3)n2)cc1]

9. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

10. The organic light emitting device of claim 9, wherein the organic material layer comprises a hole injection layer, and the hole injection layer comprises the heterocyclic compound.

11. The organic light emitting device of claim 9, wherein the organic material layer comprises an electron injection layer or an electron transfer layer, and the electron injection layer or the electron transfer layer comprises the heterocyclic compound.

12. The organic light emitting device of claim 9, wherein the organic material layer comprises an electron blocking layer or a hole blocking layer, and the electron blocking layer or the hole blocking layer comprises the heterocyclic compound.

13. The organic light emitting device of claim 9, further comprising one, two or more layers selected from the group consisting of a light emitting layer, a hole injection layer, a hole transfer layer, an electron injection layer, an electron transfer layer, an electron blocking layer and a hole blocking layer.

14. The organic light emitting device of claim 9, comprising:
a first electrode;
a first stack provided on the first electrode and comprising a first light emitting layer;
a charge generation layer provided on the first stack;
a second stack provided on the charge generation layer and comprising a second light emitting layer; and
a second electrode provided on the second stack.

15. The organic light emitting device of claim 14, wherein the charge generation layer comprises the heterocyclic compound.

16. The organic light emitting device of claim 15, wherein the charge generation layer is an N-type charge generation layer, and the charge generation layer comprises the heterocyclic compound.
